(19) United States
(12) Patent Application Publication
(10) Pub. No.: US 2021/0069356 A1
(43) Pub. Date: Mar. 11, 2021

(54) ISOTOPOLOGUES OF 2-(4-CHLOROPHENYL)-N-((2-(2,6-DIOXOPIPERIDIN-3-YL)-1-OXOISOINDOLIN-5-YL)METHYL)-2,2-DIFLUOROACETAMIDE

(71) Applicant: Celgene Corporation, Summit, NJ (US)

(72) Inventors: Joshua Hansen, La Jolla, CA (US); Weihong Zhang, Highland Park, NJ (US)

(21) Appl. No.: 16/959,560

(22) PCT Filed: Dec. 31, 2018
(86) PCT No.: PCT/US18/68102

§ 371 Date: Jul. 1, 2020

Related U.S. Application Data
(60) Provisional application No. 62/612,926, filed on Jan. 2, 2018.

(51) Int. Cl. A61K 51/04; A61K 45/06; C07D 401/04; C07B 59/00; A61P 35/02

(52) U.S. Cl. CPC A61K 51/0455; A61K 45/06; A61P 35/02; C07B 59/002; C07D 401/04

(57) ABSTRACT

Provided herein are isotopologues of Compound A, which are enriched with isotopes such as, for example, deuterium. Pharmaceutical compositions comprising the isotope-enriched compounds, and methods of using such compounds are also provided.

### 1 RELATED APPLICATIONS

This application claims the benefit of U.S. provisional application No. 62/612,926, filed Jan. 2, 2018, the disclosure of which is incorporated by reference in its entirety.

### 2 FIELD

Provided herein are isotopologues of certain compounds, compositions comprising the isotopologues, methods of making the isotopologues, and methods of their use for treatment or prevention of diseases and conditions including cancers. Also provided herein are such isotopologues for use in such methods of treatment or prevention.

### 3 BACKGROUND

2-(4-Chlorophenyl)-N-((2-(2,6-dioxopiperidin-3-yl)-1-oxoisoindolin-5-yl)methyl)-2,2-difluoroacetamide has been shown to have anti-cancer activities. The compound, solid forms of the compound, exemplary formulations of the compound and methods of use thereof are disclosed in U.S. Pat. Nos. 9,499,514 and 9,808,451 and US Application Publication Nos. 2017/1097934; 2017/0196847; and U.S. application Ser. No. 15/614,434, filed on Jan. 6, 2017, the entireties of each of which are incorporated by reference herein.

A need exists for developing isotopologues of 2-(4-chlorophenyl)-N-((2-(2,6-dioxopiperidin-3-yl)-1-oxoisoindolin-5-yl)methyl)-2,2-difluoroacetamide that are more metabolically stable, more therapeutically effective, or can be prepared by more efficient and scalable processes.

### 4 SUMMARY

Embodiments provided herein encompass isotopologues of Compound 1:

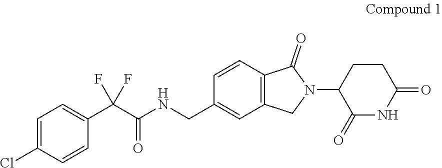 [SMILES: O=C1CCC(N2Cc3cc(CNC(=O)C(F)(F)c4ccc(Cl)cc4)ccc3C2=O)C(=O)N1]

and its stereoisomers or mixture of stereoisomers, pharmaceutically acceptable salts, tautomers, solvates, hydrates, co-crystals, clathrates, or polymorphs thereof (collectively referred to herein as “Compound A”). In one embodiment, Compound A is 2-(4-chlorophenyl)-N-((2-(2,6-dioxopiperidin-3-yl)-1-oxoisoindolin-5-yl)methyl)-2,2-difluoroacetamide.

Certain embodiments encompass mixtures of isotopologues of Compound A. Certain embodiments encompass methods of synthesizing, isolating, or characterizing an isotopologue of Compound A. In certain embodiments, the isotopologues of Compound A are deuterium, carbon-13, nitrogen-15, or oxygen-18 enriched, or combinations thereof. In certain embodiments, the isotopologues of Compound A are deuterium enriched. In certain embodiments, the isotopologues of Compound A are carbon-14 radiolabeled.

Also provided herein are pharmaceutical compositions that encompass the isotopologues of Compound A and a pharmaceutically acceptable carrier. Further provided herein are methods for treating, preventing, managing, and/or ameliorating cancers, including solid tumors and hematological cancers, or one or more symptoms or causes thereof by administering an isotopologue of Compound A.

These and other aspects of the subject matter described herein will become evident upon reference to the following detailed description.

### 5 DETAILED DESCRIPTION

The descriptions of the terminology provided below apply to the terms as used herein, unless otherwise specified.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. As used herein, an “isotopologue” is an isotopically enriched compound.

The term “isotopic enrichment” refers to the percentage of incorporation of an amount of a specific isotope at a given atom in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, about 0.0156% of molecules in a sample synthesized using non-enriched starting materials will have deuterium at a given position.

The term “isotopic enrichment factor” refers to the ratio between the isotopic composition and the natural isotopic composition of a specified isotope. The isotopic enrichment and isotopic enrichment factor of the compounds provided herein can be determined using conventional analytical methods known to one of ordinary skill in the art, including mass spectrometry and nuclear magnetic resonance spectroscopy.

It should also be noted that an isotopologue of Compound A can contain unnatural proportions of atomic isotopes at one or more of the atoms. For example, an isotopologue of Compound A may be radiolabeled at one or more positions with radioactive isotopes, such as for example tritium (H), and/or carbon-14 (¹⁴C), or may be isotopically enriched at one or more positions, such as with deuterium (2H), carbon-13 (¹³C), oxygen-18 (¹⁸O) and/or nitrogen-15 (¹⁵N). In certain embodiments, Compound A can be radiolabeled at one more positions with radioactive isotopes, such as for example tritium (3H), and/or carbon-14 (¹⁴C), while also being isotopically enriched at one or more positions, such as with deuterium (2H), carbon-13 (¹³C), oxygen-18 (¹⁸O) and/or nitrogen-15 (¹⁵N).

The term “isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of Compound A, whether radioactive or not, are intended to be encompassed within the scope of the embodiments provided herein. In some embodiments, there are provided isotopologues of Compound A, for example, the isotopologues are deuterium, carbon-13, or nitrogen-15 enriched Compound A.

With regard to the compounds provided herein, when a particular atomic position is designated as having deuterium or “D,” it is understood that the abundance of deuterium at that position is substantially greater than the natural abundance of deuterium, which is about 0.0156%. A position designated as having deuterium typically has a minimum isotopic enrichment factor of, in particular embodiments, at least 100 (1.56% deuterium incorporation), at least 500 (7.8% deuterium incorporation), at least 1000 (15.6% deuterium incorporation), at least 2000 (31.2% deuterium incorporation), at least 3000 (46.8% deuterium incorporation), at least 3500 (54.6% deuterium incorporation), at least 4000 (62.4% deuterium incorporation), at least 4500 (70.2% deuterium incorporation), at least 5000 (78% deuterium incorporation), at least 5500 (85.8% deuterium incorporation), at least 6000 (93.6% deuterium incorporation), at least 6089.7 (95% deuterium incorporation), at least 6217.9 (97% deuterium incorporation), at least 6346.2 (99% deuterium incorporation), or at least 6378.2 (99.5% deuterium incorporation) at each designated deuterium atom.

As used herein, unless otherwise specified, the term “pharmaceutically acceptable salt(s),” includes, but is not limited to, salts of acidic or basic moieties of Compound 1 and its stereoisomers or mixture of stereoisomers, tautomers, solvates, hydrates, co-crystals, clathrates, or polymorphs. Basic moieties are capable of forming a wide variety of salts with various inorganic and organic acids. The acids that can be used to prepare pharmaceutically acceptable acid addition salts of such basic compounds are those that form non-toxic acid addition salts, e.g., salts containing pharmacologically acceptable anions. Suitable organic acids include, but are not limited to, maleic, fumaric, benzoic, ascorbic, succinic, acetic, formic, oxalic, propionic, tartaric, salicylic, citric, gluconic, lactic, mandelic, cinnamic, oleic, tannic, aspartic, stearic, palmitic, glycolic, glutamic, gluconic, glucaronic, saccharic, isonicotinic, methanesulfonic, ethanesulfonic, p-toluenesulfonic, benzenesulfonic acids, or pamoic (e.g., 1,1′-methylene-bis-(2-hydroxy-3-naphthoate) acids. Suitable inorganic acids include, but are not limited to, hydrochloric, hydrobromic, hydroiodic, sulfuric, phosphoric, or nitric acids. Compounds that include an amine moiety can form pharmaceutically acceptable salts with various amino acids, in addition to the acids mentioned above. Chemical moieties that are acidic in nature are capable of forming base salts with various pharmacologically acceptable cations. Examples of such salts are alkali metal or alkaline earth metal salts and, particularly, calcium, magnesium, sodium, lithium, zinc, potassium, or iron salts. Others are well-known in the art, see for example, Remington's Pharmaceutical Sciences, 18th eds., Mack Publishing, Easton Pa. (1990) or Remington: The Science and Practice of Pharmacy, 19th eds., Mack Publishing, Easton Pa. (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. Compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, N Y, 1962); and Wilen, S. H., Tables of Resolving Agents and Optical Resolutions p. 268 (E. L. Eliel, Ed., Univ. of Notre Dame Press, Notre Dame, Ind., 1972).

It should also be noted the compounds can include E and Z isomers, or a mixture thereof, and cis and trans isomers or a mixture thereof. In certain embodiments, compounds are isolated as either the cis or trans isomer. In other embodiments, compounds are a mixture of the cis and trans isomers.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percent of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

Unless otherwise specified, the terms “solvate” and “solvated,” as used herein, refer to a solid form of a substance which contains solvent. The terms “hydrate” and “hydrated” refer to a solvate wherein the solvent is water. “Polymorphs of solvates” refer to the existence of more than one solid form for a particular solvate composition. Similarly, “polymorphs of hydrates” refer to the existence of more than one solid form for a particular hydrate composition. The term “desolvated solvate,” as used herein, refers to a solid form of a substance which can be made by removing the solvent from a solvate. The terms “solvate” and “solvated,” as used herein, can also refer to a solvate of a salt, crystal, or molecular complex. The terms “hydrate” and “hydrated,” as used herein, can also refer to a hydrate of a salt, co-crystal, or molecular complex.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. As readily understood by one skilled in the art, a wide variety of functional groups and other structures may exhibit tautomerism and all tautomers of the isotopologues of Compound A are within the scope of the present invention.

As readily understood by one skilled in the art, a wide variety of functional groups and other structures may exhibit tautomerism and all tautomers of the isotopologues of Compound A are within the scope of the present invention.

Unless otherwise specified, the term “composition” as used herein is intended to encompass a product comprising the specified ingredient(s) (and in the specified amount(s), if indicated), as well as any product which results, directly or indirectly, from combination of the specified ingredient(s) in the specified amount(s). By “pharmaceutically acceptable,” it is meant a diluent, excipient, or carrier in a formulation must be compatible with the other ingredient(s) of the formulation and not deleterious to the recipient thereof.

As used herein, “administer” or “administration” refers to the act of physically delivering a substance as it exists outside the body into a subject. Administration includes all forms known in the art for delivering therapeutic agents, including but not limited to topical, mucosal, injections, intradermal, intravenous, intramuscular delivery or other method of physical delivery described herein or known in the art (e.g., implantation of a slow-release device, such as a mini-osmotic pump to a subject; liposomal formulations; buccal; sublingual; palatal; gingival; nasal; vaginal; rectal; intra-arteriole; intraperitoneal; intraventricular; intracranial; or transdermal).

“Anti-cancer agents” refer to anti-metabolites (e.g., 5-fluoro-uracil, methotrexate, fludarabine), antimicrotubule agents (e.g., vinca alkaloids such as vincristine, vinblastine; taxanes such as paclitaxel, docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin, JM-216 or satraplatin, CI-973), anthracyclines (e.g., doxorubicin, daunorubicin), antitumor antibiotics (e.g., mitomycin, idarubicin, adriamycin, daunomycin), topoisomerase inhibitors (e.g., etoposide, camptothecins), anti-angiogenesis agents (e.g. Sutent®, sunitinib malate, and Bevacizumab) or any other cytotoxic agents (estramustine phosphate, prednimustine), hormones or hormone agonists, antagonists, partial agonists or partial antagonists, kinase inhibitors, checkpoint inhibitors, and radiation treatment.

An “effective amount” is an amount sufficient to achieve the effect for which it is administered (e.g., treat a disease or reduce one or more symptoms of a disease or condition). Thus, administration of an “amount” of a compound described herein to a subject refers to administration of “an amount effective,” to achieve the desired therapeutic result. A “therapeutically effective amount” of a compound described herein for purposes herein is thus determined by such considerations as are known in the art. The term “therapeutically effective amount” of a composition described herein refers to the amount of the composition that, when administered, is sufficient to treat one or more of the symptoms of a disease described herein (e.g., cancer, for example AML, ALL, MDS, MPN or solid tumors). Administration of a compound described herein can be determined according to factors such as, for example, the disease state, age, sex, and weight of the individual. A therapeutically effective amount also refers to any toxic or detrimental effects of isotopologues of Compound A are outweighed by the therapeutically beneficial effects.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a patient with such a disease or disorder. In some embodiments, the terms refer to the administration of a compound provided herein, with or without other additional active agent, after the onset of symptoms of the particular disease. In one embodiment, the disease is leukemia, including, but not limited to, chronic lymphocytic leukemia, chronic myelocytic leukemia, acute lymphoblastic leukemia, acute myeloid leukemia, and acute myeloblastic leukemia. In one embodiment, the leukemia can be relapsed, refractory or resistant to at least one anti-cancer therapy. In one embodiment, the disease is AML, including, a subtype of AML discussed herein. In one embodiment, the disease is MDS, including, a subtype of MDS discussed herein.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of diseases or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.” In one embodiment, the disease is leukemia, including, but is not limited to, chronic lymphocytic leukemia, chronic myelocytic leukemia, acute lymphoblastic leukemia, acute myeloid leukemia, and acute myeloblastic leukemia. In one embodiment, the leukemia can be relapsed, refractory or resistant to at least one anti-cancer therapy. In one embodiment, the disease is AML, including, a subtype of AML discussed herein. In one embodiment, the disease is MDS, including, a subtype of MDS discussed herein.

As used herein, and unless otherwise specified, the terms “manage,” “managing” and “management” refer to preventing or slowing the progression, spread or worsening of a disease or disorder, or of one or more symptoms thereof. Often, the beneficial effects that a patient derives from a prophylactic and/or therapeutic agent do not result in a cure of the disease or disorder. In this regard, the term “managing” encompasses treating a patient who had suffered from the particular disease in an attempt to prevent or minimize the recurrence of the disease, or lengthening the time during which the patient remains in remission. In one embodiment, the disease is leukemia, including, but not limited to, chronic lymphocytic leukemia, chronic myelocytic leukemia, acute lymphoblastic leukemia, acute myeloid leukemia, and acute myeloblastic leukemia. In one embodiment, the leukemia can be relapsed, refractory or resistant to at least one anti-cancer therapy. In one embodiment, the disease is AML, including, a subtype of AML discussed herein. In one embodiment, the disease is MDS, including a subtype of MDS discussed herein.

The terms “subject,” “patient,” “subject in need thereof,” and “patient in need thereof” are herein used interchangeably and refer to a living organism suffering from one or more of the diseases described herein (e.g., AML) that can be treated by administration of a composition described herein. Non-limiting examples of organisms include humans, other mammals, bovines, rats, mice, dogs, monkeys, goat, sheep, cows, deer, and other non-mammalian animals. In embodiments, a subject is human. A human subject can be between the ages of about 1 year old to about 100 years old. In embodiments, subjects herein can be characterized by the disease being treated (e.g., a “AML subject”, a “cancer subject”, or a “leukemia subject”).

As used herein, the term “tumor,” refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

As used herein, “hematologic malignancy” refers to cancer of the body's blood-forming and immune system—the bone marrow and lymphatic tissue. Such cancers include leukemias, lymphomas (Non-Hodgkin's Lymphoma), Hodgkin's disease (also called Hodgkin's Lymphoma) and myeloma. In one embodiment, the myeloma is multiple myeloma. In some embodiments, the leukemia is, for example, acute myelogenous leukemia (AML), acute lymphocytic leukemia (ALL), adult T-cell leukemia, chronic lymphocytic leukemia (CLL), hairy cell leukemia, myelodysplasia, myeloproliferative disorders or or myeloproliferative neoplasm (MPN), chronic myelogenous leukemia (CML), myelodysplastic syndrome (MDS), human lymphotropic virus-type 1 (HTLV-1) leukemia, mastocytosis, or B-cell acute lymphoblastic leukemia. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin lymphoma (HL), non-Hodgkin lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma. In one embodiment, the hematological cancer is AML. In another embodiment, the hematological cancer is MDS.

The term “leukemia” refers to malignant neoplasms of the blood-forming tissues. The leukemia includes, but is not limited to, chronic lymphocytic leukemia, chronic myelocytic leukemia, acute lymphoblastic leukemia, acute myeloid leukemia, and acute myeloblastic leukemia. The leukemia can be relapsed, refractory or resistant to at least one anti-cancer therapy.

In one embodiment, the subject has acute myelogenous or myeloid leukemia (AML), including, for example, the following subtypes of AML. The term “acute myelogenous or myeloid leukemia” refers to hematological conditions characterized by proliferation and accumulation of primarily undifferentiated or minimally differentiated myeloid cells in the bone marrow, and includes subtypes categorized by either the FAB (French, American, British) or WHO classification system. As described herein, the AML includes the following subtypes based on the FAB classification: M0 (AML minimally differentiated); M1 (AML with minimal maturation); M2 (AML with maturation); M3 (Acute promyelocytic leukemia); M4 (Acute myelomonocytic leukemia); M4 (eos Acute myelomonocytic leukemia with eosinophilia); M5 (Acute monocytic leukemia); M6 (Acute erythroid leukemia); and M7 (Acute megakaryoblastic leukemia). As described herein, the AML includes the following subtypes based on the WHO classification: AML with recurrent genetic abnormalities (AML with translocation between chromosomes 8 and 21; AML with translocation or inversion in chromosome 16; AML with translocation between chromosomes 9 and 11; APL (M3) with translocation between chromosomes 15 and 17; AML with translocation between chromosomes 6 and 9; AML with translocation or inversion in chromosome 3); AML (megakaryoblastic) with a translocation between chromosomes 1 and 22; AML with myelodysplasia-related changes; AML related to previous chemotherapy or radiation (Alkylating agent-related AML; Topoisomerase II inhibitor-related AML); AML not otherwise categorized (AML that does not fall into the above categories, i. e. AML minimally differentiated (M0); AML with minimal maturation (M1); AML with maturation (M2); Acute myelomonocytic leukemia (M4); Acute monocytic leukemia (M5); Acute erythroid leukemia (M6); Acute megakaryoblastic leukemia (M7); Acute basophilic leukemia; Acute panmyelosis with fibrosis); Myeloid Sarcoma (also known as granulocytic sarcoma, chloroma or extramedullary myeloblastoma); and Undifferentiated and biphenotypic acute leukemias (also known as mixed phenotype acute leukemias). (see https://www.cancer.org/cancer/acute-myeloid-leukemia/detection-diagnosis-staging/how-classified.html, last accessed May 25, 2017).

In one embodiment, the subject has myelodysplastic syndrome (MDS), including, for example, the following subtypes of MDS. The term “myelodysplastic syndrome” refers to hematological conditions characterized by abnormalities in the production of one or more of the cellular components of blood (red cells, white cells (other than lymphocytes) and platelets (or their progenitor cells, megakaryocytes)), and includes the following disorders: refractory anemia (RA); RA with ringed sideroblasts (RARS); RA with excess of blasts (RAEB); refractory cytopenia with multilineage dysplasia (RCMD), refractory cytopenia with unilineage dysplasia (RCUD); unclassifiable myelodysplastic syndrome (MDS-U), myelodysplastic syndrome associated with an isolated del(5q) chromosome abnormality, therapy-related myeloid neoplasms and chronic myelomonocytic leukemia (CMML). The MDS as used herein also includes very low risk, low risk, intermediate risk, high risk and very high risk MDS. In some embodiments, the MDS is primary or de novo MDS. In other embodiments, the MDS is secondary.

As used herein, “promyelocytic leukemia” or “acute promyelocytic leukemia” refers to a malignancy of the bone marrow in which there is a deficiency of mature blood cells in the myeloid line of cells and an excess of immature cells called promyelocytes. It is usually marked by an exchange of regions of chromosomes 15 and 17.

As used herein, “acute lymphocytic leukemia (ALL)”, also known as “acute lymphoblastic leukemia” refers to a malignant disease caused by the abnormal growth and development of early nongranular white blood cells, or lymphocytes.

As used herein, “T-cell leukemia” refers to a disease in which certain cells of the lymphoid system called T lymphocytes or T cells are malignant. T cells are white blood cells that normally can attack virus-infected cells, foreign cells, and cancer cells and produce substances that regulate the immune response.

The term “relapsed” refers to a situation where patients who have had a remission of leukemia after therapy have a return of leukemia cells in the marrow and a decrease in normal blood cells.

The term “refractory or resistant” refers to a circumstance where patients, even after intensive treatment, have residual leukemia cells in their marrow.

The term “drug resistance” refers to the condition when a disease does not respond to the treatment of a certain drug or drugs. Drug resistance can be either intrinsic, which means the disease has never been responsive to the particular drug or drugs, or it can be acquired, which means the disease ceases responding to particular a drug or drugs that the disease had previously responded to. In certain embodiments, drug resistance is intrinsic. In certain embodiments, the drug resistance is acquired.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. 1972, 11:942-944).

5.1 Compounds

Provided herein are isotopically enriched compounds, including isotopically enriched Compound A and synthetic intermediates thereof.

Isotopic enrichment (e.g., deuteration) of pharmaceuticals to improve pharmacokinetics (“PK”), pharmacodynamics (“PD”), and toxicity profiles has been demonstrated previously with some classes of drugs. (See, e.g., Lijinsky et. al., Food Cosmet. Toxicol., Vol. 20, p. 393 (1982); Lijinsky et. al., J. Nat. Cancer Inst., Vol. 69, p. 1127 (1982); Mangold et. al., Mutation Res. Vol. 308, p. 33 (1994); Gordon et. al., DrugMetab. Dispos., Vol. 15, p. 589 (1987); Zello et. al., Metabolism, Vol. 43, p. 487 (1994); Gately et. al., J. Nucl. Med., Vol. 27, p. 388 (1986); Wade D, Chem. Biol. Interact., Vol. 117, p. 191 (1999); and Dyck et al. J. of Neurochemistry, Vol. 46, No. 2, p. 399 (1986).

Without being limited by a particular theory, isotopic enrichment of a drug can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrease the production of deleterious metabolites in specific tissues and/or create a more effective drug and/or a safer drug for combination therapy, whether the combination therapy is intentional or not.

Replacement of an atom for one of its isotopes may often result in a change in the reaction rate of a chemical reaction or an enzyme catalyzed reaction. This phenomenon is known as the Kinetic Isotope Effect (“KIE”). For example, if a C—H bond is broken during a rate-determining step in a chemical reaction (i.e., the step with the highest transition state energy), substitution of a deuterium for that hydrogen can cause a decrease in the reaction rate and the process may slow down. This phenomenon is known as the Deuterium Kinetic Isotope Effect (“DKIE”). (See, e.g, Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. Without being limited by a particular theory, high DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T20. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, may lead to a similar kinetic isotope effect.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. These drugs therefore often require the administration of multiple or high daily doses.

Therefore, isotopic enrichment at certain positions of a compound provided herein may produce a detectable KIE that affects the pharmacokinetic, pharmacologic, and/or toxicological profiles of a compound provided herein in comparison with a similar compound having a natural isotopic composition. In one embodiment, the deuterium enrichment is performed on the site of C—H bond cleavage during metabolism.

In certain embodiments, provided herein are deuterated analogues of Compound A, wherein one or more atomic positions of Compound A is/are isotopically enriched with deuterium.

In certain embodiments, provided herein are radiolabeled analogues of Compound A, wherein one or more carbon atoms in Compound A is/are radiolabeled carbon-14 (¹⁴C).

Certain embodiments herein provide a compound having formula:

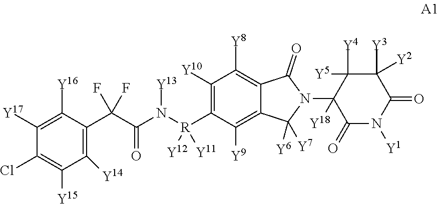

and its stereoisomers or mixture of stereoisomers, pharmaceutically acceptable salts, tautomers, solvates, hydrates, co-crystals, clathrates, or polymorphs thereof, wherein R is C or ¹⁴C; when R is C then one or more of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, and Y¹⁸ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷ and Y¹⁸ are non-enriched hydrogen atoms; and when R is ¹⁴C, then optionally one or more of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, and Y¹⁸ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷ and Y¹⁸ are non-enriched hydrogen atoms. In particular embodiments, provided herein are compounds having formula A1, wherein R is C and one or more Y atoms (i.e., Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷ and Y¹⁸) is/are hydrogen(s) isotopically enriched with deuterium, and any remaining Y atom(s) is/are non-enriched hydrogen atom(s). In particular embodiments, provided herein are compounds having formula A1, wherein R is ¹⁴C and all Y atoms are non-enriched hydrogen atoms. In certain embodiments, provided herein are compounds having formula A1, wherein one, two, three, four, five, six, seven, eight, nine, ten, eleven, twelve, thirteen, fourteen, fifteen, sixteen, seventeen or eighteen of the indicated Y atoms is/are isotopically enriched with deuterium, and any remaining Y atom(s) is/are non-enriched hydrogen(s). In one embodiment, provided herein is a compound having formula A1, wherein one of the indicated Y atoms is isotopically enriched with deuterium, and remaining Y atoms are non-enriched hydrogens. In one embodiment, provided herein is a compound having formula A1, wherein two of the indicated Y atoms are isotopically enriched with deuterium, and remaining Y atoms are non-enriched hydrogens. In one embodiment, provided herein is a compound having formula A1, wherein three of the indicated Y atoms are isotopically enriched with deuterium, and remaining Y atoms are non-enriched hydrogens. In one embodiment, provided herein is a compound having formula A1, wherein four of the indicated Y atoms are isotopically enriched with deuterium, and remaining Y atoms are non-enriched hydrogens. In one embodiment, provided herein is a compound having formula A1, wherein five of the indicated Y atoms are isotopically enriched with deuterium, and remaining Y atoms are non-enriched hydrogens. In one embodiment, provided herein is a compound having formula A1, wherein six of the indicated Y atoms are isotopically enriched with deuterium, and remaining Y atoms are non-enriched hydrogens. In one embodiment, provided herein is a compound having formula A1, wherein seven of the indicated Y atoms are isotopically enriched with deuterium, and remaining Y atoms are non-enriched hydrogens. In one embodiment, provided herein is a compound having formula A1, wherein eight of the indicated Y atoms are isotopically enriched with deuterium, and remaining Y atoms are non-enriched hydrogens. In one embodiment, provided herein is a compound having formula A1, wherein nine of the indicated Y atoms are isotopically enriched with deuterium, and remaining Y atoms are non-enriched hydrogens. In one embodiment, provided herein is a compound having formula A1, wherein ten of the indicated Y atoms are isotopically enriched with deuterium, and remaining Y atoms are non-enriched hydrogens. In one embodiment, provided herein is a compound having formula A1, wherein eleven of the indicated Y atoms are isotopically enriched with deuterium, and remaining Y atoms are non-enriched hydrogens. In one embodiment, provided herein is a compound having formula A1, wherein twelve of the indicated Y atoms are isotopically enriched with deuterium, and remaining Y atoms are non-enriched hydrogens. In one embodiment, provided herein is a compound having formula A1, wherein thirteen of the indicated Y atoms are isotopically enriched with deuterium, and remaining Y atoms are non-enriched hydrogens. In one embodiment, provided herein is a compound having formula A1, wherein fourteen of the indicated Y atoms are isotopically enriched with deuterium, and remaining Y atoms are non-enriched hydrogens. In one embodiment, provided herein is a compound having formula A1, wherein fifteen of the indicated Y atoms are isotopically enriched with deuterium, and remaining Y atoms are non-enriched hydrogens. In one embodiment, provided herein is a compound having formula A1, wherein sixteen of the indicated Y atoms are isotopically enriched with deuterium, and remaining Y atoms are non-enriched hydrogens. In one embodiment, provided herein is a compound having formula A1, wherein seventeen of the indicated Y atoms are isotopically enriched with deuterium, and remaining Y atom is non-enriched hydrogen. In one embodiment, provided herein is a compound having formula A1, wherein all of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷ and Y¹⁸ are isotopically enriched with deuterium. In one embodiment, provided herein is a compound having formula A1, wherein Y², Y³, Y⁴, Y⁵, Y⁶, Y⁷, Y⁸, Y⁹, Y¹⁰, Y¹¹, Y¹², Y¹⁴, Y¹⁵, Y¹⁶, Y¹⁷ and Y¹⁸ are isotopically enriched with deuterium.

Certain embodiments herein provide compounds having formula A2:

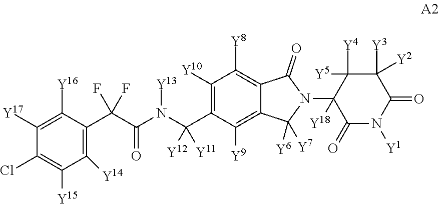

and its stereoisomers or mixture of stereoisomers, pharmaceutically acceptable salts, tautomers, solvates, hydrates, co-crystals, clathrates, or polymorphs thereof, wherein one or more Y atoms (i.e., Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷ and Y¹⁸) is/are hydrogen(s) isotopically enriched with deuterium, and any remaining Y atom(s) is/are non-enriched hydrogen atom(s). In particular embodiments, one, two, three, four, five, six, seven, eight, nine, ten, eleven, twelve, thirteen, fourteen, fifteen, sixteen, seventeen or eighteen of the indicated Y atoms is/are isotopically enriched with deuterium, and any remaining Y atom(s) is/are non-enriched hydrogen(s). In one embodiment, one of the indicated Y atoms is isotopically enriched with deuterium, and remaining Y atoms are non-enriched hydrogens. In one embodiment, two of the indicated Y atoms are isotopically enriched with deuterium, and remaining Y atoms are non-enriched hydrogens. In one embodiment, three of the indicated Y atoms are isotopically enriched with deuterium, and remaining Y atoms are non-enriched hydrogens. In one embodiment, four of the indicated Y atoms are isotopically enriched with deuterium, and remaining Y atoms are non-enriched hydrogens. In one embodiment, five of the indicated Y atoms are isotopically enriched with deuterium, and remaining Y atoms are non-enriched hydrogens. In one embodiment, six of the indicated Y atoms are isotopically enriched with deuterium, and remaining Y atoms are non-enriched hydrogens. In one embodiment, seven of the indicated Y atoms are isotopically enriched with deuterium, and remaining Y atoms are non-enriched hydrogens. In one embodiment, eight of the indicated Y atoms are isotopically enriched with deuterium, and remaining Y atoms are non-enriched hydrogens. In one embodiment, nine of the indicated Y atoms are isotopically enriched with deuterium, and remaining Y atoms are non-enriched hydrogens. In one embodiment, ten of the indicated Y atoms are isotopically enriched with deuterium, and remaining Y atoms are non-enriched hydrogens. In one embodiment, eleven of the indicated Y atoms are isotopically enriched with deuterium, and remaining Y atoms are non-enriched hydrogens. In one embodiment, twelve of the indicated Y atoms are isotopically enriched with deuterium, and remaining Y atoms are non-enriched hydrogens. In one embodiment, thirteen of the indicated Y atoms are isotopically enriched with deuterium, and remaining Y atoms are non-enriched hydrogens. In one embodiment, fourteen of the indicated Y atoms are isotopically enriched with deuterium, and remaining Y atoms are non-enriched hydrogens. In one embodiment, fifteen of the indicated Y atoms are isotopically enriched with deuterium, and remaining Y atoms are non-enriched hydrogens. In one embodiment, sixteen of the indicated Y atoms are isotopically enriched with deuterium, and remaining Y atoms are non-enriched hydrogens. In one embodiment, seventeen of the indicated Y atoms are isotopically enriched with deuterium, and remaining Y atom is non-enriched hydrogen. In one embodiment, all of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷ and Y¹⁸ are isotopically enriched with deuterium. In one embodiment, Y², Y³, Y⁴, Y⁵, Y⁶, Y⁷, Y⁸, Y⁹, Y¹⁰, Y¹¹, Y¹², Y¹⁴, Y¹⁵, Y¹⁶, Y¹⁷ and Y¹⁸ are isotopically enriched with deuterium.

Certain embodiments herein provide compounds having formula A3:

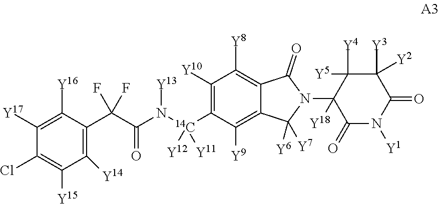

and its stereoisomers or mixture of stereoisomers, pharmaceutically acceptable salts, tautomers, solvates, hydrates, co-crystals, clathrates, or polymorphs thereof, wherein one or more Y atoms (i.e., Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷ and Y¹⁸) is/are hydrogen(s) isotopically enriched with deuterium, and any remaining Y atom(s) is/are non-enriched hydrogen atom(s). In certain embodiments, provided herein are compounds having formula A3, wherein one, two, three, four, five, six, seven, eight, nine, ten, eleven, twelve, thirteen, fourteen, fifteen, sixteen, seventeen or eighteen of the indicated Y atoms is/are isotopically enriched with deuterium, and any remaining Y atom(s) is/are non-enriched hydrogen(s). In one embodiment, provided herein is a compound having formula A3, wherein all Y atoms are non-enriched hydrogens.

In certain embodiments, one or more Y atoms on any of the rings of formula A1, A2 or A3 is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium.

In certain embodiments, one or more Y atoms on the dioxopiperidinyl portion of formula A1, A2 or A3 are deuterium-enriched. For example, particular compounds provided herein include, but are not limited to, the compounds listed in Table 1, and its stereoisomers or mixture of stereoisomers, pharmaceutically acceptable salts, tautomers, solvates, hydrates, co-crystals, clathrates, or polymorphs thereof, wherein the label “D” indicates a deuterium-enriched atomic position, i.e., a sample comprising the given compound has a deuterium enrichment at the indicated position(s) above the natural abundance of deuterium:

TABLE 1

| No. | Compound structure |
|---|---|
| 1 | 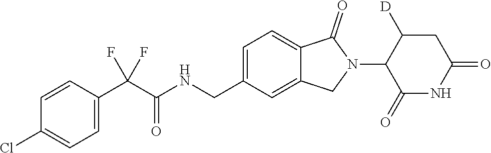 [SMILES: [2H]C1CC(=O)NC(=O)C1N1Cc2cc(CNC(=O)C(F)(F)c3ccc(Cl)cc3)ccc2C1=O] |
| 2 | 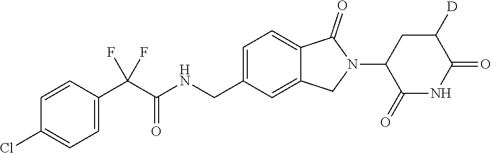 [SMILES: [2H]C1CC(N2Cc3cc(CNC(=O)C(F)(F)c4ccc(Cl)cc4)ccc3C2=O)C(=O)NC1=O] |
| 3 | 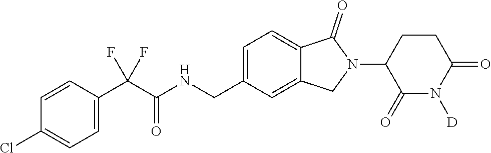 |
| 4 | 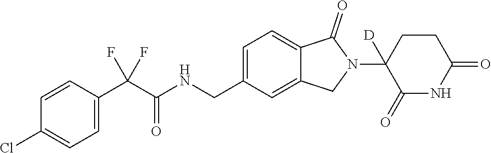 [SMILES: [2H]C1(N2Cc3cc(CNC(=O)C(F)(F)c4ccc(Cl)cc4)ccc3C2=O)CCC(=O)NC1=O] |
| 5 | 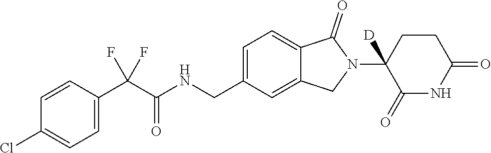 [SMILES: [2H][C@@]1(N2Cc3cc(CNC(=O)C(F)(F)c4ccc(Cl)cc4)ccc3C2=O)CCC(=O)NC1=O] |
| 6 | 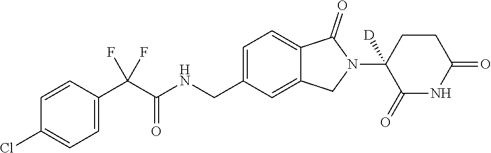 [SMILES: [2H][C@]1(N2Cc3cc(CNC(=O)C(F)(F)c4ccc(Cl)cc4)ccc3C2=O)CCC(=O)NC1=O] |
| 7 | 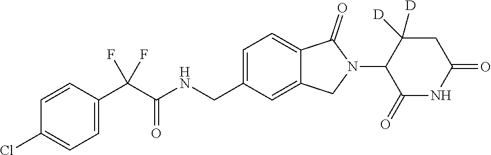 [SMILES: [2H]C1([2H])CC(=O)NC(=O)C1N1Cc2cc(CNC(=O)C(F)(F)c3ccc(Cl)cc3)ccc2C1=O] |
| 8 | 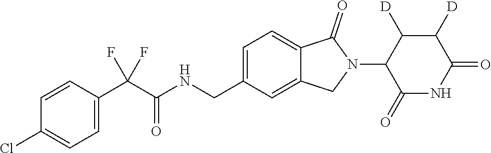 [SMILES: [2H]C1C(=O)NC(=O)C(N2Cc3cc(CNC(=O)C(F)(F)c4ccc(Cl)cc4)ccc3C2=O)C1[2H]] |
| 9 | 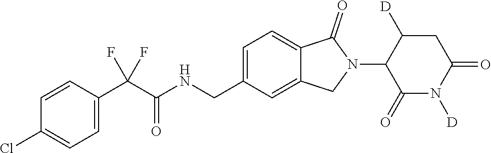 |
| 10 | 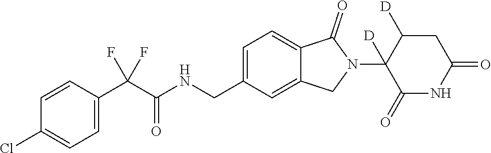 [SMILES: [2H]C1CC(=O)NC(=O)C1([2H])N1Cc2cc(CNC(=O)C(F)(F)c3ccc(Cl)cc3)ccc2C1=O] |
| 11 | 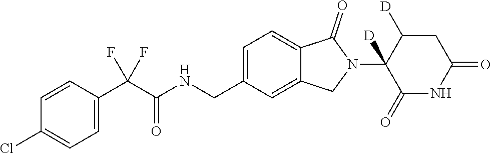 [SMILES: [2H]C1CC(=O)NC(=O)[C@]1([2H])N1Cc2cc(CNC(=O)C(F)(F)c3ccc(Cl)cc3)ccc2C1=O] |
| 12 | 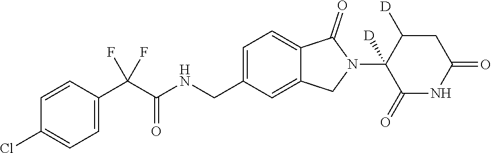 [SMILES: [2H]C1CC(=O)NC(=O)[C@@]1([2H])N1Cc2cc(CNC(=O)C(F)(F)c3ccc(Cl)cc3)ccc2C1=O] |
| 13 | 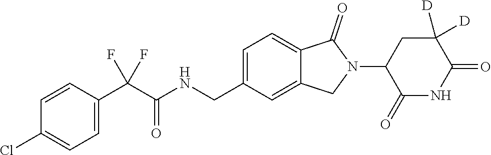 [SMILES: [2H]C1([2H])CC(N2Cc3cc(CNC(=O)C(F)(F)c4ccc(Cl)cc4)ccc3C2=O)C(=O)NC1=O] |
| 14 | 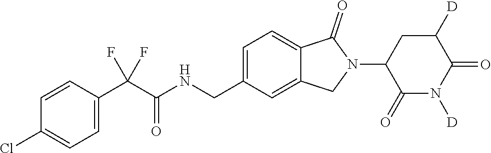 |
| 15 | 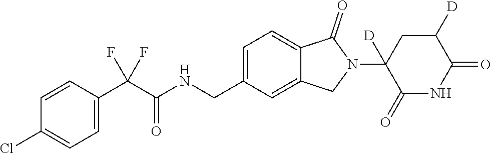 [SMILES: [2H]C1CC([2H])(N2Cc3cc(CNC(=O)C(F)(F)c4ccc(Cl)cc4)ccc3C2=O)C(=O)NC1=O] |
| 16 | 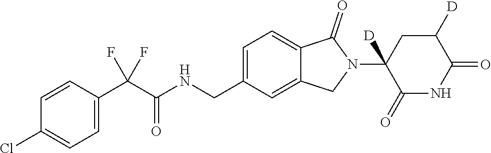 [SMILES: [2H]C1C[C@@]([2H])(N2Cc3cc(CNC(=O)C(F)(F)c4ccc(Cl)cc4)ccc3C2=O)C(=O)NC1=O] |
| 17 | 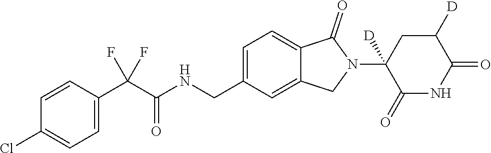 [SMILES: [2H]C1C[C@]([2H])(N2Cc3cc(CNC(=O)C(F)(F)c4ccc(Cl)cc4)ccc3C2=O)C(=O)NC1=O] |
| 18 | 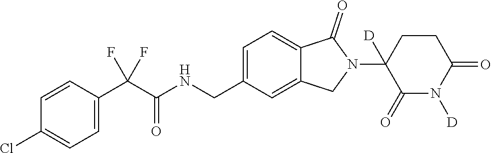 |
| 19 | 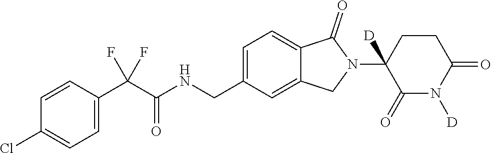 |
| 20 | 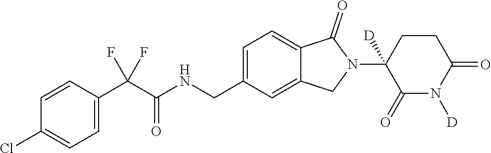 |
| 21 | 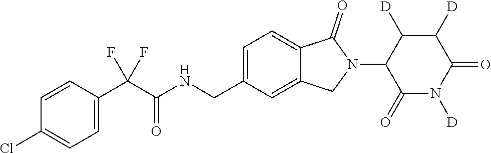 |
| 22 | 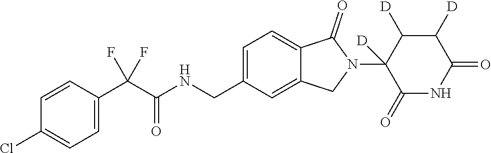 [SMILES: [2H]C1C(=O)NC(=O)C([2H])(N2Cc3cc(CNC(=O)C(F)(F)c4ccc(Cl)cc4)ccc3C2=O)C1[2H]] |
| 23 | 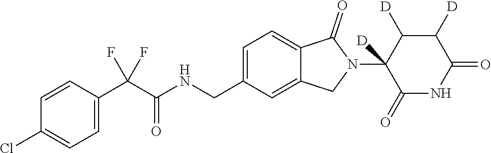 [SMILES: [2H]C1C(=O)NC(=O)[C@]([2H])(N2Cc3cc(CNC(=O)C(F)(F)c4ccc(Cl)cc4)ccc3C2=O)C1[2H]] |
| 24 | 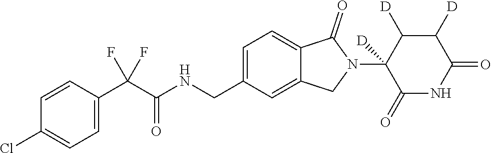 [SMILES: [2H]C1C(=O)NC(=O)[C@@]([2H])(N2Cc3cc(CNC(=O)C(F)(F)c4ccc(Cl)cc4)ccc3C2=O)C1[2H]] |
| 25 | 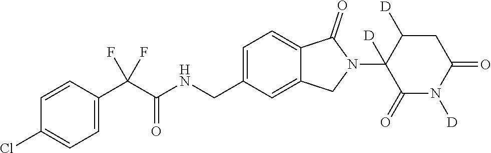 |
| 26 | 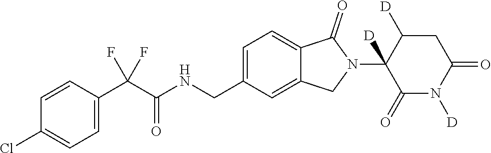 |
| 27 | 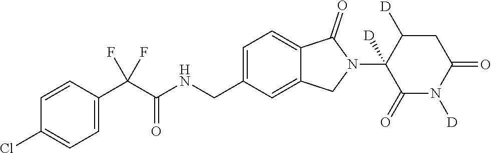 |
| 28 | 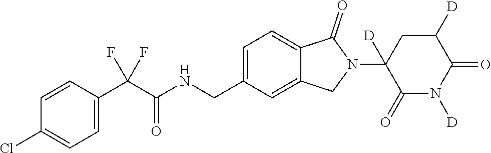 |
| 29 | 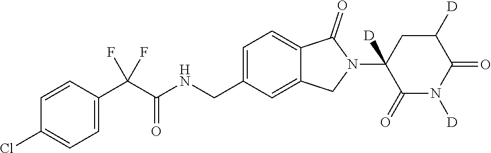 |
| 30 | 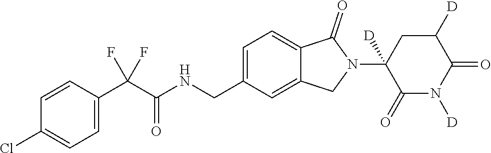 |
| 31 | 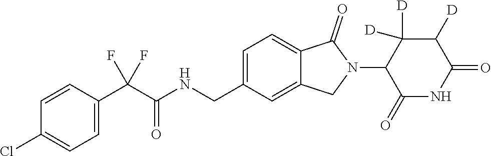 [SMILES: [2H]C1C(=O)NC(=O)C(N2Cc3cc(CNC(=O)C(F)(F)c4ccc(Cl)cc4)ccc3C2=O)C1([2H])[2H]] |
| 32 | 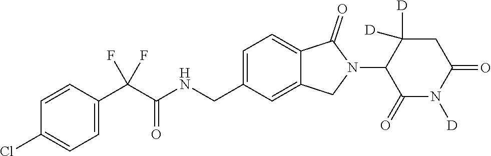 |
| 33 | 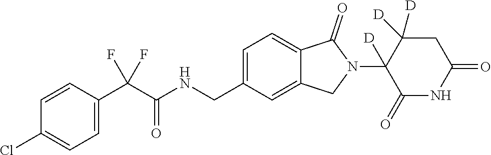 [SMILES: [2H]C1([2H])CC(=O)NC(=O)C1([2H])N1Cc2cc(CNC(=O)C(F)(F)c3ccc(Cl)cc3)ccc2C1=O] |
| 34 | 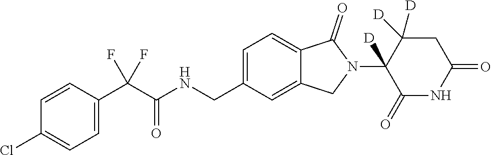 [SMILES: [2H]C1([2H])CC(=O)NC(=O)[C@]1([2H])N1Cc2cc(CNC(=O)C(F)(F)c3ccc(Cl)cc3)ccc2C1=O] |
| 35 | 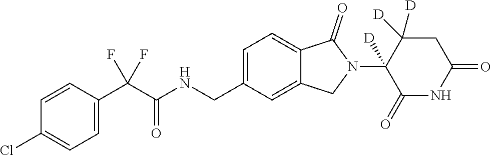 [SMILES: [2H]C1([2H])CC(=O)NC(=O)[C@@]1([2H])N1Cc2cc(CNC(=O)C(F)(F)c3ccc(Cl)cc3)ccc2C1=O] |
| 36 | 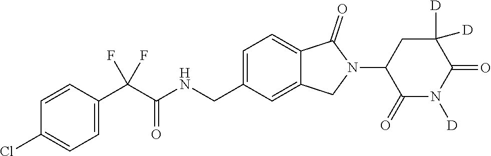 |
| 37 | 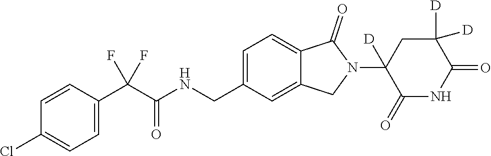 [SMILES: [2H]C1([2H])CC([2H])(N2Cc3cc(CNC(=O)C(F)(F)c4ccc(Cl)cc4)ccc3C2=O)C(=O)NC1=O] |
| 38 | 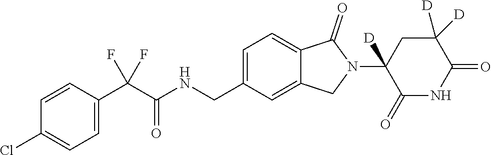 [SMILES: [2H]C1([2H])C[C@@]([2H])(N2Cc3cc(CNC(=O)C(F)(F)c4ccc(Cl)cc4)ccc3C2=O)C(=O)NC1=O] |
| 39 | 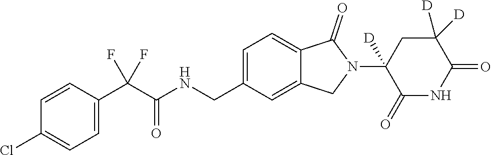 [SMILES: [2H]C1([2H])C[C@]([2H])(N2Cc3cc(CNC(=O)C(F)(F)c4ccc(Cl)cc4)ccc3C2=O)C(=O)NC1=O] |
| 40 | 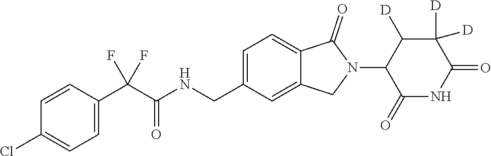 [SMILES: [2H]C1C(N2Cc3cc(CNC(=O)C(F)(F)c4ccc(Cl)cc4)ccc3C2=O)C(=O)NC(=O)C1([2H])[2H]] |
| 41 | 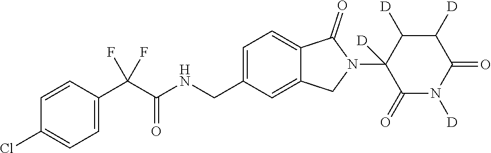 |
| 42 | 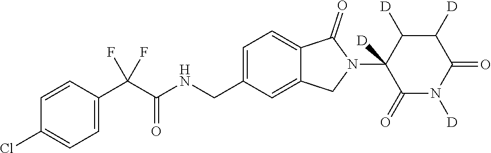 |
| 43 | 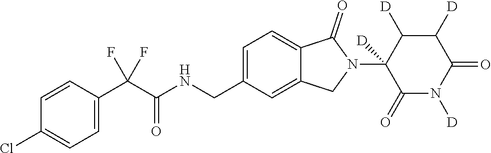 |
| 44 | 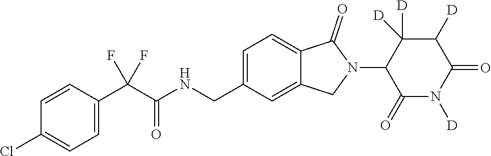 |
| 45 | 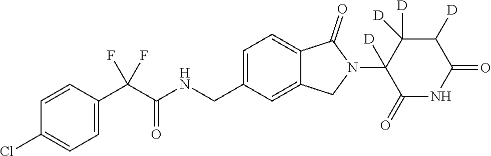 [SMILES: [2H]C1C(=O)NC(=O)C([2H])(N2Cc3cc(CNC(=O)C(F)(F)c4ccc(Cl)cc4)ccc3C2=O)C1([2H])[2H]] |
| 46 | 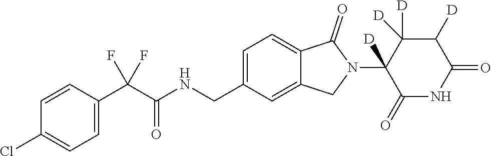 [SMILES: [2H]C1C(=O)NC(=O)[C@]([2H])(N2Cc3cc(CNC(=O)C(F)(F)c4ccc(Cl)cc4)ccc3C2=O)C1([2H])[2H]] |
| 47 | 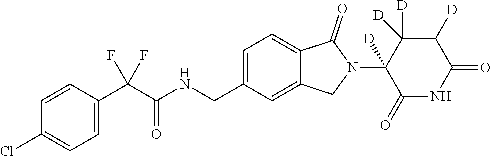 [SMILES: [2H]C1C(=O)NC(=O)[C@@]([2H])(N2Cc3cc(CNC(=O)C(F)(F)c4ccc(Cl)cc4)ccc3C2=O)C1([2H])[2H]] |
| 48 | 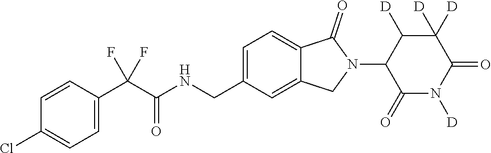 |
| 49 | 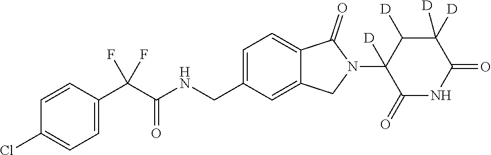 [SMILES: [2H]C1C([2H])([2H])C(=O)NC(=O)C1([2H])N1Cc2cc(CNC(=O)C(F)(F)c3ccc(Cl)cc3)ccc2C1=O] |
| 50 | 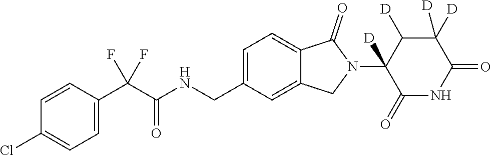 [SMILES: [2H]C1C([2H])([2H])C(=O)NC(=O)[C@]1([2H])N1Cc2cc(CNC(=O)C(F)(F)c3ccc(Cl)cc3)ccc2C1=O] |
| 51 | 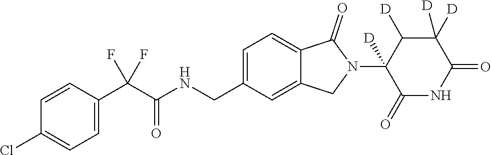 [SMILES: [2H]C1C([2H])([2H])C(=O)NC(=O)[C@@]1([2H])N1Cc2cc(CNC(=O)C(F)(F)c3ccc(Cl)cc3)ccc2C1=O] |
| 52 | 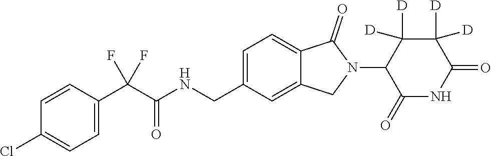 [SMILES: [2H]C1([2H])C(=O)NC(=O)C(N2Cc3cc(CNC(=O)C(F)(F)c4ccc(Cl)cc4)ccc3C2=O)C1([2H])[2H]] |
| 53 | 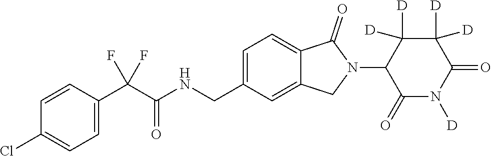 |
| 54 | 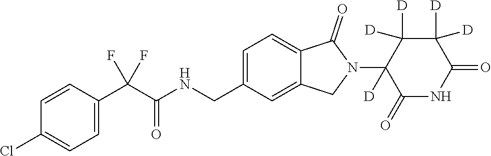 [SMILES: [2H]C1([2H])C(=O)NC(=O)C([2H])(N2Cc3cc(CNC(=O)C(F)(F)c4ccc(Cl)cc4)ccc3C2=O)C1([2H])[2H]] |
| 55 | 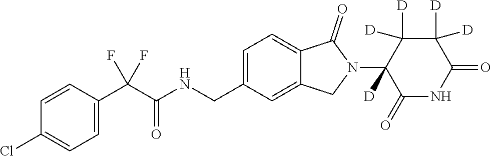 [SMILES: [2H]C1([2H])C(=O)NC(=O)[C@]([2H])(N2Cc3cc(CNC(=O)C(F)(F)c4ccc(Cl)cc4)ccc3C2=O)C1([2H])[2H]] |
| 56 | 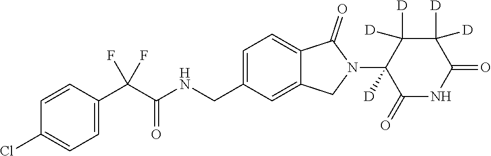 [SMILES: [2H]C1([2H])C(=O)NC(=O)[C@@]([2H])(N2Cc3cc(CNC(=O)C(F)(F)c4ccc(Cl)cc4)ccc3C2=O)C1([2H])[2H]] |
| 57 | 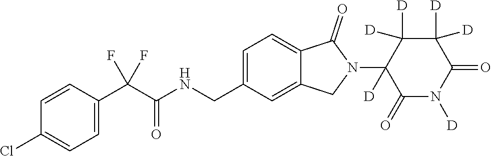 |
| 58 | 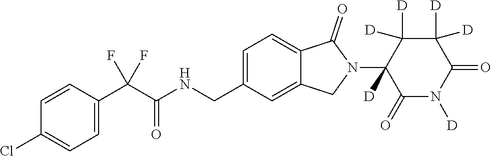 |
| 59 | 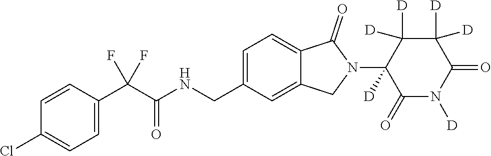 |
| 60 | 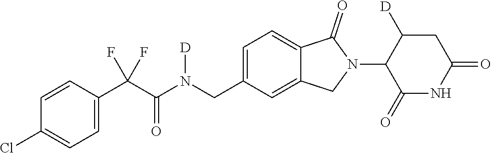 |
| 61 | 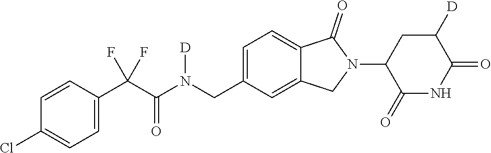 |
| 62 | 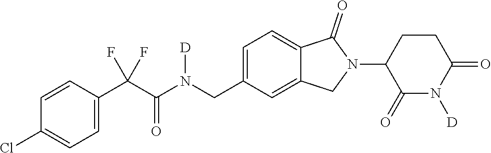 |
| 63 | 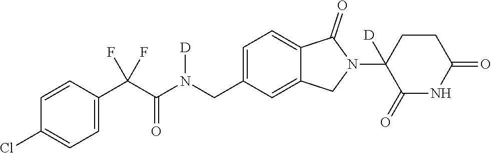 |
| 64 | 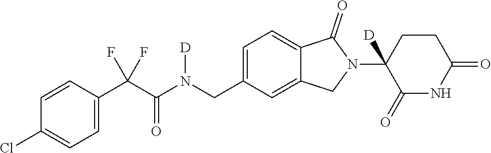 |
| 65 | 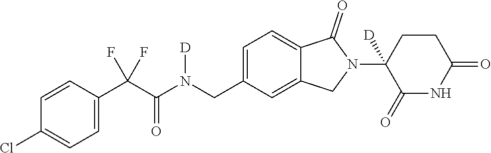 |
| 66 | 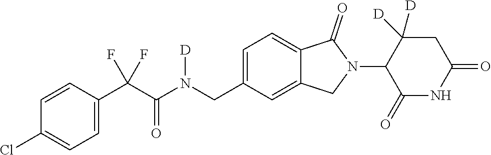 |
| 67 | 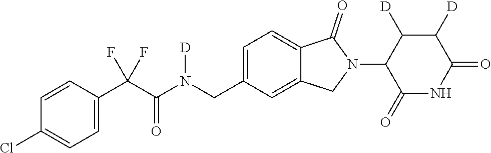 |
| 68 | 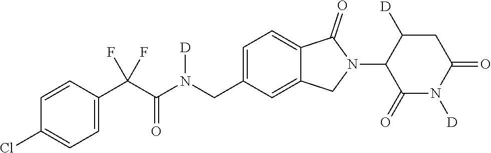 |
| 69 | 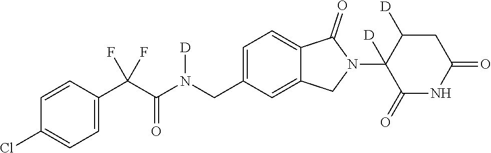 |
| 70 | 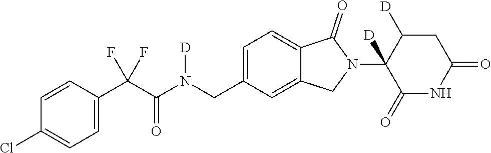 |
| 71 | 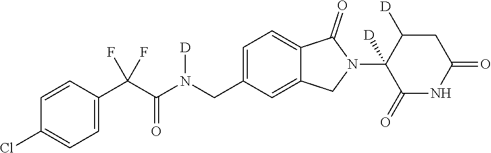 |
| 72 | 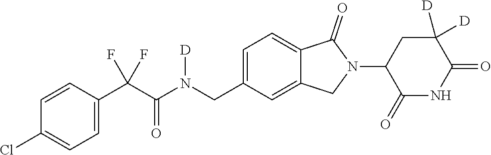 |
| 73 | 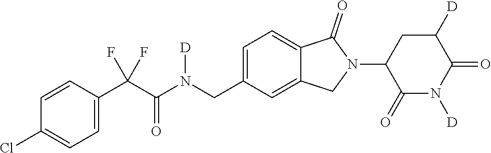 |
| 74 | 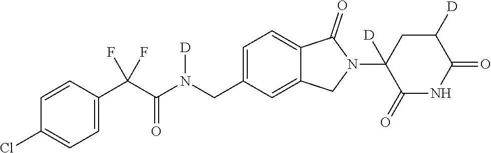 |
| 75 | 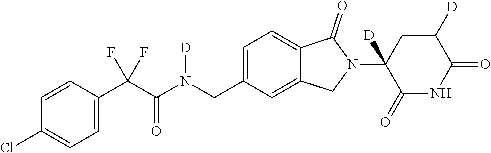 |
| 76 | 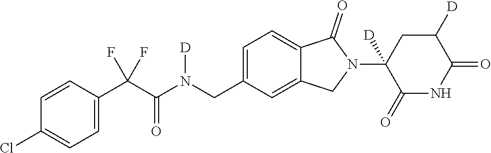 |
| 77 | 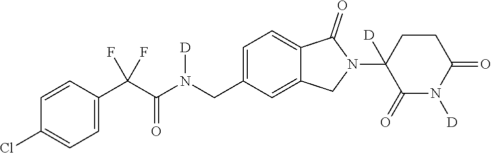 |
| 78 | 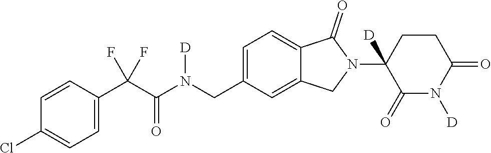 |
| 79 | 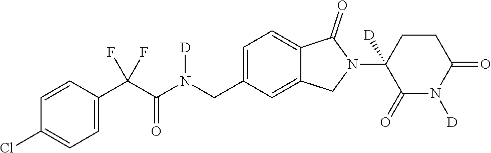 |
| 80 | 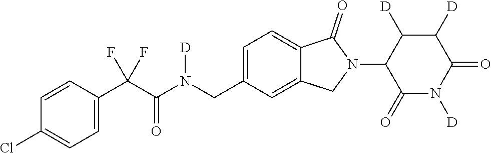 |
| 81 | 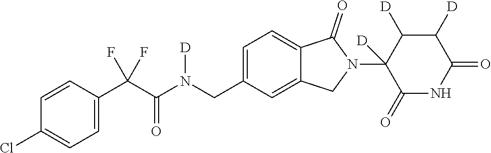 |
| 82 | 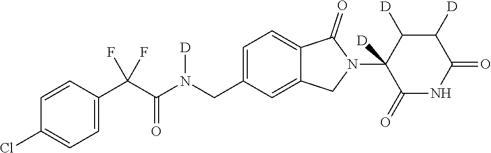 |
| 83 | 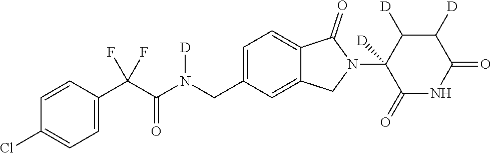 |
| 84 | 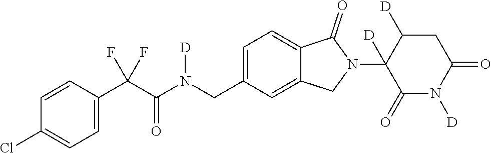 |
| 85 | 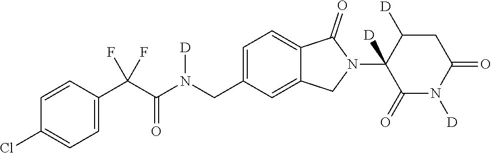 |
| 86 | 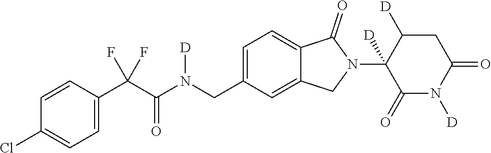 |
| 87 | 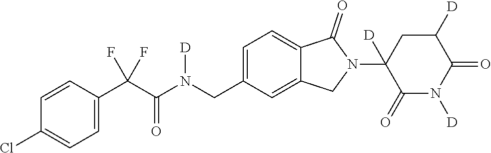 |
| 88 | 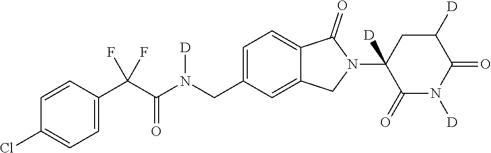 |
| 89 | 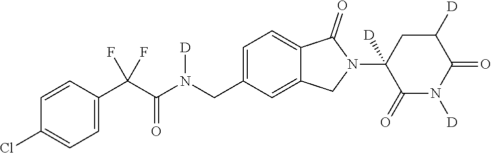 |
| 90 | 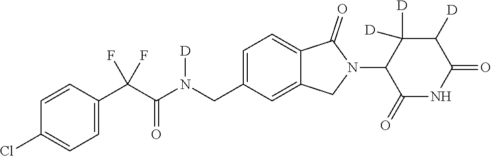 |
| 91 | 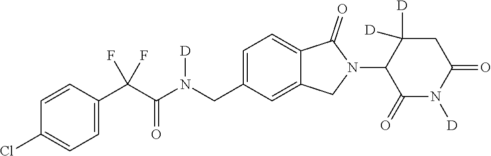 |
| 92 | 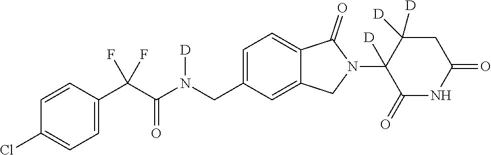 |
| 93 | 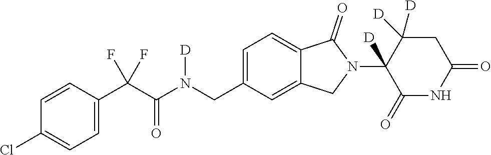 |
| 94 | 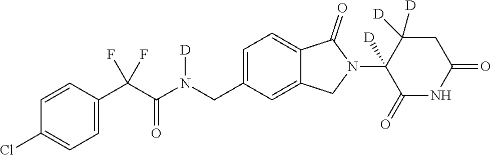 |
| 95 | 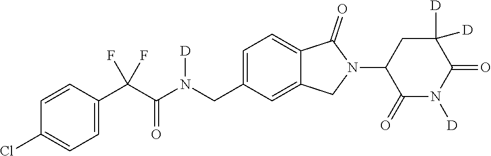 |
| 96 | 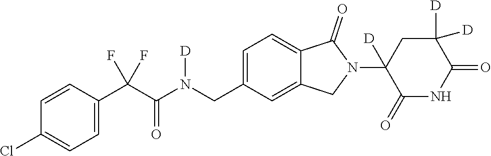 |
| 97 | 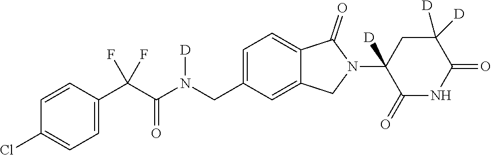 |
| 98 | 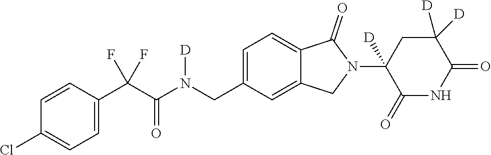 |
| 99 | 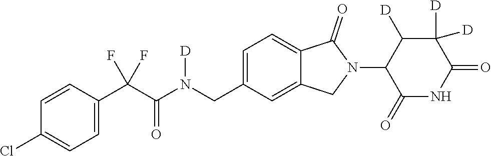 |
| 100 | 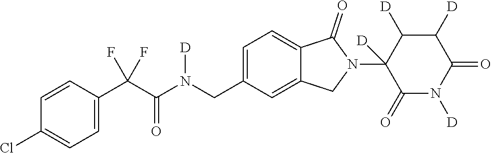 |
| 101 | 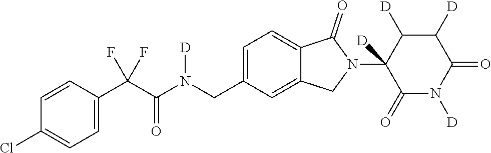 |
| 102 | 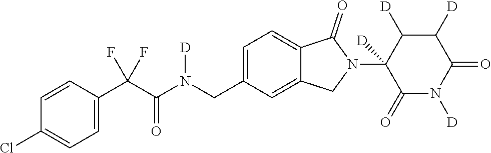 |
| 103 | 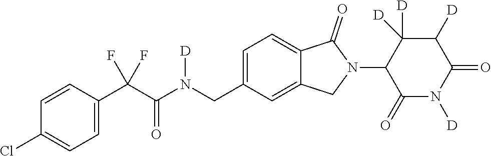 |
| 104 | 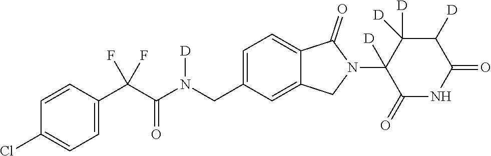 |
| 105 | 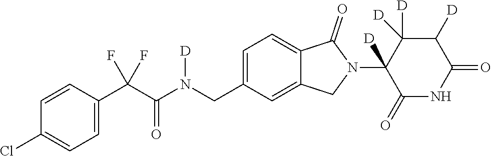 |
| 106 | 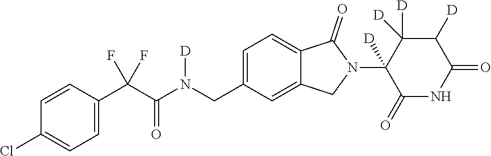 |
| 107 | 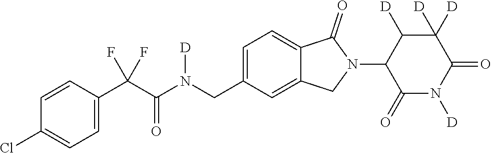 |
| 108 | 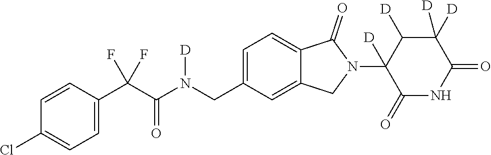 |
| 109 | 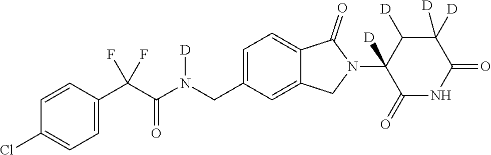 |
| 110 | 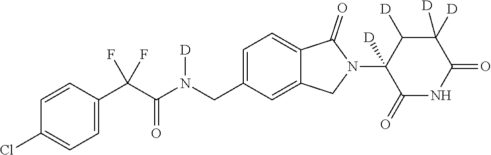 |
| 111 | 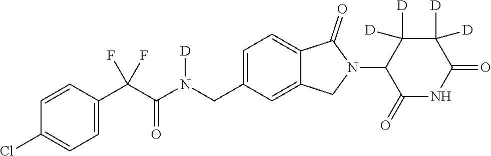 |
| 112 | 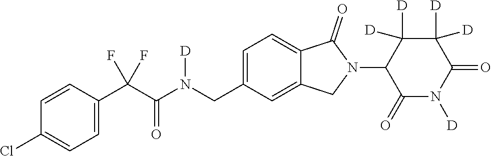 |
| 113 | 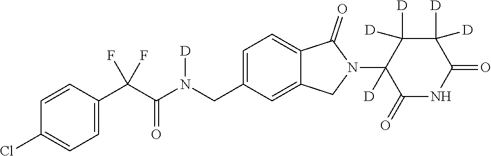 |
| 114 | 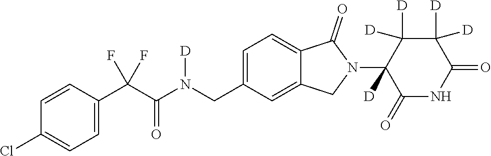 |
| 115 | 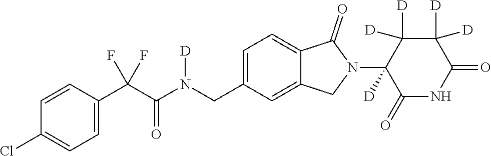 |
| 116 | 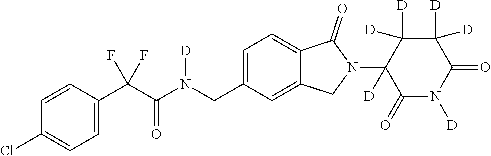 |
| 117 | 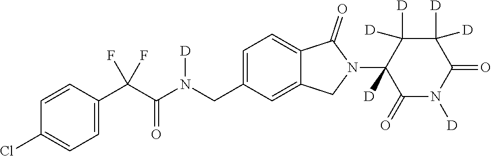 |
| 118 | 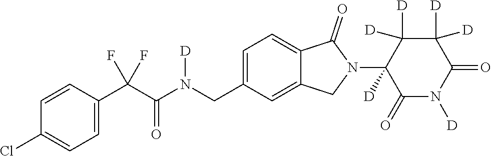 |
| 119 | 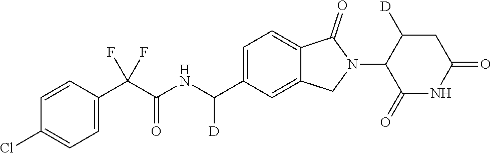 [SMILES: [2H]C(NC(=O)C(F)(F)c1ccc(Cl)cc1)c1ccc2c(c1)CN(C1C(=O)NC(=O)CC1[2H])C2=O] |
| 120 | 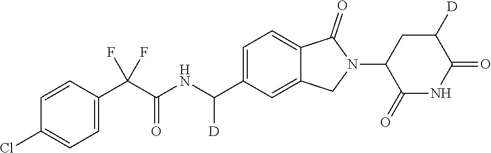 [SMILES: [2H]C1CC(N2Cc3cc(C([2H])NC(=O)C(F)(F)c4ccc(Cl)cc4)ccc3C2=O)C(=O)NC1=O] |
| 121 | 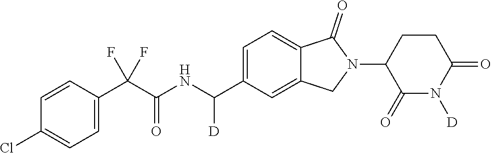 |
| 122 | 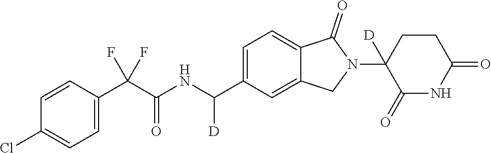 [SMILES: [2H]C(NC(=O)C(F)(F)c1ccc(Cl)cc1)c1ccc2c(c1)CN(C1([2H])CCC(=O)NC1=O)C2=O] |
| 123 | 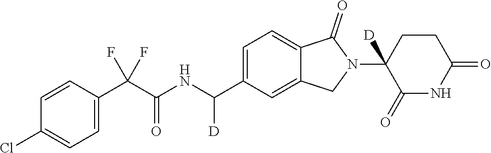 [SMILES: [2H]C(NC(=O)C(F)(F)c1ccc(Cl)cc1)c1ccc2c(c1)CN([C@]1([2H])CCC(=O)NC1=O)C2=O] |
| 124 | 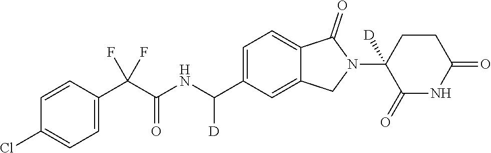 [SMILES: [2H]C(NC(=O)C(F)(F)c1ccc(Cl)cc1)c1ccc2c(c1)CN([C@@]1([2H])CCC(=O)NC1=O)C2=O] |
| 125 | 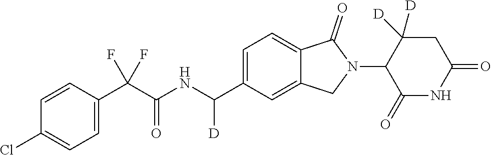 [SMILES: [2H]C(NC(=O)C(F)(F)c1ccc(Cl)cc1)c1ccc2c(c1)CN(C1C(=O)NC(=O)CC1([2H])[2H])C2=O] |
| 126 | 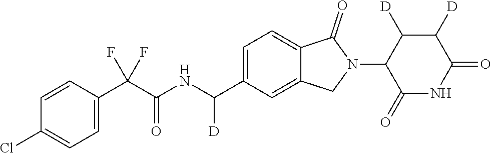 [SMILES: [2H]C(NC(=O)C(F)(F)c1ccc(Cl)cc1)c1ccc2c(c1)CN(C1C(=O)NC(=O)C([2H])C1[2H])C2=O] |
| 127 | 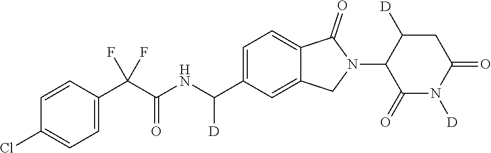 |
| 128 | 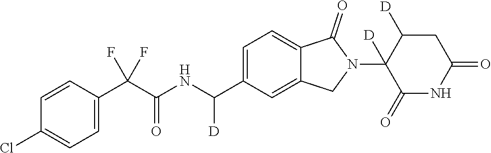 [SMILES: [2H]C(NC(=O)C(F)(F)c1ccc(Cl)cc1)c1ccc2c(c1)CN(C1([2H])C(=O)NC(=O)CC1[2H])C2=O] |
| 129 | 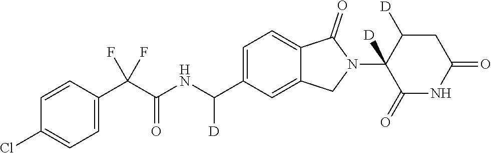 [SMILES: [2H]C(NC(=O)C(F)(F)c1ccc(Cl)cc1)c1ccc2c(c1)CN([C@@]1([2H])C(=O)NC(=O)CC1[2H])C2=O] |
| 130 | 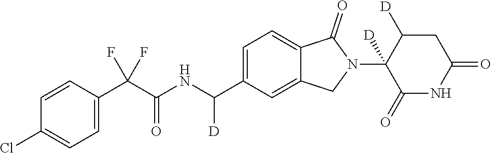 [SMILES: [2H]C(NC(=O)C(F)(F)c1ccc(Cl)cc1)c1ccc2c(c1)CN([C@]1([2H])C(=O)NC(=O)CC1[2H])C2=O] |
| 131 | 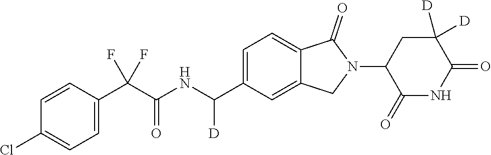 [SMILES: [2H]C(NC(=O)C(F)(F)c1ccc(Cl)cc1)c1ccc2c(c1)CN(C1CC([2H])([2H])C(=O)NC1=O)C2=O] |
| 132 | 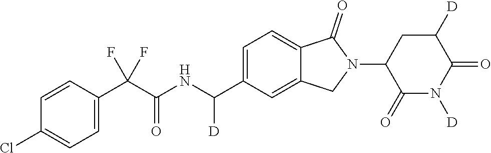 |
| 133 | 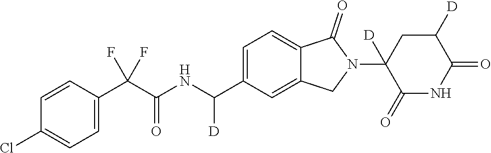 [SMILES: [2H]C1CC([2H])(N2Cc3cc(C([2H])NC(=O)C(F)(F)c4ccc(Cl)cc4)ccc3C2=O)C(=O)NC1=O] |
| 134 | 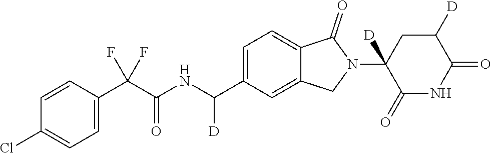 [SMILES: [2H]C1C[C@@]([2H])(N2Cc3cc(C([2H])NC(=O)C(F)(F)c4ccc(Cl)cc4)ccc3C2=O)C(=O)NC1=O] |
| 135 | 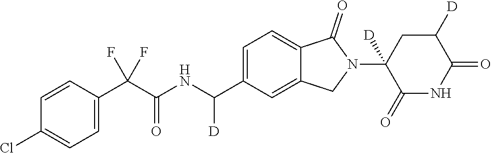 [SMILES: [2H]C1C[C@]([2H])(N2Cc3cc(C([2H])NC(=O)C(F)(F)c4ccc(Cl)cc4)ccc3C2=O)C(=O)NC1=O] |
| 136 | 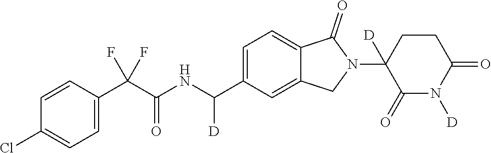 |
| 137 | 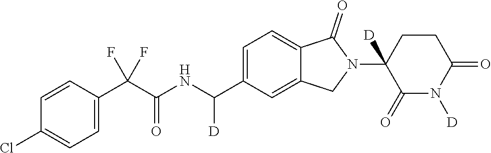 |
| 138 | 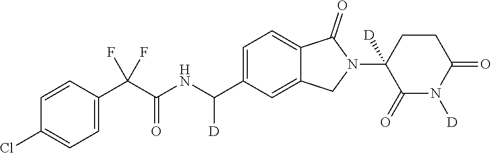 |
| 139 | 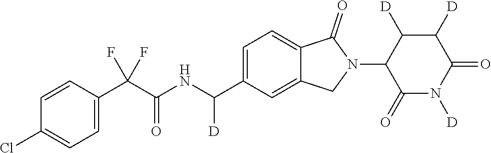 |
| 140 | 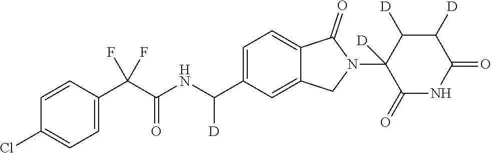 [SMILES: [2H]C(NC(=O)C(F)(F)c1ccc(Cl)cc1)c1ccc2c(c1)CN(C1([2H])C(=O)NC(=O)C([2H])C1[2H])C2=O] |
| 141 | 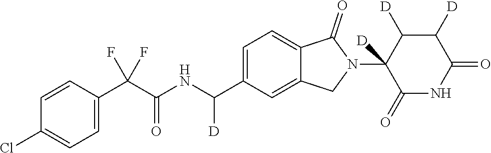 [SMILES: [2H]C(NC(=O)C(F)(F)c1ccc(Cl)cc1)c1ccc2c(c1)CN([C@@]1([2H])C(=O)NC(=O)C([2H])C1[2H])C2=O] |
| 142 | 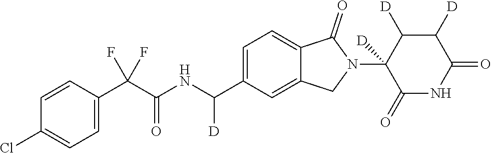 [SMILES: [2H]C(NC(=O)C(F)(F)c1ccc(Cl)cc1)c1ccc2c(c1)CN([C@]1([2H])C(=O)NC(=O)C([2H])C1[2H])C2=O] |
| 143 | 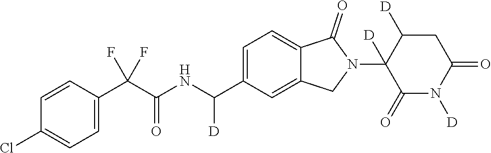 |
| 144 | 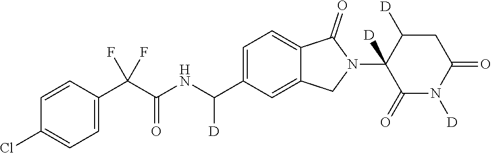 |
| 145 | 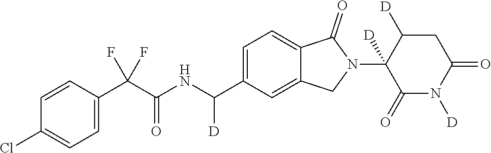 |
| 146 | 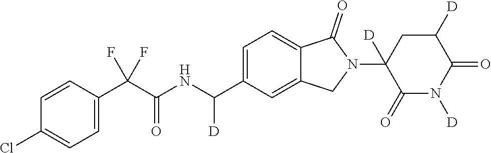 |
| 147 | 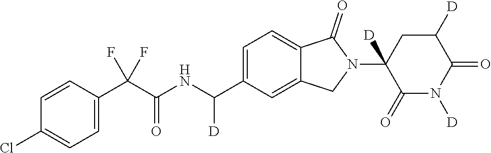 |
| 148 | 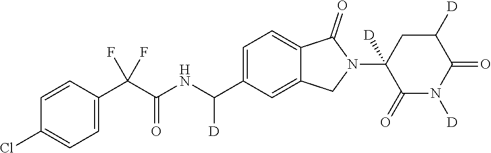 |
| 149 | 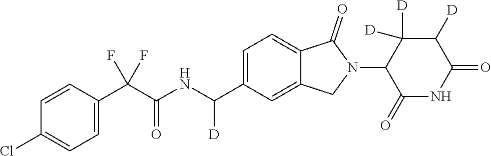 [SMILES: [2H]C(NC(=O)C(F)(F)c1ccc(Cl)cc1)c1ccc2c(c1)CN(C1C(=O)NC(=O)C([2H])C1([2H])[2H])C2=O] |
| 150 | 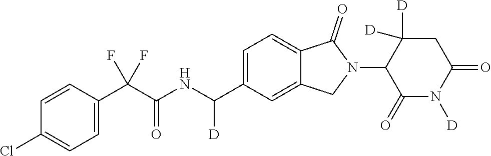 |
| 151 | 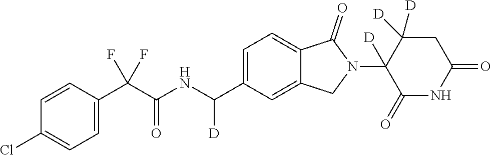 [SMILES: [2H]C(NC(=O)C(F)(F)c1ccc(Cl)cc1)c1ccc2c(c1)CN(C1([2H])C(=O)NC(=O)CC1([2H])[2H])C2=O] |
| 152 | 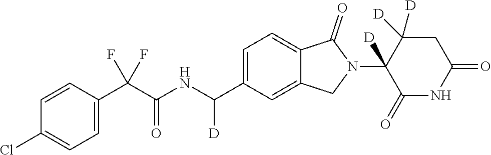 [SMILES: [2H]C(NC(=O)C(F)(F)c1ccc(Cl)cc1)c1ccc2c(c1)CN([C@@]1([2H])C(=O)NC(=O)CC1([2H])[2H])C2=O] |
| 153 | 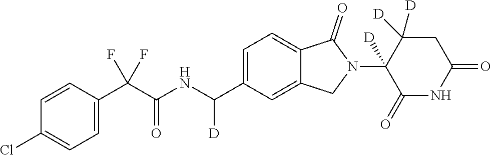 [SMILES: [2H]C(NC(=O)C(F)(F)c1ccc(Cl)cc1)c1ccc2c(c1)CN([C@]1([2H])C(=O)NC(=O)CC1([2H])[2H])C2=O] |
| 154 | 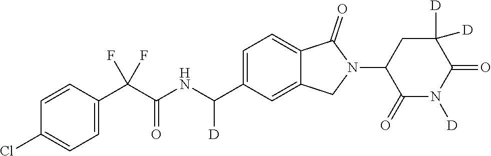 |
| 155 | 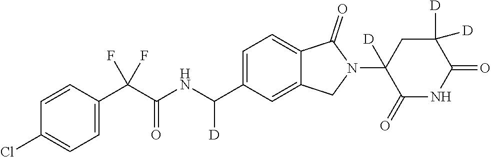 [SMILES: [2H]C(NC(=O)C(F)(F)c1ccc(Cl)cc1)c1ccc2c(c1)CN(C1([2H])CC([2H])([2H])C(=O)NC1=O)C2=O] |
| 156 | 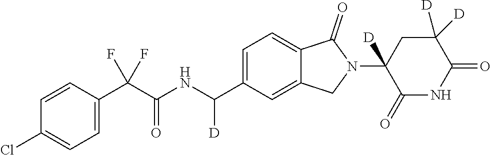 [SMILES: [2H]C(NC(=O)C(F)(F)c1ccc(Cl)cc1)c1ccc2c(c1)CN([C@]1([2H])CC([2H])([2H])C(=O)NC1=O)C2=O] |
| 157 | 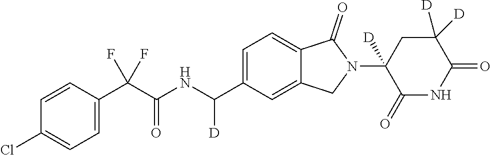 [SMILES: [2H]C(NC(=O)C(F)(F)c1ccc(Cl)cc1)c1ccc2c(c1)CN([C@@]1([2H])CC([2H])([2H])C(=O)NC1=O)C2=O] |
| 158 | 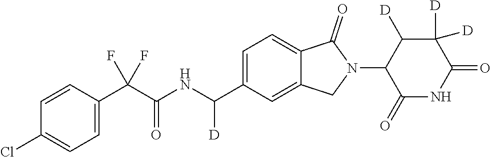 [SMILES: [2H]C(NC(=O)C(F)(F)c1ccc(Cl)cc1)c1ccc2c(c1)CN(C1C(=O)NC(=O)C([2H])([2H])C1[2H])C2=O] |
| 159 | 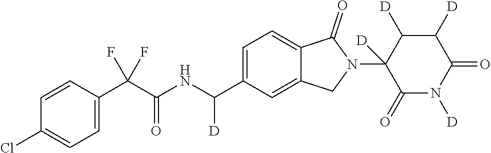 |
| 160 | 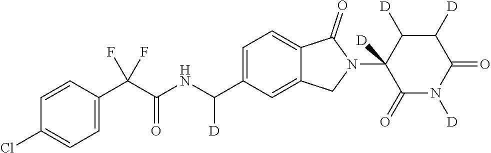 |
| 161 | 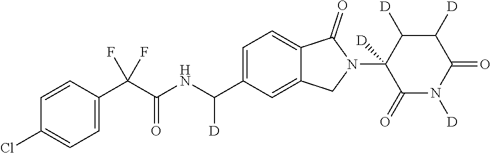 |
| 162 | 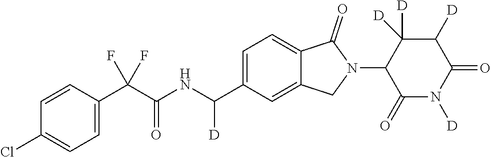 |
| 163 | 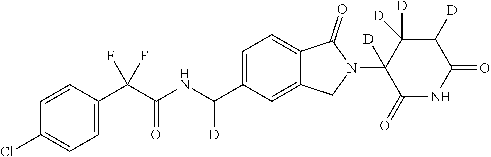 [SMILES: [2H]C(NC(=O)C(F)(F)c1ccc(Cl)cc1)c1ccc2c(c1)CN(C1([2H])C(=O)NC(=O)C([2H])C1([2H])[2H])C2=O] |
| 164 | 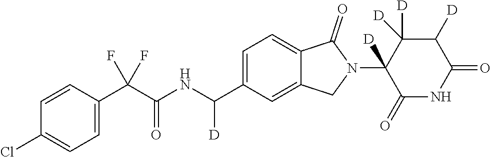 [SMILES: [2H]C(NC(=O)C(F)(F)c1ccc(Cl)cc1)c1ccc2c(c1)CN([C@@]1([2H])C(=O)NC(=O)C([2H])C1([2H])[2H])C2=O] |
| 165 | 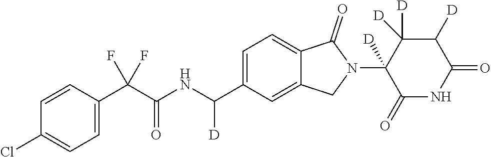 [SMILES: [2H]C(NC(=O)C(F)(F)c1ccc(Cl)cc1)c1ccc2c(c1)CN([C@]1([2H])C(=O)NC(=O)C([2H])C1([2H])[2H])C2=O] |
| 166 | 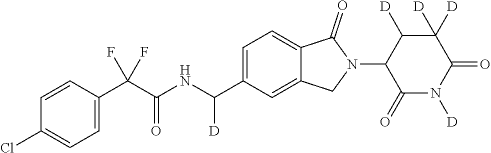 |
| 167 | 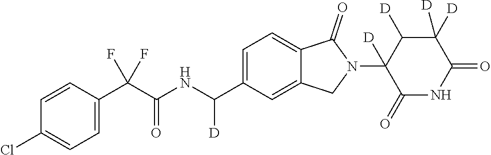 [SMILES: [2H]C(NC(=O)C(F)(F)c1ccc(Cl)cc1)c1ccc2c(c1)CN(C1([2H])C(=O)NC(=O)C([2H])([2H])C1[2H])C2=O] |
| 168 | 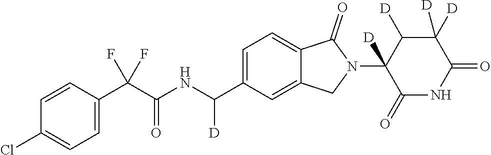 [SMILES: [2H]C(NC(=O)C(F)(F)c1ccc(Cl)cc1)c1ccc2c(c1)CN([C@@]1([2H])C(=O)NC(=O)C([2H])([2H])C1[2H])C2=O] |
| 169 | 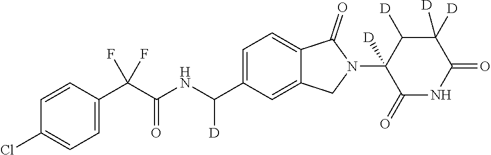 [SMILES: [2H]C(NC(=O)C(F)(F)c1ccc(Cl)cc1)c1ccc2c(c1)CN([C@]1([2H])C(=O)NC(=O)C([2H])([2H])C1[2H])C2=O] |
| 170 | 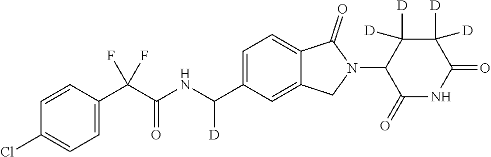 [SMILES: [2H]C(NC(=O)C(F)(F)c1ccc(Cl)cc1)c1ccc2c(c1)CN(C1C(=O)NC(=O)C([2H])([2H])C1([2H])[2H])C2=O] |
| 171 | 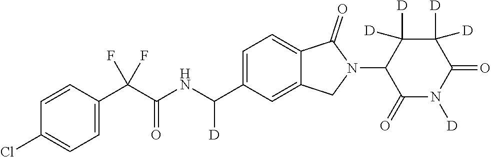 |
| 172 | 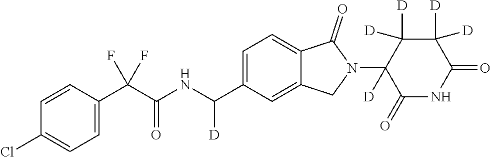 [SMILES: [2H]C(NC(=O)C(F)(F)c1ccc(Cl)cc1)c1ccc2c(c1)CN(C1([2H])C(=O)NC(=O)C([2H])([2H])C1([2H])[2H])C2=O] |
| 173 | 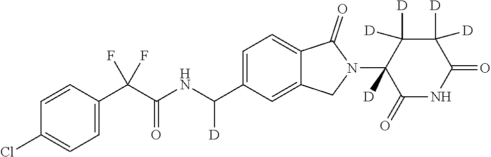 [SMILES: [2H]C(NC(=O)C(F)(F)c1ccc(Cl)cc1)c1ccc2c(c1)CN([C@@]1([2H])C(=O)NC(=O)C([2H])([2H])C1([2H])[2H])C2=O] |
| 174 | 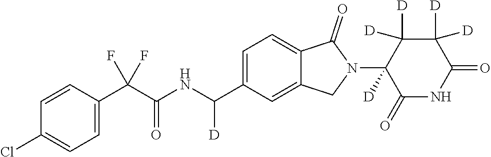 [SMILES: [2H]C(NC(=O)C(F)(F)c1ccc(Cl)cc1)c1ccc2c(c1)CN([C@]1([2H])C(=O)NC(=O)C([2H])([2H])C1([2H])[2H])C2=O] |
| 175 | 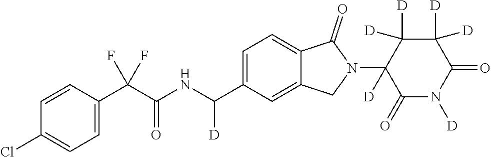 |
| 176 | 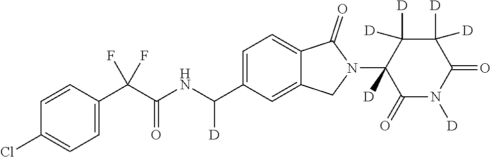 |
| 177 | 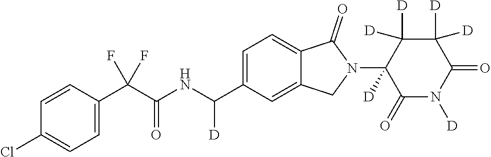 |
| 178 | 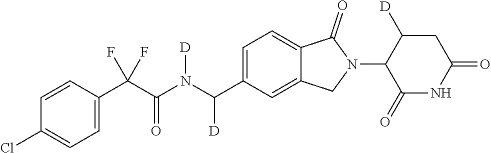 |
| 179 | 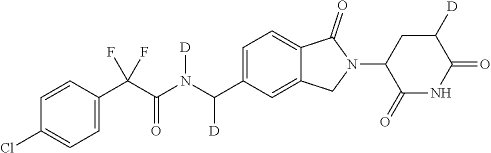 |
| 180 | 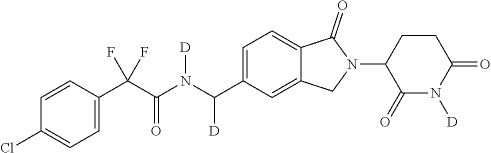 |
| 181 | 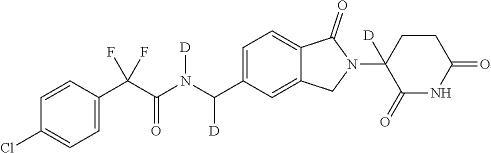 |
| 182 | 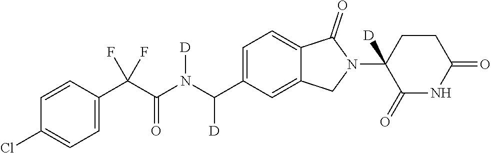 |
| 183 | 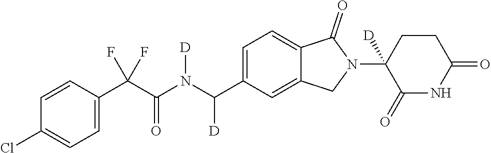 |
| 184 | 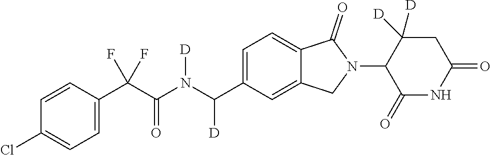 |
| 185 | 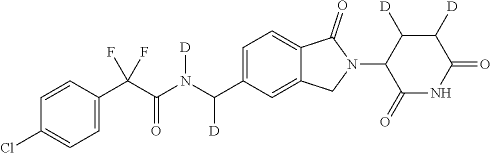 |
| 186 | 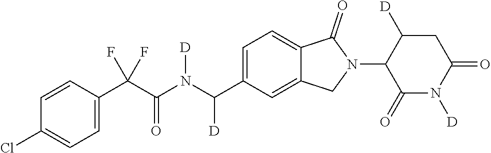 |
| 187 | 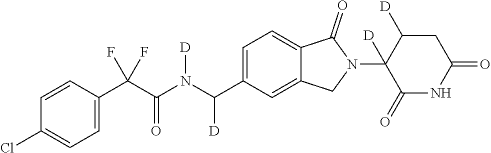 |
| 188 | 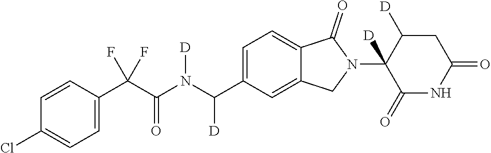 |
| 189 | 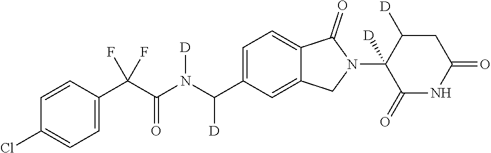 |
| 190 | 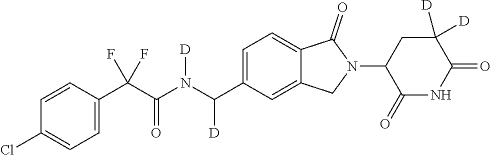 |
| 191 | 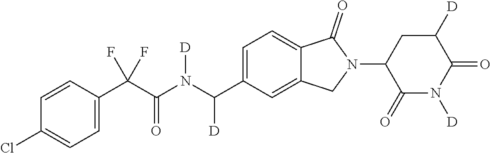 |
| 192 | 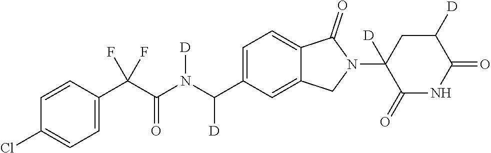 |
| 193 | 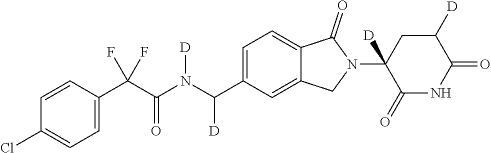 |
| 194 | 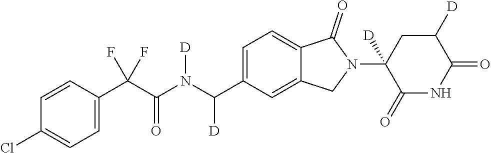 |
| 195 | 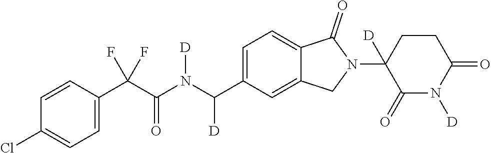 |
| 196 | 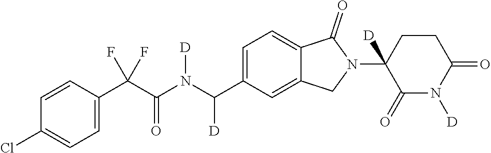 |
| 197 | 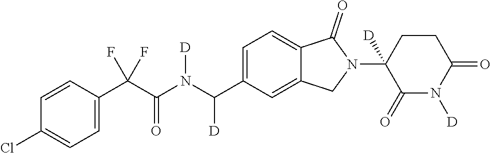 |
| 298 | 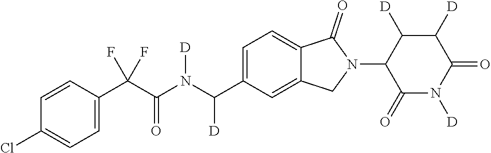 |
| 199 | 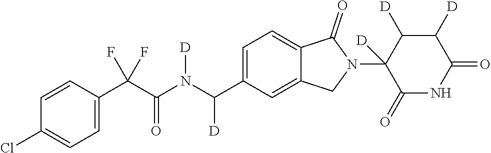 |
| 200 | 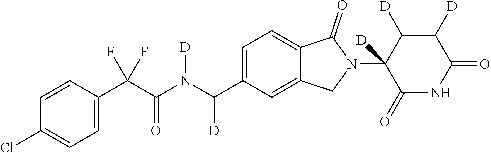 |
| 201 | 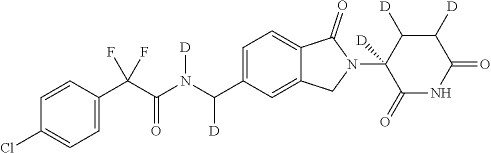 |
| 202 | 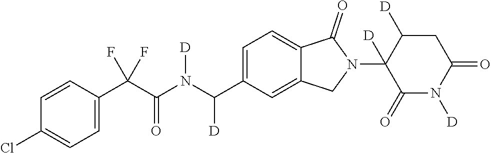 |
| 203 | 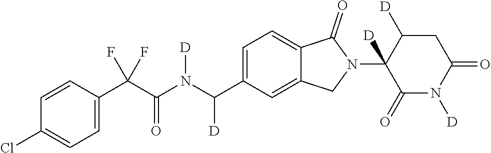 |
| 204 | 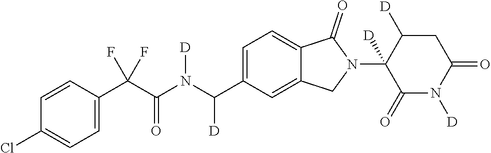 |
| 205 | 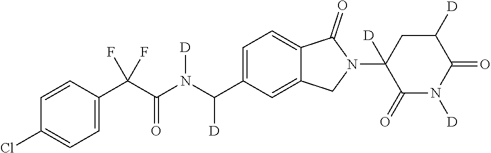 |
| 206 | 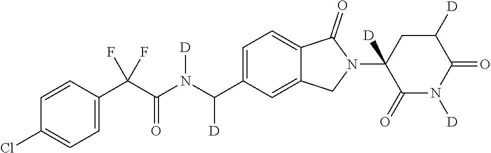 |
| 207 | 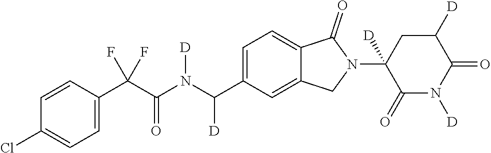 |
| 208 | 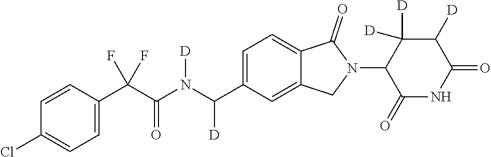 |
| 209 | 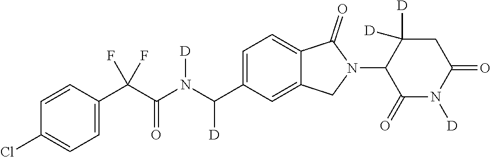 |
| 210 | 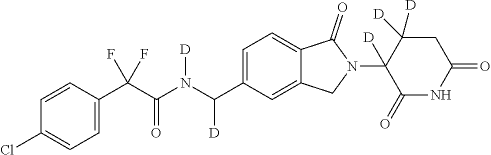 |
| 211 | 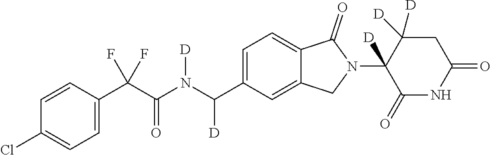 |
| 212 | 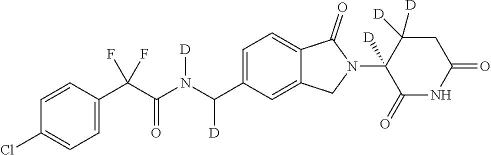 |
| 213 | 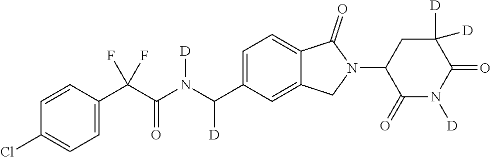 |
| 214 | 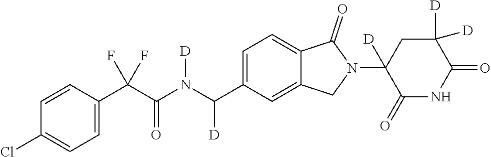 |
| 215 | 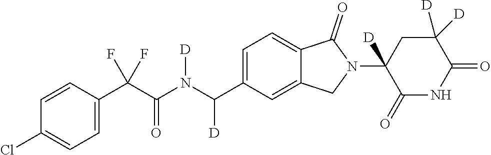 |
| 216 | 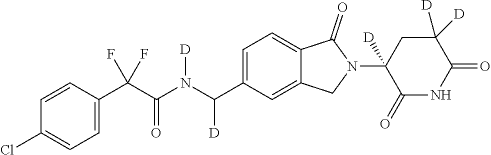 |
| 217 | 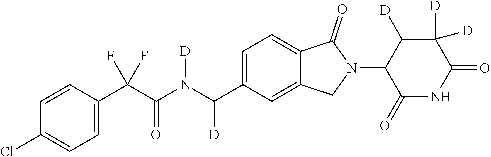 |
| 218 | 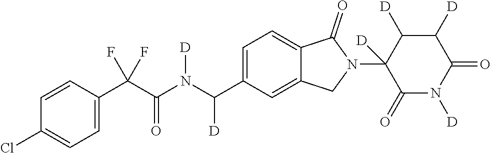 |
| 219 | 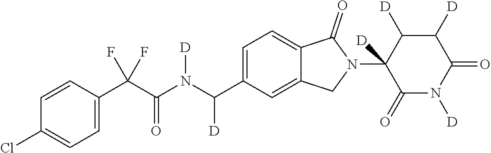 |
| 220 | 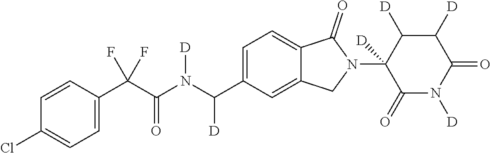 |
| 221 | 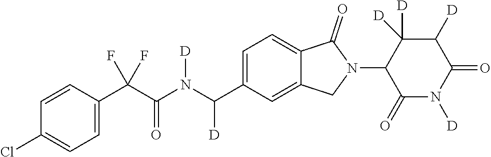 |
| 222 | 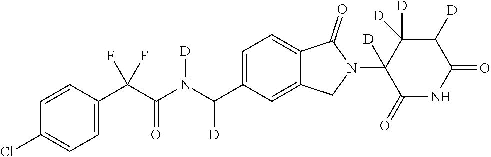 |
| 223 | 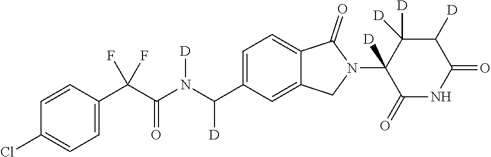 |
| 224 | 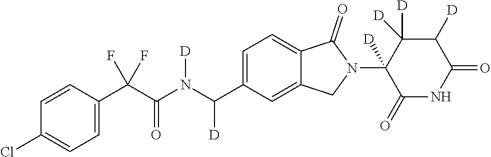 |
| 225 | 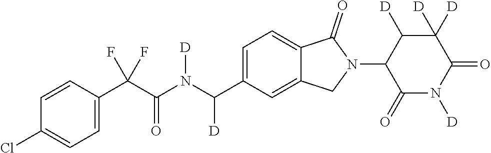 |
| 226 | 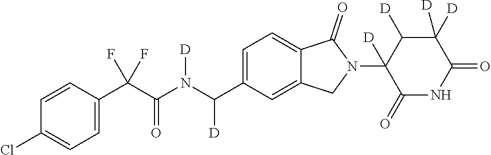 |
| 227 | 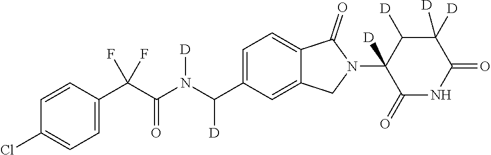 |
| 228 | 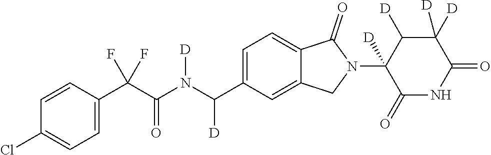 |
| 229 | 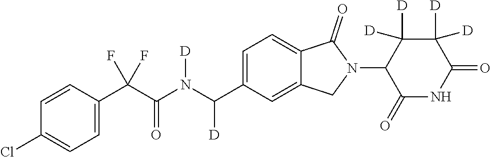 |
| 230 | 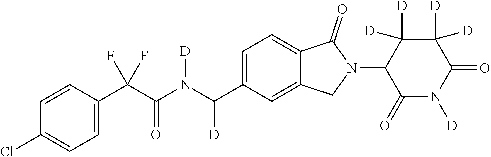 |
| 231 | 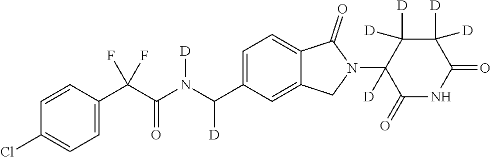 |
| 232 | 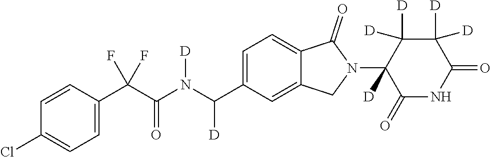 |
| 233 | 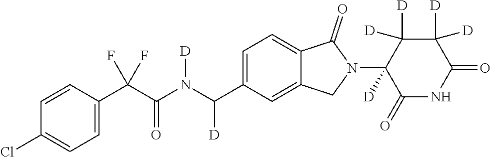 |
| 234 | 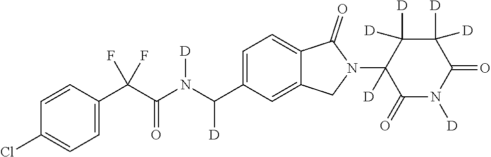 |
| 235 | 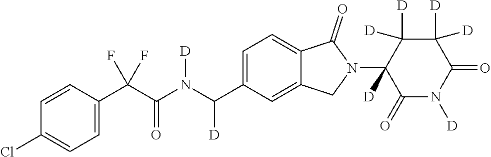 |
| 236 | 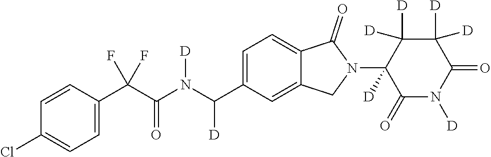 |
| 237 | 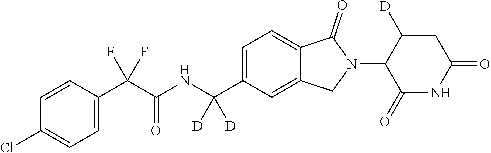 [SMILES: [2H]C1CC(=O)NC(=O)C1N1Cc2cc(C([2H])([2H])NC(=O)C(F)(F)c3ccc(Cl)cc3)ccc2C1=O] |
| 238 | 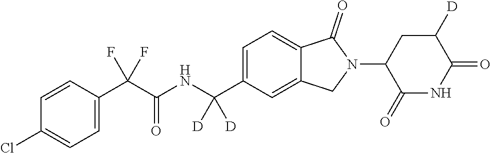 [SMILES: [2H]C1CC(N2Cc3cc(C([2H])([2H])NC(=O)C(F)(F)c4ccc(Cl)cc4)ccc3C2=O)C(=O)NC1=O] |
| 239 | 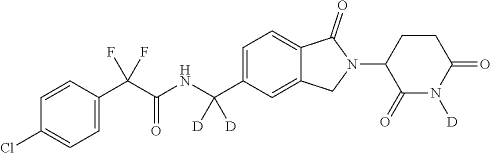 |
| 240 | 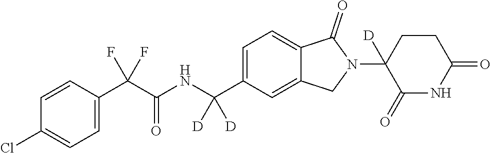 [SMILES: [2H]C([2H])(NC(=O)C(F)(F)c1ccc(Cl)cc1)c1ccc2c(c1)CN(C1([2H])CCC(=O)NC1=O)C2=O] |
| 241 | 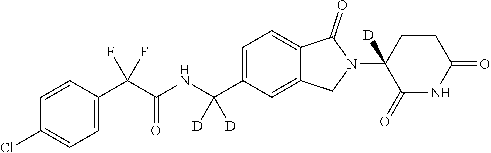 [SMILES: [2H]C([2H])(NC(=O)C(F)(F)c1ccc(Cl)cc1)c1ccc2c(c1)CN([C@]1([2H])CCC(=O)NC1=O)C2=O] |
| 242 | 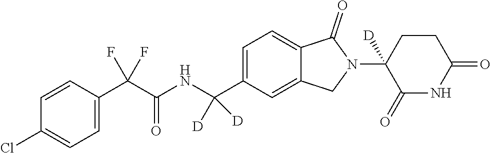 [SMILES: [2H]C([2H])(NC(=O)C(F)(F)c1ccc(Cl)cc1)c1ccc2c(c1)CN([C@@]1([2H])CCC(=O)NC1=O)C2=O] |
| 243 | 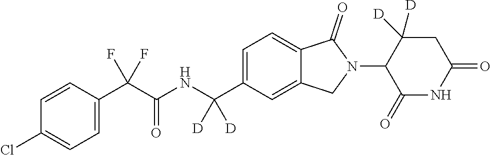 [SMILES: [2H]C([2H])(NC(=O)C(F)(F)c1ccc(Cl)cc1)c1ccc2c(c1)CN(C1C(=O)NC(=O)CC1([2H])[2H])C2=O] |
| 244 | 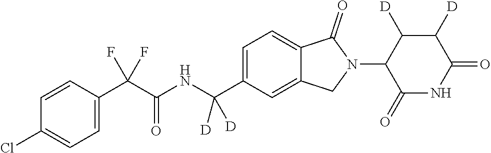 [SMILES: [2H]C1C(=O)NC(=O)C(N2Cc3cc(C([2H])([2H])NC(=O)C(F)(F)c4ccc(Cl)cc4)ccc3C2=O)C1[2H]] |
| 245 | 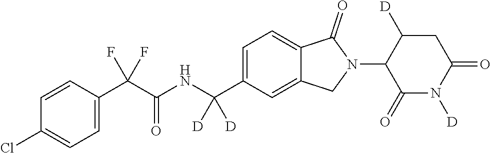 |
| 246 | 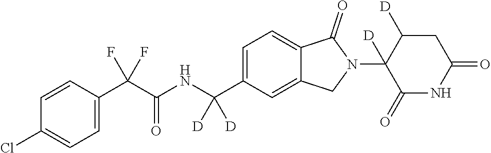 [SMILES: [2H]C1CC(=O)NC(=O)C1([2H])N1Cc2cc(C([2H])([2H])NC(=O)C(F)(F)c3ccc(Cl)cc3)ccc2C1=O] |
| 247 | 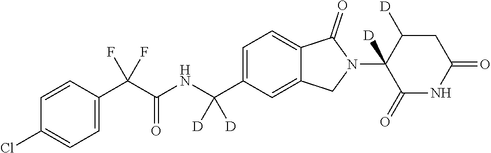 [SMILES: [2H]C1CC(=O)NC(=O)[C@]1([2H])N1Cc2cc(C([2H])([2H])NC(=O)C(F)(F)c3ccc(Cl)cc3)ccc2C1=O] |
| 248 | 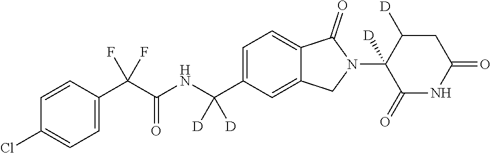 [SMILES: [2H]C1CC(=O)NC(=O)[C@@]1([2H])N1Cc2cc(C([2H])([2H])NC(=O)C(F)(F)c3ccc(Cl)cc3)ccc2C1=O] |
| 249 | 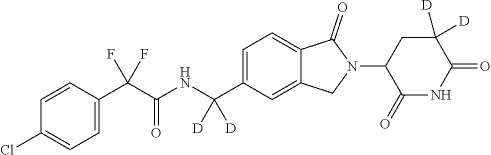 [SMILES: [2H]C1([2H])CC(N2Cc3cc(C([2H])([2H])NC(=O)C(F)(F)c4ccc(Cl)cc4)ccc3C2=O)C(=O)NC1=O] |
| 250 | 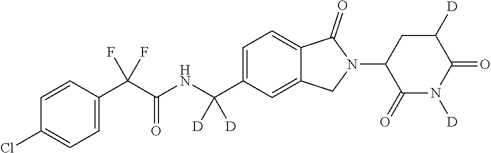 |
| 251 | 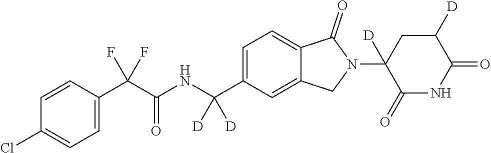 [SMILES: [2H]C1CC([2H])(N2Cc3cc(C([2H])([2H])NC(=O)C(F)(F)c4ccc(Cl)cc4)ccc3C2=O)C(=O)NC1=O] |
| 252 | 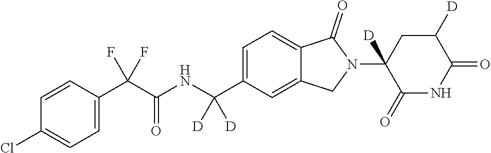 [SMILES: [2H]C1C[C@@]([2H])(N2Cc3cc(C([2H])([2H])NC(=O)C(F)(F)c4ccc(Cl)cc4)ccc3C2=O)C(=O)NC1=O] |
| 253 | 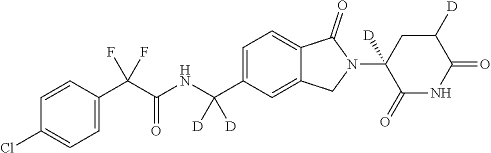 [SMILES: [2H]C1C[C@]([2H])(N2Cc3cc(C([2H])([2H])NC(=O)C(F)(F)c4ccc(Cl)cc4)ccc3C2=O)C(=O)NC1=O] |
| 254 | 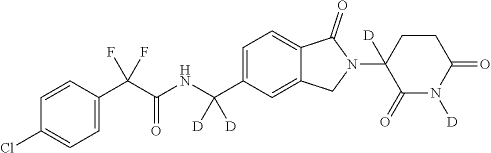 |
| 255 | 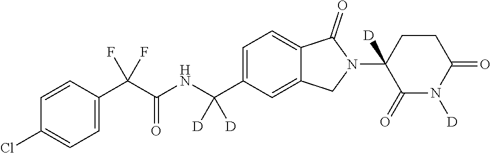 |
| 256 | 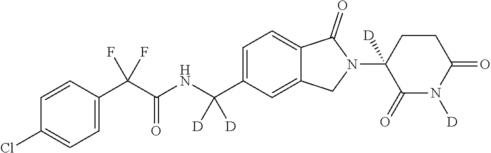 |
| 257 | 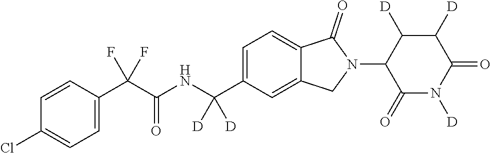 |
| 258 | 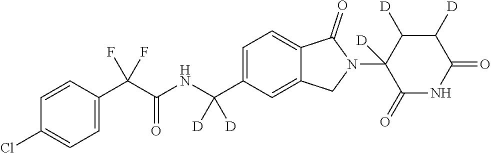 [SMILES: [2H]C1C(=O)NC(=O)C([2H])(N2Cc3cc(C([2H])([2H])NC(=O)C(F)(F)c4ccc(Cl)cc4)ccc3C2=O)C1[2H]] |
| 259 | 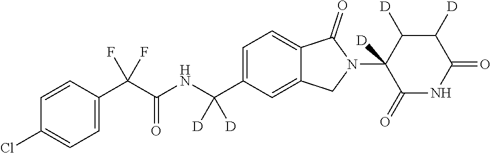 [SMILES: [2H]C1C(=O)NC(=O)[C@]([2H])(N2Cc3cc(C([2H])([2H])NC(=O)C(F)(F)c4ccc(Cl)cc4)ccc3C2=O)C1[2H]] |
| 260 | 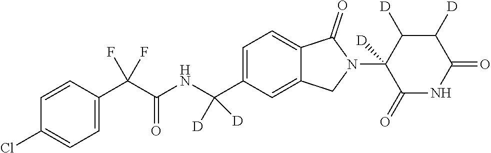 [SMILES: [2H]C1C(=O)NC(=O)[C@@]([2H])(N2Cc3cc(C([2H])([2H])NC(=O)C(F)(F)c4ccc(Cl)cc4)ccc3C2=O)C1[2H]] |
| 261 | 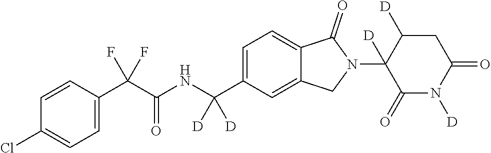 |
| 262 | 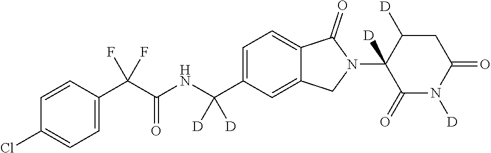 |
| 263 | 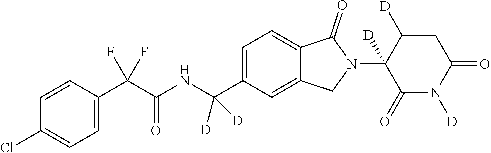 |
| 264 | 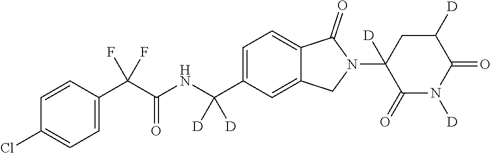 |
| 265 | 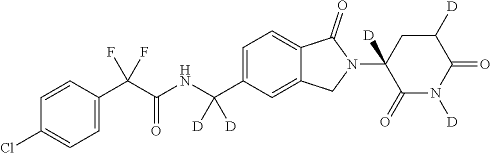 |
| 266 | 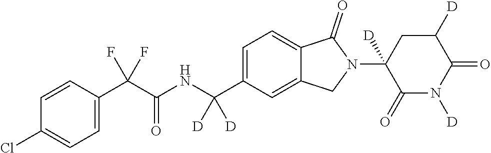 |
| 267 | 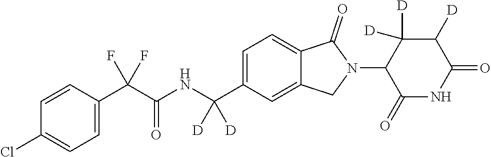 [SMILES: [2H]C1C(=O)NC(=O)C(N2Cc3cc(C([2H])([2H])NC(=O)C(F)(F)c4ccc(Cl)cc4)ccc3C2=O)C1([2H])[2H]] |
| 268 | 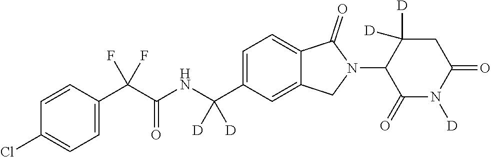 |
| 269 | 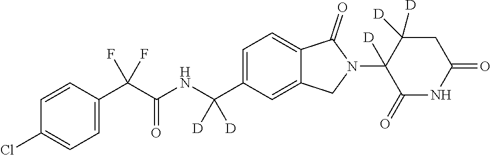 [SMILES: [2H]C([2H])(NC(=O)C(F)(F)c1ccc(Cl)cc1)c1ccc2c(c1)CN(C1([2H])C(=O)NC(=O)CC1([2H])[2H])C2=O] |
| 270 | 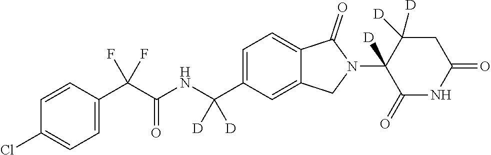 [SMILES: [2H]C([2H])(NC(=O)C(F)(F)c1ccc(Cl)cc1)c1ccc2c(c1)CN([C@@]1([2H])C(=O)NC(=O)CC1([2H])[2H])C2=O] |
| 271 | 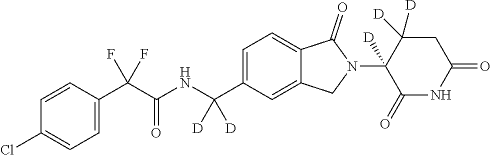 [SMILES: [2H]C([2H])(NC(=O)C(F)(F)c1ccc(Cl)cc1)c1ccc2c(c1)CN([C@]1([2H])C(=O)NC(=O)CC1([2H])[2H])C2=O] |
| 272 | 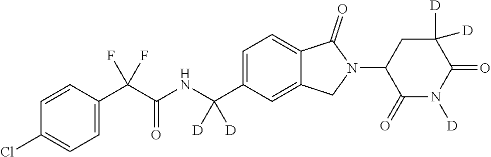 |
| 273 | 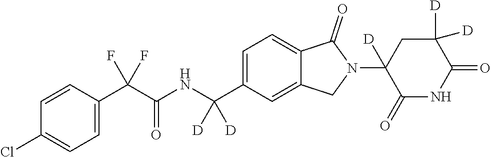 [SMILES: [2H]C1([2H])CC([2H])(N2Cc3cc(C([2H])([2H])NC(=O)C(F)(F)c4ccc(Cl)cc4)ccc3C2=O)C(=O)NC1=O] |
| 274 | 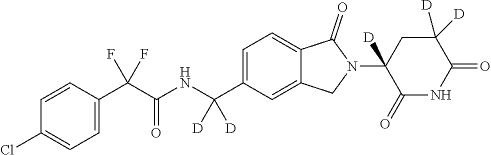 [SMILES: [2H]C1([2H])C[C@@]([2H])(N2Cc3cc(C([2H])([2H])NC(=O)C(F)(F)c4ccc(Cl)cc4)ccc3C2=O)C(=O)NC1=O] |
| 275 | 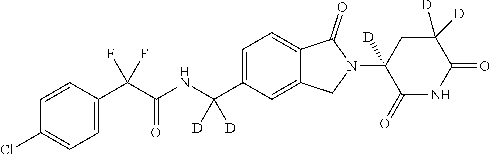 [SMILES: [2H]C1([2H])C[C@]([2H])(N2Cc3cc(C([2H])([2H])NC(=O)C(F)(F)c4ccc(Cl)cc4)ccc3C2=O)C(=O)NC1=O] |
| 276 | 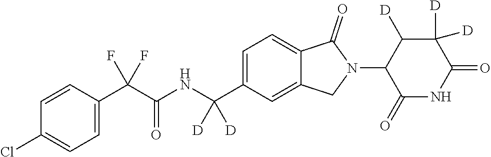 [SMILES: [2H]C1C(N2Cc3cc(C([2H])([2H])NC(=O)C(F)(F)c4ccc(Cl)cc4)ccc3C2=O)C(=O)NC(=O)C1([2H])[2H]] |
| 277 | 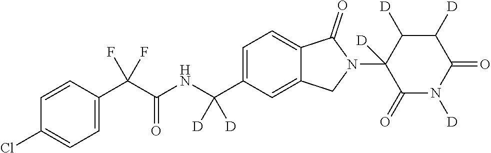 |
| 278 | 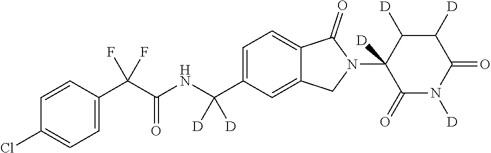 |
| 279 | 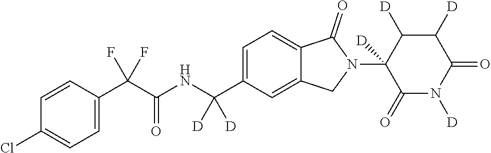 |
| 280 | 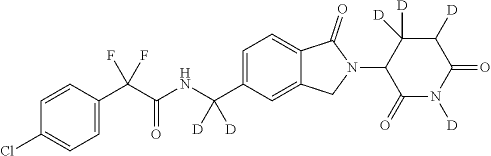 |
| 281 | 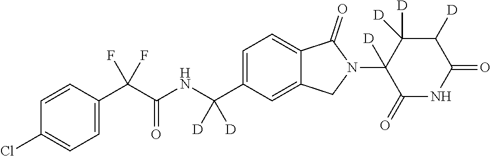 [SMILES: [2H]C1C(=O)NC(=O)C([2H])(N2Cc3cc(C([2H])([2H])NC(=O)C(F)(F)c4ccc(Cl)cc4)ccc3C2=O)C1([2H])[2H]] |
| 282 | 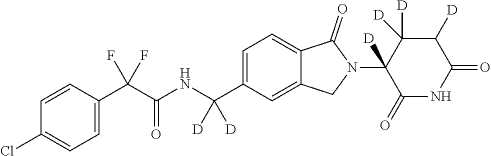 [SMILES: [2H]C1C(=O)NC(=O)[C@]([2H])(N2Cc3cc(C([2H])([2H])NC(=O)C(F)(F)c4ccc(Cl)cc4)ccc3C2=O)C1([2H])[2H]] |
| 283 | 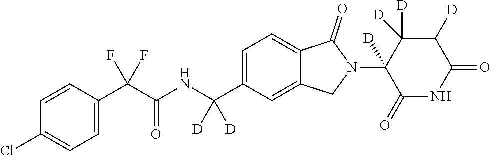 [SMILES: [2H]C1C(=O)NC(=O)[C@@]([2H])(N2Cc3cc(C([2H])([2H])NC(=O)C(F)(F)c4ccc(Cl)cc4)ccc3C2=O)C1([2H])[2H]] |
| 284 | 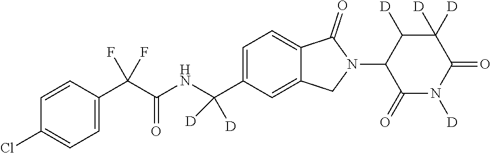 |
| 285 | 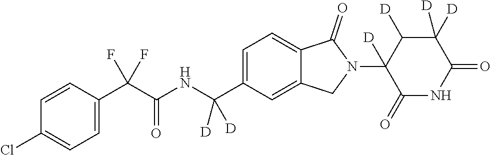 [SMILES: [2H]C1C([2H])([2H])C(=O)NC(=O)C1([2H])N1Cc2cc(C([2H])([2H])NC(=O)C(F)(F)c3ccc(Cl)cc3)ccc2C1=O] |
| 286 | 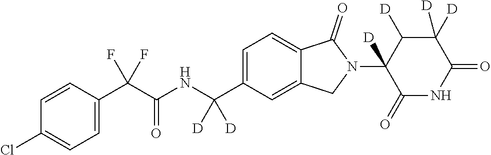 [SMILES: [2H]C1C([2H])([2H])C(=O)NC(=O)[C@]1([2H])N1Cc2cc(C([2H])([2H])NC(=O)C(F)(F)c3ccc(Cl)cc3)ccc2C1=O] |
| 287 | 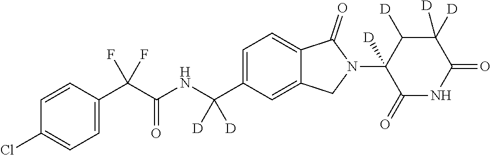 [SMILES: [2H]C1C([2H])([2H])C(=O)NC(=O)[C@@]1([2H])N1Cc2cc(C([2H])([2H])NC(=O)C(F)(F)c3ccc(Cl)cc3)ccc2C1=O] |
| 288 | 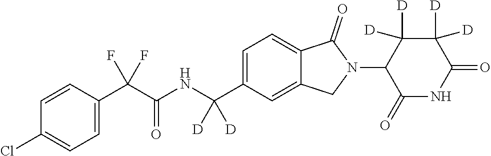 [SMILES: [2H]C([2H])(NC(=O)C(F)(F)c1ccc(Cl)cc1)c1ccc2c(c1)CN(C1C(=O)NC(=O)C([2H])([2H])C1([2H])[2H])C2=O] |
| 289 | 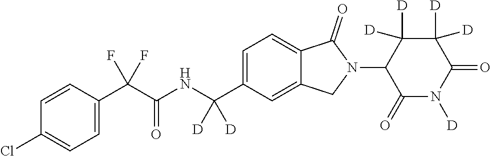 |
| 290 | 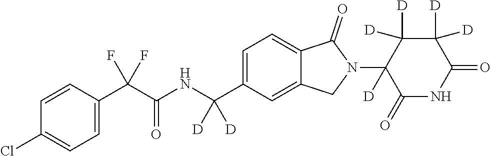 [SMILES: [2H]C([2H])(NC(=O)C(F)(F)c1ccc(Cl)cc1)c1ccc2c(c1)CN(C1([2H])C(=O)NC(=O)C([2H])([2H])C1([2H])[2H])C2=O] |
| 291 | 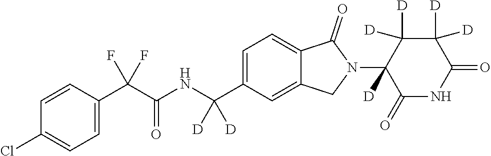 [SMILES: [2H]C([2H])(NC(=O)C(F)(F)c1ccc(Cl)cc1)c1ccc2c(c1)CN([C@@]1([2H])C(=O)NC(=O)C([2H])([2H])C1([2H])[2H])C2=O] |
| 292 | 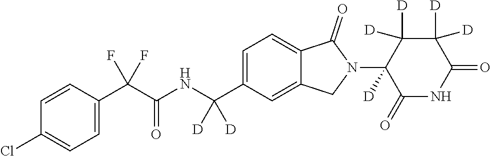 [SMILES: [2H]C([2H])(NC(=O)C(F)(F)c1ccc(Cl)cc1)c1ccc2c(c1)CN([C@]1([2H])C(=O)NC(=O)C([2H])([2H])C1([2H])[2H])C2=O] |
| 293 | 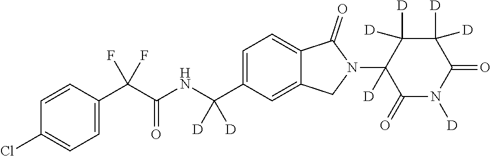 |
| 294 | 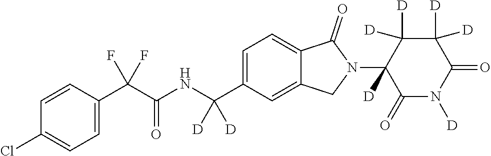 |
| 295 | 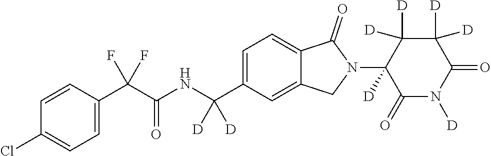 |
| 296 | 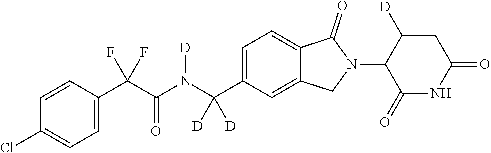 |
| 297 | 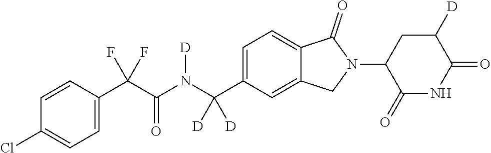 |
| 298 | 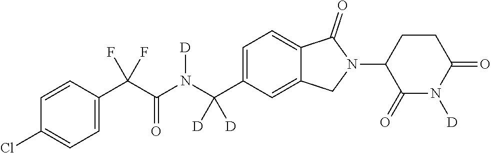 |
| 299 | 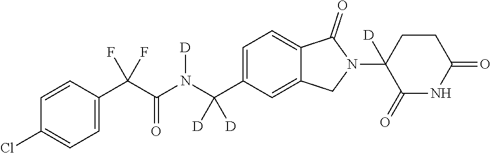 |
| 300 | 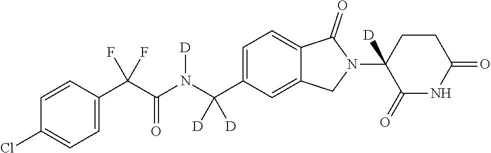 |
| 301 | 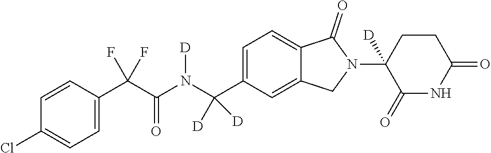 |
| 302 | 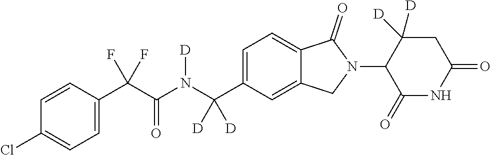 |
| 303 | 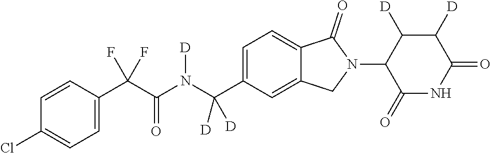 |
| 304 | 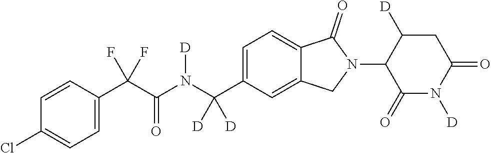 |
| 305 | 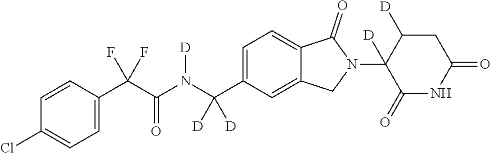 |
| 306 | 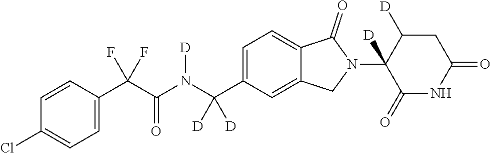 |
| 307 | 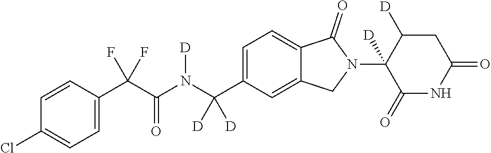 |
| 308 | 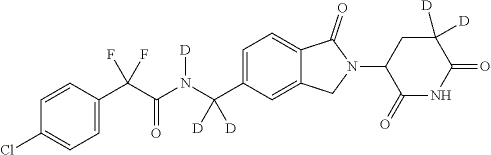 |
| 309 | 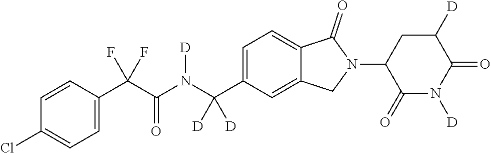 |
| 310 | 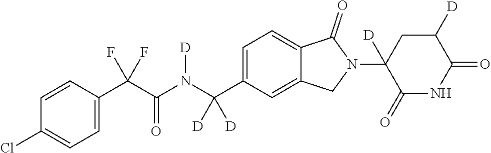 |
| 311 | 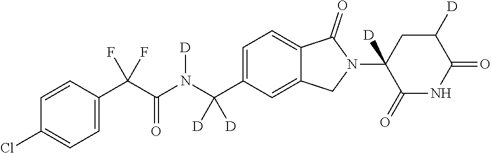 |
| 312 | 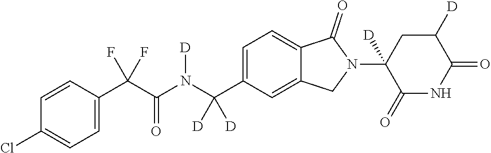 |
| 313 | 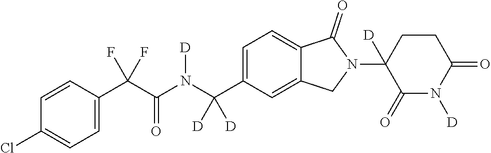 |
| 314 | 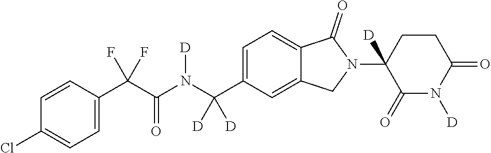 |
| 315 | 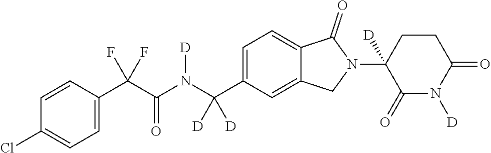 |
| 316 | 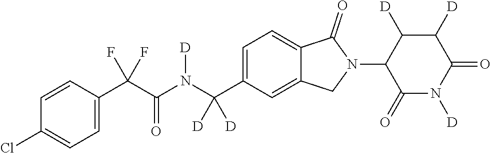 |
| 317 | 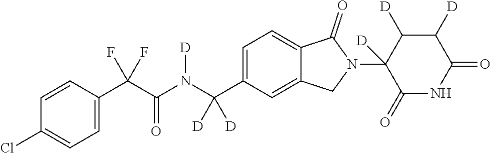 |
| 318 | 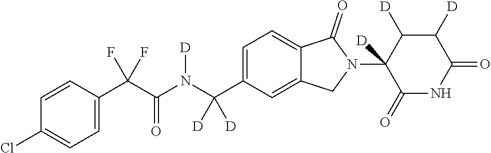 |
| 319 | 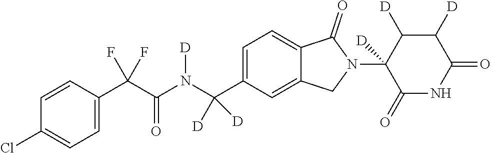 |
| 320 | 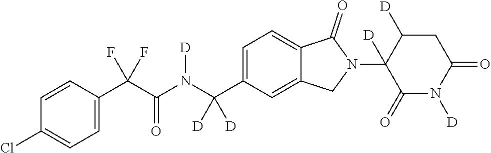 |
| 321 | 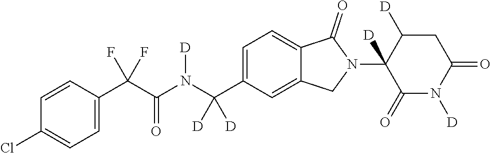 |
| 322 | 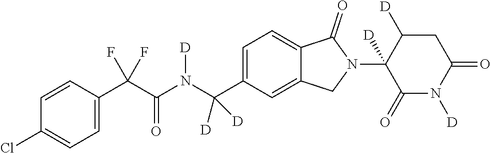 |
| 323 | 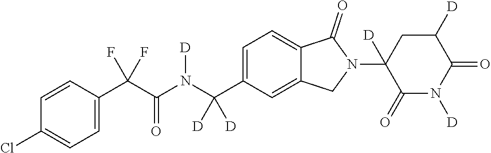 |
| 324 | 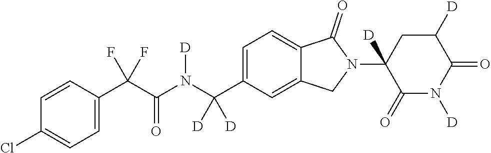 |
| 325 | 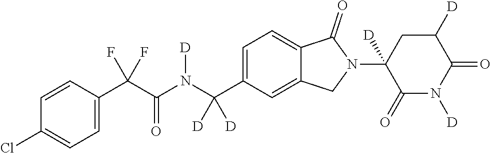 |
| 326 | 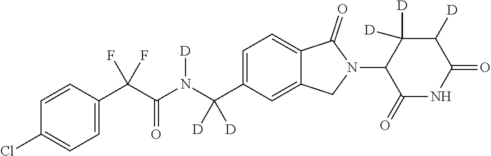 |
| 327 | 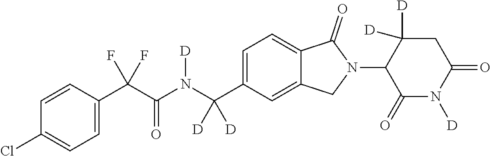 |
| 328 | 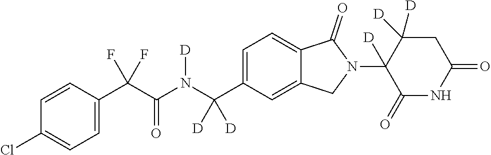 |
| 329 | 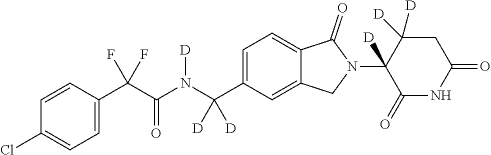 |
| 330 | 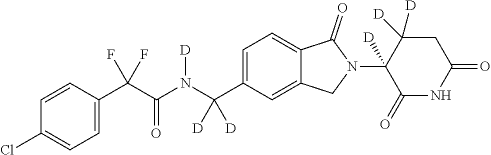 |
| 331 | 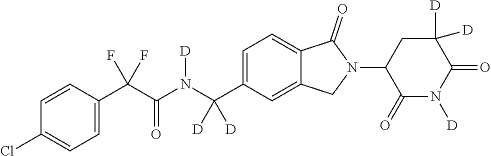 |
| 332 | 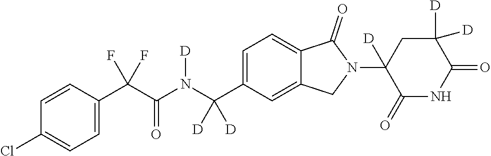 |
| 333 | 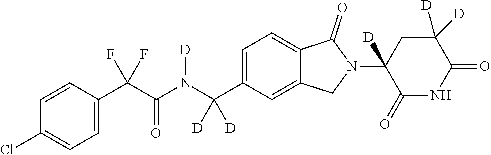 |
| 334 | 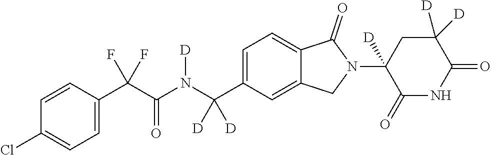 |
| 335 | 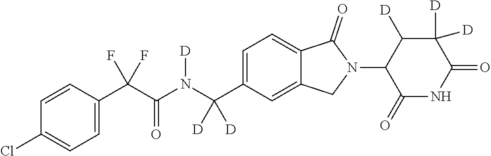 |
| 336 | 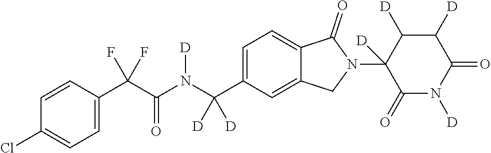 |
| 337 | 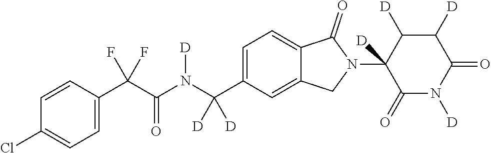 |
| 338 | 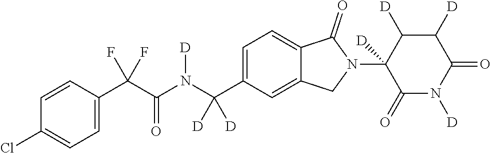 |
| 339 | 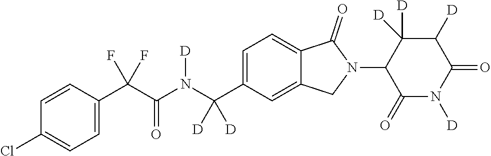 |
| 340 | 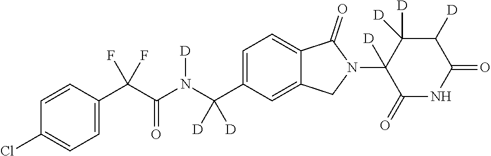 |
| 341 | 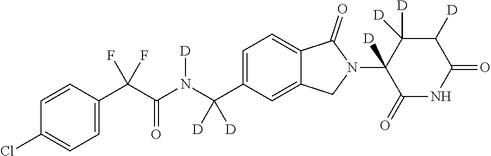 |
| 342 | 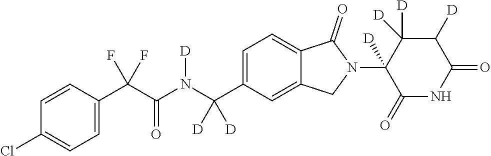 |
| 343 | 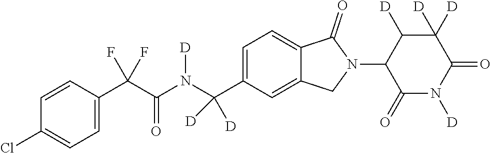 |
| 344 | 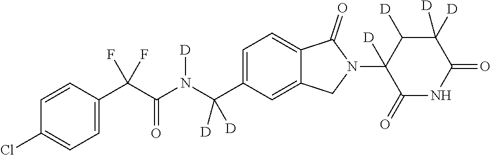 |
| 345 | 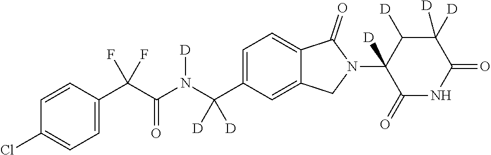 |
| 346 | 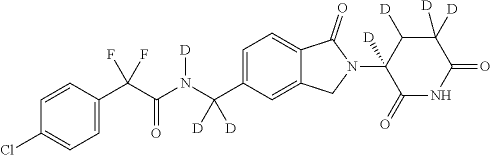 |
| 347 | 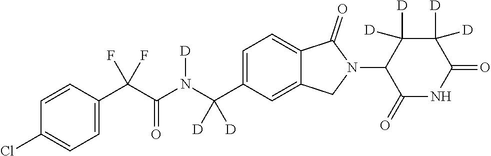 |
| 348 | 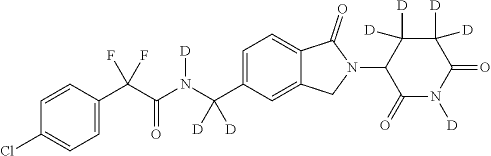 |
| 349 | 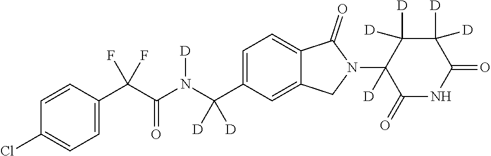 |
| 350 | 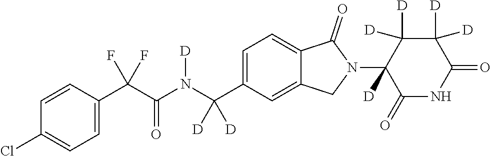 |
| 351 | 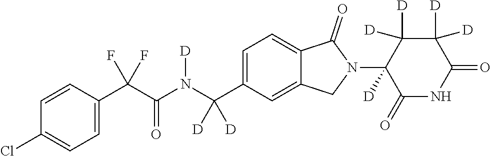 |
| 352 | 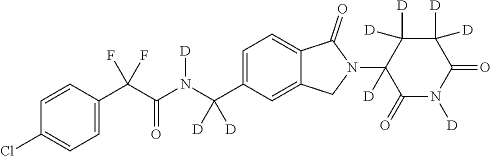 |
| 353 | 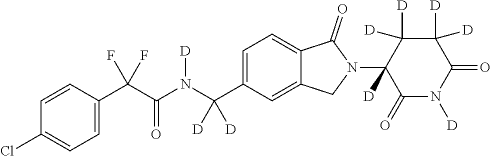 |
| 354 | 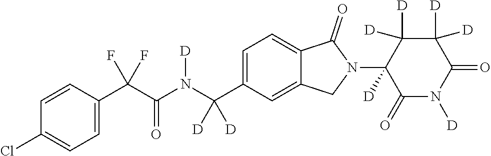 |

In one embodiment, the compound provided herein is a compound listed in Table 1, wherein one or more carbon atoms is/are radiolabeled carbon-14 (¹⁴C).

In one embodiment, the compound provided herein is a compound listed in Table 1, wherein one or more hydrogen atoms on the oxoisoindolinyl and/or phenyl ring are isotopically enriched with deuterium. In one embodiment, the compound provided herein is a compound listed in Table 1, wherein one, two, three, four or more hydrogen atoms on the oxoisoindolinyl and/or phenyl ring are isotopically enriched with deuterium.

In one embodiment, the compound provided herein is a compound listed in Table 1, wherein one or more hydrogen atoms on the oxoisoindolinyl ring are hydrogens that are isotopically enriched with deuterium. In one embodiment, the compound provided herein is a compound listed in Table 1, wherein one hydrogen atom on the oxoisoindolinyl ring is isotopically enriched with deuterium. In one embodiment, the compound provided herein is a compound listed in Table 1, wherein two hydrogen atoms on the oxoisoindolinyl ring are isotopically enriched with deuterium. In one embodiment, the compound provided herein is a compound listed in Table 1, wherein three hydrogen atoms on the oxoisoindolinyl ring are isotopically enriched with deuterium. In one embodiment, the compound provided herein is a compound listed in Table 1, wherein four hydrogen atoms on the oxoisoindolinyl ring are isotopically enriched with deuterium. In one embodiment, the compound provided herein is a compound listed in Table 1, wherein five hydrogen atoms on the oxoisoindolinyl ring are isotopically enriched with deuterium.

In one embodiment, the compound provided herein is a compound listed in Table 1, wherein one or more hydrogen atoms on the phenyl ring are isotopically enriched with deuterium. In one embodiment, the compound provided herein is a compound listed in Table 1, wherein one hydrogen atom on the phenyl ring is isotopically enriched with deuterium. In one embodiment, the compound provided herein is a compound listed in Table 1, wherein two hydrogen atoms on the phenyl ring are isotopically enriched with deuterium. In one embodiment, the compound provided herein is a compound listed in Table 1, wherein three hydrogen atoms on the phenyl ring are isotopically enriched with deuterium. In one embodiment, the compound provided herein is a compound listed in Table 1, wherein four hydrogen atoms on the phenyl ring are isotopically enriched with deuterium.

In certain embodiments, one or more Y atoms on the oxoisoindolinyl portion of A1, A2 or A3 are deuterium-enriched. For example, particular compounds provided herein include, but are not limited to, the compounds listed in Table 2, and its stereoisomers or mixture of stereoisomers, pharmaceutically acceptable salts, tautomers, solvates, hydrates, co-crystals, clathrates, or polymorphs thereof, wherein the label “D” indicates a deuterium-enriched atomic position, i.e., a sample comprising the given compound has a deuterium enrichment at the indicated position(s) above the natural abundance of deuterium:

TABLE 2

| No. | Compound structure |
|---|---|
| 355 | 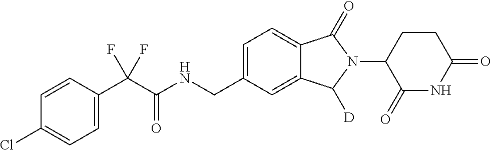 [SMILES: [2H]C1c2cc(CNC(=O)C(F)(F)c3ccc(Cl)cc3)ccc2C(=O)N1C1CCC(=O)NC1=O] |
| 356 | 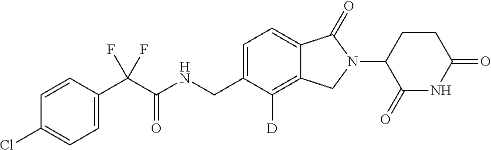 [SMILES: [2H]c1c(CNC(=O)C(F)(F)c2ccc(Cl)cc2)ccc2c1CN(C1CCC(=O)NC1=O)C2=O] |
| 357 | 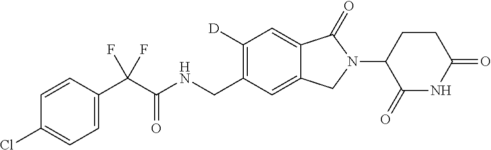 [SMILES: [2H]c1cc2c(cc1CNC(=O)C(F)(F)c1ccc(Cl)cc1)CN(C1CCC(=O)NC1=O)C2=O] |
| 358 | 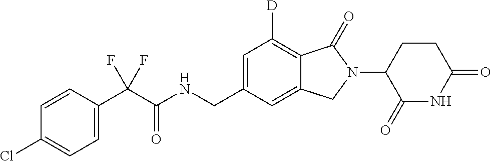 [SMILES: [2H]c1cc(CNC(=O)C(F)(F)c2ccc(Cl)cc2)cc2c1C(=O)N(C1CCC(=O)NC1=O)C2] |
| 359 | 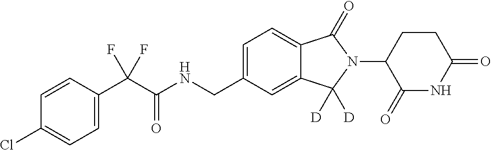 [SMILES: [2H]C1([2H])c2cc(CNC(=O)C(F)(F)c3ccc(Cl)cc3)ccc2C(=O)N1C1CCC(=O)NC1=O] |
| 360 | 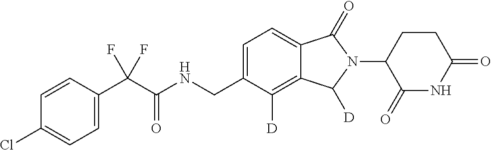 [SMILES: [2H]c1c(CNC(=O)C(F)(F)c2ccc(Cl)cc2)ccc2c1C([2H])N(C1CCC(=O)NC1=O)C2=O] |
| 361 | 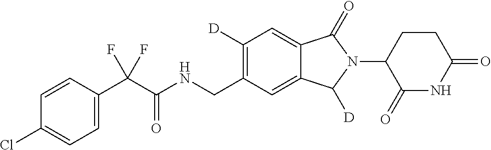 [SMILES: [2H]c1cc2c(cc1CNC(=O)C(F)(F)c1ccc(Cl)cc1)C([2H])N(C1CCC(=O)NC1=O)C2=O] |
| 362 | 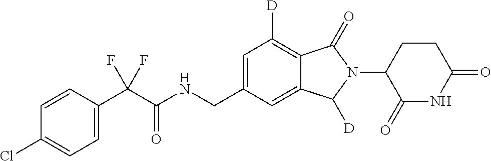 [SMILES: [2H]c1cc(CNC(=O)C(F)(F)c2ccc(Cl)cc2)cc2c1C(=O)N(C1CCC(=O)NC1=O)C2[2H]] |
| 363 | 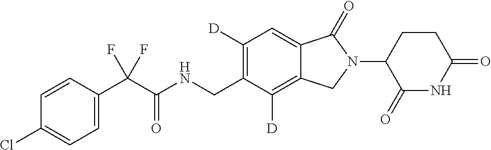 [SMILES: [2H]c1cc2c(c([2H])c1CNC(=O)C(F)(F)c1ccc(Cl)cc1)CN(C1CCC(=O)NC1=O)C2=O] |
| 364 | 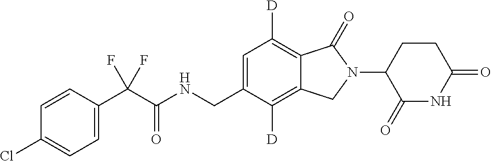 [SMILES: [2H]c1cc(CNC(=O)C(F)(F)c2ccc(Cl)cc2)c([2H])c2c1C(=O)N(C1CCC(=O)NC1=O)C2] |
| 365 | 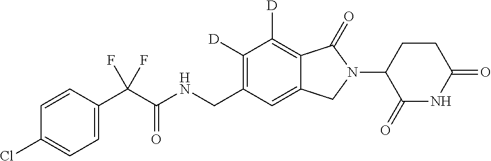 [SMILES: [2H]c1c(CNC(=O)C(F)(F)c2ccc(Cl)cc2)cc2c(c1[2H])C(=O)N(C1CCC(=O)NC1=O)C2] |
| 366 | 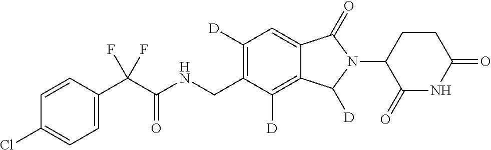 [SMILES: [2H]c1cc2c(c([2H])c1CNC(=O)C(F)(F)c1ccc(Cl)cc1)C([2H])N(C1CCC(=O)NC1=O)C2=O] |
| 367 | 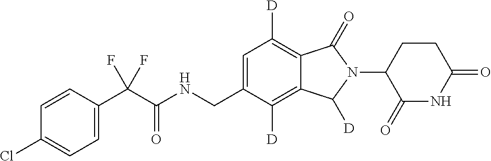 [SMILES: [2H]c1cc(CNC(=O)C(F)(F)c2ccc(Cl)cc2)c([2H])c2c1C(=O)N(C1CCC(=O)NC1=O)C2[2H]] |
| 368 | 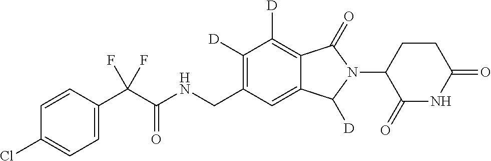 [SMILES: [2H]c1c(CNC(=O)C(F)(F)c2ccc(Cl)cc2)cc2c(c1[2H])C(=O)N(C1CCC(=O)NC1=O)C2[2H]] |
| 369 | 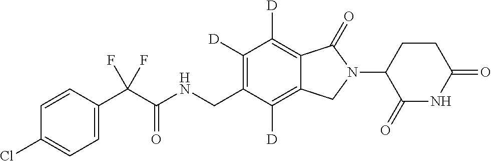 [SMILES: [2H]c1c([2H])c2c(c([2H])c1CNC(=O)C(F)(F)c1ccc(Cl)cc1)CN(C1CCC(=O)NC1=O)C2=O] |
| 370 | 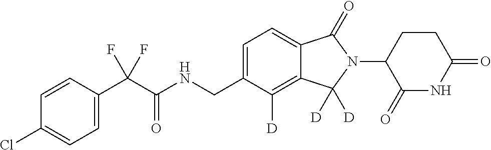 [SMILES: [2H]c1c(CNC(=O)C(F)(F)c2ccc(Cl)cc2)ccc2c1C([2H])([2H])N(C1CCC(=O)NC1=O)C2=O] |
| 371 | 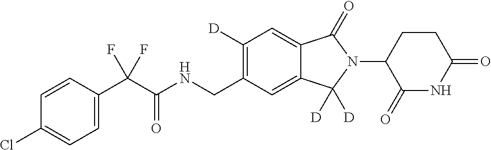 [SMILES: [2H]c1cc2c(cc1CNC(=O)C(F)(F)c1ccc(Cl)cc1)C([2H])([2H])N(C1CCC(=O)NC1=O)C2=O] |
| 372 | 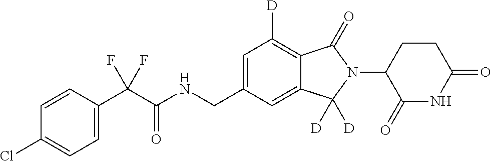 [SMILES: [2H]c1cc(CNC(=O)C(F)(F)c2ccc(Cl)cc2)cc2c1C(=O)N(C1CCC(=O)NC1=O)C2([2H])[2H]] |
| 373 | 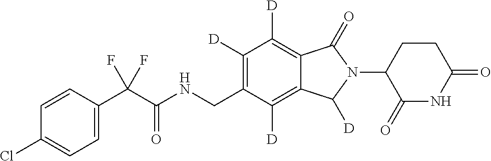 [SMILES: [2H]c1c([2H])c2c(c([2H])c1CNC(=O)C(F)(F)c1ccc(Cl)cc1)C([2H])N(C1CCC(=O)NC1=O)C2=O] |
| 374 | 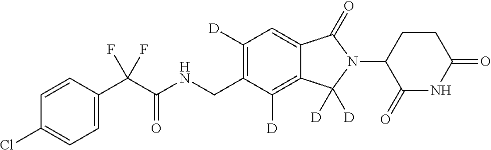 [SMILES: [2H]c1cc2c(c([2H])c1CNC(=O)C(F)(F)c1ccc(Cl)cc1)C([2H])([2H])N(C1CCC(=O)NC1=O)C2=O] |
| 375 | 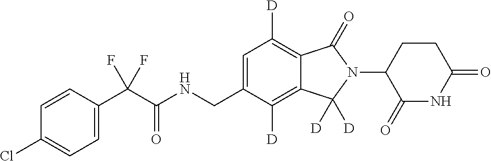 [SMILES: [2H]c1cc(CNC(=O)C(F)(F)c2ccc(Cl)cc2)c([2H])c2c1C(=O)N(C1CCC(=O)NC1=O)C2([2H])[2H]] |
| 376 | 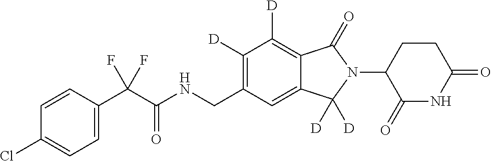 [SMILES: [2H]c1c(CNC(=O)C(F)(F)c2ccc(Cl)cc2)cc2c(c1[2H])C(=O)N(C1CCC(=O)NC1=O)C2([2H])[2H]] |
| 377 | 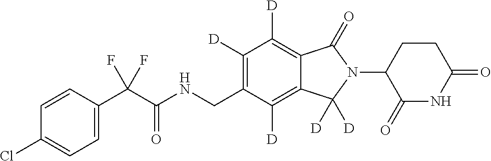 [SMILES: [2H]c1c([2H])c2c(c([2H])c1CNC(=O)C(F)(F)c1ccc(Cl)cc1)C([2H])([2H])N(C1CCC(=O)NC1=O)C2=O] |
| 378 | 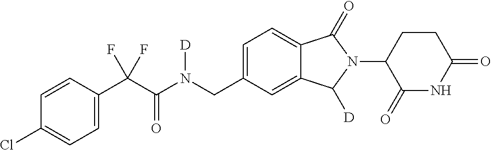 |
| 379 | 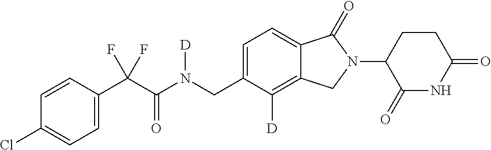 |
| 380 | 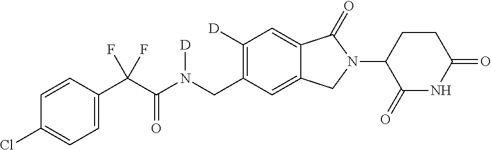 |
| 381 | 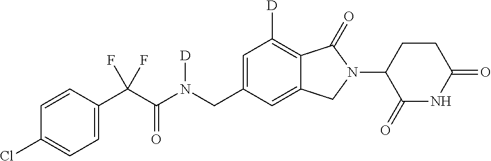 |
| 382 | 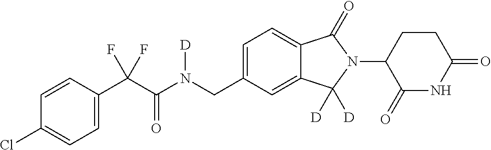 |
| 383 | 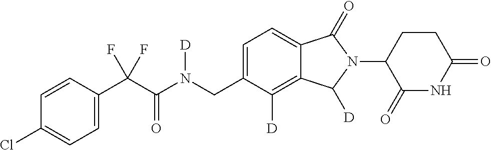 |
| 384 | 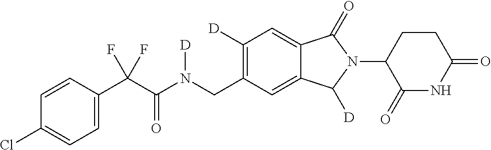 |
| 385 | 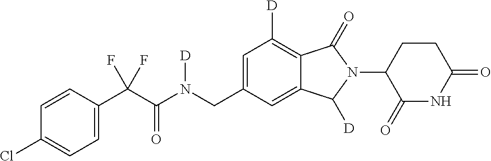 |
| 386 | 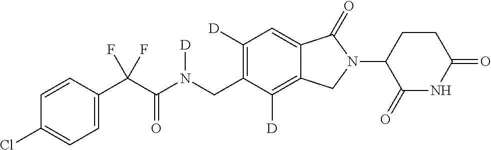 |
| 387 | 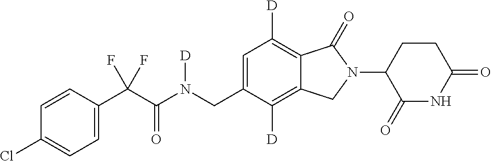 |
| 388 | 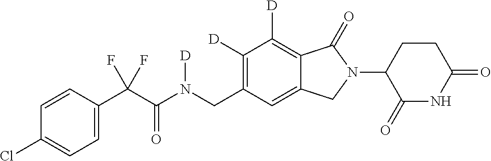 |
| 389 | 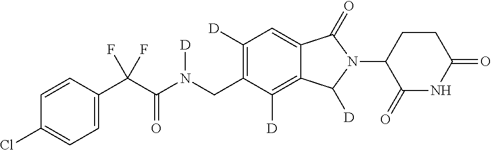 |
| 390 | 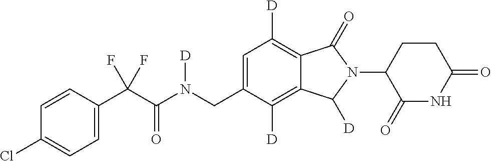 |
| 391 | 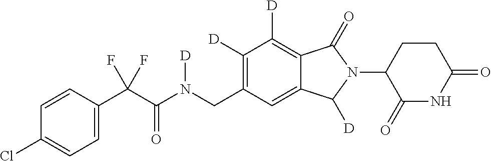 |
| 392 | 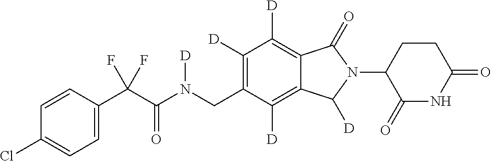 |
| 393 | 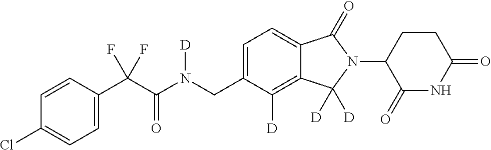 |
| 394 | 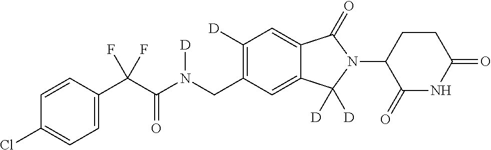 |
| 395 | 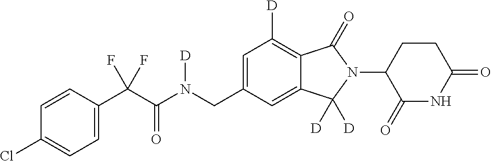 |
| 396 | 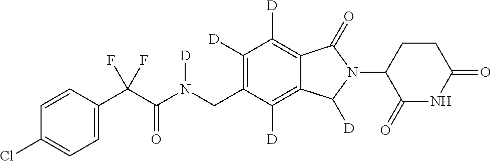 |
| 397 | 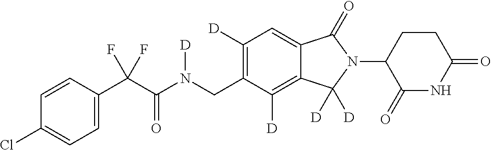 |
| 398 | 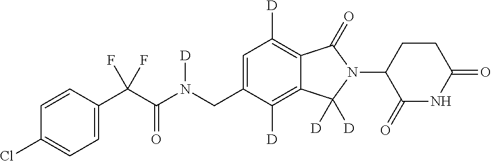 |
| 399 | 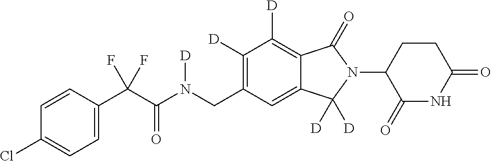 |
| 400 | 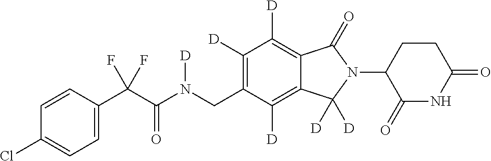 |
| 401 | 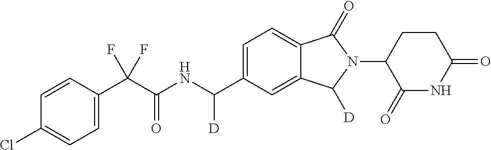 [SMILES: [2H]C(NC(=O)C(F)(F)c1ccc(Cl)cc1)c1ccc2c(c1)C([2H])N(C1CCC(=O)NC1=O)C2=O] |
| 402 | 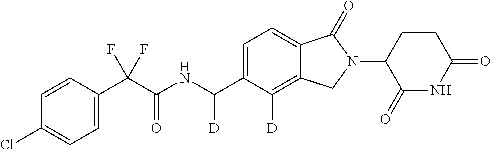 [SMILES: [2H]c1c(C([2H])NC(=O)C(F)(F)c2ccc(Cl)cc2)ccc2c1CN(C1CCC(=O)NC1=O)C2=O] |
| 403 | 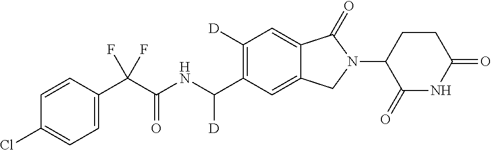 [SMILES: [2H]c1cc2c(cc1C([2H])NC(=O)C(F)(F)c1ccc(Cl)cc1)CN(C1CCC(=O)NC1=O)C2=O] |
| 404 | 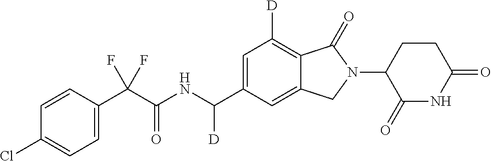 [SMILES: [2H]c1cc(C([2H])NC(=O)C(F)(F)c2ccc(Cl)cc2)cc2c1C(=O)N(C1CCC(=O)NC1=O)C2] |
| 405 | 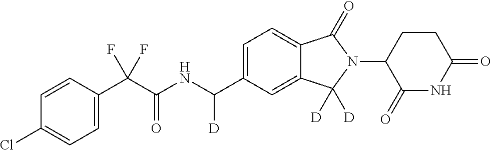 [SMILES: [2H]C(NC(=O)C(F)(F)c1ccc(Cl)cc1)c1ccc2c(c1)C([2H])([2H])N(C1CCC(=O)NC1=O)C2=O] |
| 406 | 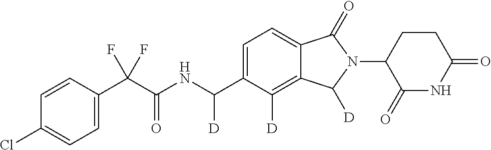 [SMILES: [2H]c1c(C([2H])NC(=O)C(F)(F)c2ccc(Cl)cc2)ccc2c1C([2H])N(C1CCC(=O)NC1=O)C2=O] |
| 407 | 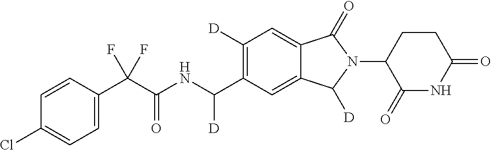 [SMILES: [2H]c1cc2c(cc1C([2H])NC(=O)C(F)(F)c1ccc(Cl)cc1)C([2H])N(C1CCC(=O)NC1=O)C2=O] |
| 408 | 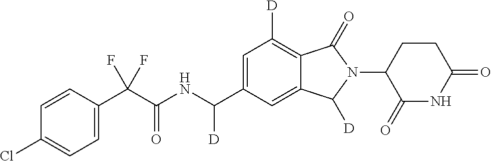 [SMILES: [2H]c1cc(C([2H])NC(=O)C(F)(F)c2ccc(Cl)cc2)cc2c1C(=O)N(C1CCC(=O)NC1=O)C2[2H]] |
| 409 | 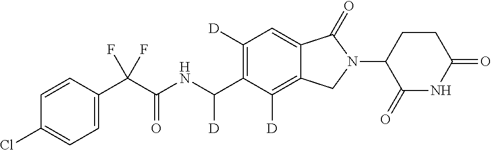 [SMILES: [2H]c1cc2c(c([2H])c1C([2H])NC(=O)C(F)(F)c1ccc(Cl)cc1)CN(C1CCC(=O)NC1=O)C2=O] |
| 410 | 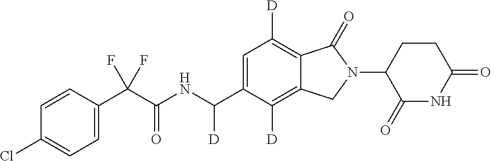 [SMILES: [2H]c1cc(C([2H])NC(=O)C(F)(F)c2ccc(Cl)cc2)c([2H])c2c1C(=O)N(C1CCC(=O)NC1=O)C2] |
| 411 | 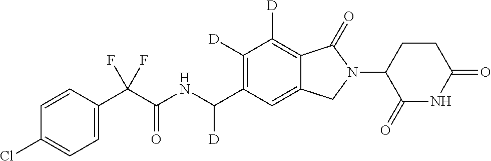 [SMILES: [2H]c1c(C([2H])NC(=O)C(F)(F)c2ccc(Cl)cc2)cc2c(c1[2H])C(=O)N(C1CCC(=O)NC1=O)C2] |
| 412 | 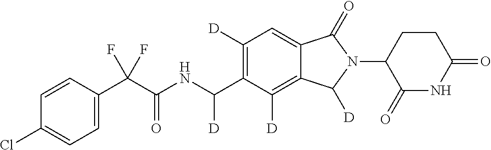 [SMILES: [2H]c1cc2c(c([2H])c1C([2H])NC(=O)C(F)(F)c1ccc(Cl)cc1)C([2H])N(C1CCC(=O)NC1=O)C2=O] |
| 413 | 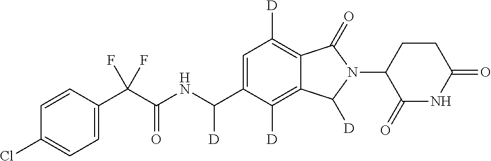 [SMILES: [2H]c1cc(C([2H])NC(=O)C(F)(F)c2ccc(Cl)cc2)c([2H])c2c1C(=O)N(C1CCC(=O)NC1=O)C2[2H]] |
| 414 | 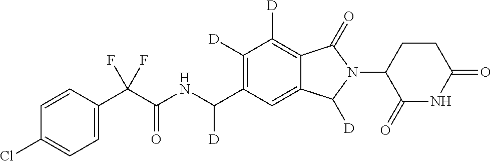 [SMILES: [2H]c1c(C([2H])NC(=O)C(F)(F)c2ccc(Cl)cc2)cc2c(c1[2H])C(=O)N(C1CCC(=O)NC1=O)C2[2H]] |
| 415 | 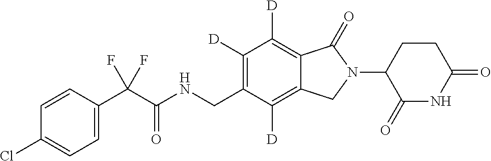 [SMILES: [2H]c1c([2H])c2c(c([2H])c1CNC(=O)C(F)(F)c1ccc(Cl)cc1)CN(C1CCC(=O)NC1=O)C2=O] |
| 416 | 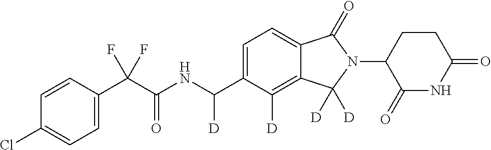 [SMILES: [2H]c1c(C([2H])NC(=O)C(F)(F)c2ccc(Cl)cc2)ccc2c1C([2H])([2H])N(C1CCC(=O)NC1=O)C2=O] |
| 417 | 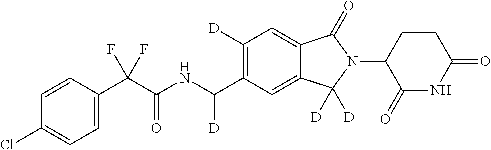 [SMILES: [2H]c1cc2c(cc1C([2H])NC(=O)C(F)(F)c1ccc(Cl)cc1)C([2H])([2H])N(C1CCC(=O)NC1=O)C2=O] |
| 418 | 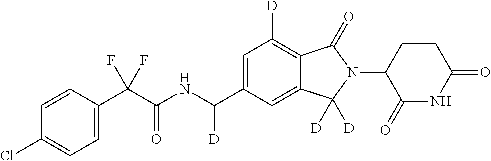 [SMILES: [2H]c1cc(C([2H])NC(=O)C(F)(F)c2ccc(Cl)cc2)cc2c1C(=O)N(C1CCC(=O)NC1=O)C2([2H])[2H]] |
| 419 | 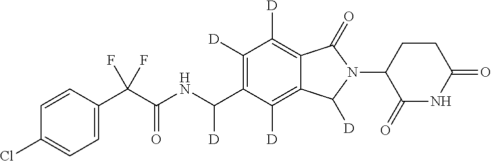 [SMILES: [2H]c1c([2H])c(C([2H])NC(=O)C(F)(F)c2ccc(Cl)cc2)c([2H])c2c1C(=O)N(C1CCC(=O)NC1=O)C2[2H]] |
| 420 | 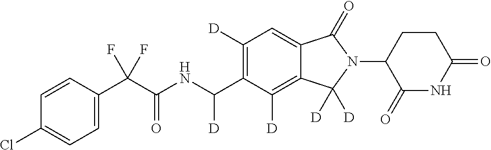 [SMILES: [2H]c1cc2c(c([2H])c1C([2H])NC(=O)C(F)(F)c1ccc(Cl)cc1)C([2H])([2H])N(C1CCC(=O)NC1=O)C2=O] |
| 421 | 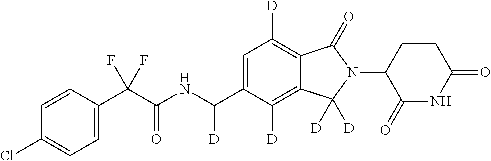 [SMILES: [2H]c1cc(C([2H])NC(=O)C(F)(F)c2ccc(Cl)cc2)c([2H])c2c1C(=O)N(C1CCC(=O)NC1=O)C2([2H])[2H]] |
| 422 | 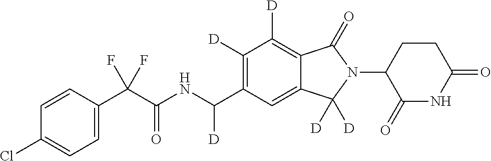 [SMILES: [2H]c1c(C([2H])NC(=O)C(F)(F)c2ccc(Cl)cc2)cc2c(c1[2H])C(=O)N(C1CCC(=O)NC1=O)C2([2H])[2H]] |
| 423 | 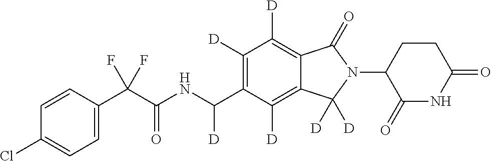 [SMILES: [2H]c1c([2H])c(C([2H])NC(=O)C(F)(F)c2ccc(Cl)cc2)c([2H])c2c1C(=O)N(C1CCC(=O)NC1=O)C2([2H])[2H]] |
| 424 | 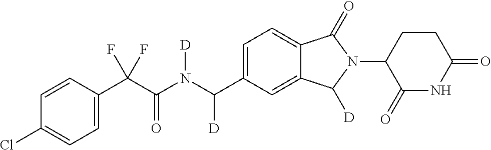 |
| 425 | 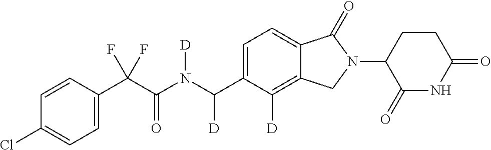 |
| 426 | 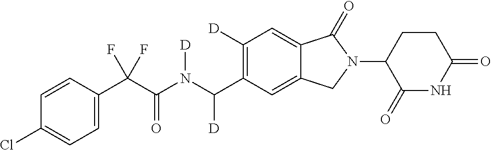 |
| 427 | 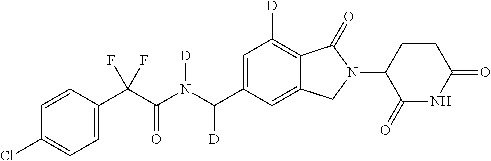 |
| 428 | 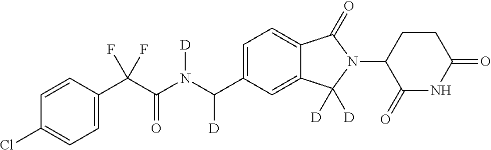 |
| 429 | 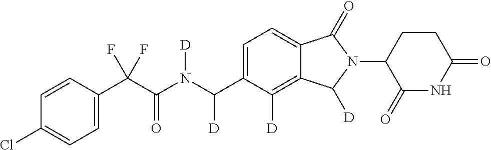 |
| 430 | 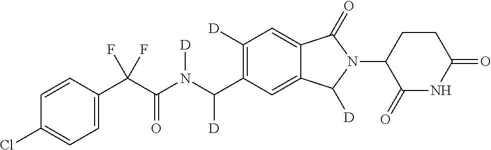 |
| 431 | 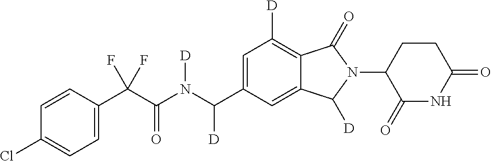 |
| 432 | 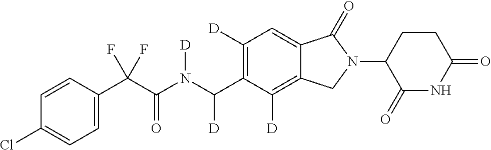 |
| 433 | 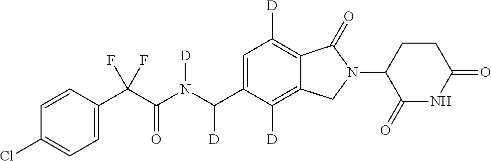 |
| 434 | 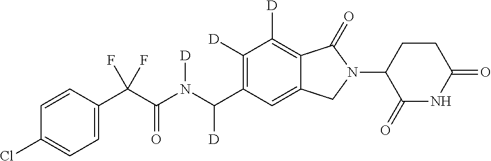 |
| 435 | 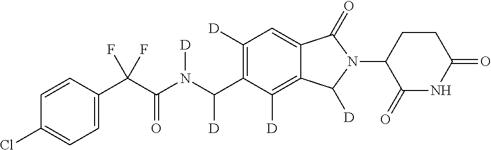 |
| 436 | 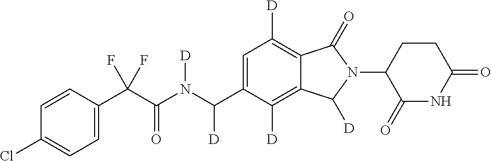 |
| 437 | 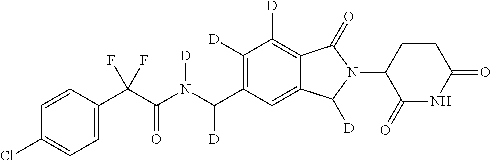 |
| 438 | 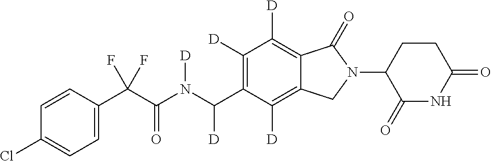 |
| 439 | 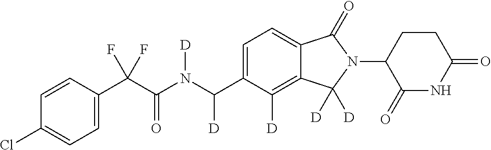 |
| 440 | 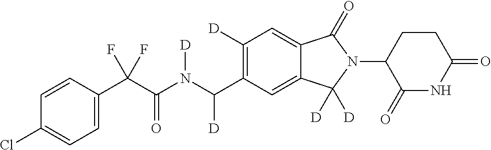 |
| 441 | 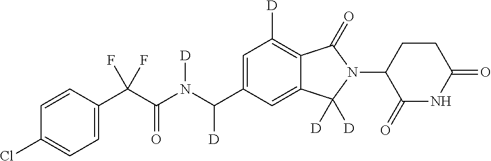 |
| 442 | 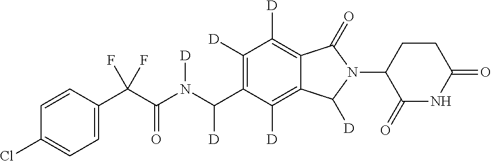 |
| 443 | 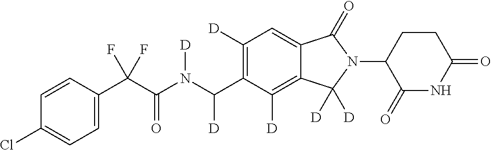 |
| 444 | 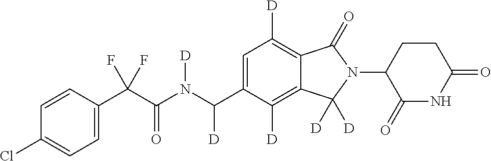 |
| 445 | 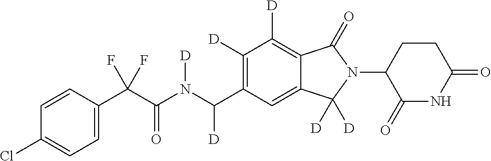 |
| 446 | 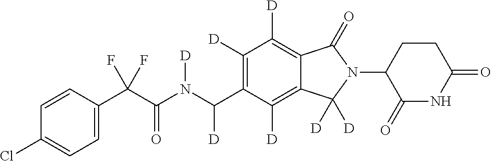 |
| 447 | 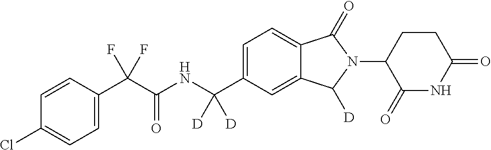 [SMILES: [2H]C1c2cc(C([2H])([2H])NC(=O)C(F)(F)c3ccc(Cl)cc3)ccc2C(=O)N1C1CCC(=O)NC1=O] |
| 448 | 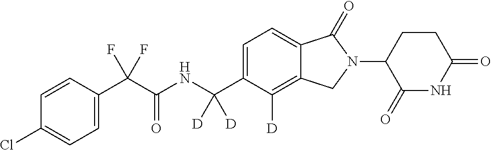 [SMILES: [2H]c1c(C([2H])([2H])NC(=O)C(F)(F)c2ccc(Cl)cc2)ccc2c1CN(C1CCC(=O)NC1=O)C2=O] |
| 449 | 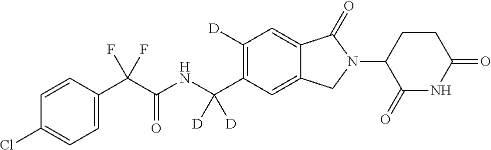 [SMILES: [2H]c1cc2c(cc1C([2H])([2H])NC(=O)C(F)(F)c1ccc(Cl)cc1)CN(C1CCC(=O)NC1=O)C2=O] |
| 450 | 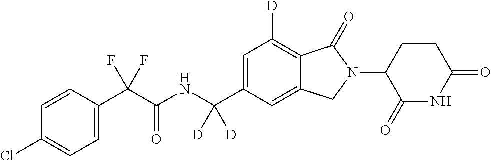 [SMILES: [2H]c1cc(C([2H])([2H])NC(=O)C(F)(F)c2ccc(Cl)cc2)cc2c1C(=O)N(C1CCC(=O)NC1=O)C2] |
| 451 | 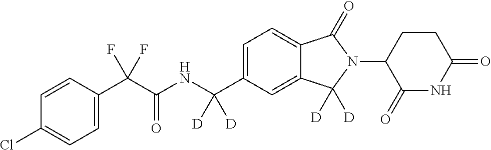 [SMILES: [2H]C([2H])(NC(=O)C(F)(F)c1ccc(Cl)cc1)c1ccc2c(c1)C([2H])([2H])N(C1CCC(=O)NC1=O)C2=O] |
| 452 | 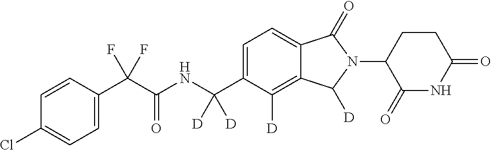 [SMILES: [2H]c1c(C([2H])([2H])NC(=O)C(F)(F)c2ccc(Cl)cc2)ccc2c1C([2H])N(C1CCC(=O)NC1=O)C2=O] |
| 453 | 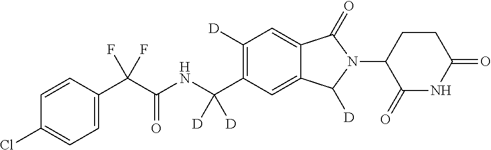 [SMILES: [2H]c1cc2c(cc1C([2H])([2H])NC(=O)C(F)(F)c1ccc(Cl)cc1)C([2H])N(C1CCC(=O)NC1=O)C2=O] |
| 454 | 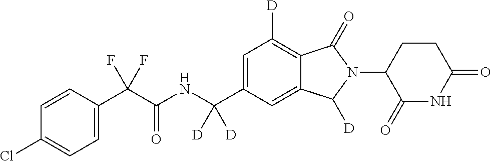 [SMILES: [2H]c1cc(C([2H])([2H])NC(=O)C(F)(F)c2ccc(Cl)cc2)cc2c1C(=O)N(C1CCC(=O)NC1=O)C2[2H]] |
| 455 | 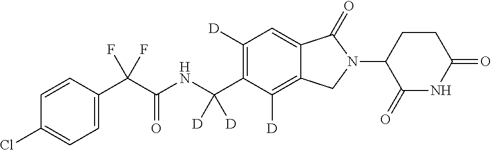 [SMILES: [2H]c1cc2c(c([2H])c1C([2H])([2H])NC(=O)C(F)(F)c1ccc(Cl)cc1)CN(C1CCC(=O)NC1=O)C2=O] |
| 456 | 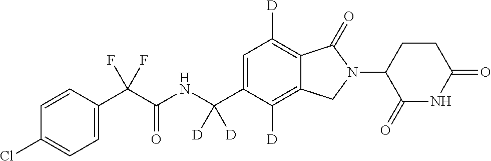 [SMILES: [2H]c1cc(C([2H])([2H])NC(=O)C(F)(F)c2ccc(Cl)cc2)c([2H])c2c1C(=O)N(C1CCC(=O)NC1=O)C2] |
| 457 | 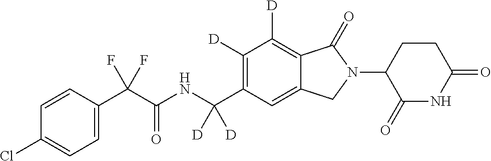 [SMILES: [2H]c1c(C([2H])([2H])NC(=O)C(F)(F)c2ccc(Cl)cc2)cc2c(c1[2H])C(=O)N(C1CCC(=O)NC1=O)C2] |
| 458 | 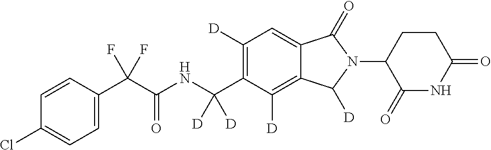 [SMILES: [2H]c1cc2c(c([2H])c1C([2H])([2H])NC(=O)C(F)(F)c1ccc(Cl)cc1)C([2H])N(C1CCC(=O)NC1=O)C2=O] |
| 459 | 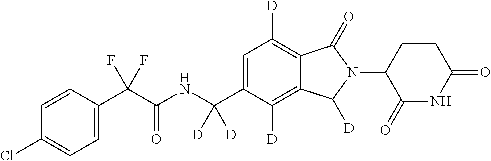 [SMILES: [2H]c1cc(C([2H])([2H])NC(=O)C(F)(F)c2ccc(Cl)cc2)c([2H])c2c1C(=O)N(C1CCC(=O)NC1=O)C2[2H]] |
| 460 | 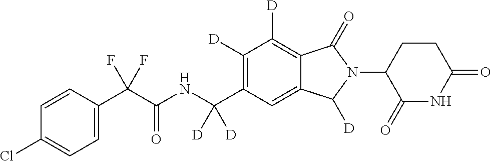 [SMILES: [2H]c1c(C([2H])([2H])NC(=O)C(F)(F)c2ccc(Cl)cc2)cc2c(c1[2H])C(=O)N(C1CCC(=O)NC1=O)C2[2H]] |
| 461 | 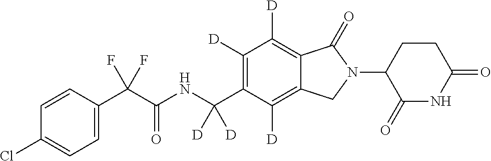 [SMILES: [2H]c1c([2H])c(C([2H])([2H])NC(=O)C(F)(F)c2ccc(Cl)cc2)c([2H])c2c1C(=O)N(C1CCC(=O)NC1=O)C2] |
| 461 | 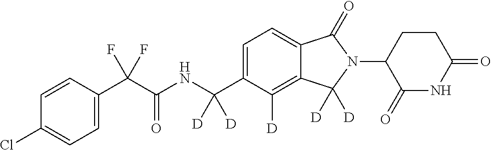 [SMILES: [2H]c1c(C([2H])([2H])NC(=O)C(F)(F)c2ccc(Cl)cc2)ccc2c1C([2H])([2H])N(C1CCC(=O)NC1=O)C2=O] |
| 463 | 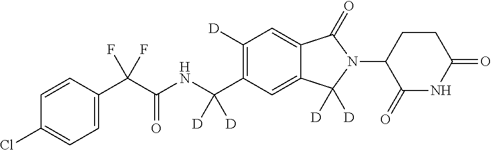 [SMILES: [2H]c1cc2c(cc1C([2H])([2H])NC(=O)C(F)(F)c1ccc(Cl)cc1)C([2H])([2H])N(C1CCC(=O)NC1=O)C2=O] |
| 464 | 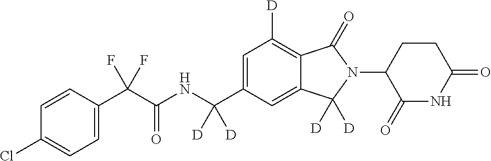 [SMILES: [2H]c1cc(C([2H])([2H])NC(=O)C(F)(F)c2ccc(Cl)cc2)cc2c1C(=O)N(C1CCC(=O)NC1=O)C2([2H])[2H]] |
| 465 | 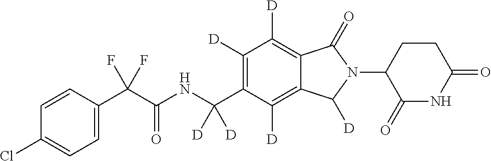 [SMILES: [2H]c1c([2H])c(C([2H])([2H])NC(=O)C(F)(F)c2ccc(Cl)cc2)c([2H])c2c1C(=O)N(C1CCC(=O)NC1=O)C2[2H]] |
| 466 | 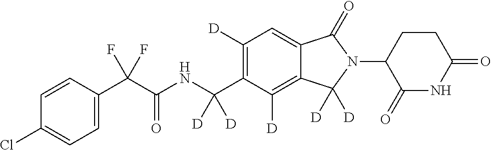 [SMILES: [2H]c1cc2c(c([2H])c1C([2H])([2H])NC(=O)C(F)(F)c1ccc(Cl)cc1)C([2H])([2H])N(C1CCC(=O)NC1=O)C2=O] |
| 467 | 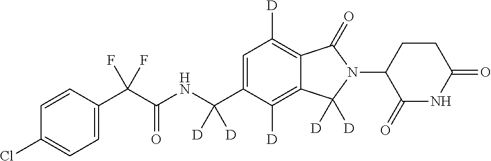 [SMILES: [2H]c1cc(C([2H])([2H])NC(=O)C(F)(F)c2ccc(Cl)cc2)c([2H])c2c1C(=O)N(C1CCC(=O)NC1=O)C2([2H])[2H]] |
| 468 | 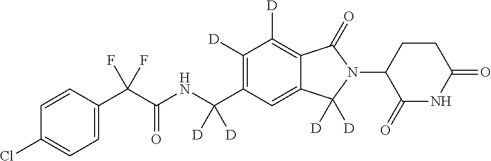 [SMILES: [2H]c1c(C([2H])([2H])NC(=O)C(F)(F)c2ccc(Cl)cc2)cc2c(c1[2H])C(=O)N(C1CCC(=O)NC1=O)C2([2H])[2H]] |
| 469 | 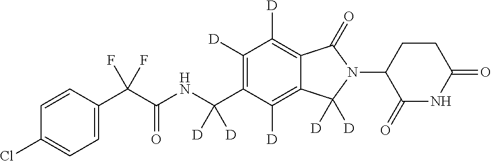 [SMILES: [2H]c1c([2H])c(C([2H])([2H])NC(=O)C(F)(F)c2ccc(Cl)cc2)c([2H])c2c1C(=O)N(C1CCC(=O)NC1=O)C2([2H])[2H]] |
| 470 | 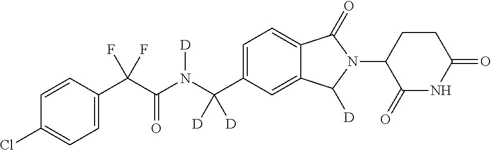 |
| 471 | 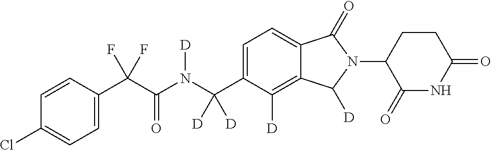 |
| 472 | 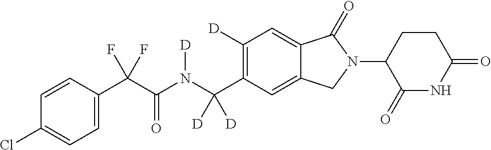 |
| 473 | 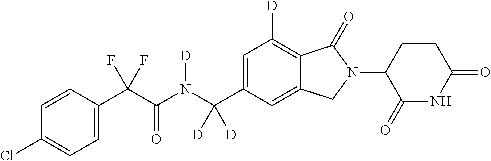 |
| 474 | 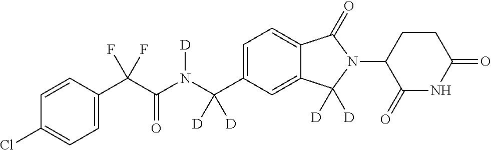 |
| 475 | 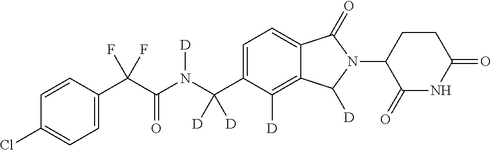 |
| 476 | 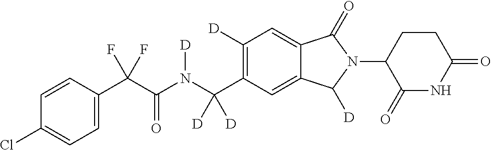 |
| 477 | 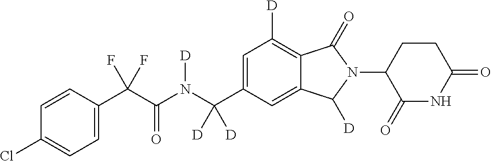 |
| 478 | 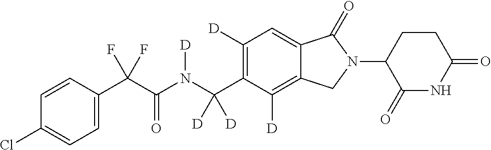 |
| 479 | 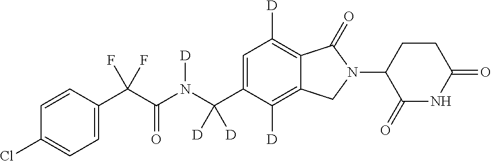 |
| 480 | 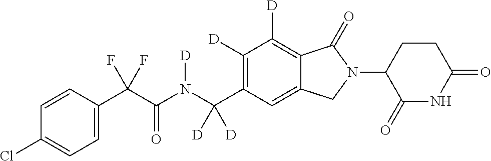 |
| 481 | 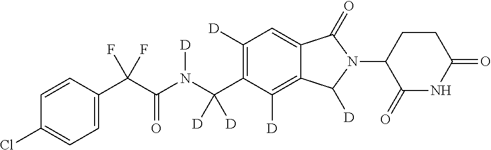 |
| 482 | 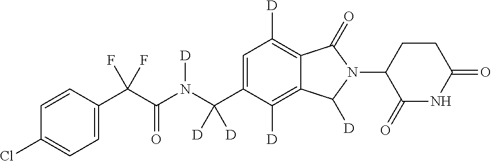 |
| 483 | 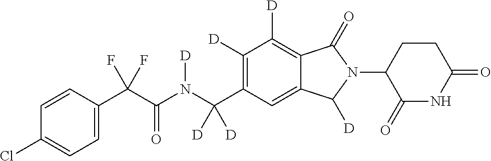 |
| 484 | 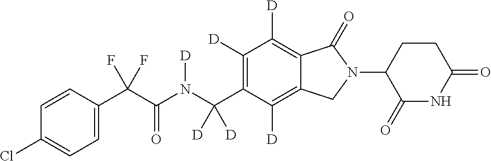 |
| 485 | 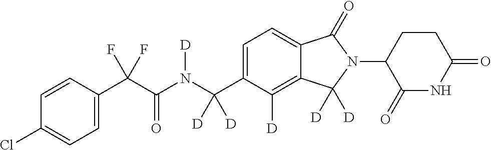 |
| 486 | 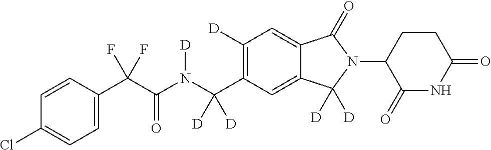 |
| 487 | 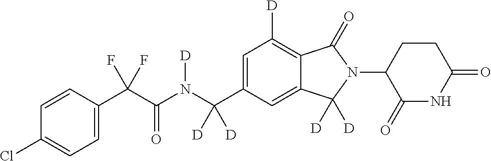 |
| 488 | 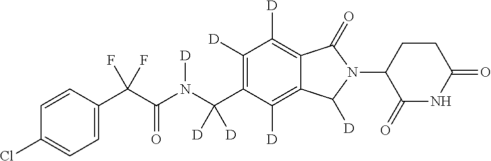 |
| 489 | 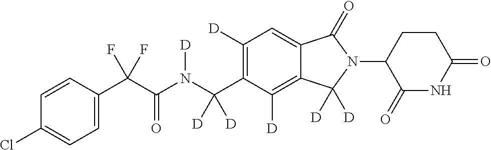 |
| 490 | 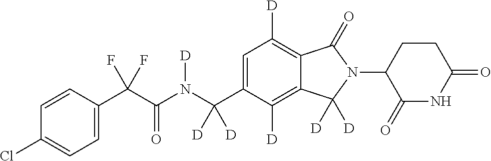 |
| 491 | 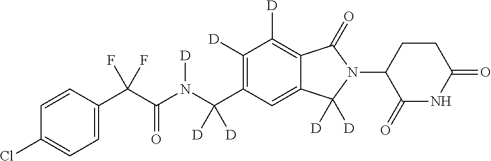 |
| 492 | 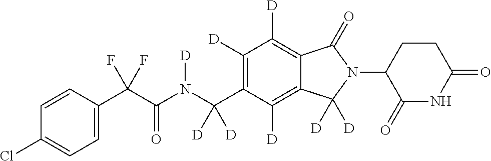 |

In one embodiment, the compound provided herein is a compound listed in Table 2, wherein one or more carbon atoms is/are radiolabeled carbon-14 (¹⁴C).

In one embodiment, the compound provided herein is a compound listed in Table 2, wherein one or more hydrogen atoms on the dioxopiperidinyl and/or phenyl ring are isotopically enriched with deuterium. In one embodiment, the compound provided herein is a compound listed in Table 2, wherein one, two, three, four or more hydrogen atoms on the dioxopiperidinyl and/or phenyl ring are isotopically enriched with deuterium.

In one embodiment, the compound provided herein is a compound listed in Table 2, wherein one or more hydrogen atoms on the dioxopiperidinyl ring are hydrogens that are isotopically enriched with deuterium. In one embodiment, the compound provided herein is a compound listed in Table 2, wherein one hydrogen atom on the dioxopiperidinyl ring is isotopically enriched with deuterium. In one embodiment, the compound provided herein is a compound listed in Table 2, wherein two hydrogen atoms on the dioxopiperidinyl ring are isotopically enriched with deuterium. In one embodiment, the compound provided herein is a compound listed in Table 2, wherein three hydrogen atoms on the dioxopiperidinyl ring are isotopically enriched with deuterium. In one embodiment, the compound provided herein is a compound listed in Table 2, wherein four hydrogen atoms on the dioxopiperidinyl ring are isotopically enriched with deuterium. In one embodiment, the compound provided herein is a compound listed in Table 2, wherein five hydrogen atoms on the dioxopiperidinyl ring are isotopically enriched with deuterium. In one embodiment, the compound provided herein is a compound listed in Table 2, wherein six hydrogen atoms on the dioxopiperidinyl ring are isotopically enriched with deuterium.

In one embodiment, the compound provided herein is a compound listed in Table 2, wherein one or more hydrogen atoms on the phenyl ring are isotopically enriched with deuterium. In one embodiment, the compound provided herein is a compound listed in Table 2, wherein one hydrogen atom on the phenyl ring is isotopically enriched with deuterium. In one embodiment, the compound provided herein is a compound listed in Table 2, wherein two hydrogen atoms on the phenyl ring are isotopically enriched with deuterium. In one embodiment, the compound provided herein is a compound listed in Table 2, wherein three hydrogen atoms on the phenyl ring are isotopically enriched with deuterium. In one embodiment, the compound provided herein is a compound listed in Table 2, wherein four hydrogen atoms on the phenyl ring are isotopically enriched with deuterium.

In certain embodiments, one or more Y atoms on the phenyl portion of A1, A2 or A3 are deuterium-enriched. For example, particular compounds provided herein include, but are not limited to, the compounds listed in Table 3, and its stereoisomers or mixture of stereoisomers, pharmaceutically acceptable salts, tautomers, solvates, hydrates, co-crystals, clathrates, or polymorphs thereof, wherein the label “D” indicates a deuterium-enriched atomic position, i.e., a sample comprising the given compound has a deuterium enrichment at the indicated position(s) above the natural abundance of deuterium:

TABLE 3

| No. | Compound structure |
|---|---|
| 493 | 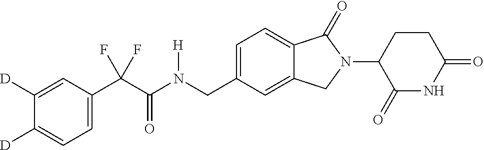 [SMILES: [2H]c1ccc(C(F)(F)C(=O)NCc2ccc3c(c2)CN(C2CCC(=O)NC2=O)C3=O)cc1[2H]] |
| 494 | 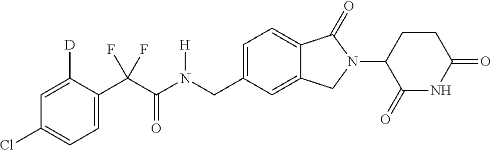 [SMILES: [2H]c1cc(Cl)ccc1C(F)(F)C(=O)NCc1ccc2c(c1)CN(C1CCC(=O)NC1=O)C2=O] |
| 495 | 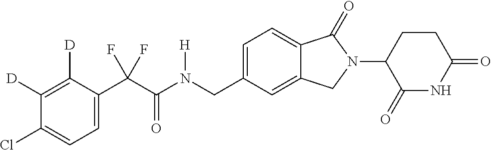 [SMILES: [2H]c1c(Cl)ccc(C(F)(F)C(=O)NCc2ccc3c(c2)CN(C2CCC(=O)NC2=O)C3=O)c1[2H]] |
| 496 | 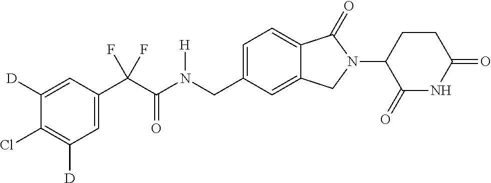 [SMILES: [2H]c1cc(C(F)(F)C(=O)NCc2ccc3c(c2)CN(C2CCC(=O)NC2=O)C3=O)cc([2H])c1Cl] |
| 497 | 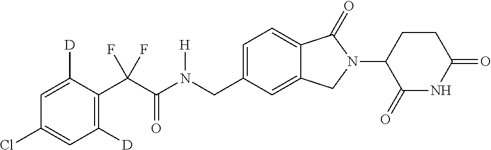 [SMILES: [2H]c1cc(Cl)cc([2H])c1C(F)(F)C(=O)NCc1ccc2c(c1)CN(C1CCC(=O)NC1=O)C2=O] |
| 498 | 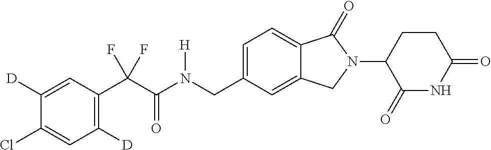 [SMILES: [2H]c1cc(C(F)(F)C(=O)NCc2ccc3c(c2)CN(C2CCC(=O)NC2=O)C3=O)c([2H])cc1Cl] |
| 499 | 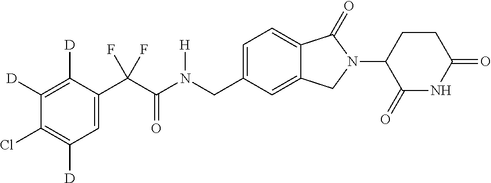 [SMILES: [2H]c1cc(C(F)(F)C(=O)NCc2ccc3c(c2)CN(C2CCC(=O)NC2=O)C3=O)c([2H])c([2H])c1Cl] |
| 500 | 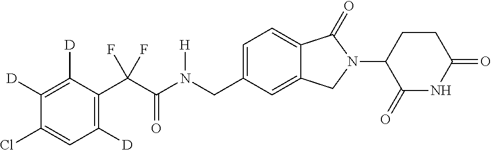 [SMILES: [2H]c1cc(Cl)c([2H])c([2H])c1C(F)(F)C(=O)NCc1ccc2c(c1)CN(C1CCC(=O)NC1=O)C2=O] |
| 501 | 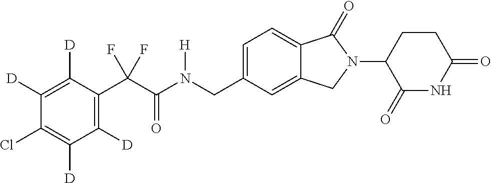 [SMILES: [2H]c1c([2H])c(C(F)(F)C(=O)NCc2ccc3c(c2)CN(C2CCC(=O)NC2=O)C3=O)c([2H])c([2H])c1Cl] |
| 502 | 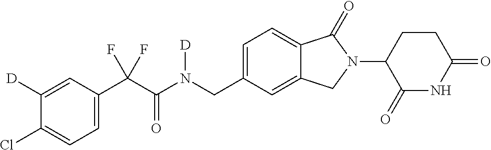 |
| 503 | 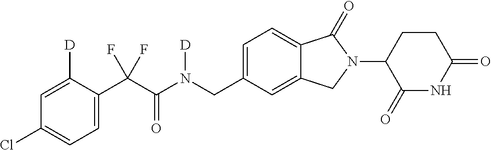 |
| 504 | 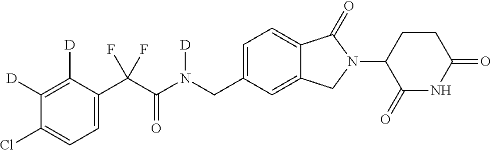 |
| 505 | 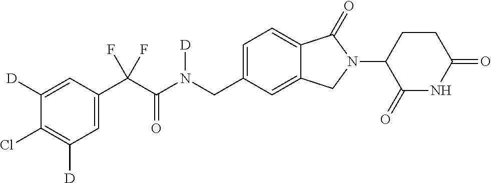 |
| 506 | 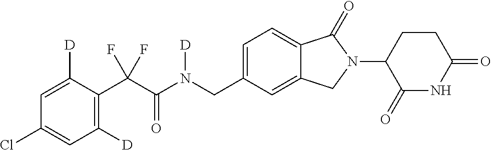 |
| 507 | 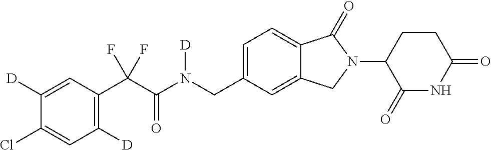 |
| 508 | 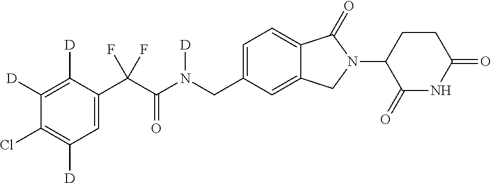 |
| 509 | 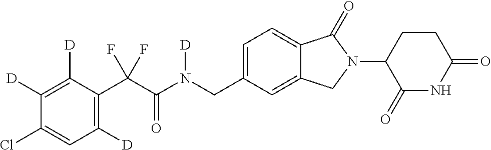 |
| 510 | 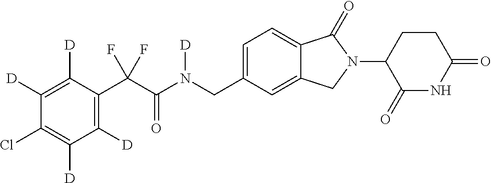 |
| 511 | 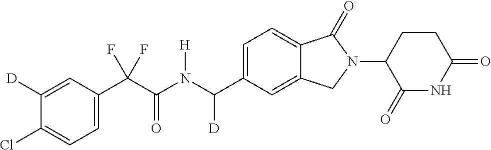 [SMILES: [2H]c1cc(C(F)(F)C(=O)NC([2H])c2ccc3c(c2)CN(C2CCC(=O)NC2=O)C3=O)ccc1Cl] |
| 512 | 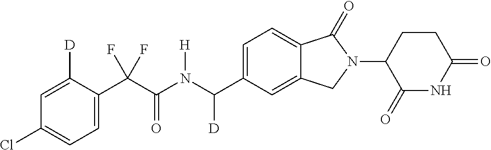 [SMILES: [2H]c1cc(Cl)ccc1C(F)(F)C(=O)NC([2H])c1ccc2c(c1)CN(C1CCC(=O)NC1=O)C2=O] |
| 513 | 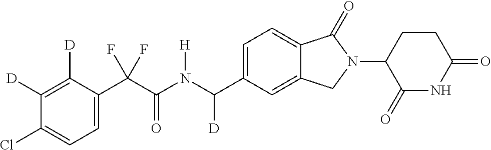 [SMILES: [2H]c1c(Cl)ccc(C(F)(F)C(=O)NC([2H])c2ccc3c(c2)CN(C2CCC(=O)NC2=O)C3=O)c1[2H]] |
| 514 | 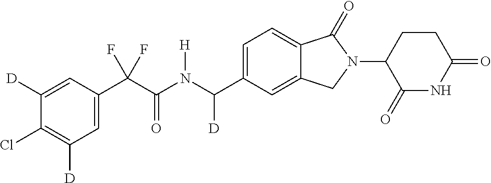 [SMILES: [2H]c1cc(C(F)(F)C(=O)NC([2H])c2ccc3c(c2)CN(C2CCC(=O)NC2=O)C3=O)cc([2H])c1Cl] |
| 515 | 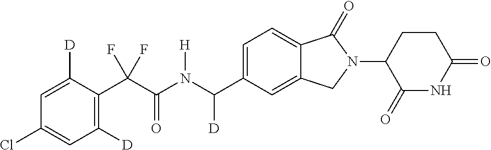 [SMILES: [2H]c1cc(Cl)cc([2H])c1C(F)(F)C(=O)NC([2H])c1ccc2c(c1)CN(C1CCC(=O)NC1=O)C2=O] |
| 516 | 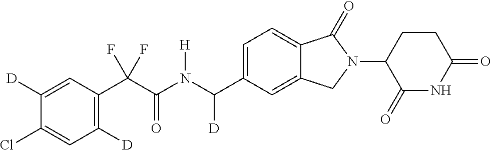 [SMILES: [2H]c1cc(C(F)(F)C(=O)NC([2H])c2ccc3c(c2)CN(C2CCC(=O)NC2=O)C3=O)c([2H])cc1Cl] |
| 517 | 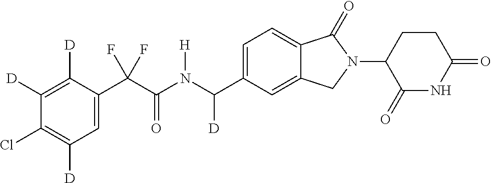 [SMILES: [2H]c1cc(C(F)(F)C(=O)NC([2H])c2ccc3c(c2)CN(C2CCC(=O)NC2=O)C3=O)c([2H])c([2H])c1Cl] |
| 518 | 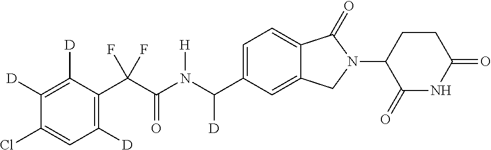 [SMILES: [2H]c1cc(Cl)c([2H])c([2H])c1C(F)(F)C(=O)NC([2H])c1ccc2c(c1)CN(C1CCC(=O)NC1=O)C2=O] |
| 519 | 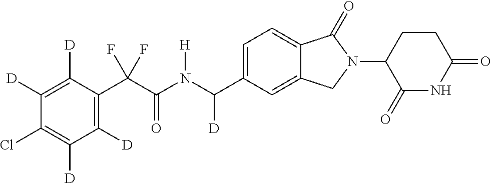 [SMILES: [2H]c1c([2H])c(C(F)(F)C(=O)NC([2H])c2ccc3c(c2)CN(C2CCC(=O)NC2=O)C3=O)c([2H])c([2H])c1Cl] |
| 520 | 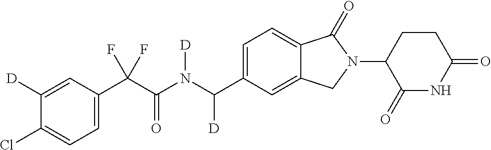 |
| 521 | 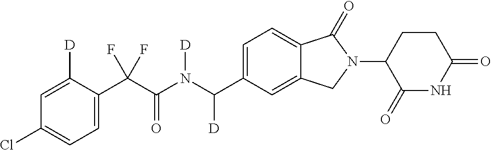 |
| 522 | 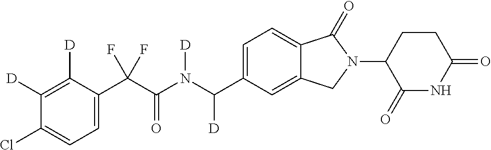 |
| 523 | 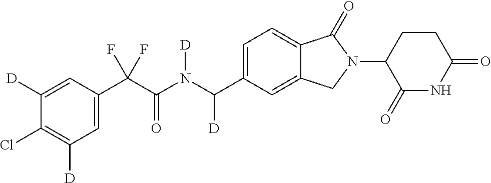 |
| 524 | 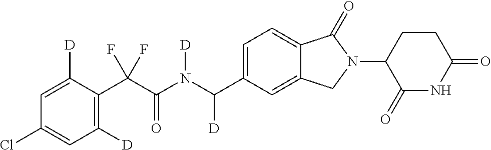 |
| 525 | 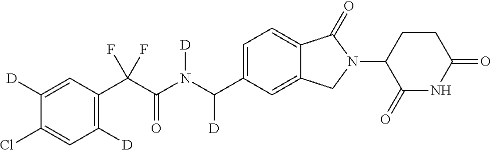 |
| 526 | 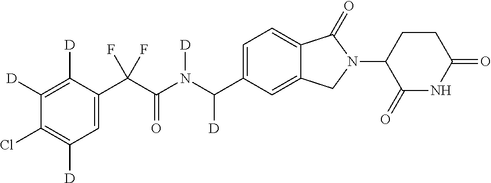 |
| 527 | 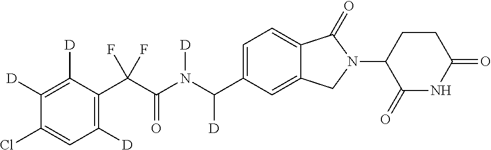 |
| 528 | 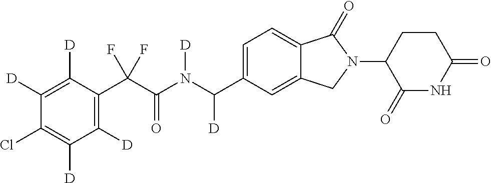 |
| 529 | 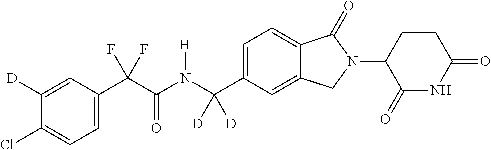 [SMILES: [2H]c1cc(C(F)(F)C(=O)NC([2H])([2H])c2ccc3c(c2)CN(C2CCC(=O)NC2=O)C3=O)ccc1Cl] |
| 530 | 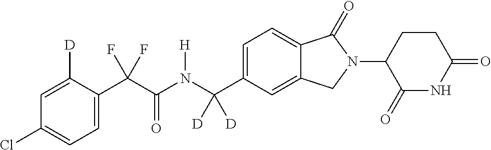 [SMILES: [2H]c1cc(Cl)ccc1C(F)(F)C(=O)NC([2H])([2H])c1ccc2c(c1)CN(C1CCC(=O)NC1=O)C2=O] |
| 531 | 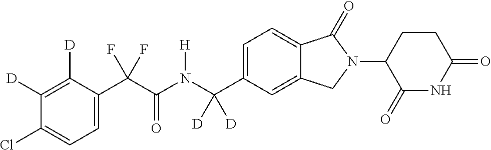 [SMILES: [2H]c1c(Cl)ccc(C(F)(F)C(=O)NC([2H])([2H])c2ccc3c(c2)CN(C2CCC(=O)NC2=O)C3=O)c1[2H]] |
| 532 | 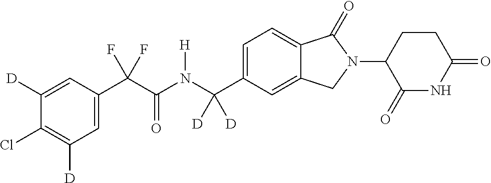 [SMILES: [2H]c1cc(C(F)(F)C(=O)NC([2H])([2H])c2ccc3c(c2)CN(C2CCC(=O)NC2=O)C3=O)cc([2H])c1Cl] |
| 533 | 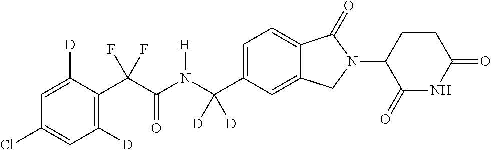 [SMILES: [2H]c1cc(Cl)cc([2H])c1C(F)(F)C(=O)NC([2H])([2H])c1ccc2c(c1)CN(C1CCC(=O)NC1=O)C2=O] |
| 534 | 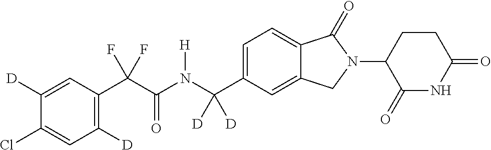 [SMILES: [2H]c1cc(C(F)(F)C(=O)NC([2H])([2H])c2ccc3c(c2)CN(C2CCC(=O)NC2=O)C3=O)c([2H])cc1Cl] |
| 535 | 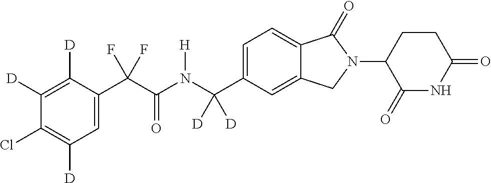 [SMILES: [2H]c1cc(C(F)(F)C(=O)NC([2H])([2H])c2ccc3c(c2)CN(C2CCC(=O)NC2=O)C3=O)c([2H])c([2H])c1Cl] |
| 536 | 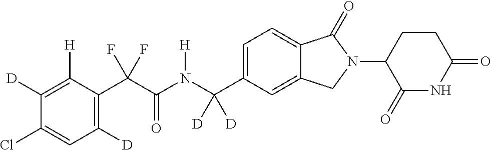 [SMILES: [2H]c1cc(C(F)(F)C(=O)NC([2H])([2H])c2ccc3c(c2)CN(C2CCC(=O)NC2=O)C3=O)c([2H])cc1Cl] |
| 537 | 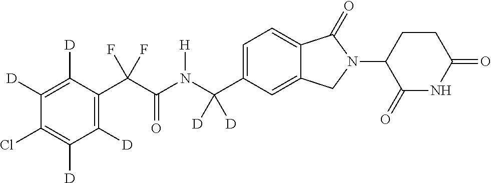 [SMILES: [2H]c1c([2H])c(C(F)(F)C(=O)NC([2H])([2H])c2ccc3c(c2)CN(C2CCC(=O)NC2=O)C3=O)c([2H])c([2H])c1Cl] |
| 538 | 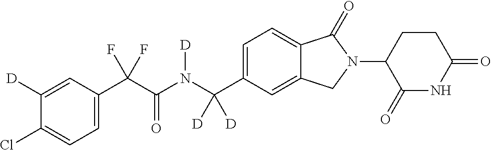 |
| 539 | 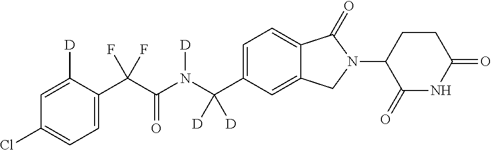 |
| 540 | 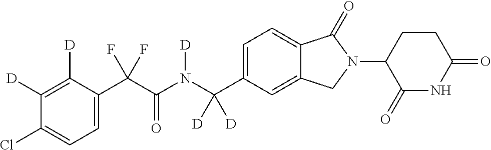 |
| 541 | 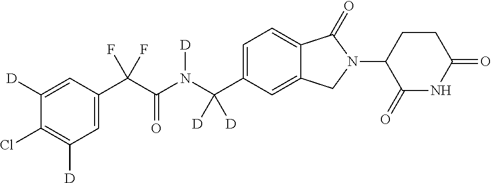 |
| 542 | 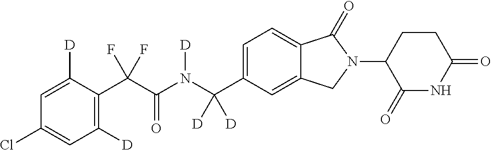 |
| 543 | 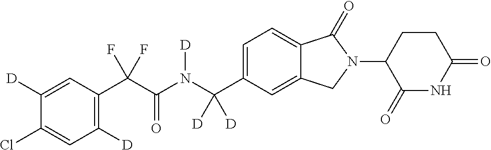 |
| 544 | 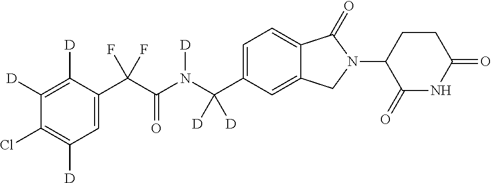 |
| 545 | 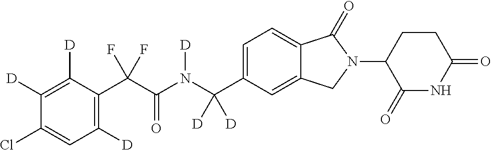 |
| 546 | 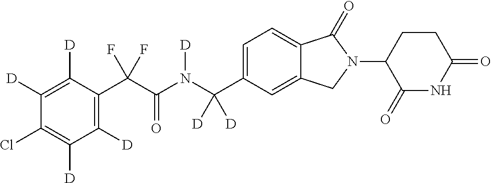 |

In one embodiment, the compound provided herein is a compound listed in Table 3, wherein one or more carbon atoms is/are radiolabeled carbon-14 (¹⁴C).

In one embodiment, the compound provided herein is a compound listed in Table 3, wherein one or more hydrogen atoms on the oxoisoindolinyl and/or dioxopiperidinyl ring are isotopically enriched with deuterium. In one embodiment, the compound provided herein is a compound listed in Table 3, wherein one, two, three, four, five or more hydrogen atoms on the oxoisoindolinyl and/or dioxopiperidinyl ring are isotopically enriched with deuterium.

In one embodiment, the compound provided herein is a compound listed in Table 3, wherein one or more hydrogen atoms on the oxoisoindolinyl ring are isotopically enriched with deuterium. In one embodiment, the compound provided herein is a compound listed in Table 3, wherein one hydrogen atom on the oxoisoindolinyl ring is isotopically enriched with deuterium. In one embodiment, the compound provided herein is a compound listed in Table 3, wherein two hydrogen atoms on the oxoisoindolinyl ring are isotopically enriched with deuterium. In one embodiment, the compound provided herein is a compound listed in Table 3, wherein three hydrogen atoms on the oxoisoindolinyl ring are isotopically enriched with deuterium. In one embodiment, the compound provided herein is a compound listed in Table 3, wherein four hydrogen atoms on the oxoisoindolinyl ring are isotopically enriched with deuterium. In one embodiment, the compound provided herein is a compound listed in Table 3, wherein five hydrogen atoms on the oxoisoindolinyl ring are isotopically enriched with deuterium. In one embodiment, the compound provided herein is a compound listed in Table 3, wherein six hydrogen atoms on the oxoisoindolinyl ring are isotopically enriched with deuterium.

In one embodiment, the compound provided herein is a compound listed in Table 3, wherein one or more hydrogen atoms on the dioxopiperidinyl ring are hydrogens that are isotopically enriched with deuterium. In one embodiment, the compound provided herein is a compound listed in Table 3, wherein one hydrogen atom on the dioxopiperidinyl ring is isotopically enriched with deuterium. In one embodiment, the compound provided herein is a compound listed in Table 3, wherein two hydrogen atoms on the dioxopiperidinyl ring are isotopically enriched with deuterium. In one embodiment, the compound provided herein is a compound listed in Table 3, wherein three hydrogen atoms on the dioxopiperidinyl ring are isotopically enriched with deuterium. In one embodiment, the compound provided herein is a compound listed in Table 3, wherein four hydrogen atoms on the dioxopiperidinyl ring are isotopically enriched with deuterium. In one embodiment, the compound provided herein is a compound listed in Table 3, wherein five hydrogen atoms on the dioxopiperidinyl ring are isotopically enriched with deuterium. In one embodiment, the compound provided herein is a compound listed in Table 3, wherein six hydrogen atoms on the dioxopiperidinyl ring are isotopically enriched with deuterium.

In certain embodiments, one or more Y atoms on the acetamide portion of A1, A2 or A3 are deuterium-enriched. For example, particular compounds provided herein include, but are not limited to, the compounds listed in Table 4, and its stereoisomers or mixture of stereoisomers, pharmaceutically acceptable salts, tautomers, solvates, hydrates, co-crystals, clathrates, or polymorphs thereof, wherein the label “D” indicates a deuterium-enriched atomic position, i.e., a sample comprising the given compound has a deuterium enrichment at the indicated position(s) above the natural abundance of deuterium:

TABLE 4

| No. | Compound structure |
|---|---|
| 547 | 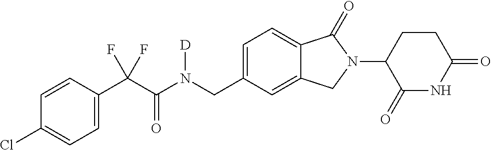 |
| 548 | 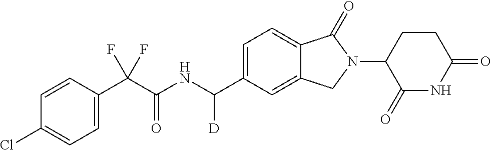 [SMILES: [2H]C(NC(=O)C(F)(F)c1ccc(Cl)cc1)c1ccc2c(c1)CN(C1CCC(=O)NC1=O)C2=O] |
| 549 | 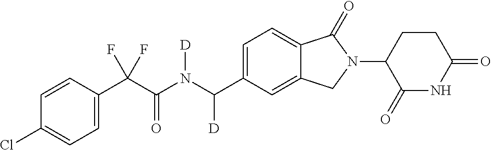 |
| 550 | 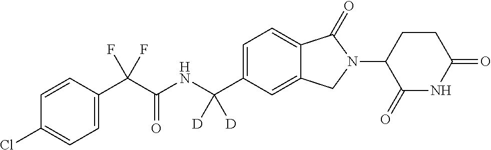 [SMILES: [2H]C([2H])(NC(=O)C(F)(F)c1ccc(Cl)cc1)c1ccc2c(c1)CN(C1CCC(=O)NC1=O)C2=O] |
| 551 | 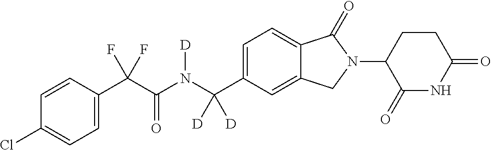 |

In one embodiment, the compound provided herein is a compound listed in Table 4, wherein one or more carbon atoms is/are radiolabeled carbon-14 (¹⁴C).

In certain embodiments, one or more Y atoms on the dioxopiperidinyl, oxoisoindolinyl or phenyl portion of A1, A2 or A3 are deuterium-enriched. For example, particular compounds provided herein include, but are not limited to, the compounds listed in Table 5, and its stereoisomers or mixture of stereoisomers, pharmaceutically acceptable salts, tautomers, solvates, hydrates, co-crystals, clathrates, or polymorphs thereof, wherein the label “D” indicates a deuterium-enriched atomic position, i.e., a sample comprising the given compound has a deuterium enrichment at the indicated position(s) above the natural abundance of deuterium:

TABLE 5

| No. | Compound structure |
|---|---|
| 552 | 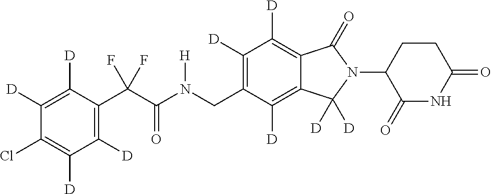 [SMILES: [2H]c1c([2H])c(C(F)(F)C(=O)NCc2c([2H])c([2H])c3c(c2[2H])C([2H])([2H])N(C2CCC(=O)NC2=O)C3=O)c([2H])c([2H])c1Cl] |
| 553 | 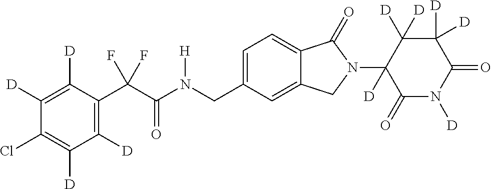 |
| 554 | 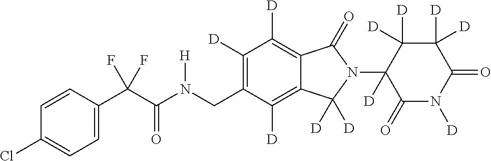 |
| 555 | 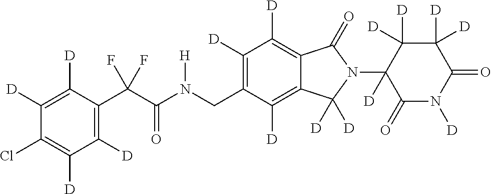 |
| 556 | 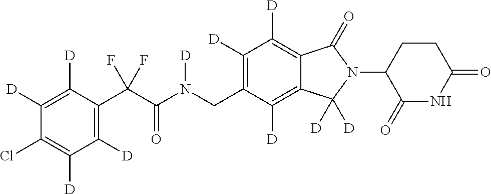 |
| 557 | 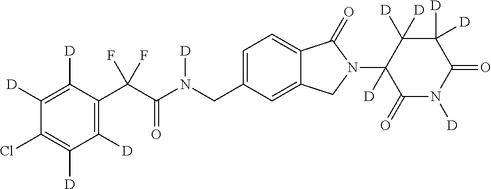 |
| 558 | 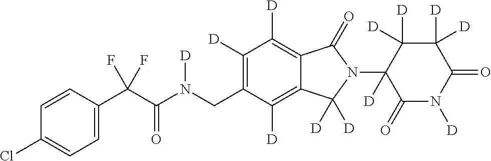 |
| 559 | 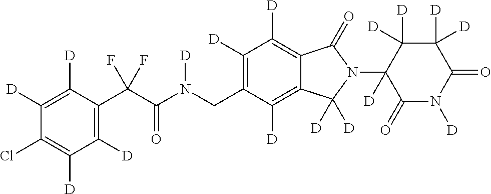 |
| 560 | 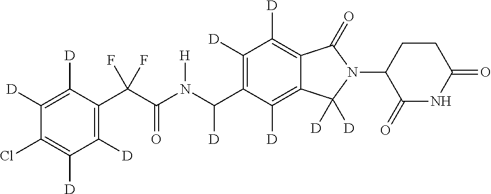 [SMILES: [2H]c1c([2H])c(C(F)(F)C(=O)NC([2H])c2c([2H])c([2H])c3c(c2[2H])C([2H])([2H])N(C2CCC(=O)NC2=O)C3=O)c([2H])c([2H])c1Cl] |
| 561 | 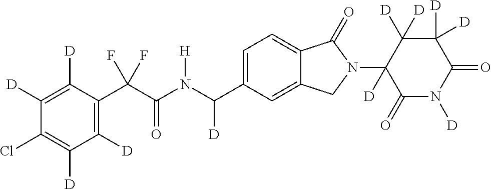 |
| 562 | 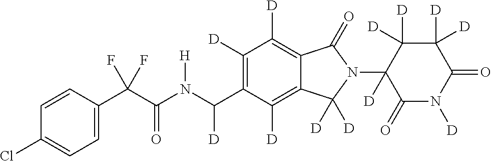 |
| 563 | 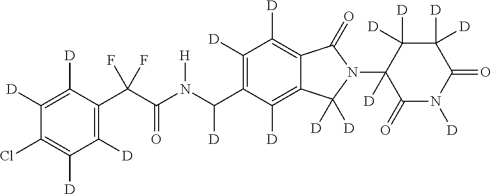 |
| 564 | 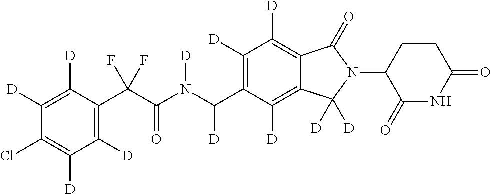 |
| 565 | 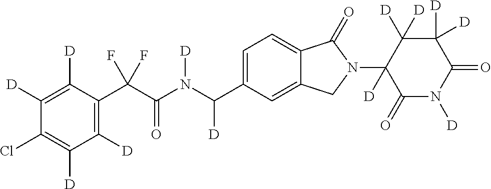 |
| 566 | 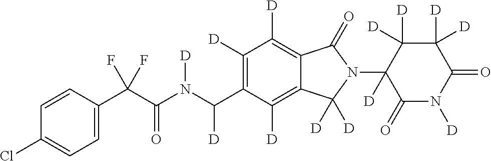 |
| 567 | 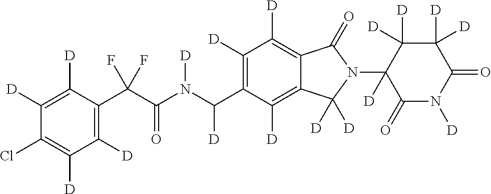 |
| 568 | 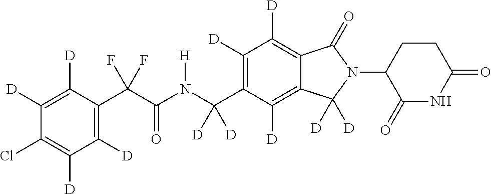 [SMILES: [2H]c1c([2H])c(C(F)(F)C(=O)NC([2H])([2H])c2c([2H])c([2H])c3c(c2[2H])C([2H])([2H])N(C2CCC(=O)NC2=O)C3=O)c([2H])c([2H])c1Cl] |
| 569 | 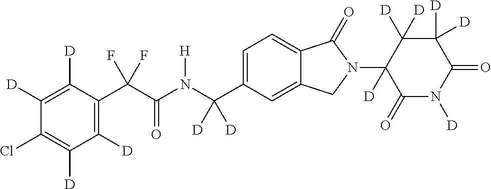 |
| 570 | 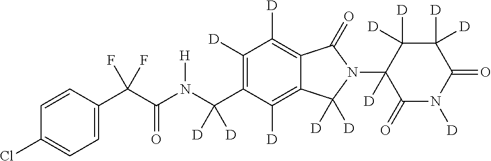 |
| 571 | 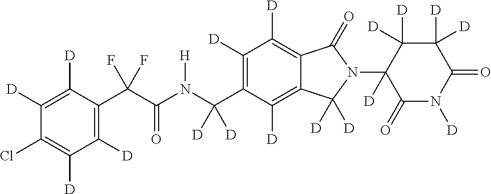 |
| 572 | 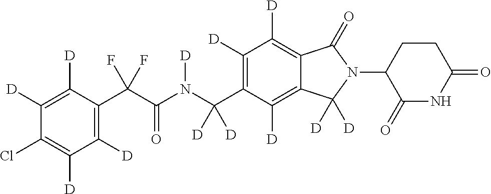 |
| 573 | 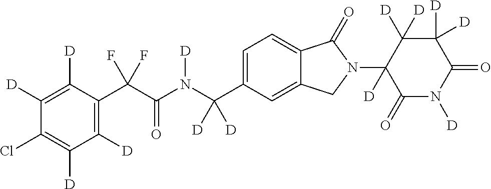 |
| 574 | 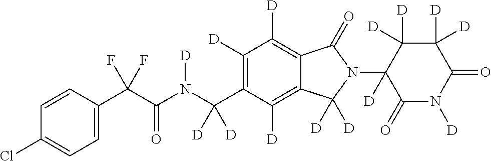 |
| 575 | 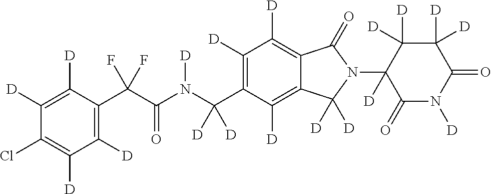 |
| 576 | 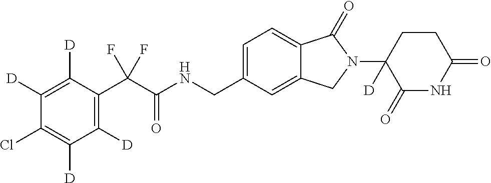 [SMILES: [2H]c1c([2H])c(C(F)(F)C(=O)NCc2ccc3c(c2)CN(C2([2H])CCC(=O)NC2=O)C3=O)c([2H])c([2H])c1Cl] |

It is understood that one or more deuteriums may exchange with hydrogen under physiological conditions.

In one embodiment, the compound provided herein is a compound listed in Table 5, wherein one or more carbon atoms is/are radiolabeled carbon-14 (¹⁴C).

In one embodiment, the compound provided herein is

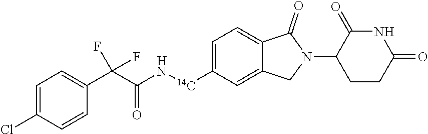 [SMILES: O=C1CCC(N2Cc3cc([14CH2]NC(=O)C(F)(F)c4ccc(Cl)cc4)ccc3C2=O)C(=O)N1]

or a stereoisomer, a mixture of stereoisomers, a pharmaceutically acceptable salt, a tautomer, a solvate, a hydrate, a co-crystal, a clathrate, or a polymorph thereof.

5.2 Synthesis

The compounds described herein may be synthesized using methods known to those of ordinary skill in the art. For example, particular compounds described herein are synthesized using standard synthetic organic chemistry techniques known to those of ordinary skill in the art.

In some embodiments, known procedures for the synthesis of compounds of A1, A2 or A3 are employed, wherein one or more of the reagents, starting materials, precursors, or intermediates are replaced by one or more isotopically-enriched reagents or intermediates, including but not limited to one or more deuterium-enriched reagents, starting materials, precursors, or intermediates. Such known procedures for the synthesis of compounds of A1, A2 or A3 and tautomers thereof include, but are not limited to, those described in U.S. Pat. No. 9,499,514, the entirety of which is incorporated by reference herein. Isotopically enriched reagents, starting materials, precursors, and intermediates are commercially available or may be prepared by routine chemical reactions known to one of skill in the art.

Exemplary schemes for preparation of an isotopologue of compounds of A1, A2 or A3 are provided below:

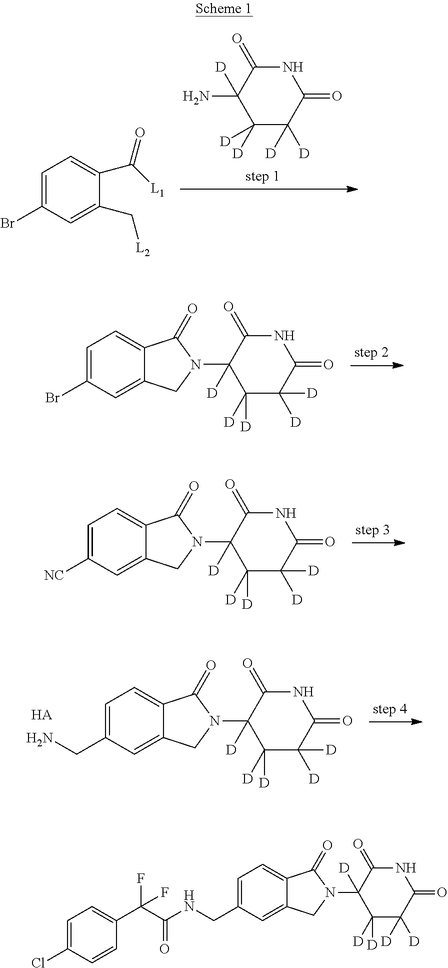

where L¹ and L² are leaving groups. Exemplary leaving groups include, but are not limited to halogen, —OR, —OCOR, —OSO₂R, and —OPO₃R; where each R is independently C₁₋₁₀alkyl, C₂₋₁₀alkenyl, C₂₋₁₀alkynyl, 5 to 10 membered aryl or 5 to 10 membered heteroaryl, and each R group is optionally independently substituted with one, two, three, four or more halogens. In one embodiment, the heteroaryl group contains 1-3 heteroatoms selected from N, O and S. In one embodiment, L₁ is O-methyl, and L₂ is Cl, Br, O-mesylate, or O-tosylate.

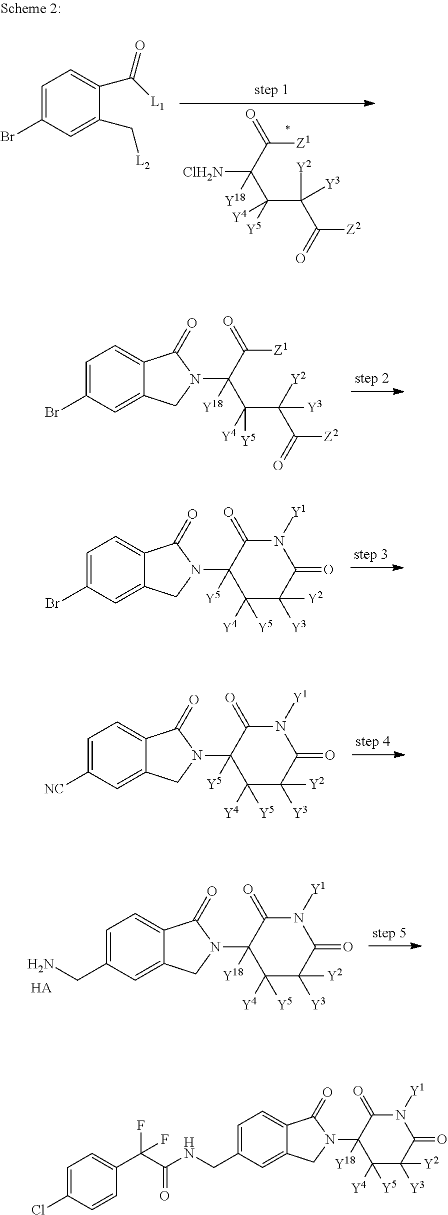

wherein one or more Y atoms (i.e., Y¹, Y², Y³, Y⁴, Y⁵, and Y¹⁸) is/are hydrogen(s) isotopically enriched with deuterium, and any remaining Y atom(s) is/are non-enriched hydrogen atom(s), L¹ and L² are leaving groups, Z¹ and Z² are selected as follows: a) Z¹ is NHZ³, and Z² is OR; b) Z¹ is OR, and Z² is NHZ³; or c) Z¹ and Z² are both OH; Z³ is hydrogen, or a suitable amino protecting group; each R is independently C₁₋₁₀alkyl, C₂₋₁₀alkenyl, C₂₋₁₀alkynyl, 5 to 10 membered aryl, or 5 to 10 membered heteroaryl, and each R group is optionally independently substituted with one, two, three, four or more halogens. Exemplary amino protecting groups include, but are not limited to Boc (t-butyloxy carbamate), Fmoc (9-fluorenylmethyl carbamate), Alloc (allyl carbamate), Troc (trichloethyl carbamate), and Cbz (benzylcarboxy carbamate). Exemplary leaving groups include, but are not limited to halogen, —OR, —OCOR, —OSO₂R, and —OPO₃R; where each R is independently C₁₋₁₀alkyl, C₂₋₁₀alkenyl, C₂₋₁₀alkynyl, 5 to 10 membered aryl or 5 to 10 membered heteroaryl, and each R group is optionally independently substituted with one, two, three, four or more halogens. In one embodiment, the heteroaryl group contains 1-3 heteroatoms selected from N, O and S. In one embodiment, L₁ is O-methyl, and L₂ is Cl, Br, O-mesylate, or O-tosylate.

* Deuterium-enriched (2,3,3,4,4-d5) L-glutamine is available from Aldrich. Other partially deuterium-enriched L-glutamine are either commercially available or can be prepared using literature procedure.

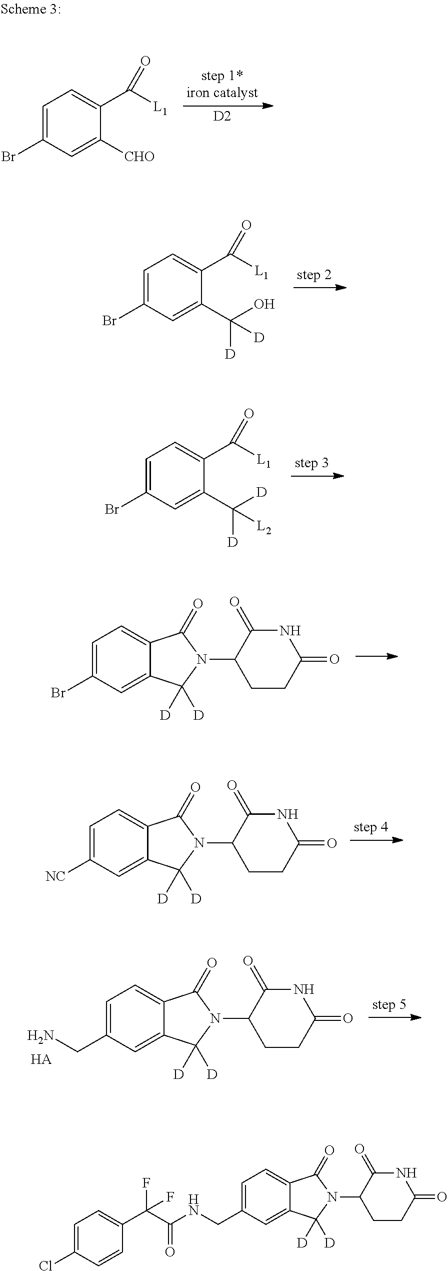

where L¹ and L² are leaving groups. Exemplary leaving groups include, but are not limited to halogen, —OR, —OCOR, —OSO₂R, and —OPO₃R; where each R is independently C₁₋₁₀alkyl, C₂₋₁₀alkenyl, C₂₋₁₀alkynyl, 5 to 10 membered aryl or 5 to 10 membered heteroaryl, and each R group is optionally independently substituted with one, two, three, four or more halogens. In one embodiment, the heteroaryl group contains 1-3 heteroatoms selected from N, O and S. In one embodiment, L₁ is O-methyl, and L₂ is Cl, Br, O-mesylate, or O-tosylate.

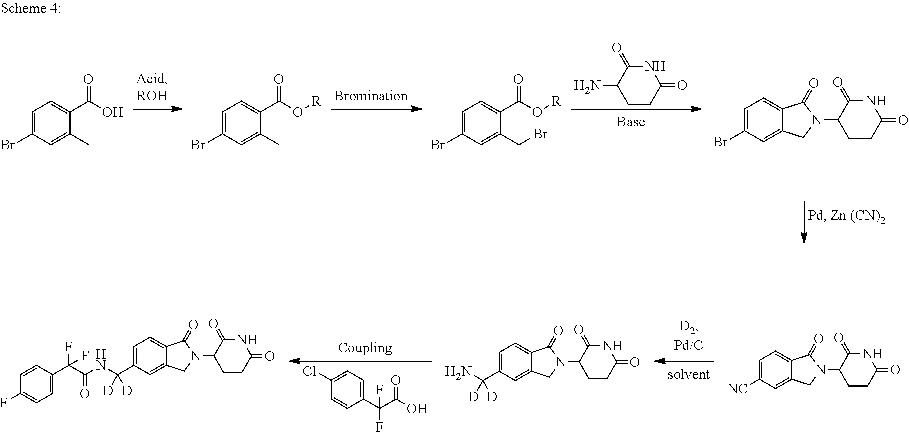

where R is alkyl.

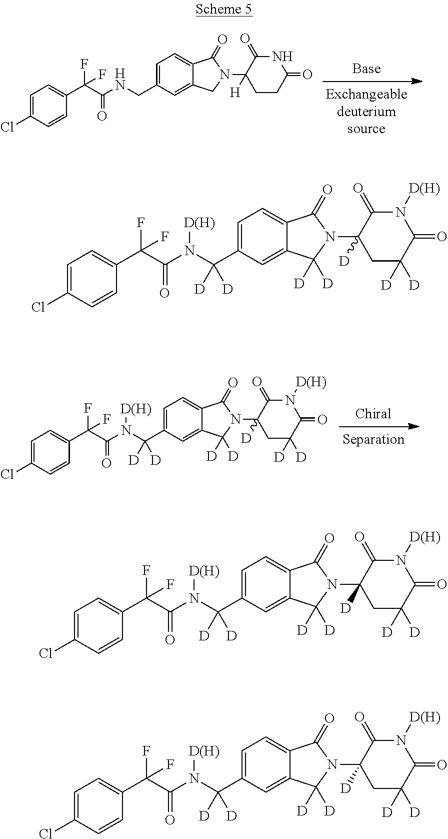

wherein the exchangeable deuterium source is selected from D₂O, C₁₋₁₄ alkyl-OD, C₁₋₁₄ alkyl-COOD, aryl-OD, heteroaryl-OD, aryl-SO₃D, deuterium chloride, deuterium bromide, deuterium iodide, sulfuric acid-d2, and nitric acid-d₁.

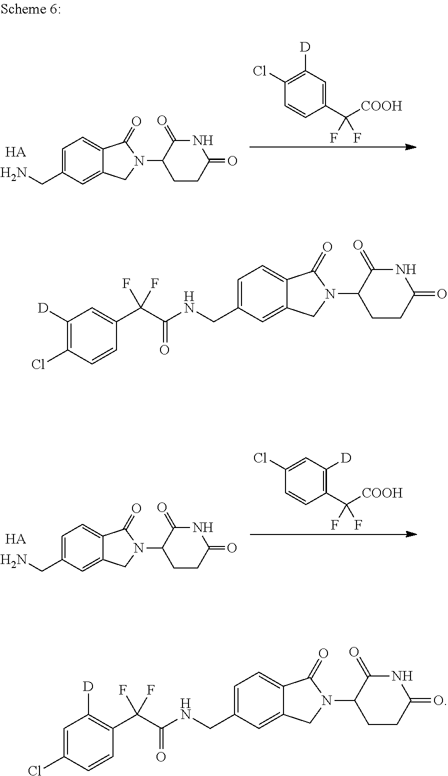

In certain embodiments, the methods described in Schemes 1-6 are employed. In particular embodiments, the methods of Schemes 1-6 are employed, wherein deuterium-enriched reagents are used, similar to above.

5.3 Methods of Use

In one embodiment, provided herein are methods of treating, preventing, managing, and/or ameliorating cancers, including solid tumors and hematological cancers, or one or more symptoms or causes thereof, by administering an isotopologue of Compound A as provided herein. In one embodiment, provided herein are methods of treating such cancers or one or more symptoms or causes thereof, by administering an isotopologue of Compound A as provided herein. In one embodiment, provided herein are methods of preventing such cancers or one or more symptoms or causes thereof, by administering an isotopologue of Compound A as provided herein. In one embodiment, provided herein are methods of managing such cancers or one or more symptoms or causes thereof, by administering an isotopologue of Compound A as provided herein. In one embodiment, provided herein are methods of ameliorating such cancers or one or more symptoms or causes thereof, by administering an isotopologue of Compound A as provided herein.

Also provided herein are methods of treating patients who have been previously treated for cancer but are non-responsive to cancer therapies, as well as those who have not previously been treated. Also encompassed are methods of treating patients regardless of patient's age, although some diseases or disorders are more common in certain age groups. Further encompassed are methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

In certain embodiments, the cancer is a solid tumor or a hematological cancer.

In certain embodiments, the cancer is a solid tumor. In certain embodiments, the solid tumor is metastatic. In certain embodiments, the solid tumor is drug-resistant.

In certain embodiments, cancer refers to a disease of skin tissues, organs, blood, and vessels. In certain embodiments, the cancer is a solid tumor, including, but not limited to, cancers of the bladder, bone, blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, colorectal cancer, including stage 3 and stage 4, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, carcinoma, including papillary thyroid carcinoma, follicular thyroid carcinoma, and medullary thyroid carcinoma, and leiomyoma.

In certain embodiments, the solid tumor is hepatocellular carcinoma, prostate cancer, ovarian cancer, or glioblastoma.

In certain embodiments, the solid tumor is breast cancer, kidney cancer, pancreatic cancer, gastrointestinal cancer, lung cancer, neuroendocrine tumor (NET), or renal cell carcinoma (RCC).

In certain embodiments, the cancer is a hematological cancer. In certain embodiments, the hematological cancer is metastatic. In certain embodiments, the hematological cancer is drug resistant to at least one anti-cancer therapy. In certain embodiments the hematological cancer is relapsed or refractory to at least one anti-cancer therapy.

In one embodiment, the hematological cancer is multiple myeloma (MM). In one embodiment, the hematological cancer is relapsed/refractory (R/R) multiple myeloma. In one embodiment, the patient having R/R multiple myeloma has impaired renal function.

In one embodiment, the hematological cancer is acute myelogenous leukemia (AML). In one embodiment, the hematological cancer is acute lymphocytic leukemia (ALL). In one embodiment, the hematological cancer is adult T-cell leukemia. In one embodiment, the hematological cancer is chronic lymphocytic leukemia (CLL). In one embodiment, the hematological cancer is hairy cell leukemia. In one embodiment, the hematological cancer is myelodysplasia. In one embodiment, the hematological cancer is a myeloproliferative disorder or myeloproliferative neoplasm (MPN). In one embodiment, the hematological cancer is chronic myelogenous leukemia (CML). In one embodiment, the hematological cancer is myelodysplastic syndrome (MDS). In one embodiment, the hematological cancer is, human lymphotropic virus-type 1 (HTLV-1) leukemia. In one embodiment, the hematological cancer is mastocytosis. In one embodiment, the hematological cancer is B-cell acute lymphoblastic leukemia. In one embodiment, the hematological cancer is CLL.

In one embodiment, provided herein are methods of treating, preventing, managing, and/or ameliorating a cancer selected from diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, mantle cell lymphoma (MCL), Hodgkin lymphoma (HL), non-Hodgkin lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, and ALK-positive large B-cell lymphoma in a subject, comprising the step of administering to the subject an amount of an isotopologue of Compound A provided herein effective to treat, prevent and/or manage the cancer. In some embodiments, the methods comprise the step of administering to the subject an isotopologue of Compound A provided herein in combination with a second active agent in amounts effective to treat, prevent and/or manage the cancer. In one embodiment, the hematological cancer is HL. In one embodiment, the hematological cancer is NHL. In one embodiment, the hematological cancer is indolent lymphoma including, for example, DLBCL, follicular lymphoma, and marginal zone lymphoma.

In one embodiment, provided herein are methods of treating, preventing, managing, and/or ameliorating leukemia by administering an isotopologue of Compound A to a subject. In one embodiment, the leukemia is acute myeloid leukemia (AML). In one embodiment, the AML is relapsed or refractory AML. In one embodiment, the AML is newly diagnosed AML. In another embodiment, the AML has FAB classification M0/1. In another embodiment, the AML has FAB classification M2. In another embodiment, the AML has FAB classification M3. In another embodiment, the AML has FAB classification M4. In another embodiment, the AML has FAB classification M5. In one embodiment, the AML is AML with at least one recurrent genetic abnormality (for example, AML with translocation between chromosomes 8 and 21; AML with translocation or inversion in chromosome 16; AML with translocation between chromosomes 9 and 11; APL (M3) with translocation between chromosomes 15 and 17; AML with translocation between chromosomes 6 and 9; AML with translocation or inversion in chromosome 3); AML (megakaryoblastic) with a translocation between chromosomes 1 and 22; AML with myelodysplasia-related changes; AML related to previous chemotherapy or radiation (for example, alkylating agent-related AML; or Topoisomerase II inhibitor-related AML); AML not otherwise categorized (for example, AML that does not fall into the above categories, i. e. AML minimally differentiated (M0); AML with minimal maturation (M1); AML with maturation (M2); Acute myelomonocytic leukemia (M4); Acute monocytic leukemia (M5); Acute erythroid leukemia (M6); Acute megakaryoblastic leukemia (M7); Acute basophilic leukemia; or Acute panmyelosis with fibrosis); Myeloid Sarcoma (also known as granulocytic sarcoma, chloroma or extramedullary myeloblastoma); or Undifferentiated and biphenotypic acute leukemias (also known as mixed phenotype acute leukemias). In certain embodiments, the methods of treating, preventing and/or managing acute myeloid leukemia in a subject comprise the step of administering to the subject an amount of an isotopologue of Compound A provided herein effective to treat, prevent and/or manage acute myeloid leukemia. In some embodiments, the methods comprise the step of administering to the subject an isotopologue of Compound A provided herein in combination with a second active agent in amounts effective to treat, prevent, ameliorate and/or manage acute myeloid leukemia.

In some embodiments, the methods provided herein encompass treating, preventing, ameliorating and/or managing acute lymphocytic leukemia (ALL) in a subject. In some embodiments, acute lymphocytic leukemia includes leukemia that originates in the blast cells of the bone marrow (B-cells), thymus (T-cells), and lymph nodes. The acute lymphocytic leukemia can be categorized according to the French-American-British (FAB) Morphological Classification Scheme as L1—Mature-appearing lymphoblasts (T-cells or pre-B-cells), L2—Immature and pleomorphic (variously shaped) lymphoblasts (T-cells or pre-B-cells), and L3—Lymphoblasts (B-cells; Burkitt's cells). In one embodiment, the acute lymphocytic leukemia originates in the blast cells of the bone marrow (B-cells). In one embodiment, the acute lymphocytic leukemia originates in the thymus (T-cells). In one embodiment, the acute lymphocytic leukemia originates in the lymph nodes. In one embodiment, the acute lymphocytic leukemia is L1 type characterized by mature-appearing lymphoblasts (T-cells or pre-B-cells). In one embodiment, the acute lymphocytic leukemia is L2 type characterized by immature and pleomorphic (variously shaped) lymphoblasts (T-cells or pre-B-cells). In one embodiment, the acute lymphocytic leukemia is L3 type characterized by lymphoblasts (B-cells; Burkitt's cells). In certain embodiments, the acute lymphocytic leukemia is T-cell leukemia. In one embodiment, the T-cell leukemia is peripheral T-cell leukemia. In another embodiment, the T-cell leukemia is T-cell lymphoblastic leukemia. In another embodiment, the T-cell leukemia is cutaneous T-cell leukemia. In another embodiment, the T-cell leukemia is adult T-cell leukemia. In certain embodiments, the methods of treating, preventing and/or managing acute lymphocytic leukemia in a subject comprise the step of administering to the subject an amount of an isotopologue of Compound A provided herein effective to treat, prevent and/or manage acute lymphocytic leukemia. In some embodiments, the methods comprise the step of administering to the subject an isotopologue of Compound A provided herein in combination with a second active agent in amounts effective to treat, prevent, ameliorate and/or manage acute lymphocytic leukemia.

In some embodiments, the methods provided herein encompass treating, preventing, ameliorating and/or managing chronic myelogenous leukemia (CML) in a subject. The methods comprise the step of administering to the subject an amount of an isotopologue of Compound A as provided herein, effective to treat, prevent and/or manage chronic myelogenous leukemia.

In some embodiments, the methods provided herein encompass treating, preventing, ameliorating and/or managing chronic lymphocytic leukemia (CLL) in a subject. The methods comprise the step of administering to the subject an amount of an isotopologue of Compound A, effective to treat, prevent, ameliorate and/or manage chronic lymphocytic leukemia.

In one embodiment, provided herein are methods of treating, preventing, managing, and/or ameliorating a myelodysplastic syndrome (MDS) by administering an isotopologue of Compound A to a subject. In one embodiment provided herein is a method of treating MDS. In one embodiment, the MDS is relapsed, resistant or refractory MDS. In one embodiment, MDS is refractory anemia (RA); RA with ringed sideroblasts (RARS); RA with excess of blasts (RAEB); refractory cytopenia with multilineage dysplasia (RCMD), refractory cytopenia with unilineage dysplasia (RCUD); unclassifiable myelodysplastic syndrome (MDS-U), myelodysplastic syndrome associated with an isolated del(5q) chromosome abnormality, therapy-related myeloid neoplasms or chronic myelomonocytic leukemia (CMML). In some embodiments, the MDS is very low risk, low risk, intermediate risk, high risk or very high risk MDS. In one embodiment, the MDS is very low risk. In another embodiment, the MDS is low risk. In another embodiment, the MDS is intermediate risk. In another embodiment, the MDS is high risk. In another embodiment, the MDS is very high risk MDS. In some embodiments, the MDS is primary or de novo MDS. In other embodiments, the MDS is secondary MDS.

In some embodiments, the methods provided herein encompass treating, preventing, ameliorating and/or managing a myeloproliferative neoplasm. In one embodiment, the myeloproliferative neoplasm is polycythemia vera, primary or essential thrombocythemia, primary or idiopathic myelofibrosis, chronic myelogenous leukemia, chronic neutrophilic leukemia, juvenile myelomonocytic leukemia, chronic eosinophilic leukemia, or hyper eosinophilic syndrome. In certain embodiments, the methods of treating, preventing and/or managing a myeloproliferative neoplasm in a subject comprise the step of administering to the subject an amount of an isotopologue of Compound A, effective to treat, prevent, ameliorate and/or manage myeloproliferative neoplasm.

In one embodiment, the methods of treating, preventing, ameliorating and/or managing cancer provided herein comprise intravenous administration of an isotopologue of Compound A.

In certain embodiments, provided herein are method of treating, preventing, ameliorating and/or managing cancer in patients with impaired renal function. In certain embodiments, provided herein are methods of providing appropriate dose adjustments for patients with impaired renal function due to, but not limited to, disease, aging, or other patient factors.

In certain embodiments, a therapeutically or prophylactically effective amount of an isotopologue of Compound A is from about 0.005 to about 20 mg per day, from about 0.05 to 20 mg per day, from about 0.01 to about 10 mg per day, from about 0.01 to about 7 mg per day, from about 0.01 to about 5 mg per day, from about 0.01 to about 3 mg per day, from about 0.05 to about 10 mg per day, from about 0.05 to about 7 mg per day, from about 0.05 to about 5 mg per day, from about 0.05 to about 3 mg per day, from about 0.1 to about 15 mg per day, from about 0.1 to about 10 mg per day, from about 0.1 to about 7 mg per day, from about 0.1 to about 5 mg per day, from about 0.1 to about 3 mg per day, from about 0.5 to about 10 mg per day, from about 0.05 to about 5 mg per day, from about 0.5 to about 3 mg per day, from about 0.5 to about 2 mg per day, from about 0.3 to about 10 mg per day, from about 0.3 to about 8.5 mg per day, from about 0.3 to about 8.1 mg per day, from about 0.6 to about 10 mg per day or from about 0.6 to about 5 mg per day. In one embodiment, a therapeutically or prophylactically effective amount of an isotopologue of Compound A is from about 0.1 to about 10 mg per day. In one embodiment, a therapeutically or prophylactically effective amount of an isotopologue of Compound A is from about 0.5 to about 10 mg per day. In one embodiment, a therapeutically or prophylactically effective amount of an isotopologue of Compound A is from about 0.5 to about 5 mg per day.

In certain embodiments, the therapeutically or prophylactically effective amount of an isotopologue of Compound A is about 0.1, about 0.2, about 0.5, about 1, about 2, about 3, about 4, about 5, about 6, about 7, about 8, about 9, or about 10 mg per day. In some such embodiments, the therapeutically or prophylactically effective amount is about 0.5, about 0.6, about 0.75, about 1, about 2, about 3, about 4, about 5, about 6 or about 7 mg per day. In some such embodiments, the therapeutically or prophylactically effective amount is about 0.6, about 1.2, about 1.8, about 2.4, or about 3.6 mg per day

In one embodiment, the recommended daily dose range of an isotopologue of Compound A, for the conditions described herein lies within the range of from about 0.01 mg to about 10 mg per day, in one embodiment, given as a single once-a-day dose, or in divided doses throughout a day. In some embodiments, the dosage ranges from about 0.1 mg to about 10 mg per day. In other embodiments, the dosage ranges from about 0.5 to about 5 mg per day. Specific doses per day include 0.1, 0.2, 0.5, 0.6, 1, 1.2, 1.5, 1.8, 2, 2.4, 2.5, 3, 3.5, 3.6, 4, 4.5, 5, 5.5, 6, 6.5, 7, 7.5, 8, 8.5, 9, 9.5 or 10 mg per day.

In a specific embodiment, the recommended starting dosage may be 0.1, 0.5, 0.6, 0.7, 1, 1.2, 1.5, 1.8, 2, 2.4, 2.5, 3, 3.5, 3.6, 4, 4.5, 5, 5.5, 6, 6.5 or 7 mg per day. In another embodiment, the recommended starting dosage may be 0.1, 0.5, 0.6, 1, 1.2, 1.8, 2, 2.4, 3, 3.6, 4, or 5 mg per day. The dose may be escalated to 7, 8, 9 or 10 mg/day.

In a specific embodiment, an isotopologue of Compound A is administered in an amount of about 0.1 mg/day to patients with leukemia, including AML. In a particular embodiment, an isotopologue of Compound A is administered in an amount of about 1 mg/day to patients with leukemia, including AML. In a particular embodiment, an isotopologue of Compound A is administered in an amount of about 3 mg/day to patients with leukemia, including AML. In a particular embodiment, an isotopologue of Compound A is administered in an amount of about 4 mg/day to patients with leukemia, including AML. In a particular embodiment, an isotopologue of Compound A is provided herein can be administered in an amount of about 5 mg/day to patients with leukemia, including AML. In a particular embodiment, an isotopologue of Compound A is administered in an amount of about 6 mg/day to patients with leukemia, including AML. In a particular embodiment, an isotopologue of Compound A is administered in an amount of about 7 mg/day to patients with leukemia, including AML. In a particular embodiment, an isotopologue of Compound A is administered in an amount of about 10 mg/day to patients with leukemia, including AML.

In a specific embodiment, an isotopologue of Compound A is administered in an amount of about 0.1 mg/day to patients with MDS. In a particular embodiment, an isotopologue of Compound A is administered in an amount of about 1 mg/day to patients with MDS. In a particular embodiment, an isotopologue of Compound A is administered in an amount of about 3 mg/day to patients with MDS. In a particular embodiment, an isotopologue of Compound A is administered in an amount of about 4 mg/day to patients with MDS. In a particular embodiment, an isotopologue of Compound A is administered in an amount of about 5 mg/day to patients with MDS. In a particular embodiment, an isotopologue of Compound A is administered in an amount of about 6 mg/day to patients with MDS. In a particular embodiment, an isotopologue of Compound A is administered in an amount of about 7 mg/day to patients with MDS. In a particular embodiment, an isotopologue of Compound A is administered in an amount of about 10 mg/day to patients with MDS.

In certain embodiments, the therapeutically or prophylactically effective amount is from about 0.001 to about 20 mg/kg/day, from about 0.01 to about 15 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In certain embodiments, the amount of an isotopologue of Compound A administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

As used herein, the term “plasma concentration at steady state” is the concentration reached after a period of administration of a formulation provided herein. Once steady state is reached, there are minor peaks and troughs on the time dependent curve of the plasma concentration of the solid form.

In certain embodiments, the amount of an isotopologue of Compound A administered is sufficient to provide a maximum plasma concentration (peak concentration) of the compound, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In certain embodiments, the amount of an isotopologue of Compound A administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

In certain embodiments, the patient to be treated with one of the methods provided herein has not been treated with anti-cancer therapy prior to the administration of an isotopologue of Compound A provided herein. In certain embodiments, the patient to be treated with one of the methods provided herein has been treated with anti-cancer therapy prior to the administration of an isotopologue of Compound A provided herein. In certain embodiments, the patient to be treated with one of the methods provided herein has developed drug resistance to the anti-cancer therapy.

The methods provided herein encompass treating a patient regardless of patient's age, although some diseases or disorders are more common in certain age groups.

An isotopologue of Compound A provided herein can be delivered as a single dose such as, e.g., a single bolus injection, or overtime, such as, e.g., continuous infusion overtime or divided bolus doses over time. An isotopologue of Compound A can be administered repeatedly if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205-216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

An isotopologue of Compound A provided herein can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, is administered once or more than once each day, for example, for a period of time. The term “continuous” is intended to mean that a therapeutic compound, is administered daily for an uninterrupted period of at least 10 days to 52 weeks. The term “intermittent” or “intermittently” as used herein is intended to mean stopping and starting at either regular or irregular intervals. For example, intermittent administration of an isotopologue of Compound A is administration for one to six days per week, administration in cycles (e.g., daily administration for one to ten consecutive days of a 28 day cycle, then a rest period with no administration for rest of the 28 day cycle or daily administration for two to eight consecutive weeks, then a rest period with no administration for up to one week), or administration on alternate days. Cycling therapy with an isotopologue of Compound A is discussed elsewhere herein.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, an isotopologue of Compound A is administered once a day. In another embodiment, an isotopologue of Compound A is administered twice a day. In yet another embodiment, an isotopologue of Compound A provided herein is administered three times a day. In still another embodiment, an isotopologue of Compound A provided herein is administered four times a day.

In certain embodiments, an isotopologue of Compound A provided herein is administered once per day from one day to six months, from one week to three months, from one week to four weeks, from one week to three weeks, or from one week to two weeks. In certain embodiments, an isotopologue of Compound A provided herein is administered once per day for one week, two weeks, three weeks, or four weeks. In one embodiment, an isotopologue of Compound A provided herein is administered once per day for 1 day. In one embodiment, an isotopologue of Compound A provided herein is administered once per day for 2 days. In one embodiment, an isotopologue of Compound A provided herein is administered once per day for 3 days. In one embodiment, an isotopologue of Compound A provided herein is administered once per day for 4 days. In one embodiment, an isotopologue of Compound A provided herein is administered once per day for 5 days. In one embodiment, an isotopologue of Compound A provided herein is administered once per day for 6 days. In one embodiment, an isotopologue of Compound A provided herein is administered once per day for one week. In one embodiment, an isotopologue of Compound A provided herein is administered once per day for up to 10 days. In another embodiment, an isotopologue of Compound A provided herein is administered once per day for two weeks. In yet another embodiment, an isotopologue of Compound A provided herein is administered once per day for three weeks. In still another embodiment, an isotopologue of Compound A provided herein is administered once per day for four weeks.

In one embodiment the present invention is directed to the isotopologues provided herein for use in any of the methods provided herein.

Combination Therapy

In one embodiment, provided herein is a method of treating, preventing, ameliorating and/or managing cancer, comprising administering to a patient an isotopologue of Compound A in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors, and optionally in combination with radiation therapy, blood transfusions, or surgery. Examples of second active agents are disclosed herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a patient with a disease or disorder. A first therapy (e.g., a prophylactic or therapeutic agent such an isotopologue of Compound A provided herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

In one embodiment, administration of an isotopologue of Compound A provided herein, and one or more second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the cancer being treated.

The route of administration of an isotopologue of Compound A provided herein, is independent of the route of administration of a second therapy. Thus, in one embodiment, an isotopologue of Compound A provided herein, is administered intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, an isotopologue of Compound A provided herein, and a second therapy are administered by the same mode of administration, by IV. In another embodiment, an isotopologue of Compound A provided herein, is administered by one mode of administration, e.g., by IV, whereas the second agent (an anti-cancer agent) is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated and/or managed, the severity and stage of disease, and the amount of an isotopologue of Compound A and any optional additional active agents concurrently administered to the patient.

One or more second active ingredients or agents can be used together with an isotopologue of Compound A in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins. Proteins that are particularly useful in the methods and compositions provided herein include proteins that stimulate the survival and/or proliferation of hematopoietic precursor cells and immunologically active poietic cells in vitro or in vivo. Other useful proteins stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

In certain embodiments, GM-CSF, G-CSF, SCF or EPO is administered subcutaneously during about five days in a four or six week cycle in an amount ranging from about 1 to about 750 mg/m²/day, from about 25 to about 500 mg/m²/day, from about 50 to about 250 mg/m²/day, or from about 50 to about 200 mg/m²/day. In certain embodiments, GM-CSF may be administered in an amount of from about 60 to about 500 mcg/m² intravenously over 2 hours or from about 5 to about 12 mg/m²/day subcutaneously. In certain embodiments, G-CSF may be administered subcutaneously in an amount of about 1 mcg/kg/day initially and can be adjusted depending on rise of total granulocyte counts. The maintenance dose of G-CSF may be administered in an amount of about 300 (in smaller patients) or 480 mcg subcutaneously. In certain embodiments, EPO may be administered subcutaneously in an amount of 10,000 Unit 3 times per week.

Particular proteins that can be used in the methods and compositions include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.).

Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; the entireties of which are incorporated herein by reference.

Also provided for use in combination with an isotopologue of Compound A, are native, naturally occurring, and recombinant proteins. Further encompassed are mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with an isotopologue of Compound A provided herein, include monoclonal and polyclonal antibodies. Examples of antibodies include, but are not limited to, trastuzumab (Herceptin®), rituximab (Rituxan®), bevacizumab (Avastin™), pertuzumab (Omnitarg™), tositumomab (Bexxar®), edrecolomab (Panorex®), and G250. An isotopologue of Compound A can also be combined with, or used in combination with, anti-TNF-α antibodies, and/or anti-EGFR antibodies, such as, for example, Erbitux® or panitumumab.

Large molecule active agents may be administered in the form of anti-cancer vaccines. For example, vaccines that secrete, or cause the secretion of, cytokines such as IL-2, G-CSF, and GM-CSF can be used in the methods and pharmaceutical compositions provided. See, e.g., Emens, L. A., et al., Curr. Opinion Mol. Ther. 3(1):77-84 (2001).

Second active agents that are small molecules can also be used to alleviate adverse effects associated with the administration of an isotopologue of Compound A provided herein. However, like some large molecules, many are believed to be capable of providing a synergistic effect when administered with (e.g., before, after or simultaneously) an isotopologue of Compound A provided herein. Examples of small molecule second active agents include, but are not limited to, anti-cancer agents, antibiotics, immunosuppressive agents, and steroids.

In certain embodiments, the second agent is an HSP inhibitor, a proteasome inhibitor, a FLT3 inhibitior or an mTOR inhibitor. In some embodiments, the mTOR inhibitor is a mTOR kinase inhibitor.

Examples of anti-cancer agents to be used within the methods or compositions described herein include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; clofarabine; crisnatol mesylate; cyclophosphamide; Ara-C; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; omacetaxine; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sorafenib; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs to be included within the methods herein include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; Ara-C ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®); imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In certain embodiments, the second agent is selected from one or more checkpoint inhibitors. In one embodiment, one checkpoint inhibitor is used in combination with an isotopologue of Compound A in the methods provided herein. In another embodiment, two checkpoint inhibitors are used in combination with an isotopologue of Compound A in connection with the methods provided herein. In yet another embodiment, three or more checkpoint inhibitors are used in combination with an isotopologue of Compound A in connection with the methods provided herein.

As used herein, the term “immune checkpoint inhibitor” or “checkpoint inhibitor” refers to molecules that totally or partially reduce, inhibit, interfere with or modulate one or more checkpoint proteins. Without being limited by a particular theory, checkpoint proteins regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In one embodiment, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In one embodiment, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In one embodiment, the checkpoint inhibitor is a PD-1 inhibitor. In one embodiment, the PD-1 inhibitor is an anti-PD-1 antibody. In one embodiment, the anti-PD-1 antibody is nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein.

In one embodiment, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitors is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitors is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhIL-15).

In one embodiment, the checkpoint inhibitor is an IDO inhibitor. In one embodiment, the IDO inhibitor is INCB024360. In another embodiment, the IDO inhibitor is indoximod.

In certain embodiments, the combination therapies provided herein include two or more of the checkpoint inhibitors described herein (including checkpoint inhibitors of the same or different class). Moreover, the combination therapies described herein can be used in combination with second active agents as described herein where appropriate for treating diseases described herein and understood in the art.

In certain embodiments, an isotopologue of Compound A can be used in combination with one or more immune cells expressing one or more chimeric antigen receptors (CARs) on their surface (e.g., a modified immune cell). Generally, CARs comprise an extracellular domain from a first protein e.g., an antigen-binding protein), a transmembrane domain, and an intracellular signaling domain. In certain embodiments, once the extracellular domain binds to a target protein such as a tumor-associated antigen (TAA) or tumor-specific antigen (TSA), a signal is generated via the intracellular signaling domain that activates the immune cell, e.g., to target and kill a cell expressing the target protein.

Extracellular domains: The extracellular domains of the CARs bind to an antigen of interest. In certain embodiments, the extracellular domain of the CAR comprises a receptor, or a portion of a receptor, that binds to said antigen. In certain embodiments, the extracellular domain comprises, or is, an antibody or an antigen-binding portion thereof. In specific embodiments, the extracellular domain comprises, or is, a single chain Fv (scFv) domain. The single-chain Fv domain can comprise, for example, a V_{L} linked to V_{H} by a flexible linker, wherein said V_{L} and V_{H} are from an antibody that binds said antigen.

In certain embodiments, the antigen recognized by the extracellular domain of a polypeptide described herein is a tumor-associated antigen (TAA) or a tumor-specific antigen (TSA). In various specific embodiments, the tumor-associated antigen or tumor-specific antigen is, without limitation, Her2, prostate stem cell antigen (PSCA), alpha-fetoprotein (AFP), carcinoembryonic antigen (CEA), cancer antigen-125 (CA-125), CA19-9, calretinin, MUC-1, B cell maturation antigen (BCMA), epithelial membrane protein (EMA), epithelial tumor antigen (ETA), tyrosinase, melanoma-24 associated antigen (MAGE), CD19, CD22, CD27, CD30, CD34, CD45, CD70, CD99, CD117, EGFRvIII (epidermal growth factor variant III), mesothelin, PAP (prostatic acid phosphatase), prostein, TARP (T cell receptor gamma alternate reading frame protein), Trp-p8, STEAPI (six-transmembrane epithelial antigen of the prostate 1), chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-I), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein. In certain other embodiments, the TAA or TSA recognized by the extracellular domain of a CAR is integrin avP3 (CD61), galactin, or Ral-B.

In certain embodiments, the TAA or TSA recognized by the extracellular domain of a CAR is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXBI, SPA17, SSX, SYCPI, or TPTE.

In certain other embodiments, the TAA or TSA recognized by the extracellular domain of a CAR is a carbohydrate or ganglioside, e.g., fuc-GMI, GM2 (oncofetal antigen-immunogenic-1; OFA-I-1); GD2 (OFA-I-2), GM3, GD3, and the like.

In certain other embodiments, the TAA or TSA recognized by the extracellular domain of a CAR is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-All, hsp70-2, KIAA0205, Mart2, Mum-1, 2, and 3, neo-PAP, myosin class I, OS-9, pml-RARα fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel17), tyrosinase, TRP-1, TRP-2, MAGE-1, MAGE-3, RAGE, GAGE-1, GAGE-2, p15(58), RAGE, SCP-1, Hom/Mel-40, PRAME, p53, HRas, HER-2/neu, E2A-PRL, H4-RET, IGH-IGK, MYL-RAR, human papillomavirus (HPV) antigens E6 and E7, TSP-180, MAGE-4, MAGE-5, MAGE-6, pi85erbB2, pi80erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, C0-029, FGF-5, G250, Ga733 (EpCAM), HTgp-175, M344, MA-50, MG7-Ag, MOV18, NB\70K, NY-C0-1, RCAS1, SDCCAGI6, TA-90, TAAL6, TAG72, TLP, or TPS.

In various specific embodiments, the tumor-associated antigen or tumor-specific antigen is an AML-related tumor antigens, as described in S. Anguille et al, Leukemia (2012), 26, 2186-2196.

Other tumor-associated and tumor-specific antigens are known to those in the art.

Receptors, antibodies, and scFvs that bind to TSAs and TAAs, useful in constructing chimeric antigen receptors, are known in the art, as are nucleotide sequences that encode them.

In certain specific embodiments, the antigen recognized by the extracellular domain of a chimeric antigen receptor is an antigen not generally considered to be a TSA or a TAA, but which is nevertheless associated with tumor cells, or damage caused by a tumor. In certain embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the antigen is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the antigen is a DAMP, e.g., a heat shock protein, chromatin-associated protein high mobility group box 1 (HMGB 1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

Transmembrane domain: In certain embodiments, the extracellular domain of the CAR is joined to the transmembrane domain of the polypeptide by a linker, spacer or hinge polypeptide sequence, e.g., a sequence from CD28 or a sequence from CTLA4. The transmembrane domain can be obtained or derived from the transmembrane domain of any transmembrane protein, and can include all or a portion of such transmembrane domain. In specific embodiments, the transmembrane domain can be obtained or derived from, e.g., CD8, CD16, a cytokine receptor, and interleukin receptor, or a growth factor receptor, or the like.

Intracellular signaling domains: In certain embodiments, the intracellular domain of a CAR is or comprises an intracellular domain or motif of a protein that is expressed on the surface of T cells and triggers activation and/or proliferation of said T cells. Such a domain or motif is able to transmit a primary antigen-binding signal that is necessary for the activation of a T lymphocyte in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD3ζ) or ITAM-containing portions thereof. In a specific embodiment, the intracellular domain is a CD3ζ intracellular signaling domain. In other specific embodiments, the intracellular domain is from a lymphocyte receptor chain, a TCR/CD3 complex protein, an Fe receptor subunit or an IL-2 receptor subunit. In certain embodiments, the CAR additionally comprises one or more co-stimulatory domains or motifs, e.g., as part of the intracellular domain of the polypeptide. The one or more co-stimulatory domains or motifs can be, or can comprise comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof.

The CAR may also comprise a T cell survival motif. The T cell survival motif can be any polypeptide sequence or motif that facilitates the survival of the T lymphocyte after stimulation by an antigen. In certain embodiments, the T cell survival motif is, or is derived from, CD3, CD28, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor β (TGFβ) receptor.

The modified immune cells expressing the CARs can be, e.g., T lymphocytes (T cells, e.g., CD4+ T cells or CD8+ T cells), cytotoxic lymphocytes (CTLs) or natural killer (NK) cells. T lymphocytes used in the compositions and methods provided herein may be naive T lymphocytes or MHC-restricted T lymphocytes. In certain embodiments, the T lymphocytes are tumor infiltrating lymphocytes (TILs). In certain embodiments, the T lymphocytes have been isolated from a tumor biopsy, or have been expanded from T lymphocytes isolated from a tumor biopsy. In certain other embodiments, the T cells have been isolated from, or are expanded from T lymphocytes isolated from, peripheral blood, cord blood, or lymph. Immune cells to be used to generate modified immune cells expressing a CAR can be isolated using art-accepted, routine methods, e.g., blood collection followed by apheresis and optionally antibody-mediated cell isolation or sorting.

The modified immune cells are preferably autologous to an individual to whom the modified immune cells are to be administered. In certain other embodiments, the modified immune cells are allogeneic to an individual to whom the modified immune cells are to be administered. Where allogeneic T lymphocytes or NK cells are used to prepare modified T lymphocytes, it is preferable to select T lymphocytes or NK cells that will reduce the possibility of graft-versus-host disease (GVHD) in the individual. For example, in certain embodiments, virus-specific T lymphocytes are selected for preparation of modified T lymphocytes; such lymphocytes will be expected to have a greatly reduced native capacity to bind to, and thus become activated by, any recipient antigens. In certain embodiments, recipient-mediated rejection of allogeneic T lymphocytes can be reduced by co-administration to the host of one or more immunosuppressive agents, e.g., cyclosporine, tacrolimus, sirolimus, cyclophosphamide, or the like.

T lymphocytes, e.g., unmodified T lymphocytes, or T lymphocytes expressing CD3 and CD28, or comprising a polypeptide comprising a CD3 signaling domain and a CD28 co-stimulatory domain, can be expanded using antibodies to CD3 and CD28, e.g., antibodies attached to beads; see, e.g., U.S. Pat. Nos. 5,948,893; 6,534,055; 6,352,694; 6,692,964; 6,887,466; and 6,905,681.

The modified immune cells, e.g., modified T lymphocytes, can optionally comprise a “suicide gene” or “safety switch” that enables killing of substantially all of the modified immune cells when desired. For example, the modified T lymphocytes, in certain embodiments, can comprise an HSV thymidine kinase gene (HSV-TK), which causes death of the modified T lymphocytes upon contact with gancyclovir. In another embodiment, the modified T lymphocytes comprise an inducible caspase, e.g., an inducible caspase 9 (icaspase9), e.g., a fusion protein between caspase 9 and human FK506 binding protein allowing for dimerization using a specific small molecule pharmaceutical. See Straathof et al., Blood 105(11):4247-4254 (2005).

Specific second active agents useful in the methods or compositions include, but are not limited to, rituximab, oblimersen (Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Arisa, taxol, taxotere, fluorouracil, leucovorin, irinotecan, xeloda, interferon alpha, pegylated interferon alpha (e.g., PEG INTRON-A), capecitabine, cisplatin, thiotepa, fludarabine, carboplatin, liposomal daunorubicin, Ara-C, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

In certain embodiments of the methods provided herein, use of a second active agent in combination with an isotopologue of Compound A provided herein, may be modified or delayed during or shortly following administration of an isotopologue of Compound A provided herein, as deemed appropriate by the practitioner of skill in the art. In certain embodiments, subjects being administered an isotopologue of Compound A provided herein, alone or in combination with other therapies may receive supportive care including antiemetics, myeloid growth factors, and transfusions of platelets, when appropriate. In some embodiments, subjects being administered an isotopologue of Compound A provided herein, may be administered a growth factor as a second active agent according to the judgment of the practitioner of skill in the art. In some embodiments, provided is administration of an isotopologue of Compound A provided herein, in combination with erythropoietin or darbepoetin (Aranesp).

In one aspect, provided herein is a method of treating, preventing, managing, and/or ameliorating locally advanced or metastatic transitional cell bladder cancer comprising administering an isotopologue of Compound A with gemcitabine, cisplatinum, 5-fluorouracil, mitomycin, methotrexate, vinblastine, doxorubicin, carboplatin, thiotepa, paclitaxel, docetaxel, atezolizumab, avelumab, durvalumab, keytruda (pembrolizumab) and/or nivolumab.

In one aspect, methods of treating, preventing, managing, and/or ameliorating a cancer provided herein comprise administering an isotopologue of Compound A in combination with a second active ingredient as follows: temozolomide to pediatric patients with relapsed or progressive brain tumors or recurrent neuroblastoma; celecoxib, etoposide and cyclophosphamide for relapsed or progressive CNS cancer; temodar to patients with recurrent or progressive meningioma, malignant meningioma, hemangiopericytoma, multiple brain metastases, relapsed brain tumors, or newly diagnosed glioblastoma multiforms; irinotecan to patients with recurrent glioblastoma; carboplatin to pediatric patients with brain stem glioma; procarbazine to pediatric patients with progressive malignant gliomas; cyclophosphamide to patients with poor prognosis malignant brain tumors, newly diagnosed or recurrent glioblastoma multiforms; Gliadel® for high grade recurrent malignant gliomas; temozolomide and tamoxifen for anaplastic astrocytoma; or topotecan for gliomas, glioblastoma, anaplastic astrocytoma or anaplastic oligodendroglioma.

In one aspect, methods of treating, preventing, managing, and/or ameliorating a metastatic breast cancer provided herein comprise administering an isotopologue of Compound A with methotrexate, cyclophosphamide, capecitabine, 5-fluorouracil, taxane, temsirolimus, ABRAXANE® (paclitaxel protein-bound particles for injectable suspension) (albumin-bound)), lapatinib, herceptin, pamidronate disodium, eribulin mesylate, everolimus, gemcitabine, palbociclib, ixabepilone, kadcyla, pertuzumab, theotepa, anastrozole, docetaxel, doxorubicin hydrochloride, epirubicin hydrochloride, toremifene, fulvestrant, goserelin acetate, ribociclib, megestrol acetate, vinblastin, aromatase inhibitors, such as letrozole, exemestane, selective estrogen modulators, estrogen receptor antagonists, anthracyclines, emtansine, and/or pexidartinib to patients with metastatic breast cancer.

In one aspect, methods of treating, preventing, managing, and/or ameliorating a neuroendocrine tumors provided herein comprise administering an isotopologue of Compound A with at least one of everolimus, avelumab, sunitinib, nexavar, leucovorin, oxaliplatin, temozolomide, capecitabine, bevacizumab, doxorubicin (Adriamycin), fluorouracil (Adrucil, 5-fluorouracil), streptozocin (Zanosar), dacarbazine, sandostatin, lanreotide, and/or pasireotide to patients with neuroendocrine tumors.

In one aspect, methods of treating, preventing, managing, and/or ameliorating a metastatic breast cancer provided herein comprise administering an isotopologue of Compound A with methotrexate, gemcitabine, cisplatin, cetuximab, 5-fluorouracil, bleomycin, docetaxel, carboplatin, hydroxyurea, pembrolizumab and/or nivolumab to patients with recurrent or metastatic head or neck cancer.

In one aspect, methods of treating, preventing, managing, and/or ameliorating a pancreatic cancer provided herein comprise administering an isotopologue of Compound A with gemcitabine, ABRAXANE®, 5-fluorouracil, afinitor, irinotecan, mitomycin C, sunitinib, sunitinibmalate, and/or tarceva to patients with pancreatic cancer.

In one aspect, methods of treating, preventing, managing, and/or ameliorating a colon or rectal cancer provided herein comprise administering an isotopologue of Compound A with ARISA®, avastatin, oxaliplatin, 5-fluorouracil, irinotecan, capecitabine, cetuximab, ramucirumab, panitumumab, bevacizumab, leucovorin calcium, lonsurf, regorafenib, ziv-aflibercept, taxol, and/or taxotere.

In one aspect, methods of treating, preventing, managing, and/or ameliorating a refractory colorectal cancer provided herein comprise administering an isotopologue of Compound A with capecitabine and/or vemurafenib to patients with refractory colorectal cancer, or patients who fail first line therapy or have poor performance in colon or rectal adenocarcinoma.

In one aspect, methods of treating, preventing, managing, and/or ameliorating a colorectal cancer provided herein comprise administering an isotopologue of Compound A with fluorouracil, leucovorin, and/or irinotecan to patients with colorectal cancer, including stage 3 and stage 4, or to patients who have been previously treated for metastatic colorectal cancer.

In certain embodiments, an isotopologue of Compound A provided herein is administered to patients with refractory colorectal cancer in combination with capecitabine, xeloda, and/or irinotecan.

In certain embodiments, an isotopologue of Compound A provided herein is administered with capecitabine and irinotecan to patients with refractory colorectal cancer or to patients with unresectable or metastatic colorectal carcinoma.

In one aspect, the methods provided herein comprise administering an isotopologue of Compound A with interferon alpha or capecitabine to patients with unresectable or metastatic hepatocellular carcinoma; or with cisplatin and thiotepa, or with sorafenib tosylate to patients with primary or metastatic liver cancer.

In one aspect, the methods provided herein comprise administering an isotopologue of Compound A with doxorubicin, paclitaxel, vinblastine, pegylated interferon alpha and/or recombinant interferon alpha-2b to patients with Kaposi's sarcoma.

In one aspect, the methods provided herein comprise administering an isotopologue of Compound A with at least one of enasidenib, arsenic trioxide, fludarabine, carboplatin, daunorubicin, cyclophosphamide, cytarabine, doxorubicin, idarubicin, mitoxantrone hydrochloride, thioguanine, vincritine, midostaurin and/or topotecan to patients with acute myeloid leukemia, including refractory or relapsed or high-risk acute myeloid leukemia.

In one aspect, the methods provided herein comprise administering an isotopologue of Compound A with at least one of enasidenib, liposomal daunorubicin, topotecan and/or cytarabine to patients with unfavorable karyotype acute myeloblastic leukemia.

In one aspect, the methods provided herein comprise administering an isotopologue of Compound A with methotrexate, mechlorethamine hydrochloride, afatinib dimaleate, pemetrexed, bevacizumab, carboplatin, cisplatin, ceritinib, crizotinib, ramucirumab, pembrolizumab, docetaxel, vinorelbine tartrate, gemcitabine, ABRAXANE®, erlotinib, geftinib, irinotecan, everolimus, alectinib, brigatinib, nivolumab, osimertinib, atezolizumab, necitumumab and/or to patients with non-small cell lung cancer.

In one aspect, the methods provided herein comprise administering an isotopologue of Compound A with carboplatin and irinotecan to patients with non-small cell lung cancer.

In one aspect, the methods provided herein comprise administering an isotopologue of Compound A with doxetaxol to patients with non-small cell lung cancer who have been previously treated with carbo/etoposide and radiotherapy.

In one aspect, the methods provided herein comprise administering an isotopologue of Compound A with carboplatin and/or taxotere, or in combination with carboplatin, pacilitaxel and/or thoracic radiotherapy to patients with non-small cell lung cancer.

In one aspect, the methods provided herein comprise administering an isotopologue of Compound A with taxotere to patients with stage IIIB or IV non-small cell lung cancer.

In one aspect, the methods provided herein comprise administering an isotopologue of Compound A with oblimersen (Genasense®), methotrexate, mechlorethamine hydrochloride, etoposide, topotecan and/or doxorubicin to patients with small cell lung cancer.

In one aspect, the methods provided herein comprise administering an isotopologue of Compound A with ABT-737 (Abbott Laboratories) and/or obatoclax (GX15-070) to patients with lymphoma and other blood cancers.

In one aspect, the methods provided herein comprise administering an isotopologue of Compound A with a second active ingredient such as vinblastine or fludarabine adcetris, ambochlorin, becenum, bleomycin, brentuximab vedotin, carmustinem chlorambucil, cyclophosphamide, dacarbazine, doxorubicin, lomustine, matulane, mechlorethamine hydrochloride, prednisone, procarbazine hydrochloride, vincristine, methotrexate, nelarabin, belinostat, bendamustine HCl, tositumomab, and iodine 131 tositumomab, denileukin diftitox, dexamethasone, pralatrexate, prelixafor, obinutuzumab, ibritumomab, tiuxefan, ibritinib, idelasib, intron A, romidepsin, lenalidomide, rituximab, and/or vorinostat to patients with various types of lymphoma, including, but not limited to, Hodgkin's lymphoma, non-Hodgkin's lymphoma, cutaneous T-Cell lymphoma, cutaneous B-Cell lymphoma, diffuse large B-Cell lymphoma or relapsed or refractory low grade follicular lymphoma.

In one aspect, the methods provided herein comprise administering an isotopologue of Compound A with taxotere, dabrafenib, imlygic, ipilimumab, pembrolizumab, nivolumab, trametinib, vemurafenib, talimogene laherparepvec, IL-2, IFN, GM-CSF, and/or dacarbazine, aldesleukin, cobimetinib, Intron A®, peginterferon Alfa-2b, and/or trametinib to patients with various types or stages of melanoma.

In one aspect, the methods provided herein comprise administering an isotopologue of Compound A with vinorelbine or pemetrexed disodium to patients with malignant mesothelioma, or stage IIIB non-small cell lung cancer with pleural implants or malignant pleural effusion mesothelioma syndrome.

In one aspect, the methods of treating patients with various types or stages of multiple myeloma provided herein comprise administering an isotopologue of Compound A with dexamethasone, zoledronic acid, palmitronate, GM-CSF, biaxin, vinblastine, melphalan, busulphan, cyclophosphamide, IFN, prednisone, bisphosphonate, celecoxib, arsenic trioxide, PEG INTRON-A, vincristine, becenum, bortezomib, carfilzomib, doxorubicin, panobinostat, lenalidomide, pomalidomide, thalidomide, mozobil, carmustine, daratumumab, elotuzumab, ixazomib citrate, plerixafor or a combination thereof.

In certain embodiments, an isotopologue of Compound A provided herein is administered to patients with various types or stages of multiple myeloma in combination with chimeric antigen receptor (CAR) T-cells.

In certain embodiments, an isotopologue of Compound A provided herein is administered to patients with relapsed or refractory multiple myeloma in combination with doxorubicin (Doxil®), vincristine and/or dexamethasone (Decadron®).

In certain embodiments, the methods provided herein comprise administering an isotopologue of Compound A to patients with various types or stages of ovarian cancer such as peritoneal carcinoma, papillary serous carcinoma, refractory ovarian cancer or recurrent ovarian cancer, in combination with taxol, carboplatin, doxorubicin, gemcitabine, cisplatin, xeloda, paclitaxel, dexamethasone, avastin, cyclophosphamide, topotecan, olaparib, thiotepa, melphalan, niraparib tosylate monohydrate, rubraca or a combination thereof.

In certain embodiments, the methods provided herein comprise administering an isotopologue of Compound A to patients with various types or stages of prostate cancer, in combination with xeloda, 5 FU/LV, gemcitabine, irinotecan plus gemcitabine, cyclophosphamide, vincristine, dexamethasone, GM-CSF, celecoxib, taxotere, ganciclovir, paclitaxel, adriamycin, docetaxel, estramustine, Emcyt, denderon, zytiga, bicalutamide, cabazitaxel, degarelix, enzalutamide, zoladex, leuprolide acetate, mitoxantrone hydrochloride, prednisone, sipuleucel-T, radium 223 dichloride, or a combination thereof.

In certain embodiments, the methods provided herein comprise administering an isotopologue of Compound A to patients with various types or stages of renal cell cancer, in combination with capecitabine, IFN, tamoxifen, IL-2, GM-CSF, Celebrex®, flutamide, goserelin acetate, nilutamide or a combination thereof.

In certain embodiments, the methods provided herein comprise administering an isotopologue of Compound A to patients with various types or stages of gynecologic, uterus or soft tissue sarcoma cancer in combination with IFN, dactinomycin, doxorubicin, imatinib mesylate, pazopanib, hydrochloride, trabectedin, eribulin mesylate, olaratumab, a COX-2 inhibitor such as celecoxib, and/or sulindac.

In one aspect, the methods provided herein comprise administering an isotopologue of Compound A to patients with various types or stages of solid tumors in combination with celecoxib, etoposide, cyclophosphamide, docetaxel, apecitabine, IFN, tamoxifen, IL-2, GM-CSF, or a combination thereof.

In one aspect, the methods provided herein comprise administering an isotopologue of Compound A to patients with scleroderma or cutaneous vasculitis in combination with celebrex, etoposide, cyclophosphamide, docetaxel, apecitabine, IFN, tamoxifen, IL-2, GM-CSF, or a combination thereof.

In one aspect, the methods provided herein comprise administering an isotopologue of Compound A to patients with MDS in combination with azacitidine, cytarabine, daunorubicin, decitabine, idarubicin, lenalidomide, enasidenib, or a combination thereof.

In one aspect, the methods provided herein comprise administering an isotopologue of Compound A to patients with hematological cancer in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one aspect, the methods provided herein comprise administering an isotopologue of Compound A to patients with leukemia in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one aspect, the methods provided herein comprise administering an isotopologue of Compound A to patients with AML in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one aspect, the methods provided herein comprise administering an isotopologue of Compound A to patients with leukemia in combination with an mTOR inhibitor. In certain embodiments, the mTOR inhibitor is selected from everolimus, MLN-0128 and AZD8055. In other aspects, the methods provided herein comprise administering an isotopologue of Compound A to patients with leukemia in combination with an mTOR kinase inhibitor. In certain embodiments, the mTOR kinase inhibitor is selected from 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223) and 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, an isotopologue of Compound A is administered to patients with leukemia in combination with 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223). In certain embodiments, an isotopologue of Compound A is administered to patients with leukemia in combination with 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, an isotopologue of Compound A is administered to patients with leukemia in combination with everolimus. In certain embodiments, an isotopologue of Compound A is administered to patients with leukemia in combination with MLN-0128. In certain embodiments, an isotopologue of Compound A is administered to patients with leukemia in combination with AZD8055.

In one aspect, the methods provided herein comprise administering an isotopologue of Compound A to patients with AML in combination with an mTOR inhibitor. In certain embodiments, the mTOR inhibitor is selected from everolimus, MLN-0128 and AZD8055. In other aspects, the methods provided herein comprise administering an isotopologue of Compound A to patients with AML in combination with an mTOR kinase inhibitor. In certain embodiments, the mTOR kinase inhibitor is selected from 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223) and 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, an isotopologue of Compound A is administered to patients with AML in combination with 1-ethyl-7-(2-methyl-6-(1H-1,2,4-triazol-3-yl)pyridin-3-yl)-3,4-dihydropyrazino[2,3-b]pyrazin-2(1H)-one. In certain embodiments, an isotopologue of Compound A is administered to patients with AML in combination with everolimus. In certain embodiments, an isotopologue of Compound A is administered to patients with AML in combination with MLN-0128. In certain embodiments, an isotopologue of Compound A is administered to patients with AML in combination with AZD8055.

In one aspect, the methods provided herein comprise administering an isotopologue of Compound A to patients with MPN in combination with a JAK inhibitor. In one aspect the JAK inhibitor is selected from a JAK1 inhibitor, a JAK2 inhibitor and a JAK3 inhibitor. In certain embodiments, the JAK inhibitor is selected from momelotinib, filgotinib, decernotinib, barcitinib, ruxolitinib, fedratinib, NS-018 and pacritinib. In certain embodiments, an isotopologue of Compound A is administered to patients with MPN in combination with momelotinib. In certain embodiments, an isotopologue of Compound A is administered to patients with MPN in combination with filgotinib. In certain embodiments, an isotopologue of Compound A is administered to patients with MPN in combination with decernotinib. In certain embodiments, an isotopologue of Compound A is administered to patients with MPN in combination with barcitinib. In certain embodiments, an isotopologue of Compound A is administered to patients with MPN in combination with ruxolitinib. In certain embodiments, an isotopologue of Compound A is administered to patients with MPN in combination with fedratinib. In certain embodiments, an isotopologue of Compound A is administered to patients with MPN in combination with NS-018. In certain embodiments, an isotopologue of Compound A is administered to patients with MPN in combination with pacritinib. In certain embodiments, the patient carries a JAK2^{V617F} mutation.

In one aspect, the methods provided herein comprise administering an isotopologue of Compound A to patients with leukemia in combination with a JAK inhibitor. In one aspect the JAK inhibitor is selected from a JAK1 inhibitor, a JAK2 inhibitor and a JAK3 inhibitor. In certain embodiments, the JAK inhibitor is selected from momelotinib, filgotinib, decernotinib, barcitinib, ruxolitinib, fedratinib, NS-018 and pacritinib. In certain embodiments, an isotopologue of Compound A is administered to patients with leukemia in combination with momelotinib. In certain embodiments, an isotopologue of Compound A is administered to patients with leukemia in combination with filgotinib. In certain embodiments, an isotopologue of Compound A is administered to patients with leukemia in combination with decernotinib. In certain embodiments, an isotopologue of Compound A is administered to patients with leukemia in combination with barcitinib. In certain embodiments, an isotopologue of Compound A is administered to patients with leukemia in combination with ruxolitinib. In certain embodiments, an isotopologue of Compound A is administered to patients with leukemia in combination with fedratinib. In certain embodiments, an isotopologue of Compound A is administered to patients with leukemia in combination with NS-018. In certain embodiments, an isotopologue of Compound A is administered to patients with leukemia in combination with pacritinib. In certain embodiments, the patient carries a JAK2^{V617F} mutation.

In one aspect, the methods provided herein comprise administering an isotopologue of Compound A to patients with AML in combination with a JAK inhibitor. In one aspect the JAK inhibitor is selected from a JAK1 inhibitor, a JAK2 inhibitor and a JAK3 inhibitor. In certain embodiments, the JAK inhibitor is selected from momelotinib, filgotinib, decernotinib, barcitinib, ruxolitinib, fedratinib, NS-018 and pacritinib. In certain embodiments, an isotopologue of Compound A is administered to patients with ANL in combination with momelotinib. In certain embodiments, an isotopologue of Compound A is administered to patients with ANL in combination with filgotinib. In certain embodiments, an isotopologue of Compound A is administered to patients with AML in combination with decernotinib. In certain embodiments, an isotopologue of Compound A is administered to patients with AML in combination with barcitinib. In certain embodiments, an isotopologue of Compound A is administered to patients with AML in combination with ruxolitinib. In certain embodiments, an isotopologue of Compound A is administered to patients with AML in combination with fedratinib. In certain embodiments, an isotopologue of Compound A is administered to patients with AML in combination with NS-018. In certain embodiments, an isotopologue of Compound A is administered to patients with AML in combination with pacritinib. In certain embodiments, the patient carries a JAK2^{V617F} mutation.

In one aspect, the methods provided herein comprise administering an isotopologue of Compound A to patients with leukemia in combination with a FLT3 kinase inhibitor. In certain embodiments, the FLT3 kinase inhibitor is selected from quizartinib, sunitinib, sunitinib malate, midostaurin, pexidartinib, lestaurtinib, tandutinib, and crenolanib. In certain embodiments, an isotopologue of Compound A is administered to patients with leukemia in combination with quizartinib. In certain embodiments, an isotopologue of Compound A is administered to patients with leukemia in combination with sunitinib. In certain embodiments, an isotopologue of Compound A is administered to patients with leukemia in combination with midostaurin. In certain embodiments, an isotopologue of Compound A is administered to patients with leukemia in combination with pexidartinib. In certain embodiments, an isotopologue of Compound A is administered to patients with leukemia in combination with lestaurtinib. In certain embodiments, an isotopologue of Compound A is administered to patients with leukemia in combination with tandutinib. In certain embodiments, an isotopologue of Compound A is administered to patients with leukemia in combination with crenolanib. In certain embodiments, the patient carries a FLT3-ITD mutation.

In one aspect, the methods provided herein comprise administering an isotopologue of Compound A to patients with AML in combination with a FLT3 kinase inhibitor. In certain embodiments, the FLT3 kinase inhibitor is selected from quizartinib, sunitinib, sunitinib malate, midostaurin, pexidartinib, lestaurtinib, tandutinib, quizartinib and crenolanib. In certain embodiments, an isotopologue of Compound A is administered to patients with AML in combination with quizartinib. In certain embodiments an isotopologue of Compound A is administered to patients with AML in combination with sunitinib. In certain embodiments, an isotopologue of Compound A is administered to patients with AML in combination with midostaurin. In certain embodiments, an isotopologue of Compound A is administered to patients with AML in combination with pexidartinib. In certain embodiments, an isotopologue of Compound A is administered to patients with AML in combination with lestaurtinib. In certain embodiments, an isotopologue of Compound A is administered to patients with AML in combination with tandutinib. In certain embodiments, an isotopologue of Compound A is administered to patients with AML in combination with crenolanib. In certain embodiments, the patient carries a FLT3-ITD mutation.

In certain embodiments, an isotopologue of Compound A is administered to patients with leukemia in combination with a spliceosome inhibitor. In certain embodiments, an isotopologue of Compound A is administered to patients with AML in combination with a spliceosome inhibitor. In certain embodiments, the spliceosome inhibitor is pladienolide B.

In one aspect, the methods provided herein comprise administering an isotopologue of Compound A to patients with leukemia in combination with an SMG1 kinase inhibitor. In one aspect, the methods provided herein comprise administering an isotopologue of Compound A to patients with AML in combination with an SMG1 kinase inhibitor. In certain embodiments, the SMG1 inhibitor is 1-ethyl-7-(2-methyl-6-(1H-1,2,4-triazol-3-yl)pyridin-3-yl)-3,4-dihydropyrazino[2,3-b]pyrazin-2(1H)-one, chloro-N,N-diethyl-5-((4-(2-(4-(3-methylureido)phenyl)pyridin-4-yl)pyrimidin-2-yl)amino)benzenesulfonamide (compound Ii), or a compound disclosed in Gopalsamy et al, Bioorg. Med Chem Lett. 2012, 22:6636-66412 (for example, chloro-N,N-diethyl-5-((4-(2-(4-(3-methylureido)phenyl)pyridin-4-yl)pyrimidin-2-yl)amino)benzenesulfonamide.

In one aspect, the methods provided herein comprise administering an isotopologue of Compound A to patients with leukemia in combination with a BCL2 inhibitor. In certain embodiments, an isotopologue of Compound A is administered to patients with AML in combination with a BCL2 inhibitor, for example, venetoclax or navitoclax. In certain embodiments, the BCL2 inhibitor is venetoclax.

In one aspect, the methods provided herein comprise administering an isotopologue of Compound A to patients with leukemia in combination with a topoisomerase inhibitor. In certain embodiments, an isotopologue of Compound A is administered to patients with AML in combination with a topoisomerase inhibitor. In one embodiment, the topoisomerase inhibitor is irinotecan, topotecan, camptothecin, lamellarin D, etoposide, teniposide, doxorubicin, daunorubicin, mitoxantrone, amsacrine, ellipticines, aurintricarboxylic acid, or HU-331. In certain embodiments, the topoisomerase inhibitor is topotecan.

In one aspect, the methods provided herein comprise administering an isotopologue of Compound A to patients with leukemia in combination with one or more agents selected from triptolide, retaspimycin, alvespimycin, 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223), 1-ethyl-7-(2-methyl-6-(1H-1,2,4-triazol-3-yl)pyridin-3-yl)-3,4-dihydropyrazino[2,3-b]pyrazin-2(1H)-one (CC-115), rapamycin, MLN-0128, everolimus, AZD8055, pladienolide B, topotecan, thioguanine, mitoxantrone, etoposide, decitabine, daunorubicin, clofarabine, cladribine, 6-mercaptopurine, chloro-N,N-diethyl-5-((4-(2-(4-(3-methylureido)phenyl)pyridin-4-yl)pyrimidin-2-yl)amino)benzenesulfonamide (compound Ii), fedratinib, sunitinib, pexidartinib, midostaurin, lestaurtinib, momelotinib, quizartinib, and crenolanib.

In one aspect, the methods provided herein comprise administering an isotopologue of Compound A to patients with AML in combination with one or more agents selected from triptolide, retaspimycin, alvespimycin, 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223), 1-ethyl-7-(2-methyl-6-(1H-1,2,4-triazol-3-yl)pyridin-3-yl)-3,4-dihydropyrazino[2,3-b]pyrazin-2(1H)-one (CC-115), rapamycin, MLN-0128, everolimus, AZD8055, pladienolide B, topotecan, thioguanine, mitoxantrone, etoposide, decitabine, daunorubicin, clofarabine, cladribine, 6-mercaptopurine, chloro-N,N-diethyl-5-((4-(2-(4-(3-methylureido)phenyl)pyridin-4-yl)pyrimidin-2-yl)amino)benzenesulfonamide (compound Ii), fedratinib, sunitinib, pexidartinib, midostaurin, lestaurtinib, momelotinib, quizartinib, and crenolanib.

In one aspect, the methods provided herein comprise administering an isotopologue of Compound A to patients with cancer in combination with an mTOR inhibitor. In certain embodiments, the mTOR inhibitor is selected from everolimus, MLN-0128 and AZD8055. In other aspects, the methods provided herein comprise administering an isotopologue of Compound A to patients with cancer in combination with an an mTOR kinase inhibitor. In certain embodiments, the mTOR kinase inhibitor is selected from 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223) and 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, the cancer is selected from breast cancer, kidney cancer, pancreatic cancer, gastrointestinal cancer, lung cancer, neuroendocrine tumor (NET), and renal cell carcinoma. In one embodiment, the mTOR kinase inhibitor is 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223). In one embodiment, the mTOR kinase inhibitor is 1-ethyl-7-(2-methyl-6-(1H-1,2,4-triazol-3-yl)pyridin-3-yl)-3,4-dihydropyrazino[2,3-b]pyrazin-2(1H)-one (CC-15). In one embodiment, the mTOR inhibitor is everolimus. In one embodiment, the mTOR inhibitor is temsirolimus. In one embodiment, the mTOR kinase inhibitor is MLN-0128. In one embodiment, the mTOR kinase inhibitor is AZD8055.

In certain embodiments, an isotopologue of Compound A is administered to breast cancer patients in combination with everolimus.

In certain embodiments, an isotopologue of Compound A is administered to kidney cancer patients in combination with everolimus.

In certain embodiments, an isotopologue of Compound A is administered to pancreatic cancer patients in combination with everolimus.

In certain embodiments, an isotopologue of Compound A is administered to gastrointestinal cancer patients in combination with everolimus.

In certain embodiments, an isotopologue of Compound A is administered to lung cancer patients in combination with everolimus.

In certain embodiments, an isotopologue of Compound A is administered to neuroendocrine tumor patients in combination with everolimus.

In certain embodiments, an isotopologue of Compound A is administered to renal cell carcinoma patients in combination with everolimus.

In one embodiment, provided herein is a method comprising administering an isotopologue of Compound A in combination with an anti-cancer drug or agent to a patient (e.g., a human), wherein the method comprises increasing the dosage of an anti-cancer drug or agent that can be safely and effectively administered to a patient herein. Patients that can benefit by this method are those likely to suffer from an adverse effect associated with anti-cancer drugs for treating a specific cancer of the skin, subcutaneous tissue, lymph nodes, brain, lung, liver, bone, intestine, colon, heart, pancreas, adrenal, kidney, prostate, breast, colorectal, or combinations thereof. The administration of an isotopologue of Compound A provided herein, alleviates or reduces adverse effects which are of such severity that it would otherwise limit the amount of anti-cancer drug.

In one embodiment, provided herein is a method comprising administering an isotopologue of Compound A in combination with an anti-cancer drug or agent to a patient (e.g., a human), wherein the method comprises decreasing the dosage of an anti-cancer drug or agent that can be safely and effectively administered to a patient herein. Patients that can benefit by this method are those likely to suffer from an adverse effect associated with anti-cancer drugs for treating a specific cancer of the skin, subcutaneous tissue, lymph nodes, brain, lung, liver, bone, intestine, colon, heart, pancreas, adrenal, kidney, prostate, breast, colorectal, or combinations thereof. The administration of an isotopologue of Compound A provided herein, potentiates the activity of the anti-cancer drug, which allows for a reduction in dose of the anti-cancer drug while maintaining efficacy, which in turn can alleviate or reduce the adverse effects which are of such severity that it limited the amount of anti-cancer drug.

In one embodiment, an isotopologue of Compound A is administered daily in an amount ranging from about 0.1 to about 20 mg, from about 1 to about 15 mg, from about 1 to about 10 mg, or from about 1 to about 15 mg prior to, during, or after the occurrence of the adverse effect associated with the administration of an anti-cancer drug to a patient. In certain embodiments, an isotopologue of Compound A is administered in combination with specific agents such as heparin, aspirin, coumadin, or G-CSF to avoid adverse effects that are associated with anti-cancer drugs such as but not limited to neutropenia or thrombocytopenia.

In one embodiment, an isotopologue of Compound A provided herein, is administered to patients with diseases and disorders associated with or characterized by, undesired angiogenesis in combination with additional active ingredients, including, but not limited to, anti-cancer drugs, anti-inflammatories, antihistamines, antibiotics, and steroids.

In another embodiment, encompassed herein is a method of treating, preventing, ameliorating and/or managing cancer, which comprises administering an isotopologue of Compound A provided herein, in conjunction with (e.g. before, during, or after) at least one anti-cancer therapy including, but not limited to, surgery, immunotherapy, biological therapy, radiation therapy, or other non-drug based therapy presently used to treat, prevent, ameliorate and/or manage cancer. The combined use of the compound provided herein and other anti-cancer therapy may provide a unique treatment regimen that is unexpectedly effective in certain patients. Without being limited by theory, it is believed that an isotopologue of Compound A may provide additive or synergistic effects when given concurrently with at least one anti-cancer therapy.

As discussed elsewhere herein, encompassed herein is a method of reducing, treating and/or preventing adverse or undesired effects associated with other anti-cancer therapy including, but not limited to, surgery, chemotherapy, radiation therapy, hormonal therapy, biological therapy and immunotherapy. An isotopologue of Compound A provided herein, and other active ingredient can be administered to a patient prior to, during, or after the occurrence of the adverse effect associated with other anti-cancer therapy.

In certain embodiments, the methods provided herein comprise administration of one or more of calcium, calcitriol, and vitamin D supplementation with an isotopologue of Compound A. In certain embodiments, the methods provided herein comprise administration of calcium, calcitriol, and vitamin D supplementation prior to the treatment with an isotopologue of Compound A. In certain embodiments, the methods provided herein comprise administration of calcium, calcitriol, and vitamin D supplementation prior to the administration of first dose of an isotopologue of Compound A in each cycle. In certain embodiments, the methods provided herein comprise administration of calcium, calcitriol, and vitamin D supplementation at least up to 3 days prior to the treatment with an isotopologue of Compound A. In certain embodiments, the methods provided herein comprise administration of calcium, calcitriol, and vitamin D supplementation prior to the administration of first dose of an isotopologue of Compound A in each cycle. In certain embodiments, the methods provided herein comprise administration of calcium, calcitriol, and vitamin D supplementation at least up to 3 days prior to the administration of first dose of an isotopologue of Compound A in each cycle. In certain embodiments, the methods provided herein comprise administration of calcium, calcitriol, and vitamin D supplementation prior to administration of first dose of an isotopologue of Compound A in each cycle and continues after administration of the last dose of an isotopologue of Compound A in each cycle. In certain embodiments, the methods provided herein comprise administration of calcium, calcitriol, and vitamin D supplementation at least up to 3 days prior to administration of first dose of an isotopologue of Compound A in each cycle and continues until at least up to 3 days after administration of the last dose of an isotopologue of Compound A in each cycle (e.g., at least up to day 8 when an isotopologue of Compound A is administered on Days 1-5).

In certain embodiments, calcium supplementation is administered to deliver at least 1200 mg of elemental calcium per day given in divided doses. In certain embodiments, calcium supplementation is administered as calcium carbonate in a dose of 500 mg administered three times a day per orally (PO).

In certain embodiments, calcitriol supplementation is administered to deliver 0.25 pg calcitriol (PO) once daily.

In certain embodiments, vitamin D supplementation is administered to deliver about 500 IU to about 50,000 IU vitamin D once daily. In certain embodiments, vitamin D supplementation is administered to deliver about 1000 IU vitamin D once daily. In certain embodiments, vitamin D supplementation is administered to deliver about 50,000 IU vitamin D weekly. In certain embodiments, vitamin D supplementation is administered to deliver about 1000 IU vitamin D2 or D3 once daily. In certain embodiments, vitamin D supplementation is administered to deliver about 500 IU vitamin D once daily. In certain embodiments, vitamin D supplementation is administered to deliver about 50,000 IU vitamin D weekly. In certain embodiments, vitamin D supplementation is administered to deliver about 20,000 IU vitamin D weekly. In certain embodiments, vitamin D supplementation is administered to deliver about 1000 IU vitamin D2 or D3 once daily. In certain embodiments, vitamin D supplementation is administered to deliver about 50,000 IU vitamin D2 or D3 weekly. In certain embodiments, vitamin D supplementation is administered to deliver about 20,000 IU vitamin D2 or D3 weekly.

In certain embodiments, an isotopologue of Compound A provided herein and doxetaxol are administered to patients with non-small cell lung cancer who were previously treated with carbo/VP 16 and radiotherapy.

Use with Transplantation Therapy

An isotopologue of Compound A provided herein, can be used to reduce the risk of Graft Versus Host Disease (GVHD). Therefore, encompassed herein is a method of treating, preventing and/or managing cancer, which comprises administering an isotopologue of Compound A provided herein, in conjunction with transplantation therapy.

As those of ordinary skill in the art are aware, the treatment of cancer is often based on the stages and mechanism of the disease. For example, as inevitable leukemic transformation develops in certain stages of cancer, transplantation of peripheral blood stem cells, hematopoietic stem cell preparation or bone marrow may be necessary. The combined use of an isotopologue of Compound A provided herein, and transplantation therapy provides a unique and unexpected synergism. In particular, an isotopologue of Compound A provided herein exhibits immunomodulatory activity that may provide additive or synergistic effects when given concurrently with transplantation therapy in patients with cancer.

An isotopologue of Compound A provided herein, can work in combination with transplantation therapy reducing complications associated with the invasive procedure of transplantation and risk of GVHD. Encompassed herein is a method of treating, preventing and/or managing cancer which comprises administering to a patient (e.g., a human) an isotopologue of Compound A provided herein before, during, or after the transplantation of umbilical cord blood, placental blood, peripheral blood stem cell, hematopoietic stem cell preparation, or bone marrow. Some examples of stem cells suitable for use in the methods provided herein are disclosed in U.S. Pat. No. 7,498,171, the disclosure of which is incorporated herein by reference in its entirety.

In one embodiment, an isotopologue of Compound A provided herein, is administered to patients with acute myeloid leukemia before, during, or after transplantation.

In one embodiment, an isotopologue of Compound A provided herein, is administered to patients with multiple myeloma before, during, or after the transplantation of autologous peripheral blood progenitor cell.

In one embodiment, an isotopologue of Compound A provided herein, is administered to patients with NHL (e.g., DLBCL) before, during, or after the transplantation of autologous peripheral blood progenitor cell.

Cycling Therapy

In certain embodiments, an isotopologue of Compound A provided herein, are cyclically administered to a patient independent of the cancer treated. Cycling therapy involves the administration of an active agent for a period of time, followed by a rest for a period of time, and repeating this sequential administration. Cycling therapy can reduce the development of resistance to one or more of the therapies, avoid, or reduce the side effects of one of the therapies, and/or improves the efficacy of the treatment.

In certain embodiments, an isotopologue of Compound A provided herein, is administered daily in a single or divided dose in a four to six week cycle with a rest period of about a week or two weeks. In certain embodiments, an isotopologue of Compound A provided herein, is administered daily in a single or divided doses for one to ten consecutive days of a 28 day cycle, then a rest period with no administration for rest of the 28 day cycle. The cycling method further allows the frequency, number, and length of dosing cycles to be increased. Thus, encompassed herein in certain embodiments is the administration of an isotopologue of Compound A provided herein, for more cycles than are typical when it is administered alone. In certain embodiments, an isotopologue of Compound A provided herein, is administered for a greater number of cycles that would typically cause dose-limiting toxicity in a patient to whom a second active ingredient is not also being administered.

In one embodiment, an isotopologue of Compound A provided herein, is administered daily and continuously for three or four weeks to administer a dose of an isotopologue of Compound A from about 0.1 to about 20 mg/d followed by a break of one or two weeks.

In another embodiment, an isotopologue of Compound A provided herein, is administered intravenously and a second active ingredient is administered orally, with administration of an isotopologue of Compound A provided herein, occurring 30 to 60 minutes prior to a second active ingredient, during a cycle of four to six weeks. In certain embodiments, the combination of an isotopologue of Compound A provided herein, and a second active ingredient is administered by intravenous infusion over about 90 minutes every cycle. In certain embodiments, one cycle comprises the administration from about 0.1 to about 150 mg/day of an isotopologue of Compound A provided herein, and from about 50 to about 200 mg/m²/day of a second active ingredient daily for three to four weeks and then one or two weeks of rest. In certain embodiments, the number of cycles during which the combinatorial treatment is administered to a patient is ranging from about one to about 24 cycles, from about two to about 16 cycles, or from about four to about three cycles.

In one embodiment, a cycling therapy provided herein comprises administering an isotopologue of Compound A provided herein, in a treatment cycle which includes an administration period of up to 5 days followed by a rest period. In one embodiment, the treatment cycle includes an administration period of 5 days followed by a rest period. In one embodiment, the treatment cycle includes an administration period of up to 10 days followed by a rest period. In one embodiment, the rest period is from about 10 days up to about 40 days. In one embodiment, the treatment cycle includes an administration period of up to 10 days followed by a rest period from about 10 days up to about 40 days. In one embodiment, the treatment cycle includes an administration period of up to 10 days followed by a rest period from about 23 days up to about 37 days. In one embodiment, the rest period is from about 23 days up to about 37 days. In one embodiment, the rest period is 23 days. In one embodiment, the treatment cycle includes an administration period of up to 10 days followed by a rest period of 23 days. In one embodiment, the rest period is 37 days. In one embodiment, the treatment cycle includes an administration period of up to 10 days followed by a rest period of 37 days.

In one embodiment, the treatment cycle includes an administration of an isotopologue of Compound A provided herein, on days 1 to 5 of a 28 day cycle. In another embodiment, the treatment cycle includes an administration of an isotopologue of Compound A provided herein, on days 1-10 of a 28 day cycle. In one embodiment, the treatment cycle includes an administration on days 1 to 5 of a 42 day cycle. In another embodiment, the treatment cycle includes an administration on days 1-10 of a 42 day cycle. In another embodiment, the treatment cycle includes an administration on days 1-5 and 15-19 of a 28 day cycle.

In one embodiment, the treatment cycle includes an administration of an isotopologue of Compound A provided herein, on days 1 to 21 of a 28 day cycle. In another embodiment, the treatment cycle includes an administration on days 1 to 5 of a 7 day cycle. In another embodiment, the treatment cycle includes an administration on days 1 to 7 of a 7 day cycle.

Any treatment cycle described herein can be repeated for at least 2, 3, 4, 5, 6, 7, 8, or more cycles. In certain instances, the treatment cycle as described herein includes from 1 to about 24 cycles, from about 2 to about 16 cycles, or from about 2 to about 4 cycles. In certain instances a treatment cycle as described herein includes from 1 to about 4 cycles. In certain embodiments, cycle 1 to 4 are all 28 day cycles. In certain embodiments, cycle 1 is a 42 day cycle and cycles 2 to 4 are 28 day cycles. In some embodiments, an isotopologue of Compound A provided herein, is administered for 1 to 13 cycles of 28 days (e.g. about 1 year). In certain instances, the cycling therapy is not limited to the number of cycles, and the therapy is continued until disease progression. Cycles, can in certain instances, include varying the duration of administration periods and/or rest periods described herein.

In one embodiment the treatment cycle includes administering an isotopologue of Compound A at a dosage amount of about 0.3 mg/day, 0.6 mg/day, 1.2 mg/day, 1.8 mg/day, 2.4 mg/day, 3.6 mg/day, 5.4 mg/day, 7.2 mg/day, 8.1 mg/day, 9.0 mg/day, 10.0 mg/day, 10.8 mg/day, or 12.2 mg/day administered once per day. In one embodiment the treatment cycle includes administering an isotopologue of Compound A at a dosage amount of about 0.3 mg/day, 0.6 mg/day, 1.2 mg/day, 1.8 mg/day, 2.4 mg/day, 3.6 mg/day, 5.4 mg/day, 7.2 mg/day, 8.1 mg/day, 9.0 mg/day, 10.0 mg/day, 10.8 mg/day, 12.2 mg/day, or 20 mg/day administered once per day. In one embodiment the treatment cycle includes administering an isotopologue of Compound A at a dosage amount of about 0.6 mg/day, 1.2 mg/day, 1.8 mg/day, 2.4 mg/day, or 3.6 mg/day, administered once per day. In some such embodiments, the treatment cycle includes administering an isotopologue of Compound A at a dosage amount of about 0.6 mg, 1.2 mg, 1.8 mg, 2.4 mg, or 3.6 mg on days 1 to 3 of a 28 day cycle. In other embodiments, the treatment cycle includes administering an isotopologue of Compound A at a dosage amount of about 0.6 mg, 1.2 mg, 1.8 mg, 2.4 mg, or 3.6 mg on days 1 to 5 and 15 to 19 of a 28 day cycle. In other embodiments, the treatment cycle includes administering an isotopologue of Compound A at a dosage amount of about 0.6 mg, 1.2 mg, 1.8 mg, 2.4 mg, 3.6 mg, 5.4 mg/day, 7.2 mg/day, 8.1 mg/day, 9.0 mg/day, or 10.0 mg/day, on days 1 to 5 and 15 to 19 of a 28 day cycle.

An isotopologue of Compound A provided herein, can be administered at the same amount for all administration periods in a treatment cycle. Alternatively, in one embodiment, the compound is administered at different doses in the administration periods.

In one embodiment, an isotopologue of Compound A provided herein is administered to a subject in a cycle, wherein the cycle comprises administering the compound for at least 5 days in a 28 day cycle. In one embodiment, an isotopologue of Compound A provided herein is administered to a subject in a cycle, wherein the cycle comprises administering the compound on days 1 to 5 of a 28 day cycle. In one embodiment, an isotopologue of Compound A is administered in a dose of about 0.1 mg to about 20 mg on days 1 to 5 of a 28 day cycle. In one embodiment, an isotopologue of Compound A is administered in a dose of about 0.5 mg to about 5 mg on days 1 to 5 of a 28 day cycle. In one embodiment, an isotopologue of Compound A administered in a dose of about 0.5 mg to about 10 mg on days 1 to 5 of a 28 day cycle. In one embodiment, an isotopologue of Compound A provided herein is administered to a subject in a cycle, wherein the cycle comprises administering the compound on days 1 to 5 and 15 to 19 of a 28 day cycle. In one embodiment, an isotopologue of Compound A is administered in a dose of about 0.1 mg to about 20 mg on days 1 to 5 and 15 to 19 of a 28 day cycle. In one embodiment, an isotopologue of Compound A is administered in a dose of about 0.5 mg to about 5 mg on days 1 to 5 and 15 to 19 of a 28 day cycle. In one embodiment, an isotopologue of Compound A is administered in a dose of about 0.5 mg to about 10 mg on days 1 to 5 and 15 to 19 of a 28 day cycle.

In one embodiment, provided herein is a method of treating of AML by administering to a subject an isotopologue of Compound A in a cycle, wherein the cycle comprises administering the isotopologue of Compound A in a dose of about 0.1 mg to about 20 mg for at least 5 days in a 28 day cycle. In one embodiment, provided herein is a method of treating of AML by administering to a subject an isotopologue of Compound A in a cycle, wherein the cycle comprises administering the isotopologue of Compound A in a dose of about 0.1 mg to about 20 mg on days 1 to 5 of a 28 day cycle. In one embodiment, provided herein is a method of treating of AML by administering to a subject an isotopologue of Compound A in a cycle, wherein the cycle comprises administering the isotopologue of Compound A in a dose of about 0.1 mg to about 5 mg on days 1 to 5 of a 28 day cycle. In one embodiment, provided herein is a method of treating of AML by administering to a subject an isotopologue of Compound A in a cycle, wherein the cycle comprises administering the isotopologue of Compound A in a dose of about 0.5 mg to about 5 mg on days 1 to 5 of a 28 day cycle. In another embodiment, provided herein is a method of treating of AML by administering to a subject an isotopologue of Compound A in a cycle, wherein the cycle comprises administering the isotopologue of Compound A in a dose of about 0.1 mg to about 20 mg on days 1 to 5 and 15 to 19 of a 28 day cycle. In one embodiment, provided herein is a method of treating of AML by administering to a subject an isotopologue of Compound A in a cycle, wherein the cycle comprises administering the isotopologue of Compound A in a dose of about 0.1 mg to about 5 mg on days 1 to 5 and 15 to 19 of a 28 day cycle. In one embodiment, provided herein is a method of treating of AML by administering to a subject an isotopologue of Compound A in a cycle, wherein the cycle comprises administering the isotopologue of Compound A in a dose of about 0.5 mg to about 5 mg on days 1 to 5 and 15 to 19 of a 28 day cycle.

In one embodiment, provided herein is a method of treating of MDS by administering to a subject an isotopologue of Compound A in a cycle, wherein the cycle comprises administering the isotopologue of Compound A in a dose of about 0.1 mg to about 20 mg for at least 5 days in a 28 day cycle. In one embodiment, provided herein is a method of treating of MDS by administering to a subject an isotopologue of Compound A in a cycle, wherein the cycle comprises administering the isotopologue of Compound A in a dose of about 0.1 mg to about 20 mg on days 1 to 5 of a 28 day cycle. In one embodiment, provided herein is a method of treating of MDS by administering to a subject an isotopologue of Compound A in a cycle, wherein the cycle comprises administering the isotopologue of Compound A a dose of about 0.1 mg to about 5 mg on days 1 to 5 of a 28 day cycle. In one embodiment, provided herein is a method of treating of MDS by administering to a subject an isotopologue of Compound A in a cycle, wherein the cycle comprises administering the isotopologue of Compound A in a dose of about 0.5 mg to about 5 mg on days 1 to 5 of a 28 day cycle. In another embodiment, provided herein is a method of treating of MDS by administering to a subject an isotopologue of Compound A in a cycle, wherein the cycle comprises administering the isotopologue of Compound A in a dose of about 0.1 mg to about 20 mg on days 1 to 5 and 15 to 19 of a 28 day cycle. In one embodiment, provided herein is a method of treating of MDS by administering to a subject an isotopologue of Compound A in a cycle, wherein the cycle comprises administering the isotopologue of Compound A in a dose of about 0.1 mg to about 5 mg on days 1 to 5 and 15 to 19 of a 28 day cycle. In one embodiment, provided herein is a method of treating of MDS by administering to a subject an isotopologue of Compound A in a cycle, wherein the cycle comprises administering the isotopologue of Compound A in a dose of about 0.5 mg to about 5 mg on days 1 to 5 and 15 to 19 of a 28 day cycle.

Patient Population

In certain embodiments of the methods provided herein, the subject is an animal, in one embodiment a mammal, more preferably a non-human primate. In particular embodiments, the subject is a human. The subject can be a male or female subject.

Particularly useful subjects for the methods provided herein include human cancer patients, for example, those who have been diagnosed with leukemia, including acute myeloid leukemia, acute lymphocytic leukemia, chronic myelogenous leukemia, and chronic myelogenous leukemia. In certain embodiments, the subject has not been diagnosed with acute promyelocytic leukemia.

In some embodiments, the subject has a higher than normal blast population. In some embodiments, the subject has a blast population of at least 10%. In some embodiments, the subject has a blast population of between 10 and 15%. In some embodiments, the subject has a blast population of at least 15%. In some embodiments, the subject has a blast population of between 15 and 20%. In some embodiments, the subject has a blast population of at least 20%. In some embodiments, the subject has a blast population of about 10-15%, about 15-20%, or about 20-25%. In other embodiments, the subject has a blast population of less than 10%. In the context of the methods described herein, useful subjects having a blast population of less than 10% includes those subjects that, for any reason according to the judgment of the skilled practitioner in the art, are in need of treatment with a compound provided herein, alone or in combination with a second active agent.

In some embodiments, the subject is treated based on the Eastern Cooperative Oncology Group (ECOG) performance status score of the subject for leukemia. ECOG performance status can be scored on a scale of 0 to 5, with 0 denoting asymptomatic; 1 denoting symptomatic but completely ambulant; 2 denoting symptomatic and <50% in bed during the day; 3 denoting symptomatic and >50% in bed, but not bed bound; 4 denoting bed bound; and 5 denoting death. In some embodiments, the subject has an ECOG performance status score of 0 or 1. In some embodiments, the subject has an ECOG performance status score of 0. In some embodiments, the subject has an ECOG performance status score of 1. In other embodiments, the subject has an ECOG performance status score of 2.

In certain embodiments, the methods provided herein encompass the treatment of subjects who have not been previously treated for leukemia. In some embodiments, the subject has not undergone allogeneic bone marrow transplantation. In some embodiments, the subject has not undergone a stem cell transplantation. In some embodiments, the subject has not received hydroxyurea treatment. In some embodiments, the subject has not been treated with any investigational products for leukemia. In some embodiments, the subject has not been treated with systemic glucocorticoids.

In other embodiments, the methods encompass treating subjects who have been previously treated or are currently being treated for leukemia. For example, the subject may have been previously treated or are currently being treated with a standard treatment regimen for leukemia. The subject may have been treated with any standard leukemia treatment regimen known to the practitioner of skill in the art. In certain embodiments, the subject has been previously treated with at least one induction/reinduction or consolidation AML regimen. In some embodiments, the subject has undergone autologous bone marrow transplantation or stem cell transplantation as part of a consolidation regimen. In some embodiments, the bone marrow or stem cell transplantation occurred at least 3 months prior to treatment according to the methods provided herein. In some embodiments, the subject has undergone hydroxyurea treatment. In some embodiments, the hydroxyurea treatment occurred no later than 24 hours prior to treatment according to the methods provided herein. In some embodiments, the subject has undergone prior induction or consolidation therapy with cytarabine (Ara-C). In some embodiments, the subject has undergone treatment with systemic glucocorticosteroids. In some embodiments, the glucocorticosteroid treatment occurred no later 24 hours prior to treatment according to the methods described herein. In other embodiments, the methods encompass treating subjects who have been previously treated for cancer, but are non-responsive to standard therapies.

Also encompassed are methods of treating subjects having relapsed or refractory leukemia. In some embodiments, the subject has been diagnosed with a relapsed or refractory AML subtype, as defined by the World Health Organization (WHO). Relapsed or refractory disease may be de novo AML or secondary AML, e.g., therapy-related AML (t-AML).

In some embodiments, the methods provided herein are used to treat drug resistant leukemias, such as chronic myelogenous leukemia (CML). Thus, treatment with an isotopologue of Compound A provided herein could provide an alternative for patients who do not respond to other methods of treatment. In some embodiments, such other methods of treatment encompass treatment with Gleevec® (imatinib mesylate). In some embodiments, provided herein are methods of treatment of Philadelphia chromosome positive chronic myelogenous leukemia (Ph+CML). In some embodiments, provided herein are methods of treatment of Gleevec® (imatinib mesylate) resistant Philadelphia chromosome positive chronic myelogenous leukemia (Ph+CML).

Also encompassed are methods of treating a subject regardless of the subject's age, although some diseases or disorders are more common in certain age groups. In some embodiments, the subject is at least 18 years old. In some embodiments, the subject is more than 18, 25, 35, 40, 45, 50, 55, 60, 65, or 70 years old. In other embodiments, the subject is less than 65 years old. In some embodiments, the subject is less than 18 years old. In some embodiments, the subject is less than 18, 15, 12, 10, 9, 8 or 7 years old.

In some embodiments, the methods may find use in subjects at least 50 years of age, although younger subjects could benefit from the method as well. In other embodiments, the subjects are at least 55, at least 60, at least 65, and at least 70 years of age. In another embodiment, the subjects have adverse cytogenetics. “Adverse cytogenetics” is defined as any nondiploid karyotype, or greater than or equal to 3 chromosomal abnormalities. In another embodiment, the subjects are at least 60 years of age and have adverse cytogenetics. In another embodiment, the subjects are 60-65 years of age and have adverse cytogenetics. In another embodiment, the subjects are 65-70 years of age and have adverse cytogenetics.

In certain embodiments, the subject treated has no history of myocardial infarction within three months of treatment according to the methods provided herein. In some embodiments, the subject has no history of cerebrovascular accident or transient ischemic attack within three months of treatment according to the methods provided herein. In some embodiments, the subject has no suffered no thromboembelic event, including deep vein thrombosis or pulmonary embolus, within 28 days of treatment according to the methods provided herein. In other embodiments, the subject has not experienced or is not experiencing uncontrolled disseminated intravascular coagulation.

Because subjects with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual subject with cancer.

It will be appreciated that every suitable combination of the compounds provided herein with one or more of the aforementioned compounds and optionally one or more further pharmacologically active substances is contemplated herein.

5.4 Formulation of Pharmaceutical Compositions

The pharmaceutical compositions provided herein contain therapeutically effective amounts of one or more of compounds provided herein and a pharmaceutically acceptable carrier, diluent and/or excipient.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the compounds in the compositions are effective for delivery of an amount, upon administration, that treats, prevents, or ameliorates one or more of the symptoms and/or progression of cancer, including solid tumors and blood borne tumors.

Typically, the compositions are formulated for single dosage administration. To formulate a composition, the weight fraction of an isotopologue of Compound A is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, an isotopologue of Compound A may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of an isotopologue of Compound A provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

An isotopologue of Compound A is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of an isotopologue of Compound A in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of cancer, including solid tumors and blood borne tumors.

In certain embodiments, a therapeutically effective dosage should produce a serum concentration of active ingredient of from about 0.1 ng/mL to about 50-100 μg/mL. In one embodiment, the pharmaceutical compositions provide a dosage of from about 0.001 mg to about 2000 mg of an isotopologue of Compound A per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of an isotopologue of Compound A in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to orally, parenterally, rectally, topically and locally. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the isotopologue of Compound A in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the isotopologue of Compound A sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the isotopologue of Compound A provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non toxic carrier may be prepared. For oral administration, a pharmaceutically acceptable non toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1 85% or about 75-95%.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

Lactose-free compositions provided herein can contain excipients that are well known in the art and are listed, for example, in the U.S. Pharmocopia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing an isotopologue of Compound A provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

The pharmaceutical compositions provided herein can be used in any of the methods of treating, preventing, ameliorating and/or managing provided herein.

i. Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the isotopologue of Compound A could be provided in a composition that protects it from the acidic environment of the stomach. For example, the composition can be formulated in an enteric coating that maintains its integrity in the stomach and releases the isotopologue of Compound A in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is an isotopologue of Compound A as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non effervescent granules and effervescent preparations reconstituted from effervescent granules. Aqueous solutions include, for example, elixirs and syrups. Emulsions are either oil in-water or water in oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non aqueous liquids, emulsifying agents and preservatives. Suspensions use pharmaceutically acceptable suspending agents and preservatives. Pharmaceutically acceptable substances used in non effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the isotopologue of Compound A in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing an isotopologue of Compound A provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

ii. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, an isotopologue of Compound A provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of an isotopologue of Compound A contained in such parenteral compositions is highly dependent on the specific nature thereof, as well as the activity of the isotopologue of Compound A and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the isotopologue of Compound A is adjusted so that an injection provides an effective amount to produce the desired pharmacological effect. The exact dose depends on the age, weight and condition of the patient or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an isotopologue of Compound A is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the isotopologue of Compound A to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted overtime according to the individual need and the professional judgment of the person administering or supervising the administration of the formulations, and that the concentration ranges set forth herein are exemplary only and are not intended to limit the scope or practice of the claimed formulations.

The isotopologue of Compound A may be suspended in micronized or other suitable form or may be derivatized to produce a more soluble active product. The form of the resulting mixture depends upon a number of factors, including the intended mode of administration and the solubility of the isotopologue of Compound A in the selected carrier or vehicle. The effective concentration is sufficient for ameliorating the symptoms of the condition and may be empirically determined.

iii. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving an isotopologue of Compound A provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

iv. Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the isotopologue of Compound A alone or in combination with other pharmaceutically acceptable excipients can also be administered.

These solutions, particularly those intended for ophthalmic use, may be formulated as 0.01%-10% isotonic solutions, pH about 5-7, with appropriate salts.

v. Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

vi. Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

vii. Targeted Formulations

The compounds provided herein, or pharmaceutically acceptable salts thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of an isotopologue of Compound A provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

viii. Articles of Manufacture

The isotopologue of Compound A can be packaged as an article of manufacture containing packaging material, an isotopologue of Compound A provided herein, which is used for treatment, prevention or amelioration of one or more symptoms or progression of cancer, including solid tumors and blood borne tumors, and a label that indicates that the isotopologue of Compound A is used for treatment, prevention or amelioration of one or more symptoms or progression of cancer, including solid tumors and blood borne tumors.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

5.5 Evaluation of Activity

Standard physiological, pharmacological and biochemical procedures are available for testing the compounds to identify those that possess the desired activity.

Such assays include, for example, cell based assays, including the assay described in the Example section and in U.S. Pat. No. 9,499,514.

Embodiments provided herein may be more fully understood by reference to the following examples. These examples are meant to be illustrative of pharmaceutical compositions and dosage forms provided herein, but are not in any way limiting.

### 6 EXAMPLES

General: Isotopically enriched analogs of the compounds provided herein may generally be prepared according known procedures for the synthesis of Compound A, wherein one or more of the reagents, starting materials, precursors, or intermediates used is replaced by one or more isotopically enriched reagents, starting materials, precursors, or intermediates. Isotopically enriched reagents, starting materials, precursors, or intermediates are commercially available or may be prepared by routine procedures known to one of skill in the art. Schemes for the preparation of exemplary isotopically enriched compounds are illustrated below.

### Abbreviations

HPLC: high performance liquid chromatography
GC-MS: gas chromatography/mass spectrometry
NMR: nuclear magnetic resonance

### Example 1

### 2-(4-Chloro-5-deutero-phenyl)-N-((2-(2,6-dioxopiperidin-3-yl)-1-oxoisoindolin-5-yl)methyl)-2,2-difluoroacetamide

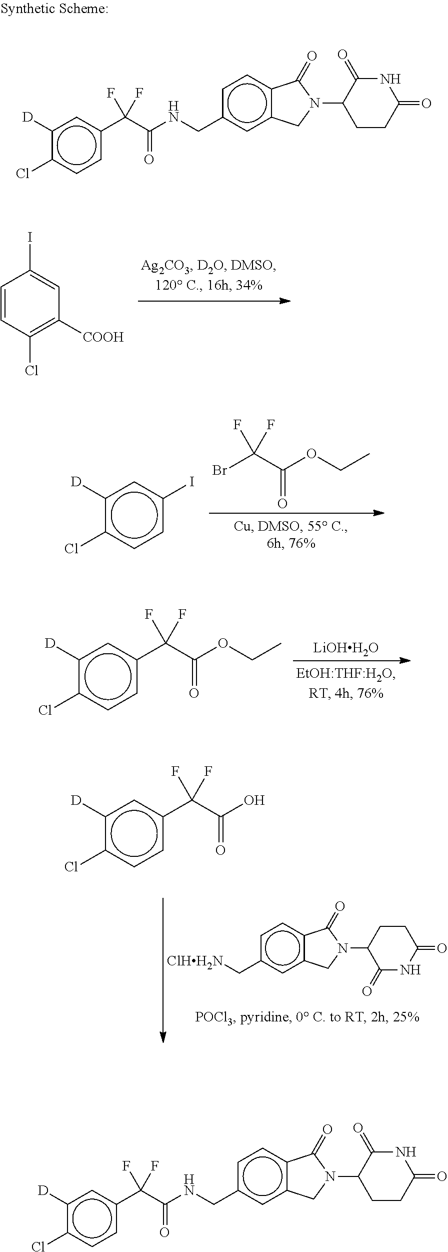

A. 1-chloro-4-iodobenzene-2-d: To a solution of 2-chloro-5-iodobenzoic acid (2.0 g, 7.08 mmol) in dimethyl sulfoxide (40 mL) was added silver carbonate (195 mg, 0.71 mmol) and deuterium oxide (7.08 g, 354.02 mmol). The reaction was stirred at 120° C. for 16 h. The reaction mixture was cooled to room temperature, poured into water and extracted with ethyl acetate (3×100 mL). The combined organic layer was washed with water (2×100 mL), brine (100 mL) and dried over sodium sulphate. Volatile organics were removed under reduced pressure to afford 1-chloro-4-iodobenzene-2-d (600 mg, 2.51 mmol, 34% yield). GCMS (m/z) 239.0 [M]⁺.

B. Ethyl 2-(4-chlorophenyl-3-d)-2,2-difluoroacetate: To a stirred solution of 1-chloro-4-iodobenzene-2-d (600 mg, 2.51 mmol) in dimethyl sulfoxide (6.5 mL) was added copper (415 mg, 6.53 mmol) and ethyl 2-bromo-2,2-difluoroacetate (608 mg, 3.01 mmol) at room temperature and stirred at 55° C. for 6 h. The reaction mixture was neutralized with aqueous saturated ammonium chloride solution and extracted with ethyl acetate (2×50 mL). The combined organic layers were washed with water (2×50 mL), brine (50 mL), dried over sodium sulphate and concentrated to afford ethyl 2-(4-chlorophenyl-3-d)-2,2-difluoroacetate (450 mg, 1.91 mmol, 76%). GCMS (m/z) 235.0 [M]⁺.

C. 2-(4-chlorophenyl-3-d)-2,2-difluoroacetic acid: To a solution of ethyl 2-(4-chlorophenyl-3-d)-2,2-difluoroacetate (450 mg, 1.91 mmol) in tetrahydrofuran:methanol:water mixture (30 mL, 1:1:1) was added lithium hydroxide monohydrate (240 mg, 3.41 mmol) and stirred at room temperature for 4 h. The reaction mixture was concentrated and the residue was neutralized with saturated potassium bisulphate (20 mL) and extracted with ethyl acetate (2×25 mL). The combined organic layers were washed with brine (25 mL), dried over sodium sulphate and concentrated to afford 2-(4-chlorophenyl-3-d)-2,2-difluoroacetic acid (300 mg, 1.44 mmol, 76% yield). MS (ESI) m/z 206.23 [M−1]⁻.

D.2-(4-chlorophenyl-3-d)-N-((2-(2,6-dioxopiperidin-3-yl)-1-oxoisoindolin-5-yl)methyl)-2,2-difluoroacetamide: To a cold (0° C.) solution of 2-(4-chlorophenyl-3-d)-2,2-difluoroacetic acid (200 mg, 0.96 mmol) in pyridine (10 mL) was added phosphoryl chloride (441 mg, 2.88 mmol) dropwise and stirred at 0-5° C. for 30 min. To this reaction mixture was then added 3-(5-(aminomethyl)-1-oxoisoindolin-2-yl)piperidine-2,6-dione hydrochloride (297 mg, 0.96 mmol) and stirred at room temperature for 1 h. The reaction mixture was neutralized with aqueous saturated sodium bicarbonate (up to pH-8) and extracted with ethyl acetate (3×50 mL). The combined organic layers were washed with water (2×50 mL), brine (50 mL), dried over sodium sulphate and concentrated. The resultant residue was purified by Reveleris C-18 reversed phase column chromatography using 42-45% acetonitrile in aqueous formic acid (0.1%) to afford 2-(4-chlorophenyl-3-d)-N-((2-(2,6-dioxopiperidin-3-yl)-1-oxoisoindolin-5-yl)methyl)-2,2-difluoroacetamide (75 mg, 0.16 mmol, 25% yield) as an off-white solid. ¹H NMR (300 MHz, DMSO-d6) δ 10.98 (s, 1H), 9.68 (t, J=6.2 Hz, 1H), 7.72-7.56 (m, 4H), 7.44-7.30 (m, 2H), 5.10 (dd, J=5.0, 13.0 Hz, 1H), 4.52-4.22 (m, 4H), 3.00-2.83 (m, 1H), 2.68-2.55 (m, 1H), 2.46-2.30 (m, 1H), 2.06-1.93 (m, 1H). MS (ESI) m/z 463.08 [M+1]⁺.

### Example 2

### 2-(4-Chloro-2-deuterophenyl)-N-((2-(2,6-dioxopiperidin-3-yl)-1-oxoisoindolin-5-yl)methyl)-2,2-difluoroacetamide

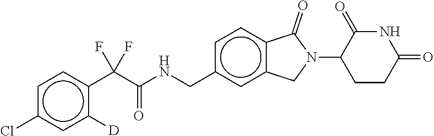

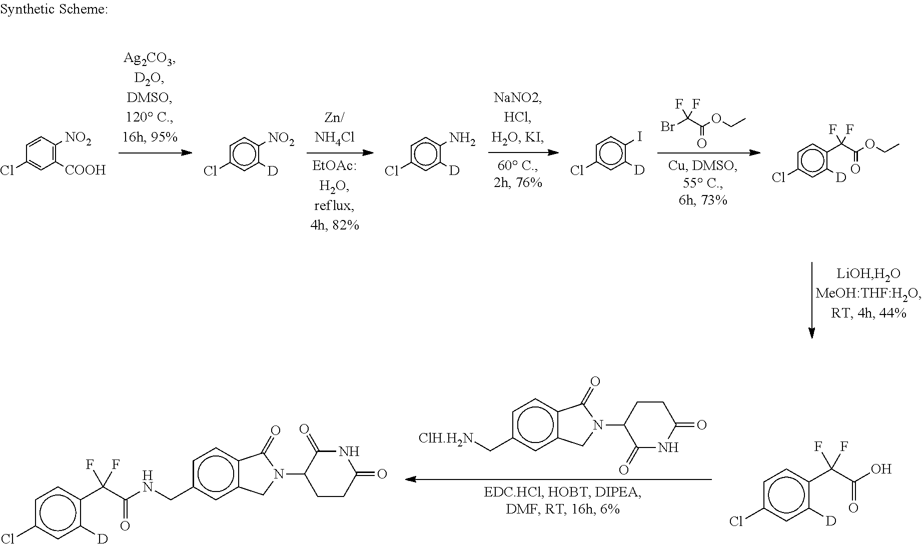

A. 1-chloro-4-nitrobenzene-3-d: To a solution of 5-chloro-2-nitrobenzoic acid (2.0 g, 9.95 mmol) in dimethylsulfoxide (50 mL) was added silver carbonate (274 mg, 0.99 mmol) and deuterium oxide (9.95 g, 497.51 mmol). The reaction was stirred at 120° C. for 16 h. The reaction mixture was cooled to room temperature, poured into water and extracted with ethyl acetate (3×100 mL). The combined organic layers were washed with water (2×100 mL), brine (100 mL), dried over sodium sulphate and concentrated to afford 1-chloro-4-nitrobenzene-3-d (1.5 g, 7.46 mmol, 95% yield). GCMS (m/z) 158.1 [M]⁺.

B. 4-chlorobenzen-2-d-amine: To a stirred solution of 1-chloro-4-nitrobenzene-3-d (1.5 g, 7.46 mmol) in ethyl acetate (40 mL) and water (10 mL) was added zinc powder (2.48 g, 37.97 mmol) followed ammonium chloride (5.1 g, 94.93 mmol) at room temperature and heated to reflux for 4 h. The reaction mixture was diluted with ethyl acetate (50 mL). The organic layer was washed with water (2×50 mL), brine (50 mL), dried over sodium sulphate and concentrated to afford 4-chlorobenzen-2-d-amine (1.0 g, 7.81 mmol, 82% yield). GCMS (m/z) 128.1 [M]⁺.

C. 1-chloro-4-iodobenzene-3-d: To a cold (0° C.) solution of 4-chlorobenzen-2-d-amine (1.0 g, 7.81 mmol) in 50% aqueous hydrochloric acid (8 mL) was added a solution of sodium nitrite (1.35 g, 19.53 mmol) and stirred at the same temperature for 30 min. Then added a solution of potassium iodide (3.24 g, 19.53 mmol) and stirred at 60° C. for 2 h. The reaction mixture was cooled to room temperature, poured into water (25 mL) and extracted with ethyl acetate (2×50 mL). The combined organic layers were washed with brine (50 mL), dried over sodium sulphate and concentrated. The resultant residue was purified by column chromatography using 0-2% ethyl acetate in petroleum ether to afford 1-chloro-4-iodobenzene-3-d (1.2 g, 5.02 mmol, 74% yield). GCMS (m/z) 238.9 [M]⁺.

D. Ethyl 2-(4-chlorophenyl-2-d)-2,2-difluoroacetate: To a stirred solution of 1-chloro-4-iodobenzene-3-d (1.2 g, 5.02 mmol) in dimethyl sulfoxide (13 mL) was added copper (829 g, 13.05 mmol) and ethyl 2-bromo-2,2-difluoroacetate (1.52 g, 7.53 mmol) at room temperature and stirred at 55° C. for 6 h. The reaction mixture was neutralized with aqueous saturated ammonium chloride solution and extracted with ethyl acetate (2×100 mL). The combined organic layers were washed with water (2×100 mL), brine (50 mL), dried over sodium sulphate and concentrated to afford ethyl 2-(4-chlorophenyl-2-d)-2,2-difluoroacetate (900 mg, 3.83 mmol, 73%). GCMS (m/z) 235.0 [M]⁺.

E. 2-(4-chlorophenyl-2-d)-2,2-difluoroacetic acid: To a stirred solution of ethyl 2-(4-chlorophenyl-2-d)-2,2-difluoroacetate (900 mg, 3.83 mmol) in tetrahydrofuran:methanol:water mixture (30 mL, 1:1:1) was added lithium hydroxide monohydrate (482 mg, 11.49 mmol) and stirred at room temperature for 4 h. The reaction mixture was concentrated and the residue was neutralized with saturated potassium bisulphate (25 mL) and extracted with ethyl acetate (2×50 mL). The combined organic layers were washed with brine (50 mL), dried over sodium sulphate and concentrated to afford 2-(4-chlorophenyl-2-d)-2,2-difluoroacetic acid (600 mg, 2.90 mmol, 76% yield). MS (ESI) m/z 206.2 [M−1]. F. 2-(4-chlorophenyl-2-d)-N-((2-(2,6-dioxopiperidin-3-yl)-1-oxoisoindolin-5-yl)methyl)-2,2-difluoroacetamide: To a stirred solution of 2-(4-chlorophenyl-2-d)-2,2-difluoroacetic acid (200 mg, 0.97 mmol) in N,N-dimethylformamide (10 mL) was added N-(3-Dimethylaminopropyl)-N′-ethylcarbodiimide hydrochloride, (278 mg, 1.45 mmol), 1-hydroxybenzotriazole (222 mg, 1.45 mmol), N,N-diisopropylethylamine (0.5 mL, 2.90 mmol) followed by 3-(5-(aminomethyl)-1-oxoisoindolin-2-yl)piperidine-2,6-dione hydrochloride and stirred at room temperature for 16 h. The reaction mixture was poured into water (20 mL) and extracted with ethyl acetate (3×50 mL). The combined organic layers were washed with water (2×50 mL), brine (50 mL) and dried over sodium sulphate and was concentrated. The resultant residue was purified by Reveleris C-18 reversed phase column chromatography using 60-65% acetonitrile in aqueous formic acid (0.1%) to afford 2-(4-chlorophenyl-2-d)-N-((2-(2,6-dioxopiperidin-3-yl)-1-oxoisoindolin-5-yl)methyl)-2,2-difluoroacetamide (27 mg, 0.06 mmol, 6% yield) white solid. ¹H NMR (400 MHz, DMSO-d6) δ 10.95 (s, 1H), 9.65 (t, J=5.9 Hz, 1H), 7.69-7.54 (m, 4H), 7.41-7.28 (m, 2H), 5.07 (dd, J=5.4, 13.2 Hz, 1H), 4.47-4.20 (m, 4H), 2.94-2.81 (m, 1H), 2.67-2.53 (m, 1H), 2.45-2.31 (m, 1H), 2.02-1.91 (m, 1H). MS (ESI) m/z 462.95 [M+1]⁺.

### Example 3

### 2-(4-chlorophenyl-2,3,5,6-d4)-N-((2-(2,6-dioxopiperidin-3-yl)-1-oxoisoindolin-5-yl)methyl)-2,2-difluoroacetamide

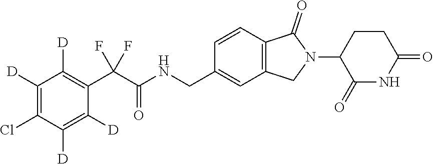 [SMILES: [2H]c1c([2H])c(C(F)(F)C(=O)NCc2ccc3c(c2)CN(C2CCC(=O)NC2=O)C3=O)c([2H])c([2H])c1Cl]

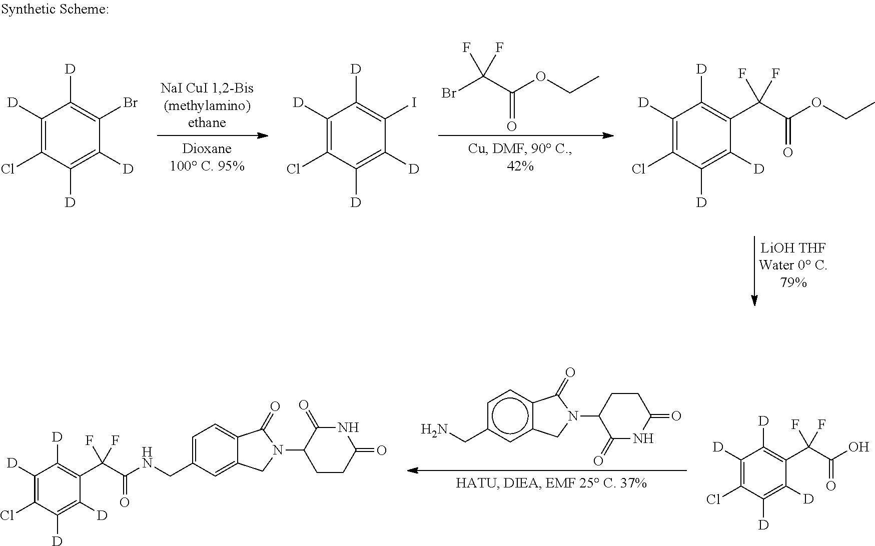

A. 1-chloro-4-iodobenzene-2,3,5,6-d4: 4-Bromochlorobenzene-d4 (7.67 g, 39.2 mmol) was placed in a vial with sodium iodide (11.76 g, 78 mmol), copper(I) iodide (0.747 g, 3.92 mmol), N¹,N²-dimethylethane-1,2-diamine (0.692 g, 7.85 mmol), and 1,4-dioxane (40.0 mL). The reaction mixture was heated to 110° C. for 22 h. After cooling, the reaction was partitioned between ethyl acetate and aqueous ammonium hydroxide solution. The organic layer was extracted with water (lx) and brine (lx). The organic layer was dried over sodium sulfate and volatile organics were removed under reduced pressure to give 1-chloro-4-iodobenzene-2,3,5,6-d4 (9.06 g, 37.4 mmol, 95% yield) as a white solid.

B. Ethyl 2-(4-chlorophenyl-2,3,5,6-d4)-2,2-difluoroacetate: 1-chloro-4-iodobenzene-2,3,5,6-d4 (9.05 g, 37.3 mmol) was placed in a vial with ethyl 2-bromo-2,2-difluoroacetate (7.58 g, 37.3 mmol), N, N-dimethylformamide (40.0 mL), and copper powder (45 micron) (6.40 g, 101 mmol). The reaction mixture was capped and stirred at 90° C. for 18 h. After cooling, the reaction mixture was diluted with ethyl acetate and filtered though celite. The filter cake was washed with more ethyl acetate. The filtrate was taken and stirred hard for 30 min with 10% potassium dihydrogen phosphate. The organic layer was taken and extracted with ethyl acetate (lx) and brine (lx). The organic layer was taken and volatile organics were removed under reduced pressure to give an orange oil. The oil was taken up in dimethyl sulfoxide and purified using reverse phase semi preparatory HPLC (50-100% acetonitrile in water+10 mM ammonium carbonate, over 30 min). Fractions containing desired product were combined and volatile organics were removed until mostly water remained and an orange oil began to fall out of solution. The mixture was partitioned between dichloromethane and brine. The organic layer was removed and the aqueous layer was extracted with dichloromethane once more. The combined organic layer was dried over sodium sulfate and volatile organics were removed under reduced pressure to give ethyl 2-(4-chlorophenyl-2,3,5,6-d4)-2,2-difluoroacetate (3.75 g, 15.71 mmol, 42.1% yield) as an orange oil.

C. 2-(4-chlorophenyl-2,3,5,6-d4)-2,2-difluoroacetic acid: Ethyl 2-(4-chlorophenyl-2,3,5,6-d4)-2,2-difluoroacetate (3.75 g, 15.71 mmol) was placed in flask with tetrahydrofuran (25 mL) and water (25.00 mL). The flask was cooled to 0° C. and lithium hydroxide (1.129 g, 47.1 mmol) was added. The reaction mixture was stirred for 90 min. The reaction mixture was transferred to a separatory funnel and more water was added along with dichloromethane. The organic layer was removed and the aqueous layer was acidified with 6N HCl to a pH of 1. The aqueous layer was extracted twice with dichloromethane. The combined organic layer was dried over sodium sulfate and volatile organics were removed under reduced pressure to give 2-(4-chlorophenyl-2,3,5,6-d4)-2,2-difluoroacetic acid (2.63 g, 12.49 mmol, 79% yield) as a light yellow crystalline solid.

D.2-(4-chlorophenyl-2,3,5,6-d4)-N-((2-(2,6-dioxopiperidin-3-yl)-1-oxoisoindolin-5-yl)methyl)-2,2-difluoroacetamide: 3-(5-(Aminomethyl)-1-oxoisoindolin-2-yl)piperidine-2,6-dione, mesylic acid (4.61 g, 12.49 mmol) was placed in a flask with N,N-dimethylformamide (25 mL), N,N-diisopropylethylamine (6.54 mL, 37.5 mmol) and 2-(4-chlorophenyl-2,3,5,6-d4)-2,2-difluoroacetic acid (2.63 g, 12.49 mmol). To the flask was added 1-[Bis(dimethyl amino)methylene]-1H-1,2,3-triazolo[4,5-b]pyridinium 3-oxide hexafluorophosphate (5.70 g, 14.99 mmol) and the reaction mixture was stirred at 25° C. for 18 h. The reaction mixture was partitioned between ethyl acetate (300 mL) and water (200 mL). The organic layer was removed and washed with saturated sodium bicarbonate solution (2×200 mL) and 1N HCl solution (2×200 mL). The organic layer was finally washed with brine (2×200 mL). The aqueous layer was removed, the organic layer was taken and volatile organics were removed under reduced pressure to give a yellow solid. Solids were slurried in water for 30 min and collected by vacuum filtration. The solid was taken up in dimethyl sulfoxide and purified using reverse-phase semi preparatory HPLC (45-65-100% acetonitrile+0.1% formic acid in water+0.1% formic acid, over 30 min). Fractions containing desired product were combined and volatile organics were removed under reduced pressure to give 2-(4-chlorophenyl-2,3,5,6-d4)-N-((2-(2,6-dioxopiperidin-3-yl)-1-oxoisoindolin-5-yl)methyl)-2,2-difluoroacetamide (2.18 g, 4.68 mmol, 37.5% yield) as a white solid. ¹H NMR (500 MHz, DMSO-d6) δ 10.97 (s, 1H), 9.67 (t, J=5.99 Hz, 1H), 7.68 (d, J=8.20 Hz, 1H), 7.40 (s, 1H), 7.36 (d, J=7.88 Hz, 1H), 5.10 (dd, J=5.20, 13.40 Hz, 1H), 4.38-4.48 (m, 3H), 4.25-4.33 (m, 1H), 2.91 (ddd, J=5.36, 13.71, 17.50 Hz, 1H), 2.56-2.64 (m, 1H), 2.38 (qd, J=4.57, 13.29 Hz, 1H), 2.00 (dtd, J=2.21, 5.24, 12.53 Hz, 1H). Anal. Calcd for C₂₂H₁₄D₄CF₂N₃O₄: C, 56.67; H, 3.86; N, 9.02. Found: C, 56.54; H, 4.09; N, 9.01. MS (ESI) m/z 466.2 [M+1]⁺.

### Example 4:2-(4-chlorophenyl)-N-((2-(2,6-dioxopiperidin-3-yl)-1-oxoisoindolin-5-yl)-12-methyl-¹⁴C)-2,2-difluoroacetamide

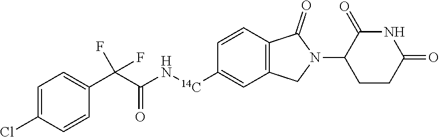 [SMILES: O=C1CCC(N2Cc3cc([14CH2]NC(=O)C(F)(F)c4ccc(Cl)cc4)ccc3C2=O)C(=O)N1]

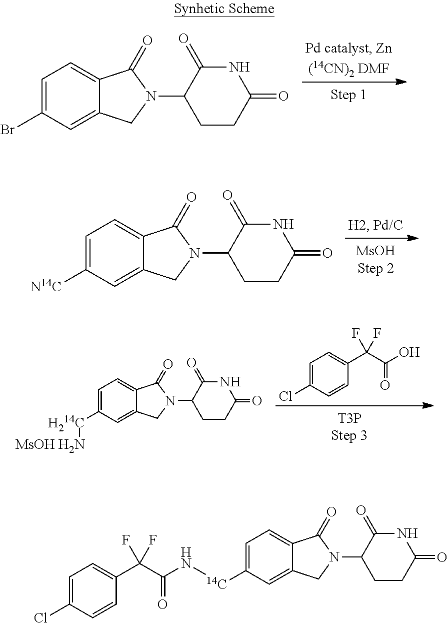

A. 2-(2,6-dioxopiperidin-3-yl)-1-oxoisoindoline-5-carbonitrile: To a mixture of 3-(5-bromo-1-oxoisoindolin-2-yl)piperidine-2,6-dione, ¹⁴C labeled Zinc cyanide, Zinc in N,N-dimethylacetamide was added 1,1′-Bis(diphenylphosphino)ferrocene-palladium(II) dichloride complex under N2 atmosphere. The reaction mixture was heated to 110° C. for 3 hours, then the reaction mixture was cooled to 10° C., and water was added. The resulting precipitate was collected by filtration to give the crude product. The crude product was washed with aqueous ammonia and dichloromethane to give 2-(2,6-dioxopiperidin-3-yl)-1-oxoisoindoline-5-carbonitrile.

B. 3-(5-(aminomethyl-1-oxoisoindolin-2-yl)piperidine-2,6-dione: Compound 2-(2,6-dioxopiperidin-3-yl)-1-oxoisoindoline-5-carbonitrile was stirred in water, n-propanol and methanesulfonic acid under hydrogen pressure. The mixture was then filtered. Filtrate was charged isopropanol. The resulting suspension was filtered and the solid was dried to provide 3-(5-(aminomethyl-1-oxoisoindolin-2-yl)piperidine-2,6-dione.

C. 2-(4-chlorophenyl)-N-((2-(2,6-dioxopiperidin-3-yl)-1-oxoisoindolin-5-yl)-12-methyl-¹⁴C)-2,2-difluoroacetamide: Compound 3-(5-(aminomethyl-1-oxoisoindolin-2-yl)piperidine-2,6-dione was stirred in DMF and n-methyl morpholine. T3P was charged slowly under hydrogen pressure. The mixture was then stirred for 12 hours. The mixture was added water and resulting suspension was filtered. The solid was dried to desired product 2-(4-chlorophenyl)-N-((2-(2,6-dioxopiperidin-3-yl)-1-oxoisoindolin-5-yl)-12-methyl-¹⁴C)-2,2-difluoroacetamide.

### Example 5: Effect of Test Compounds on KG-1 and KG-1a Cell Proliferation

The anti-proliferative activity of the test compounds was evaluated on KG-1 and KG-1a cell lines using CellTiter-Glo assay as described in U.S. Pat. No. 9,499,514 at 72 hours post-treatment. IC₅₀ values for exemplary compounds are provided in Table 6.

TABLE 6

| | FCA Prolif Cell | FCA Prolif Cell |
|---|---|---|
| | TiterGlo KG-1 72 h | TiterGlo KG-1a 72 h |
| Compound | (IC₅₀) | (IC₅₀) |
| 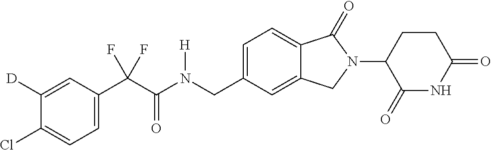 [SMILES: [2H]c1cc(C(F)(F)C(=O)NCc2ccc3c(c2)CN(C2CCC(=O)NC2=O)C3=O)ccc1Cl] | 0.01545 | 0.01815 |
| 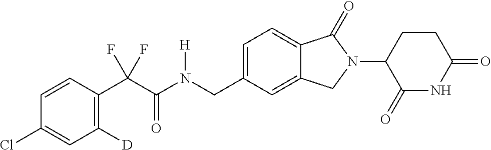 [SMILES: [2H]c1cc(Cl)ccc1C(F)(F)C(=O)NCc1ccc2c(c1)CN(C1CCC(=O)NC1=O)C2=O] | 0.036899 | 0.029865 |
| 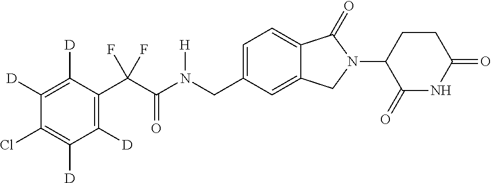 [SMILES: [2H]c1c([2H])c(C(F)(F)C(=O)NCc2ccc3c(c2)CN(C2CCC(=O)NC2=O)C3=O)c([2H])c([2H])c1Cl] | 0.026179 | 0.031302 |

### Example 6: Determination of Isotopic Enrichment

Isotopic enrichment may be confirmed and quantified by mass spectrometry and/or NMR, including, for example, proton-NMR; carbon-13 NMR; or nitrogen-15 NMR.

Isotopic enrichment may also be confirmed by single-crystal neutron diffraction. For example, the isotopic ratio at a particular hydrogen/deuterium position in a deuterated Compound A can be determined using single-crystal neutron diffraction. Neutron diffraction is advantageous because neutrons are scattered by the nucleus of an atom, therefore allowing for discrimination between isotopes, such as hydrogen and deuterium, which differ in the number of neutrons in the nucleus.

A single crystal of suitable size and quality comprising the deuterated Compound A is grown using standard methods of crystal growth. For single-crystal neutron diffraction experiments, crystals of several cubic millimeters are generally required for suitable data collection. A minimum size for a single crystal is typically about 1 cubic millimeter. Suitable single crystals are obtained by dissolving the deuterated Compound A in a solvent with appreciable solubility, then slowly evaporating or cooling the solution to yield crystals of suitable size and quality. Alternatively, suitable single crystals are obtained by dissolving the deuterated Compound Ain a solvent with appreciable solubility, then slowly diffusing into the solution of antisolvent (i.e., a solvent in which the deuterated Compound A is not appreciably soluble) to yield crystals of suitable size and quality. These and other suitable methods of crystal growth are known in the art and are described, e.g., in George H. Stout & Lyle H. Jensen, X-Ray Structure Determination: A Practical Guide 74-92 (John Wiley & Sons, Inc. 2nd ed. 1989) (the entirety of which is incorporated herein).

After isolating a suitable single crystal comprising the deuterated Compound A, the crystal is mounted in a neutron beam, neutron diffraction data is collected, and the crystal structure is solved and refined. Different neutron sources can be used, including steady-state sources and pulsed spallation sources. Examples of steady-state sources include the Grenoble ILL High Flux Reactor (Grenoble, France) and the Oak Ridge High Flux Isotope Reactor (Oak Ridge, Tenn.). Examples of pulsed spallation sources include ISIS, the spallation neutron source at Rutherford Appleton Laboratory (Oxfordshire, UK); the Intense Pulsed Neutron Source (IPNS) at Argonne National Laboratory (Argonne, Ill.), the Los Alamos Neutron Science Center (LANSCE) at Los Alamos National Laboratory (Los Alamos, N. Mex.), and the Neutron Science Laboratory (KENS) at KEK (Tsukuba, Ibaraki, Japan).

For a steady-state neutron source, four-circle diffractometer techniques are used with a monochromatic beam and a single detector, rotating the crystal and detector to measure each reflection sequentially. Diffractometer control software and step-scanning methods for intensity extraction can be adopted from routine four-circle X-ray diffractometry methods. One or more area detectors, including area detector arrays, may alternatively be used to increase the region of reciprocal space accessed in a single measurement. A broad band (white) beam used with an area detector allows for Laue or quasi-Laue diffraction with a stationary crystal and detector.

For a pulse source with a white neutron beam, time-of-flight Laue diffraction techniques are used, which allow for the determination of the velocity, energy, and wavelength of each neutron detected. This approach combines wavelength sorting with large area position-sensitive detectors, and allows for fixed scattering geometries (i.e., a stationary crystal and detector). Pulse source data collected in this fashion allows for rapid collection of data sets and good accuracy and precision in standard structural refinements. Additional details regarding steady-state and pulse source neutron diffraction experiments are well known in the art. See, e.g., Chick C. Wilson, Neutron Single Crystal Diffraction, 220 Z. Kristallogr. 385-98 (2005) (incorporated by reference herein in its entirety).

Crystal structure data, including particular isotopic ratios, are obtained from neutron diffraction data following routine structure solution and refinement processes. Structure solution is carried out using one of several methods, including direct methods and Patterson methods. For convenience, atomic coordinates from prior single crystal X-ray diffraction experiments may be used as a starting point for structure refinement using neutron diffraction data; this approach permits additional refinement of atomic positions, including hydrogen and deuterium positions. Refinement is conducted using full-matrix least-squares methods to achieve optimal agreement between the observed diffraction intensities and those calculated from the structural model. Ideally, full anisotropic refinement is carried out on all atoms, including the H/D atomic positions of interest. Data collection, structure solution and structure refinement methods, both for X-ray and neutron diffraction data, are well known in the art. See, e.g., Chick C. Wilson, Single Crystal Neutron Diffraction from Molecular Materials (World Scientific Publishing Co. 2000); George H. Stout & Lyle H. Jensen, X-Ray Structure Determination: A Practical Guide (John Wiley & Sons, Inc. 2nd ed. 1989) (both of which are incorporated herein in their entireties).

The isotopic ratio for a particular position on a deuterated Compound A is calculated by examining the neutron scattering cross sections for the H/D atomic position of interest. The scattering cross section is obtained as part of the refinement process discussed above. An example of determining the isotopic ratio for a partially deuterated compound is provided by G. A. Jeffrey et al., Neutron Diffraction Refinement of Partially Deuterated β-D-Arabinopyranose and α-L-Xylopyranose at 123 K, B36 Acta Crystallographica 373-77 (1980) (incorporated by reference herein in its entirety). Jeffrey et al. used single-crystal neutron diffraction to determine the percentage deuterium substitution for hydroxyl groups on two sugar compounds of interest. Employing the methods discussed by Jeffrey et al., one may similarly ascertain the isotopic ratio for a particular H/D position on a deuterated Compound A.

All of the cited references are incorporated herein by reference in their entirety.

1. A compound, wherein the compound is an isotopologue of a compound having the following structure: 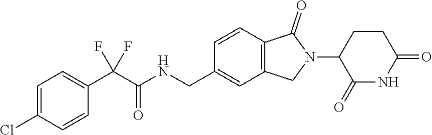 [SMILES: O=C1CCC(N2Cc3cc(CNC(=O)C(F)(F)c4ccc(Cl)cc4)ccc3C2=O)C(=O)N1]
or a stereoisomer, a mixture of stereoisomers, a pharmaceutically acceptable salt, a tautomer, a solvate, a hydrate, a co-crystal, a clathrate, or a polymorph thereof.

2. The compound of claim 1, wherein the isotopologue is an isotopologue of a compound having the following structure:

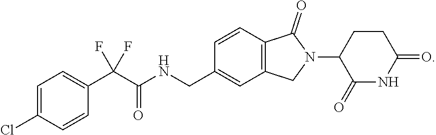 [SMILES: O=C1CCC(N2Cc3cc(CNC(=O)C(F)(F)c4ccc(Cl)cc4)ccc3C2=O)C(=O)N1]

3. The compound of claim 1, wherein the isotopologue is deuterium-enriched.

4. The compound of claim 1, wherein the isotopologue is radiolabeled with carbon-14 (¹⁴C).

5. The compound of claim 1, wherein the isotopologue is a compound having formula A1: 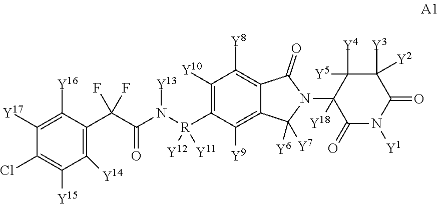
or a stereoisomer, a mixture of stereoisomers, a pharmaceutically acceptable salt, a tautomer, a solvate, a hydrate, a co-crystal, a clathrate, or a polymorph thereof, wherein
R is C or ¹⁴C; when R is C then one or more of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, and Y¹⁸ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷ and Y¹⁸ are non-enriched hydrogen atoms; and when R is ¹⁴C, then optionally one or more of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, and Y¹⁸ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷ and Y¹⁸ are non-enriched hydrogen atoms.

6. The compound of claim 1, wherein the isotopologue is a compound having formula A2: 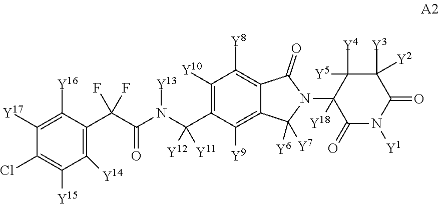
or a stereoisomer, a mixture of stereoisomers, a pharmaceutically acceptable salt, a tautomer, a solvate, a hydrate, a co-crystal, a clathrate, or a polymorph thereof, wherein one or more of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, and Y¹⁸ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷ and Y¹⁸ are non-enriched hydrogen atoms.

7. The compound of claim 1, wherein the isotopologue is a compound having formula A3: 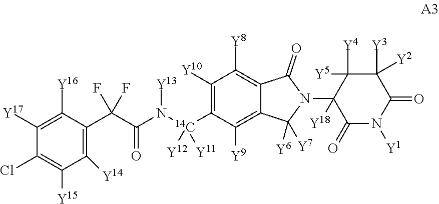
or a stereoisomer, a mixture of stereoisomers, a pharmaceutically acceptable salt, a tautomer, a solvate, a hydrate, a co-crystal, a clathrate, or a polymorph thereof, wherein optionally one or more of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, and Y¹⁸ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷ and Y¹⁸ are non-enriched hydrogen atoms.

8. The compound of claim 5, wherein one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, and Y¹⁸ is isotopically enriched with deuterium, and the others are non-enriched hydrogens.

9. The compound of claim 5, wherein two of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷ and Y¹⁸ are isotopically enriched with deuterium, and the others are non-enriched hydrogens.

10. The compound of claim 6, wherein the compound is:

| No. | Compound structure |
|---|---|
| 1 | 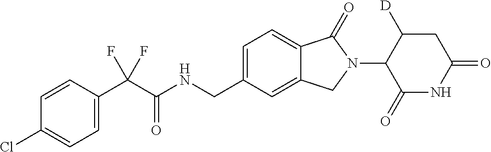 [SMILES: [2H]C1CC(=O)NC(=O)C1N1Cc2cc(CNC(=O)C(F)(F)c3ccc(Cl)cc3)ccc2C1=O] |
| 2 | 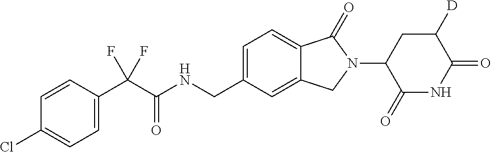 [SMILES: [2H]C1CC(N2Cc3cc(CNC(=O)C(F)(F)c4ccc(Cl)cc4)ccc3C2=O)C(=O)NC1=O] |
| 3 | 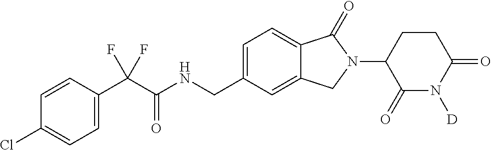 |
| 4 | 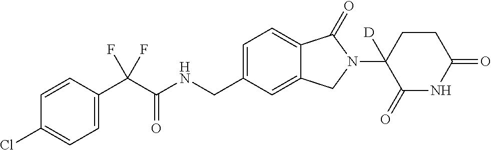 [SMILES: [2H]C1(N2Cc3cc(CNC(=O)C(F)(F)c4ccc(Cl)cc4)ccc3C2=O)CCC(=O)NC1=O] |
| 5 | 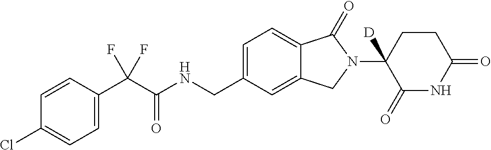 [SMILES: [2H][C@@]1(N2Cc3cc(CNC(=O)C(F)(F)c4ccc(Cl)cc4)ccc3C2=O)CCC(=O)NC1=O] |
| 6 | 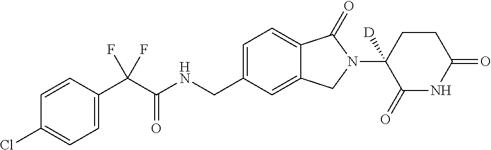 [SMILES: [2H][C@]1(N2Cc3cc(CNC(=O)C(F)(F)c4ccc(Cl)cc4)ccc3C2=O)CCC(=O)NC1=O] |
| 7 | 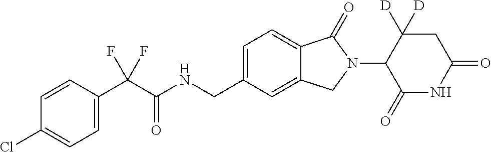 [SMILES: [2H]C1([2H])CC(=O)NC(=O)C1N1Cc2cc(CNC(=O)C(F)(F)c3ccc(Cl)cc3)ccc2C1=O] |
| 8 | 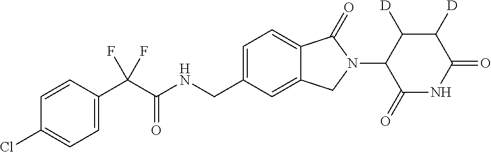 [SMILES: [2H]C1C(=O)NC(=O)C(N2Cc3cc(CNC(=O)C(F)(F)c4ccc(Cl)cc4)ccc3C2=O)C1[2H]] |
| 9 | 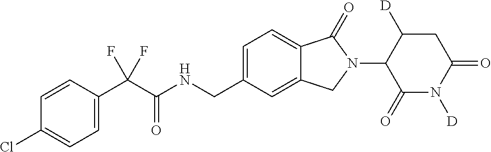 |
| 10 | 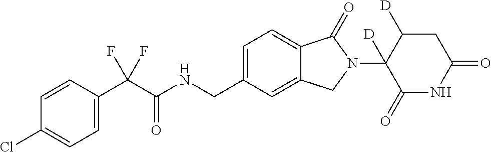 [SMILES: [2H]C1CC(=O)NC(=O)C1([2H])N1Cc2cc(CNC(=O)C(F)(F)c3ccc(Cl)cc3)ccc2C1=O] |
| 11 | 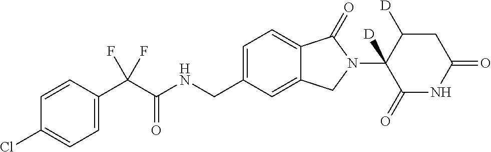 [SMILES: [2H]C1CC(=O)NC(=O)[C@]1([2H])N1Cc2cc(CNC(=O)C(F)(F)c3ccc(Cl)cc3)ccc2C1=O] |
| 12 | 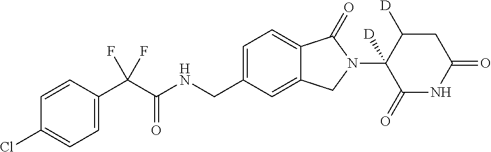 [SMILES: [2H]C1CC(=O)NC(=O)[C@@]1([2H])N1Cc2cc(CNC(=O)C(F)(F)c3ccc(Cl)cc3)ccc2C1=O] |
| 13 | 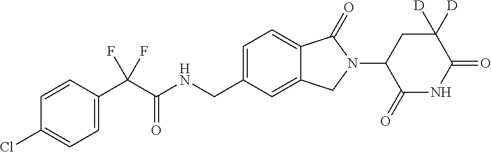 [SMILES: [2H]C1([2H])CC(N2Cc3cc(CNC(=O)C(F)(F)c4ccc(Cl)cc4)ccc3C2=O)C(=O)NC1=O] |
| 14 | 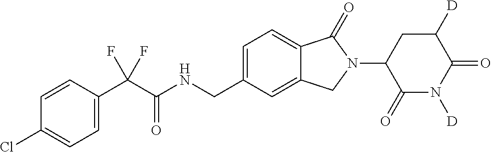 |
| 15 | 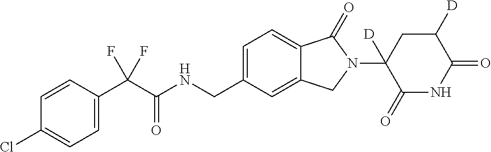 [SMILES: [2H]C1CC([2H])(N2Cc3cc(CNC(=O)C(F)(F)c4ccc(Cl)cc4)ccc3C2=O)C(=O)NC1=O] |
| 16 | 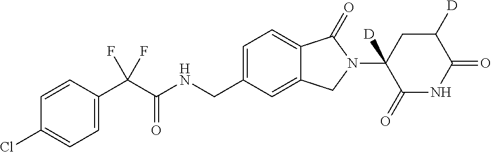 [SMILES: [2H]C1C[C@@]([2H])(N2Cc3cc(CNC(=O)C(F)(F)c4ccc(Cl)cc4)ccc3C2=O)C(=O)NC1=O] |
| 17 | 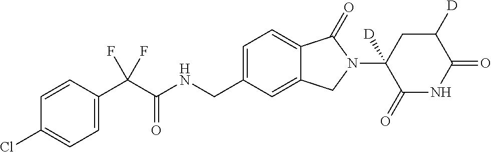 [SMILES: [2H]C1C[C@]([2H])(N2Cc3cc(CNC(=O)C(F)(F)c4ccc(Cl)cc4)ccc3C2=O)C(=O)NC1=O] |
| 18 | 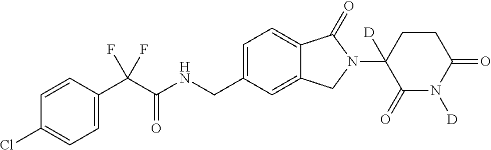 |
| 19 | 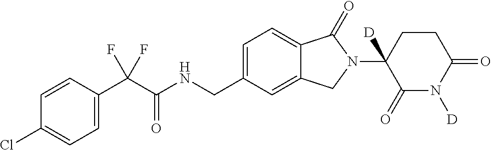 |
| 20 | 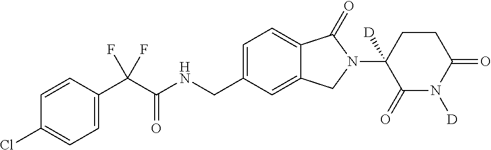 |
| 21 | 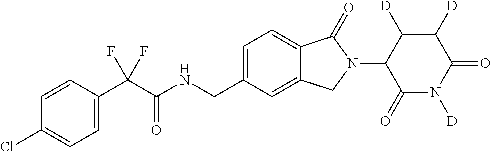 |
| 22 | 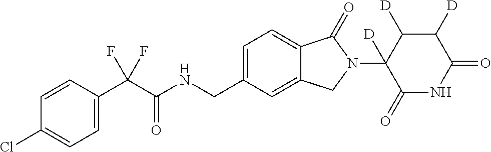 [SMILES: [2H]C1C(=O)NC(=O)C([2H])(N2Cc3cc(CNC(=O)C(F)(F)c4ccc(Cl)cc4)ccc3C2=O)C1[2H]] |
| 23 | 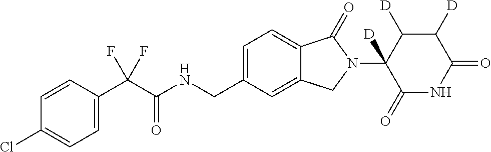 [SMILES: [2H]C1C(=O)NC(=O)[C@]([2H])(N2Cc3cc(CNC(=O)C(F)(F)c4ccc(Cl)cc4)ccc3C2=O)C1[2H]] |
| 24 | 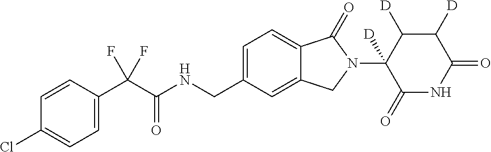 [SMILES: [2H]C1C(=O)NC(=O)[C@@]([2H])(N2Cc3cc(CNC(=O)C(F)(F)c4ccc(Cl)cc4)ccc3C2=O)C1[2H]] |
| 25 | 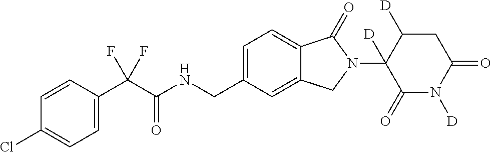 |
| 26 | 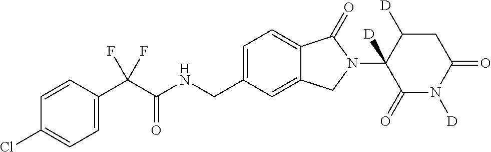 |
| 27 | 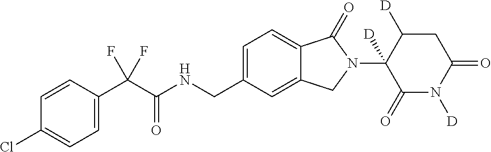 |
| 28 | 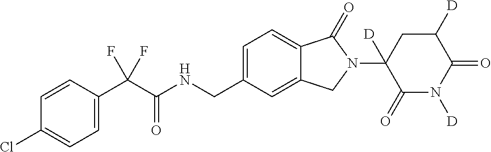 |
| 29 | 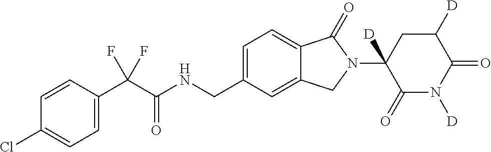 |
| 30 | 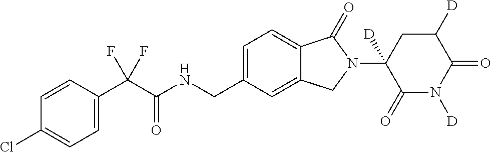 |
| 31 | 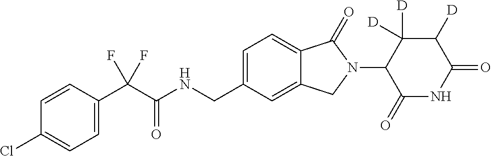 [SMILES: [2H]C1C(=O)NC(=O)C(N2Cc3cc(CNC(=O)C(F)(F)c4ccc(Cl)cc4)ccc3C2=O)C1([2H])[2H]] |
| 32 | 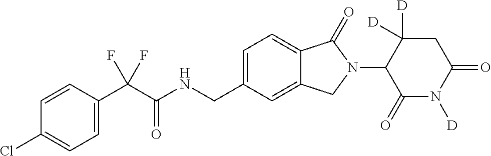 |
| 33 | 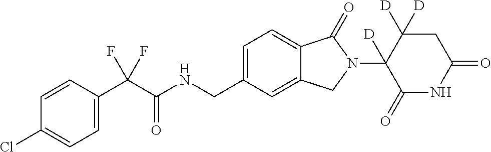 [SMILES: [2H]C1([2H])CC(=O)NC(=O)C1([2H])N1Cc2cc(CNC(=O)C(F)(F)c3ccc(Cl)cc3)ccc2C1=O] |
| 34 | 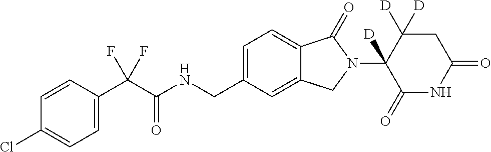 [SMILES: [2H]C1([2H])CC(=O)NC(=O)[C@]1([2H])N1Cc2cc(CNC(=O)C(F)(F)c3ccc(Cl)cc3)ccc2C1=O] |
| 35 | 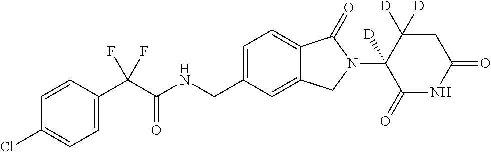 [SMILES: [2H]C1([2H])CC(=O)NC(=O)[C@@]1([2H])N1Cc2cc(CNC(=O)C(F)(F)c3ccc(Cl)cc3)ccc2C1=O] |
| 36 | 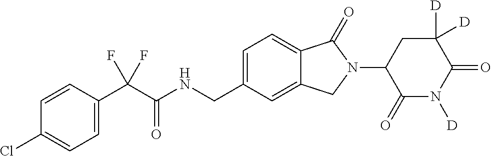 |
| 37 | 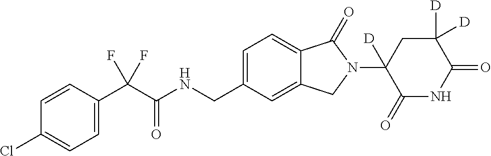 [SMILES: [2H]C1([2H])CC([2H])(N2Cc3cc(CNC(=O)C(F)(F)c4ccc(Cl)cc4)ccc3C2=O)C(=O)NC1=O] |
| 38 | 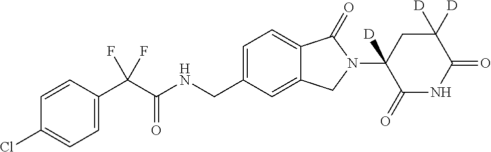 [SMILES: [2H]C1([2H])C[C@@]([2H])(N2Cc3cc(CNC(=O)C(F)(F)c4ccc(Cl)cc4)ccc3C2=O)C(=O)NC1=O] |
| 39 | 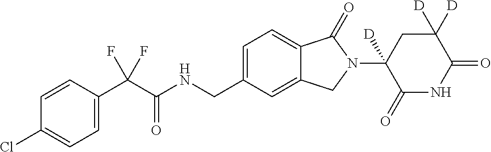 [SMILES: [2H]C1([2H])C[C@]([2H])(N2Cc3cc(CNC(=O)C(F)(F)c4ccc(Cl)cc4)ccc3C2=O)C(=O)NC1=O] |
| 40 | 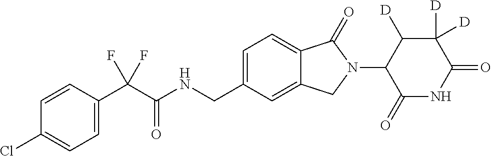 [SMILES: [2H]C1C(N2Cc3cc(CNC(=O)C(F)(F)c4ccc(Cl)cc4)ccc3C2=O)C(=O)NC(=O)C1([2H])[2H]] |
| 41 | 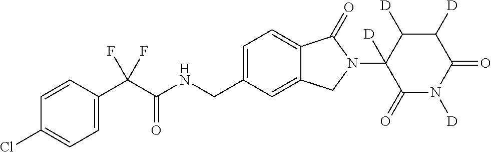 |
| 42 | 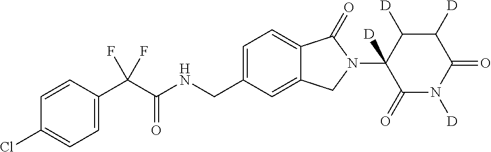 |
| 43 | 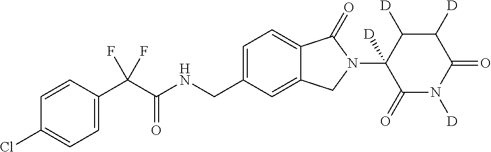 |
| 44 | 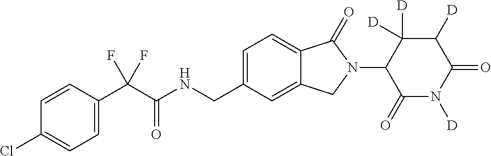 |
| 45 | 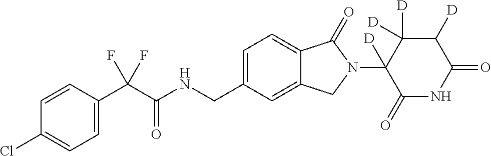 [SMILES: [2H]C1C(=O)NC(=O)C([2H])(N2Cc3cc(CNC(=O)C(F)(F)c4ccc(Cl)cc4)ccc3C2=O)C1([2H])[2H]] |
| 46 | 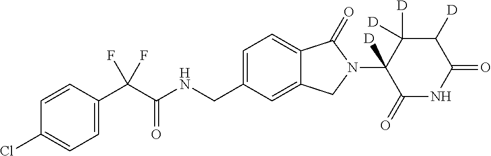 [SMILES: [2H]C1C(=O)NC(=O)[C@]([2H])(N2Cc3cc(CNC(=O)C(F)(F)c4ccc(Cl)cc4)ccc3C2=O)C1([2H])[2H]] |
| 47 | 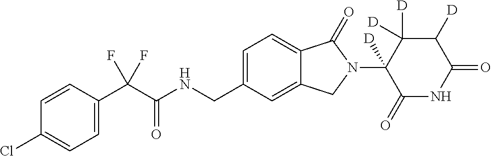 [SMILES: [2H]C1C(=O)NC(=O)[C@@]([2H])(N2Cc3cc(CNC(=O)C(F)(F)c4ccc(Cl)cc4)ccc3C2=O)C1([2H])[2H]] |
| 48 | 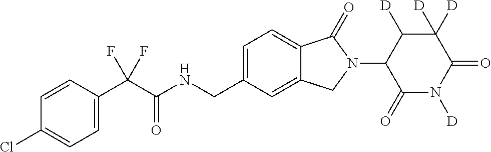 |
| 49 | 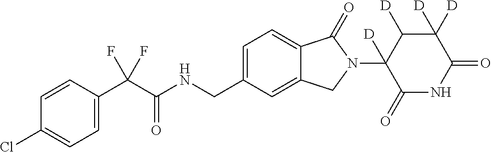 [SMILES: [2H]C1C([2H])([2H])C(=O)NC(=O)C1([2H])N1Cc2cc(CNC(=O)C(F)(F)c3ccc(Cl)cc3)ccc2C1=O] |
| 50 | 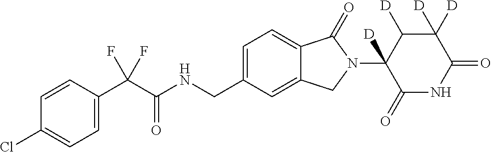 [SMILES: [2H]C1C([2H])([2H])C(=O)NC(=O)[C@]1([2H])N1Cc2cc(CNC(=O)C(F)(F)c3ccc(Cl)cc3)ccc2C1=O] |
| 51 | 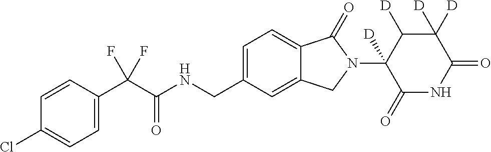 [SMILES: [2H]C1C([2H])([2H])C(=O)NC(=O)[C@@]1([2H])N1Cc2cc(CNC(=O)C(F)(F)c3ccc(Cl)cc3)ccc2C1=O] |
| 52 | 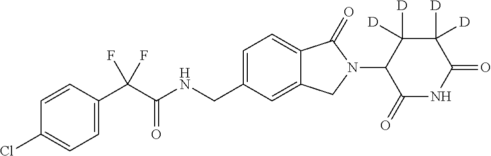 [SMILES: [2H]C1([2H])C(=O)NC(=O)C(N2Cc3cc(CNC(=O)C(F)(F)c4ccc(Cl)cc4)ccc3C2=O)C1([2H])[2H]] |
| 53 | 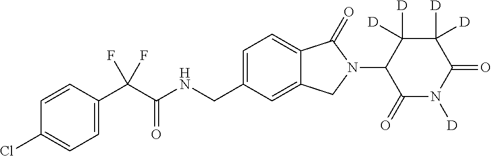 |
| 54 | 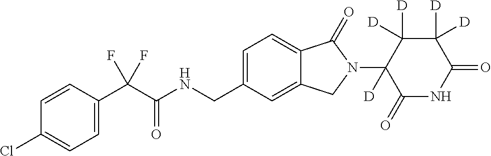 [SMILES: [2H]C1([2H])C(=O)NC(=O)C([2H])(N2Cc3cc(CNC(=O)C(F)(F)c4ccc(Cl)cc4)ccc3C2=O)C1([2H])[2H]] |
| 55 | 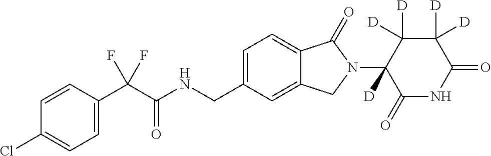 [SMILES: [2H]C1([2H])C(=O)NC(=O)[C@]([2H])(N2Cc3cc(CNC(=O)C(F)(F)c4ccc(Cl)cc4)ccc3C2=O)C1([2H])[2H]] |
| 56 | 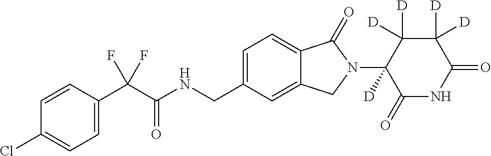 [SMILES: [2H]C1([2H])C(=O)NC(=O)[C@@]([2H])(N2Cc3cc(CNC(=O)C(F)(F)c4ccc(Cl)cc4)ccc3C2=O)C1([2H])[2H]] |
| 57 | 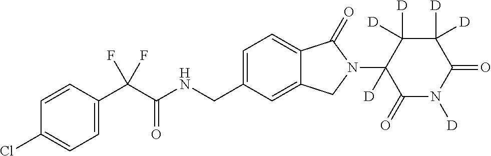 |
| 58 | 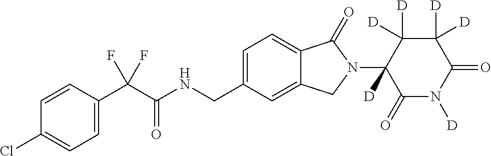 |
| 59 | 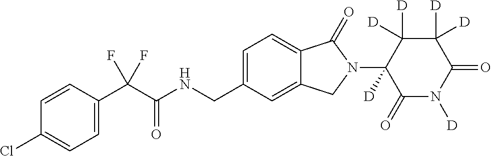 |
| 60 | 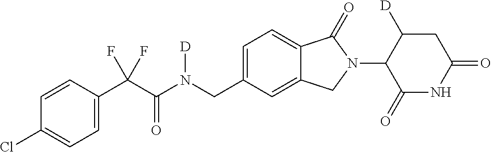 |
| 61 | 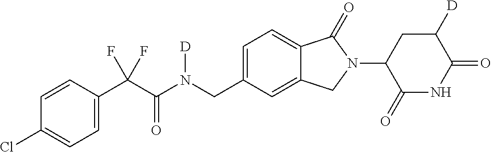 |
| 62 | 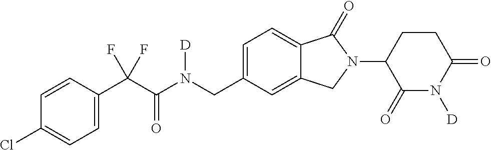 |
| 63 | 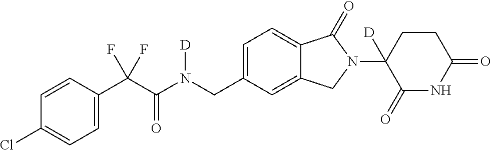 |
| 64 | 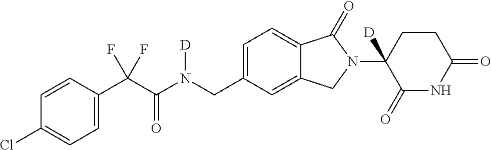 |
| 65 | 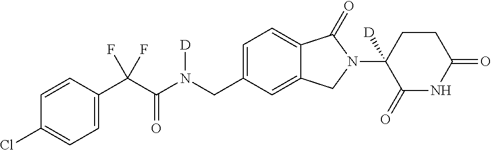 |
| 66 | 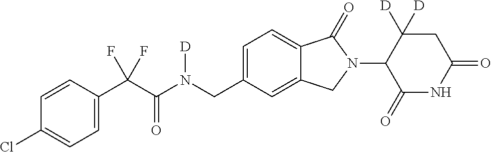 |
| 67 | 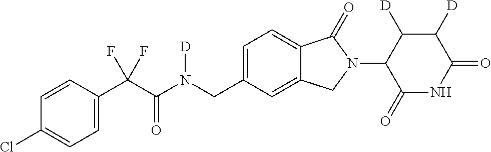 |
| 68 | 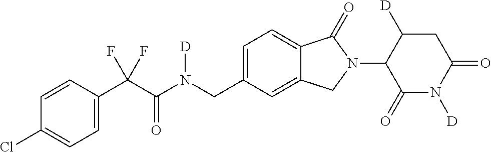 |
| 69 | 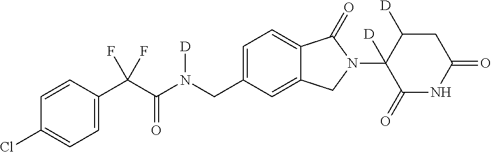 |
| 70 | 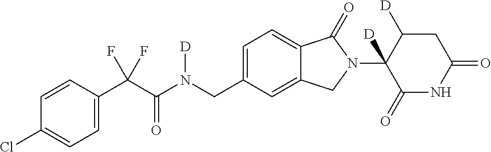 |
| 71 | 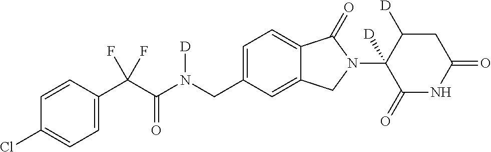 |
| 72 | 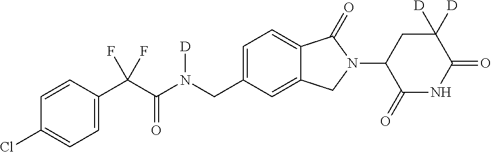 |
| 73 | 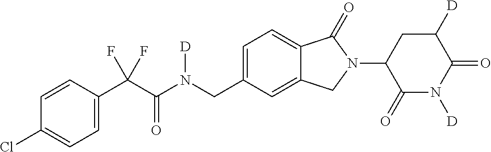 |
| 74 | 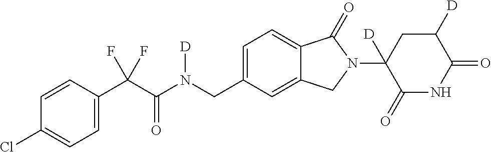 |
| 75 | 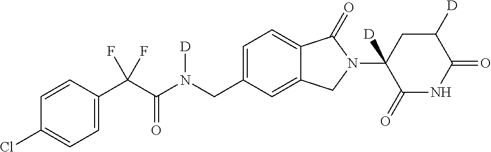 |
| 76 | 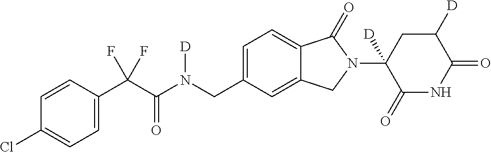 |
| 77 | 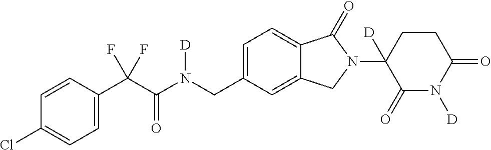 |
| 78 | 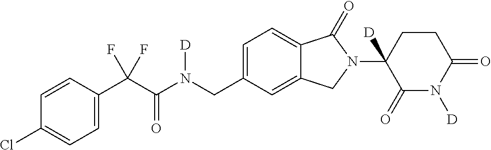 |
| 79 | 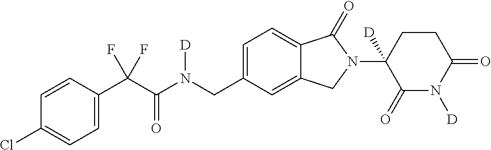 |
| 80 | 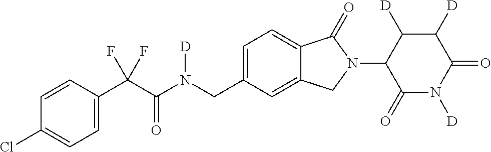 |
| 81 | 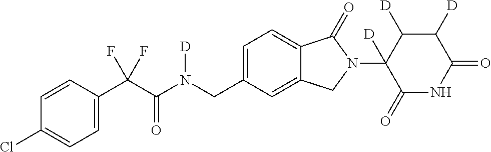 |
| 82 | 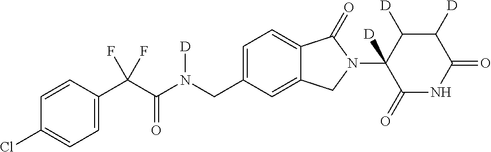 |
| 83 | 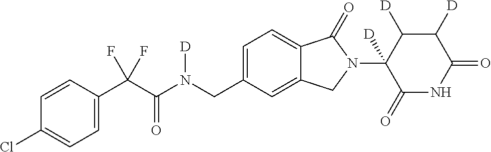 |
| 84 | 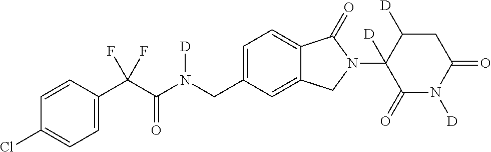 |
| 85 | 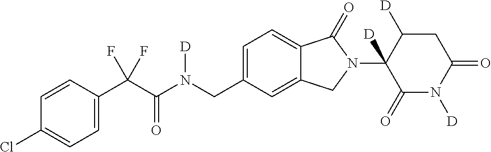 |
| 86 | 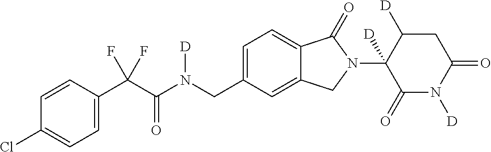 |
| 87 | 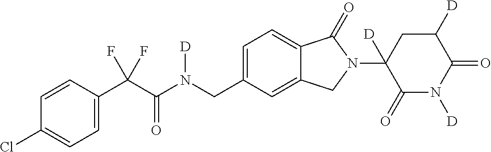 |
| 88 | 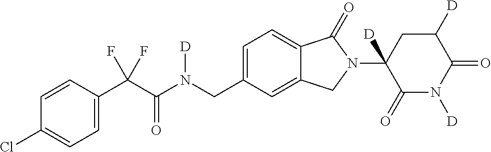 |
| 89 | 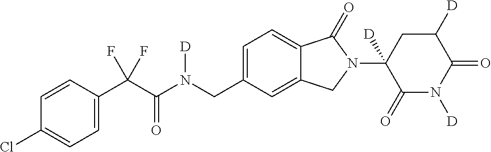 |
| 90 | 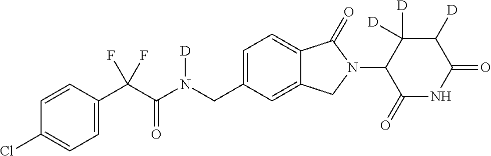 |
| 91 | 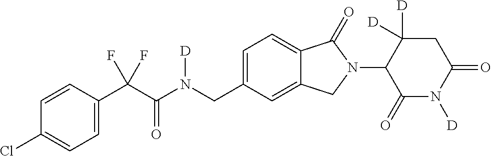 |
| 92 | 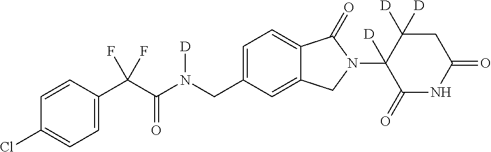 |
| 93 | 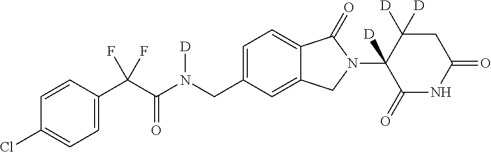 |
| 94 | 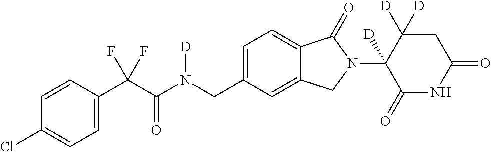 |
| 95 | 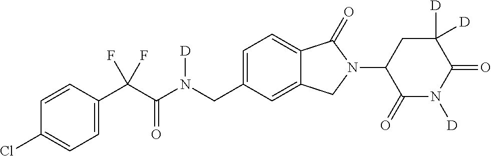 |
| 96 | 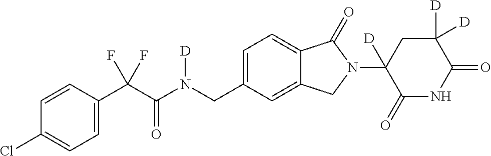 |
| 97 | 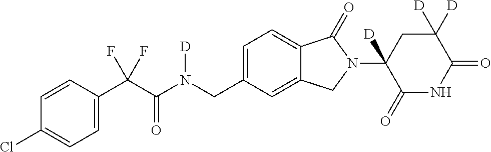 |
| 98 | 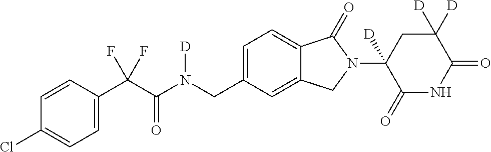 |
| 99 | 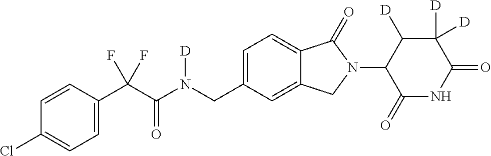 |
| 100 | 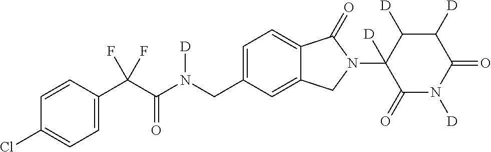 |
| 101 | 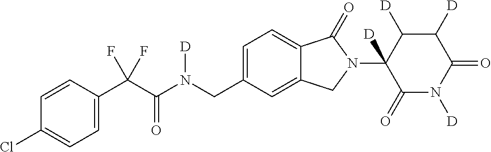 |
| 102 | 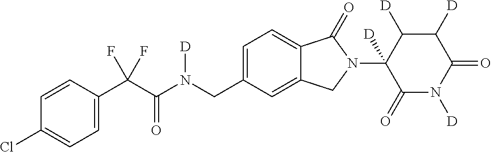 |
| 103 | 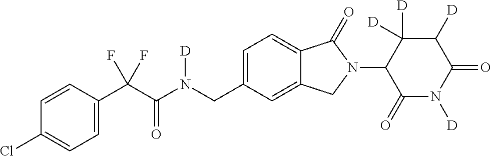 |
| 104 | 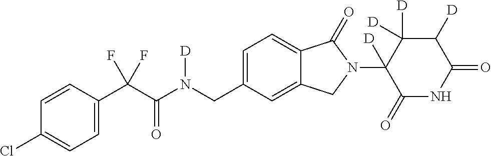 |
| 105 | 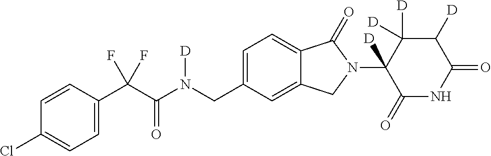 |
| 106 | 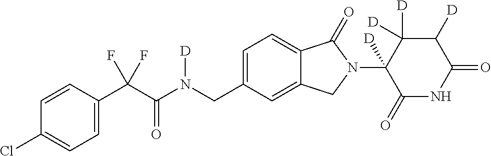 |
| 107 | 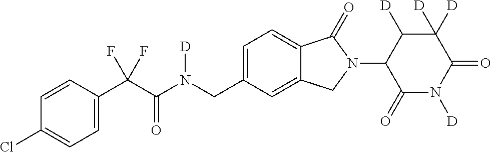 |
| 108 | 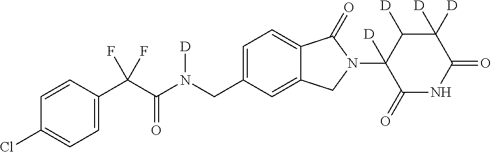 |
| 109 | 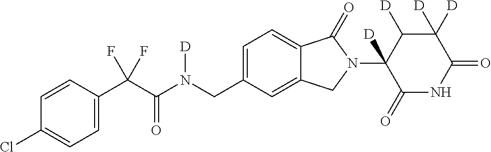 |
| 110 | 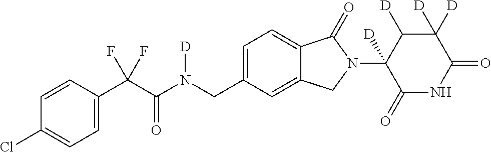 |
| 111 | 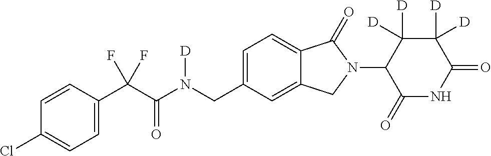 |
| 112 | 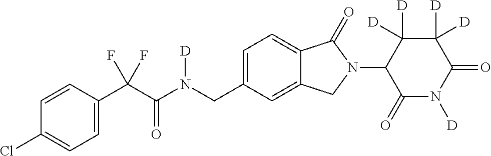 |
| 113 | 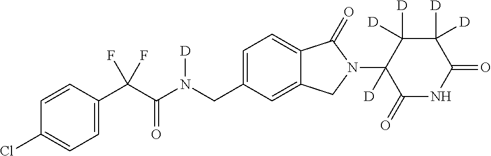 |
| 114 | 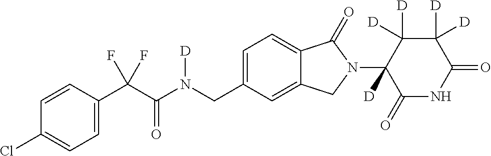 |
| 115 | 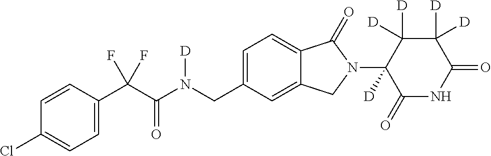 |
| 116 | 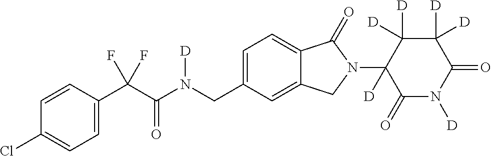 |
| 117 | 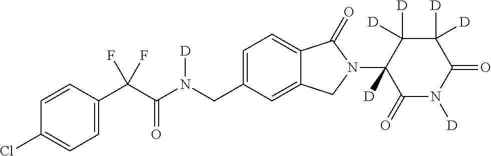 |
| 118 | 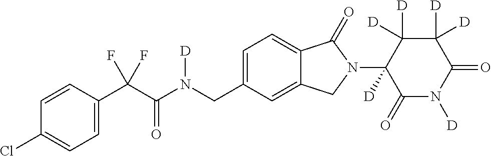 |
| 119 | 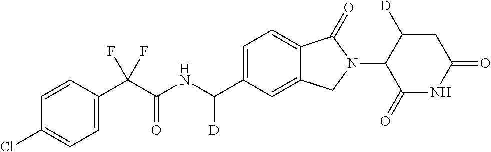 [SMILES: [2H]C(NC(=O)C(F)(F)c1ccc(Cl)cc1)c1ccc2c(c1)CN(C1C(=O)NC(=O)CC1[2H])C2=O] |
| 120 | 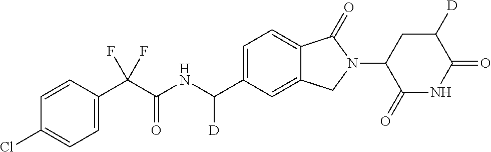 [SMILES: [2H]C1CC(N2Cc3cc(C([2H])NC(=O)C(F)(F)c4ccc(Cl)cc4)ccc3C2=O)C(=O)NC1=O] |
| 121 | 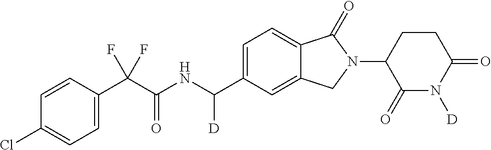 |
| 122 | 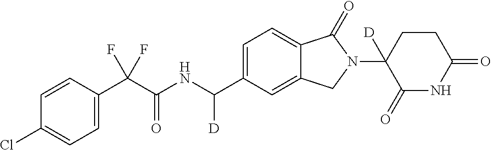 [SMILES: [2H]C(NC(=O)C(F)(F)c1ccc(Cl)cc1)c1ccc2c(c1)CN(C1([2H])CCC(=O)NC1=O)C2=O] |
| 123 | 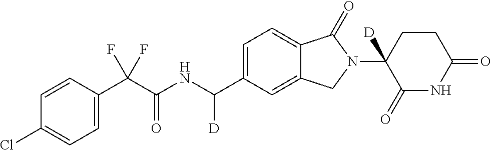 [SMILES: [2H]C(NC(=O)C(F)(F)c1ccc(Cl)cc1)c1ccc2c(c1)CN([C@]1([2H])CCC(=O)NC1=O)C2=O] |
| 124 | 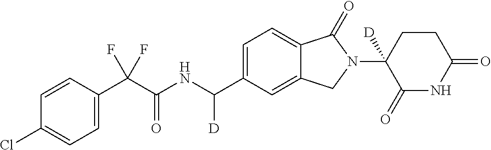 [SMILES: [2H]C(NC(=O)C(F)(F)c1ccc(Cl)cc1)c1ccc2c(c1)CN([C@@]1([2H])CCC(=O)NC1=O)C2=O] |
| 125 | 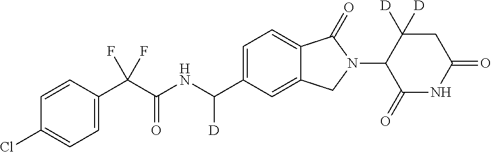 [SMILES: [2H]C(NC(=O)C(F)(F)c1ccc(Cl)cc1)c1ccc2c(c1)CN(C1C(=O)NC(=O)CC1([2H])[2H])C2=O] |
| 126 | 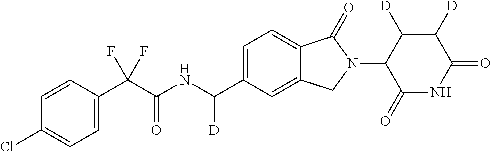 [SMILES: [2H]C(NC(=O)C(F)(F)c1ccc(Cl)cc1)c1ccc2c(c1)CN(C1C(=O)NC(=O)C([2H])C1[2H])C2=O] |
| 127 | 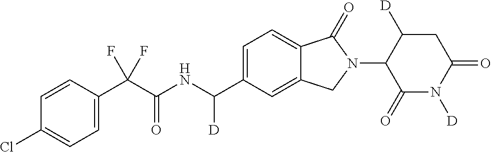 |
| 128 | 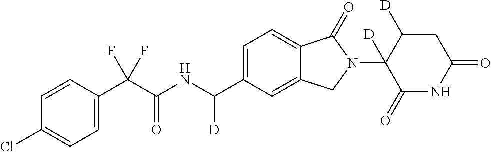 [SMILES: [2H]C(NC(=O)C(F)(F)c1ccc(Cl)cc1)c1ccc2c(c1)CN(C1([2H])C(=O)NC(=O)CC1[2H])C2=O] |
| 129 | 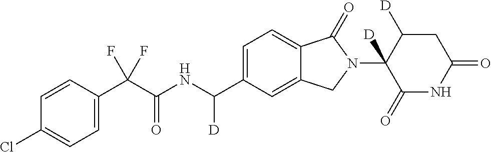 [SMILES: [2H]C(NC(=O)C(F)(F)c1ccc(Cl)cc1)c1ccc2c(c1)CN([C@@]1([2H])C(=O)NC(=O)CC1[2H])C2=O] |
| 130 | 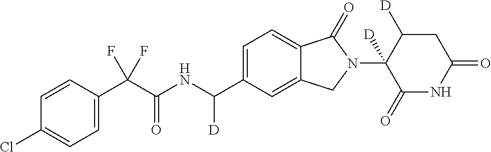 [SMILES: [2H]C(NC(=O)C(F)(F)c1ccc(Cl)cc1)c1ccc2c(c1)CN([C@]1([2H])C(=O)NC(=O)CC1[2H])C2=O] |
| 131 | 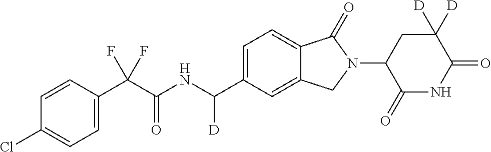 [SMILES: [2H]C(NC(=O)C(F)(F)c1ccc(Cl)cc1)c1ccc2c(c1)CN(C1CC([2H])([2H])C(=O)NC1=O)C2=O] |
| 132 | 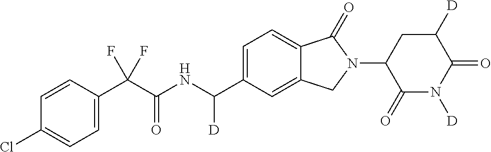 |
| 133 | 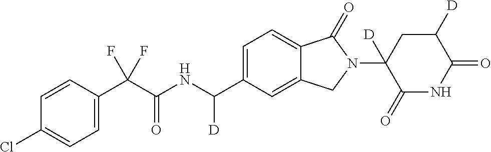 [SMILES: [2H]C1CC([2H])(N2Cc3cc(C([2H])NC(=O)C(F)(F)c4ccc(Cl)cc4)ccc3C2=O)C(=O)NC1=O] |
| 134 | 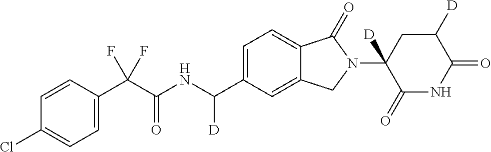 [SMILES: [2H]C1C[C@@]([2H])(N2Cc3cc(C([2H])NC(=O)C(F)(F)c4ccc(Cl)cc4)ccc3C2=O)C(=O)NC1=O] |
| 135 | 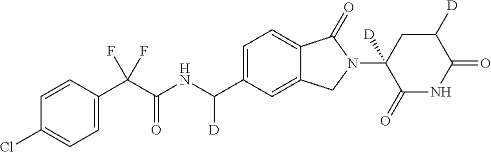 [SMILES: [2H]C1C[C@]([2H])(N2Cc3cc(C([2H])NC(=O)C(F)(F)c4ccc(Cl)cc4)ccc3C2=O)C(=O)NC1=O] |
| 136 | 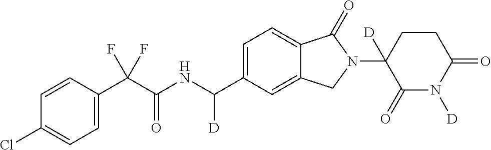 |
| 137 | 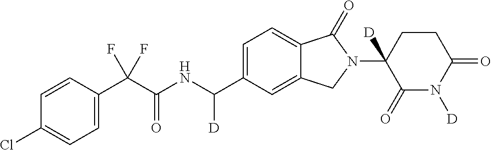 |
| 138 | 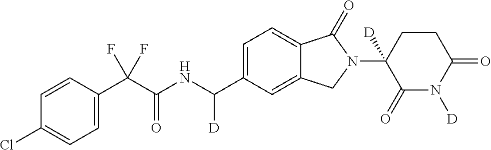 |
| 139 | 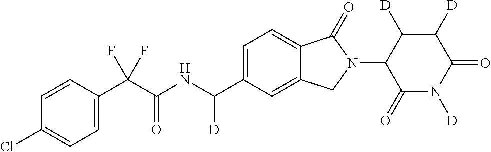 |
| 140 | 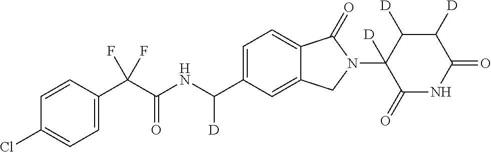 [SMILES: [2H]C(NC(=O)C(F)(F)c1ccc(Cl)cc1)c1ccc2c(c1)CN(C1([2H])C(=O)NC(=O)C([2H])C1[2H])C2=O] |
| 141 | 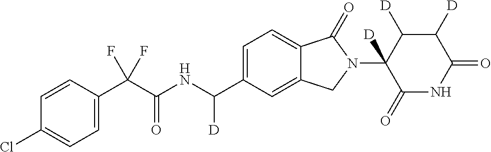 [SMILES: [2H]C(NC(=O)C(F)(F)c1ccc(Cl)cc1)c1ccc2c(c1)CN([C@@]1([2H])C(=O)NC(=O)C([2H])C1[2H])C2=O] |
| 142 | 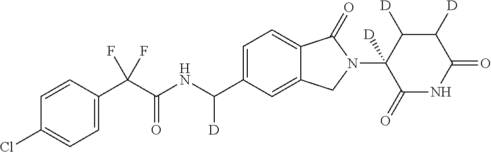 [SMILES: [2H]C(NC(=O)C(F)(F)c1ccc(Cl)cc1)c1ccc2c(c1)CN([C@]1([2H])C(=O)NC(=O)C([2H])C1[2H])C2=O] |
| 143 | 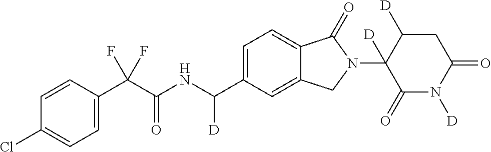 |
| 144 | 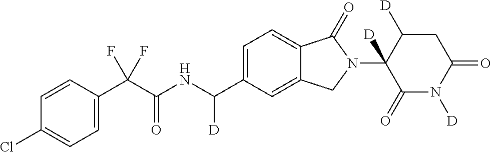 |
| 145 | 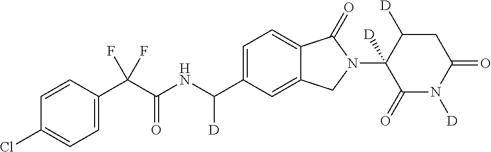 |
| 146 | 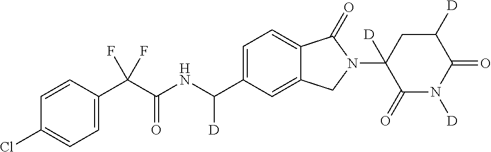 |
| 147 | 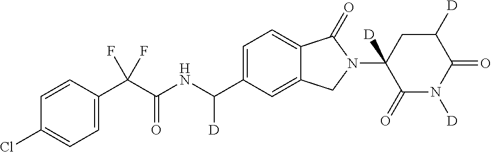 |
| 148 | 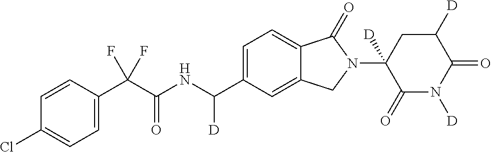 |
| 149 | 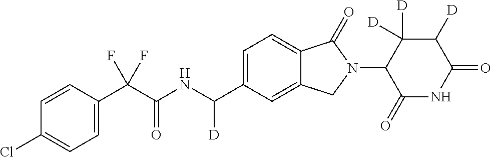 [SMILES: [2H]C(NC(=O)C(F)(F)c1ccc(Cl)cc1)c1ccc2c(c1)CN(C1C(=O)NC(=O)C([2H])C1([2H])[2H])C2=O] |
| 150 | 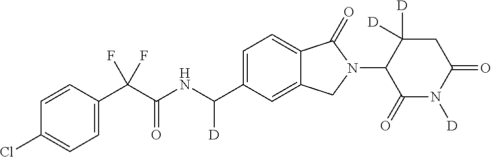 |
| 151 | 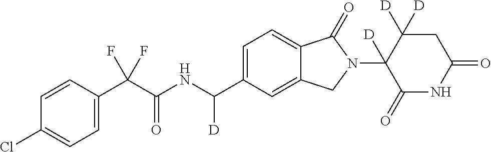 [SMILES: [2H]C(NC(=O)C(F)(F)c1ccc(Cl)cc1)c1ccc2c(c1)CN(C1([2H])C(=O)NC(=O)CC1([2H])[2H])C2=O] |
| 152 | 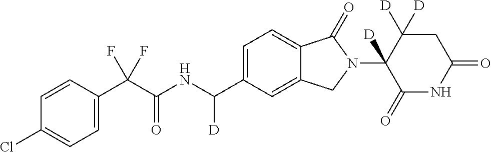 [SMILES: [2H]C(NC(=O)C(F)(F)c1ccc(Cl)cc1)c1ccc2c(c1)CN([C@@]1([2H])C(=O)NC(=O)CC1([2H])[2H])C2=O] |
| 153 | 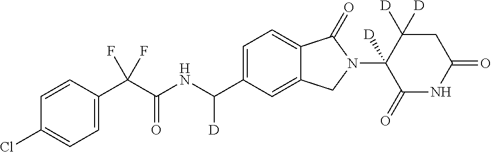 [SMILES: [2H]C(NC(=O)C(F)(F)c1ccc(Cl)cc1)c1ccc2c(c1)CN([C@]1([2H])C(=O)NC(=O)CC1([2H])[2H])C2=O] |
| 154 | 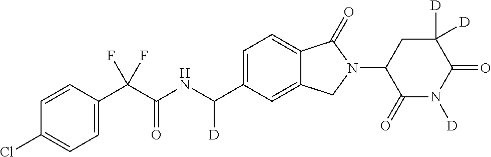 |
| 155 | 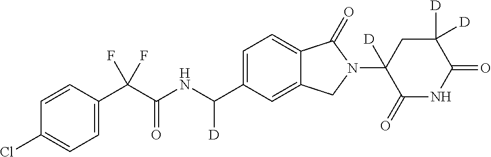 [SMILES: [2H]C(NC(=O)C(F)(F)c1ccc(Cl)cc1)c1ccc2c(c1)CN(C1([2H])CC([2H])([2H])C(=O)NC1=O)C2=O] |
| 156 | 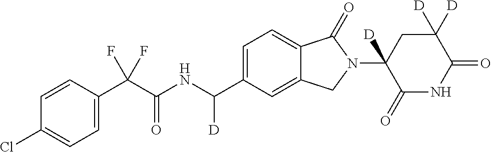 [SMILES: [2H]C(NC(=O)C(F)(F)c1ccc(Cl)cc1)c1ccc2c(c1)CN([C@]1([2H])CC([2H])([2H])C(=O)NC1=O)C2=O] |
| 157 | 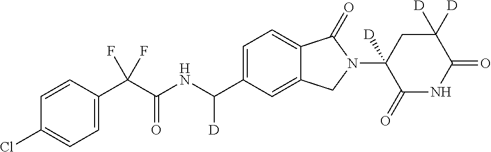 [SMILES: [2H]C(NC(=O)C(F)(F)c1ccc(Cl)cc1)c1ccc2c(c1)CN([C@@]1([2H])CC([2H])([2H])C(=O)NC1=O)C2=O] |
| 158 | 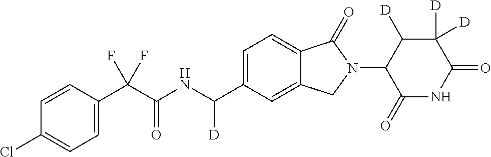 [SMILES: [2H]C(NC(=O)C(F)(F)c1ccc(Cl)cc1)c1ccc2c(c1)CN(C1C(=O)NC(=O)C([2H])([2H])C1[2H])C2=O] |
| 159 | 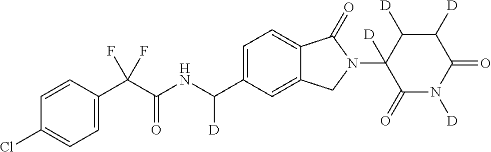 |
| 160 | 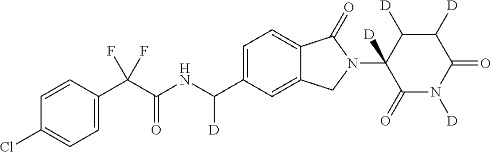 |
| 161 | 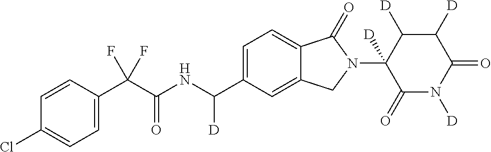 |
| 162 | 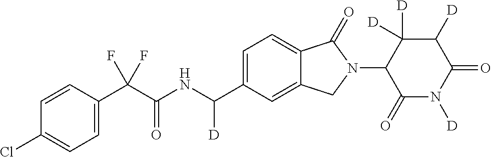 |
| 163 | 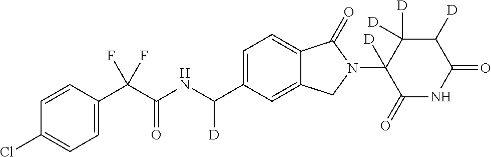 [SMILES: [2H]C(NC(=O)C(F)(F)c1ccc(Cl)cc1)c1ccc2c(c1)CN(C1([2H])C(=O)NC(=O)C([2H])C1([2H])[2H])C2=O] |
| 164 | 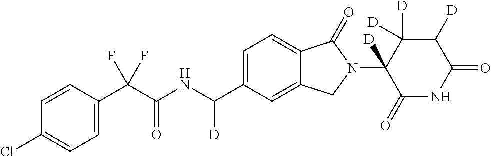 [SMILES: [2H]C(NC(=O)C(F)(F)c1ccc(Cl)cc1)c1ccc2c(c1)CN([C@@]1([2H])C(=O)NC(=O)C([2H])C1([2H])[2H])C2=O] |
| 165 | 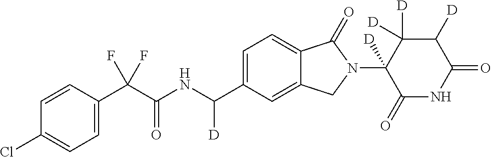 [SMILES: [2H]C(NC(=O)C(F)(F)c1ccc(Cl)cc1)c1ccc2c(c1)CN([C@]1([2H])C(=O)NC(=O)C([2H])C1([2H])[2H])C2=O] |
| 166 | 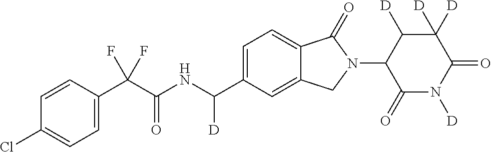 |
| 167 | 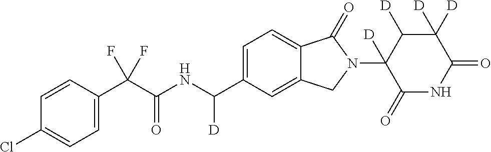 [SMILES: [2H]C(NC(=O)C(F)(F)c1ccc(Cl)cc1)c1ccc2c(c1)CN(C1([2H])C(=O)NC(=O)C([2H])([2H])C1[2H])C2=O] |
| 168 | 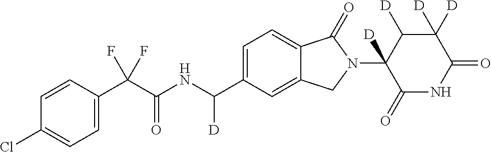 [SMILES: [2H]C(NC(=O)C(F)(F)c1ccc(Cl)cc1)c1ccc2c(c1)CN([C@@]1([2H])C(=O)NC(=O)C([2H])([2H])C1[2H])C2=O] |
| 169 | 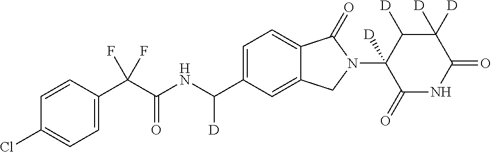 [SMILES: [2H]C(NC(=O)C(F)(F)c1ccc(Cl)cc1)c1ccc2c(c1)CN([C@]1([2H])C(=O)NC(=O)C([2H])([2H])C1[2H])C2=O] |
| 170 | 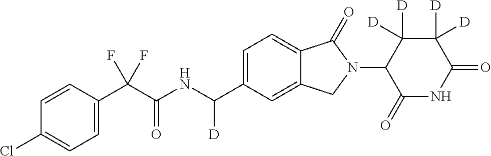 [SMILES: [2H]C(NC(=O)C(F)(F)c1ccc(Cl)cc1)c1ccc2c(c1)CN(C1C(=O)NC(=O)C([2H])([2H])C1([2H])[2H])C2=O] |
| 171 | 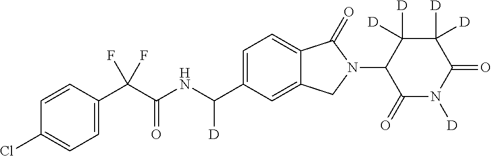 |
| 172 | 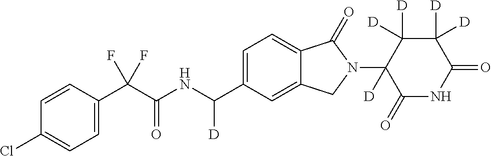 [SMILES: [2H]C(NC(=O)C(F)(F)c1ccc(Cl)cc1)c1ccc2c(c1)CN(C1([2H])C(=O)NC(=O)C([2H])([2H])C1([2H])[2H])C2=O] |
| 173 | 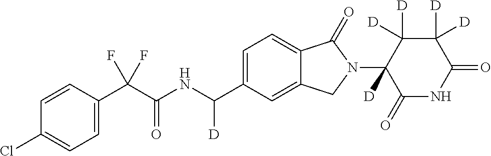 [SMILES: [2H]C(NC(=O)C(F)(F)c1ccc(Cl)cc1)c1ccc2c(c1)CN([C@@]1([2H])C(=O)NC(=O)C([2H])([2H])C1([2H])[2H])C2=O] |
| 174 | 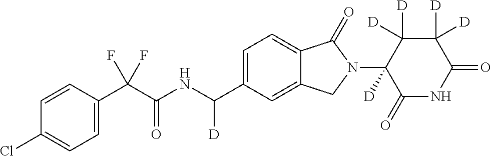 [SMILES: [2H]C(NC(=O)C(F)(F)c1ccc(Cl)cc1)c1ccc2c(c1)CN([C@]1([2H])C(=O)NC(=O)C([2H])([2H])C1([2H])[2H])C2=O] |
| 175 | 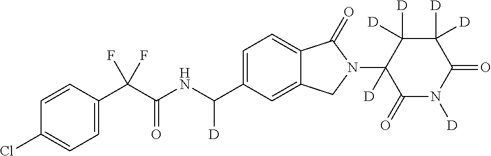 |
| 176 | 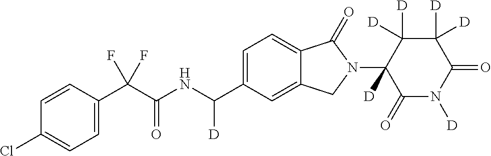 |
| 177 | 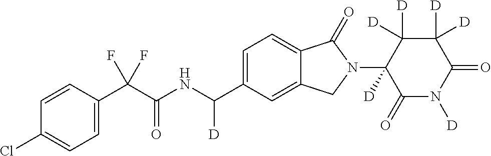 |
| 178 | 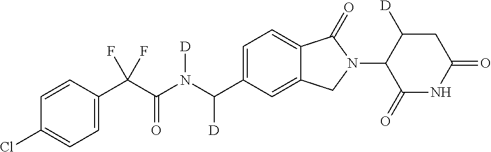 |
| 179 | 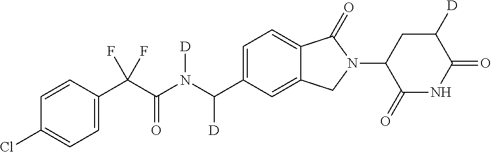 |
| 180 | 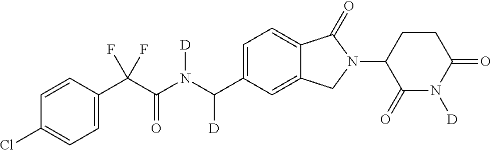 |
| 181 | 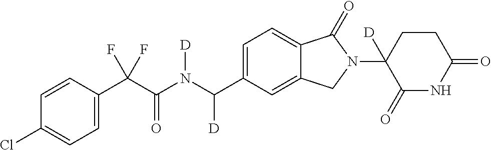 |
| 182 | 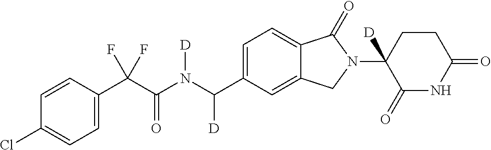 |
| 183 | 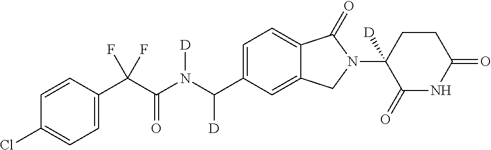 |
| 184 | 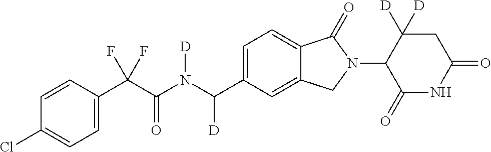 |
| 185 | 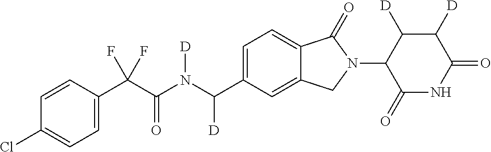 |
| 186 | 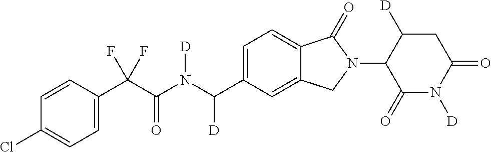 |
| 187 | 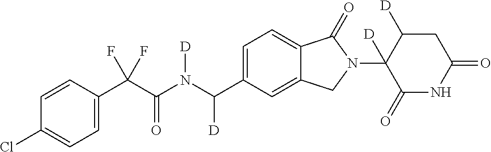 |
| 188 | 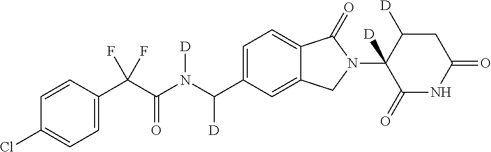 |
| 189 | 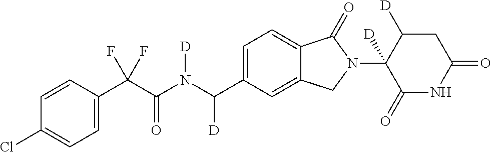 |
| 190 | 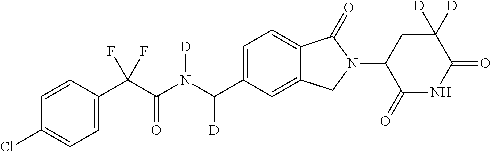 |
| 191 | 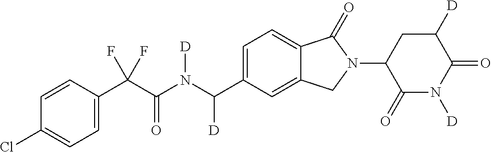 |
| 192 | 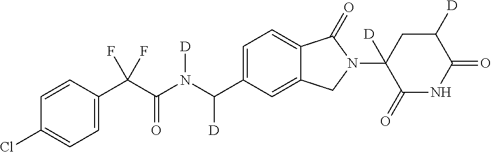 |
| 193 | 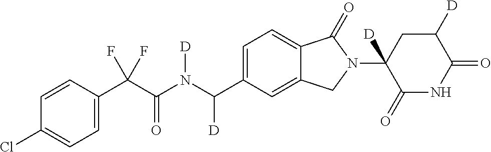 |
| 194 | 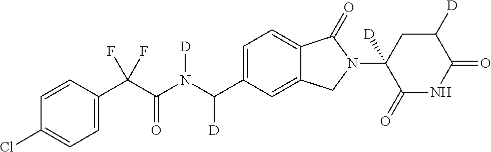 |
| 195 | 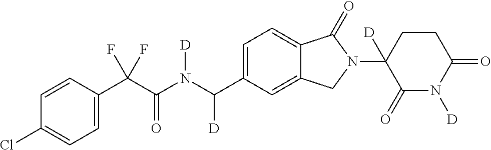 |
| 196 | 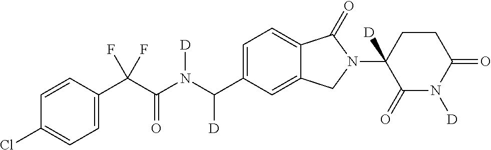 |
| 197 | 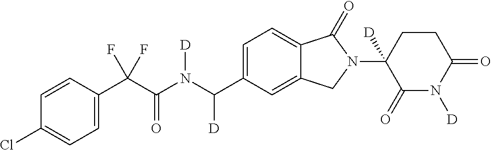 |
| 298 | 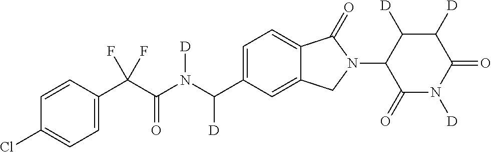 |
| 199 | 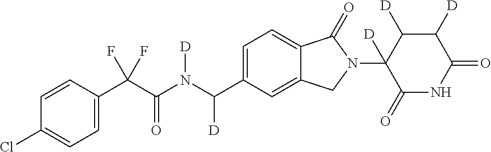 |
| 200 | 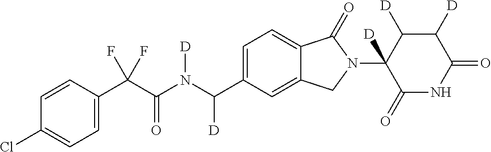 |
| 201 | 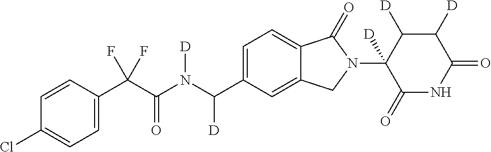 |
| 202 | 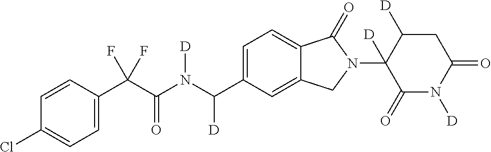 |
| 203 | 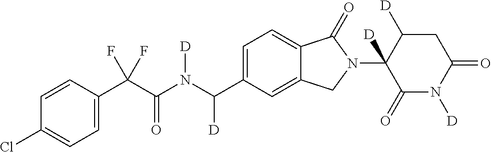 |
| 204 | 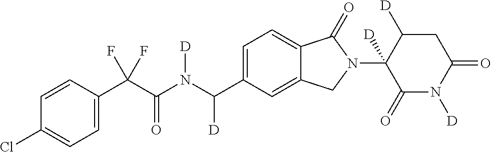 |
| 205 | 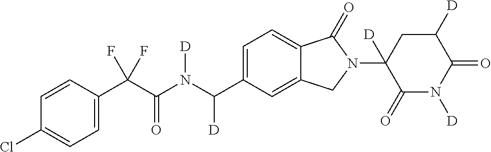 |
| 206 | 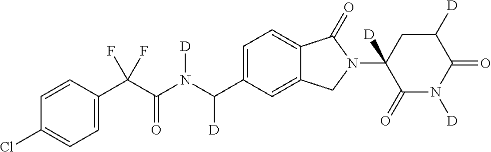 |
| 207 | 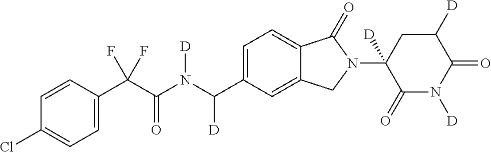 |
| 208 | 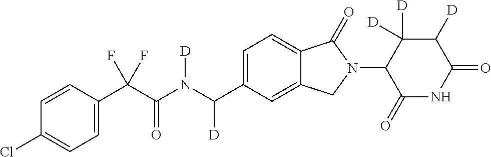 |
| 209 | 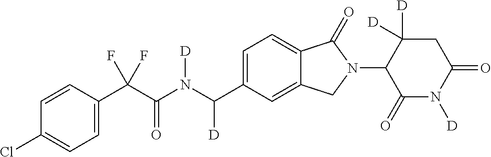 |
| 210 | 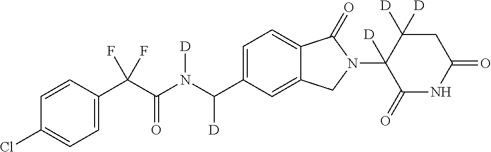 |
| 211 | 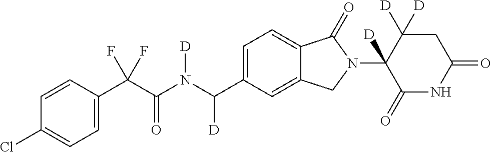 |
| 212 | 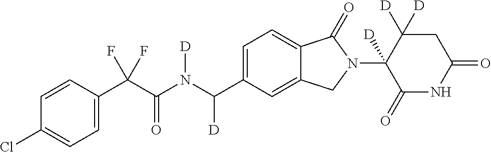 |
| 213 | 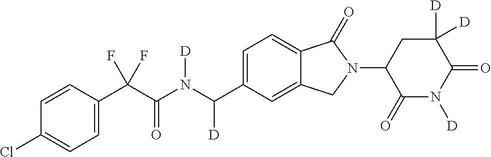 |
| 214 | 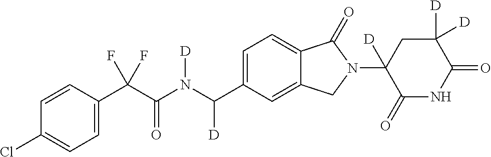 |
| 215 | 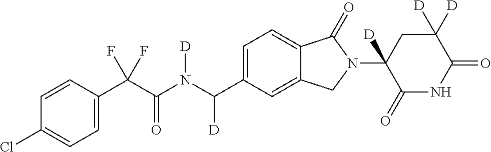 |
| 216 | 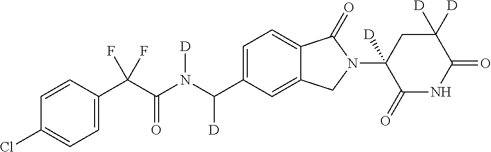 |
| 217 | 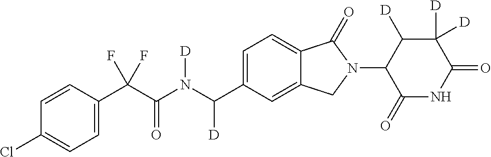 |
| 218 | 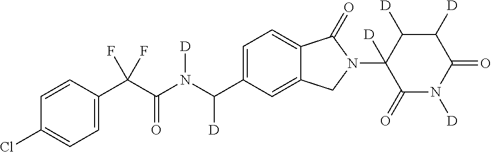 |
| 219 | 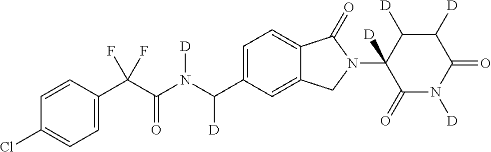 |
| 220 | 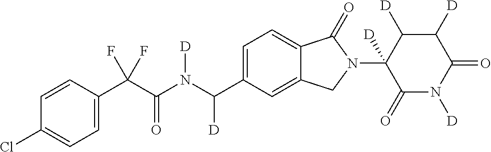 |
| 221 | 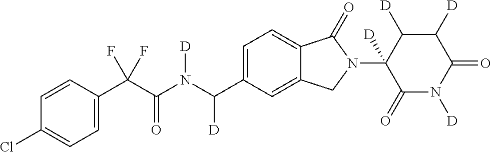 |
| 222 | 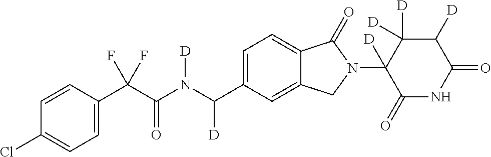 |
| 223 | 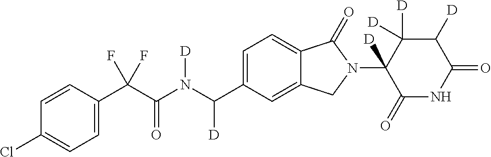 |
| 224 | 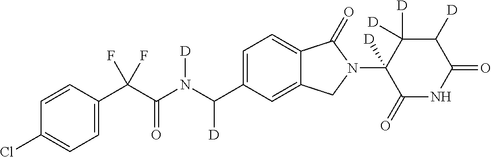 |
| 225 | 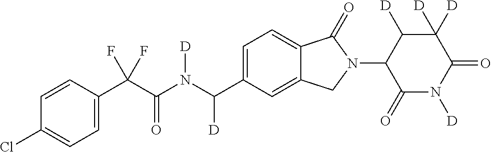 |
| 226 | 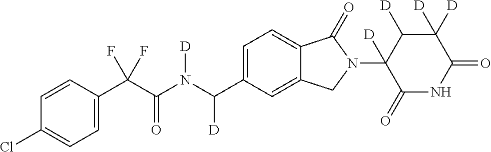 |
| 227 | 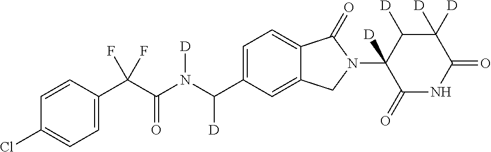 |
| 228 | 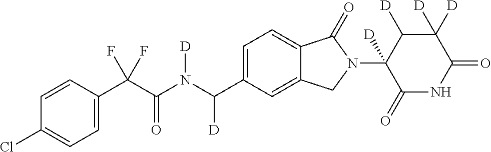 |
| 229 | 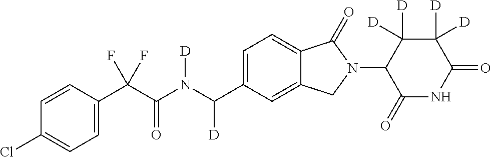 |
| 230 | 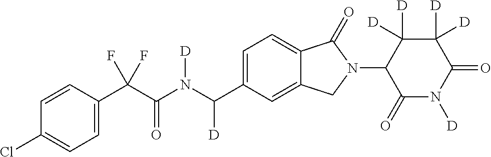 |
| 231 | 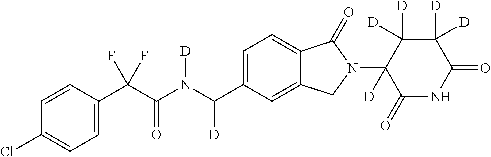 |
| 232 | 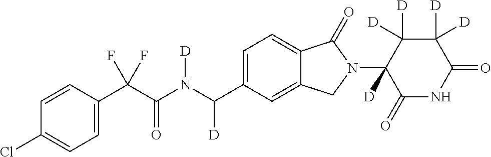 |
| 233 | 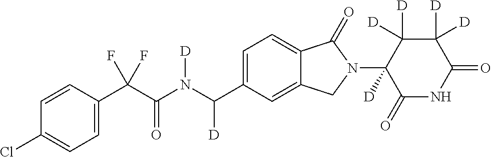 |
| 234 | 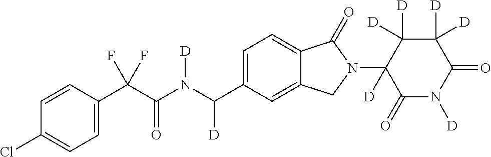 |
| 235 | 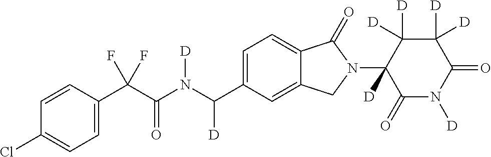 |
| 236 | 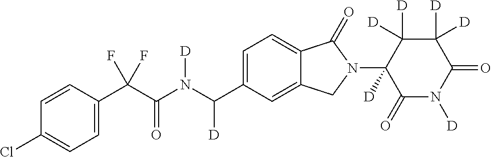 |
| 237 | 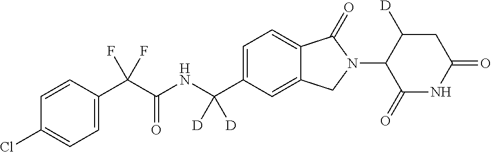 [SMILES: [2H]C1CC(=O)NC(=O)C1N1Cc2cc(C([2H])([2H])NC(=O)C(F)(F)c3ccc(Cl)cc3)ccc2C1=O] |
| 238 | 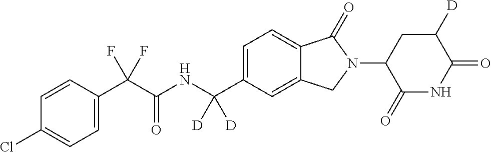 [SMILES: [2H]C1CC(N2Cc3cc(C([2H])([2H])NC(=O)C(F)(F)c4ccc(Cl)cc4)ccc3C2=O)C(=O)NC1=O] |
| 239 | 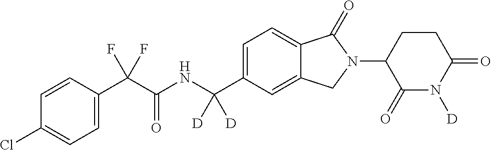 |
| 240 | 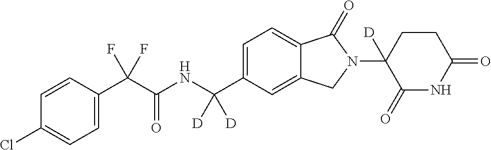 [SMILES: [2H]C([2H])(NC(=O)C(F)(F)c1ccc(Cl)cc1)c1ccc2c(c1)CN(C1([2H])CCC(=O)NC1=O)C2=O] |
| 241 | 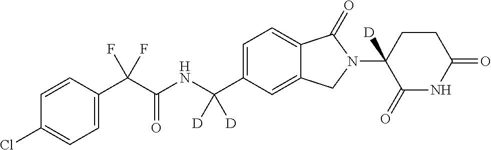 [SMILES: [2H]C([2H])(NC(=O)C(F)(F)c1ccc(Cl)cc1)c1ccc2c(c1)CN([C@]1([2H])CCC(=O)NC1=O)C2=O] |
| 242 | 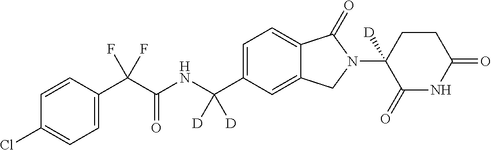 [SMILES: [2H]C([2H])(NC(=O)C(F)(F)c1ccc(Cl)cc1)c1ccc2c(c1)CN([C@@]1([2H])CCC(=O)NC1=O)C2=O] |
| 243 | 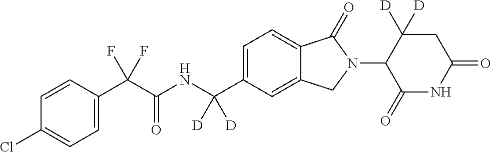 [SMILES: [2H]C([2H])(NC(=O)C(F)(F)c1ccc(Cl)cc1)c1ccc2c(c1)CN(C1C(=O)NC(=O)CC1([2H])[2H])C2=O] |
| 244 | 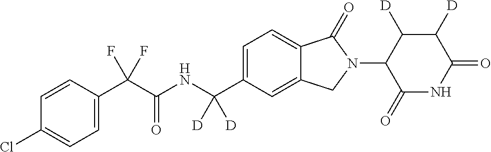 [SMILES: [2H]C1C(=O)NC(=O)C(N2Cc3cc(C([2H])([2H])NC(=O)C(F)(F)c4ccc(Cl)cc4)ccc3C2=O)C1[2H]] |
| 245 | 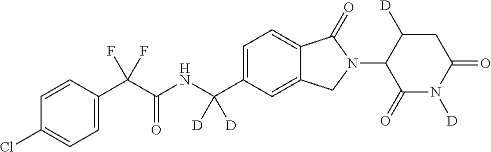 |
| 246 | 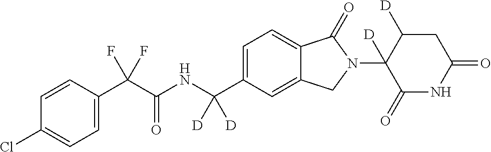 [SMILES: [2H]C1CC(=O)NC(=O)C1([2H])N1Cc2cc(C([2H])([2H])NC(=O)C(F)(F)c3ccc(Cl)cc3)ccc2C1=O] |
| 247 | 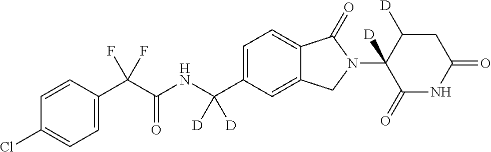 [SMILES: [2H]C1CC(=O)NC(=O)[C@]1([2H])N1Cc2cc(C([2H])([2H])NC(=O)C(F)(F)c3ccc(Cl)cc3)ccc2C1=O] |
| 248 | 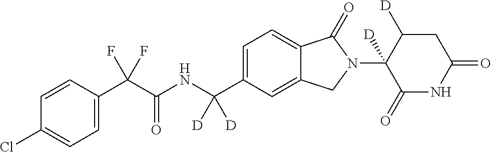 [SMILES: [2H]C1CC(=O)NC(=O)[C@@]1([2H])N1Cc2cc(C([2H])([2H])NC(=O)C(F)(F)c3ccc(Cl)cc3)ccc2C1=O] |
| 249 | 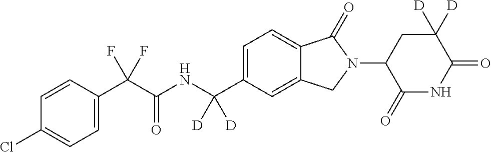 [SMILES: [2H]C1([2H])CC(N2Cc3cc(C([2H])([2H])NC(=O)C(F)(F)c4ccc(Cl)cc4)ccc3C2=O)C(=O)NC1=O] |
| 250 | 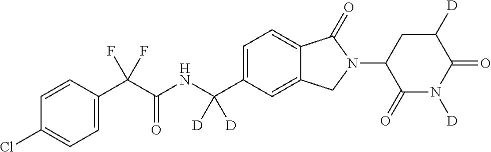 |
| 251 | 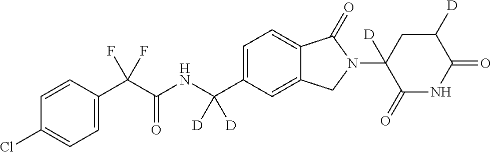 [SMILES: [2H]C1CC([2H])(N2Cc3cc(C([2H])([2H])NC(=O)C(F)(F)c4ccc(Cl)cc4)ccc3C2=O)C(=O)NC1=O] |
| 252 | 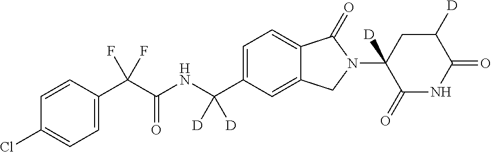 [SMILES: [2H]C1C[C@@]([2H])(N2Cc3cc(C([2H])([2H])NC(=O)C(F)(F)c4ccc(Cl)cc4)ccc3C2=O)C(=O)NC1=O] |
| 253 | 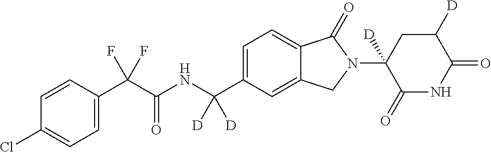 [SMILES: [2H]C1C[C@]([2H])(N2Cc3cc(C([2H])([2H])NC(=O)C(F)(F)c4ccc(Cl)cc4)ccc3C2=O)C(=O)NC1=O] |
| 254 | 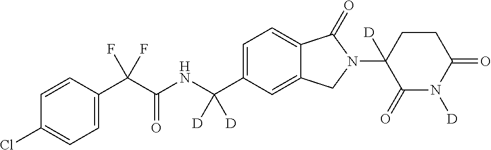 |
| 255 | 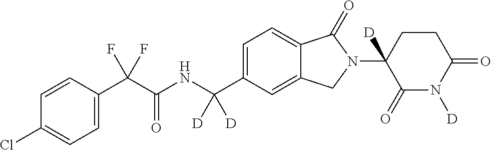 |
| 256 | 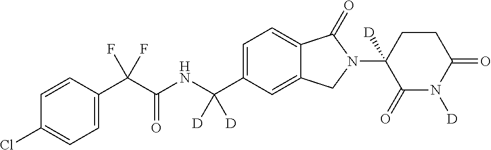 |
| 257 | 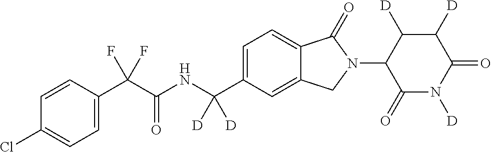 |
| 258 | 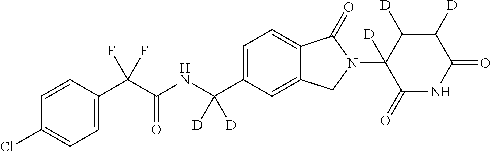 [SMILES: [2H]C1C(=O)NC(=O)C([2H])(N2Cc3cc(C([2H])([2H])NC(=O)C(F)(F)c4ccc(Cl)cc4)ccc3C2=O)C1[2H]] |
| 259 | 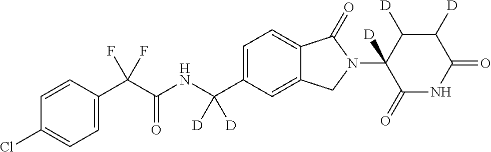 [SMILES: [2H]C1C(=O)NC(=O)[C@]([2H])(N2Cc3cc(C([2H])([2H])NC(=O)C(F)(F)c4ccc(Cl)cc4)ccc3C2=O)C1[2H]] |
| 260 | 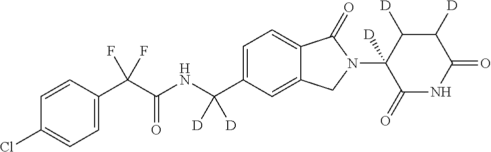 [SMILES: [2H]C1C(=O)NC(=O)[C@@]([2H])(N2Cc3cc(C([2H])([2H])NC(=O)C(F)(F)c4ccc(Cl)cc4)ccc3C2=O)C1[2H]] |
| 261 | 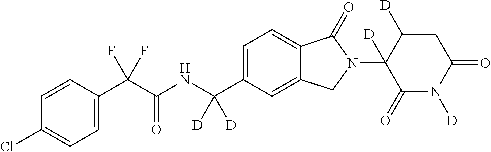 |
| 262 | 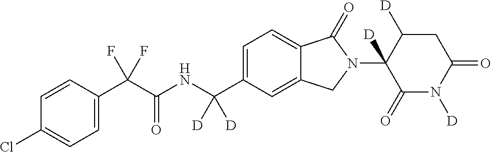 |
| 263 | 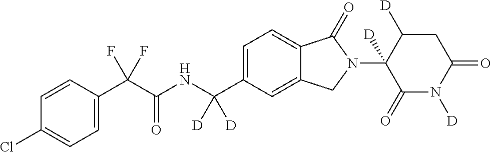 |
| 264 | 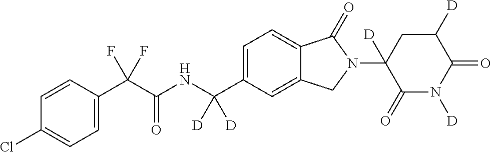 |
| 265 | 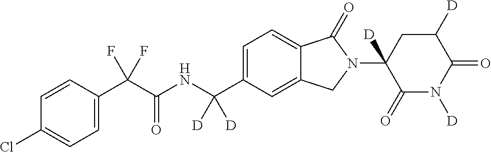 |
| 266 | 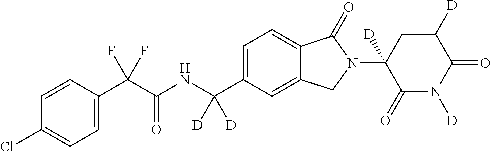 |
| 267 | 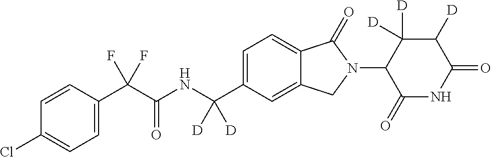 [SMILES: [2H]C1C(=O)NC(=O)C(N2Cc3cc(C([2H])([2H])NC(=O)C(F)(F)c4ccc(Cl)cc4)ccc3C2=O)C1([2H])[2H]] |
| 268 | 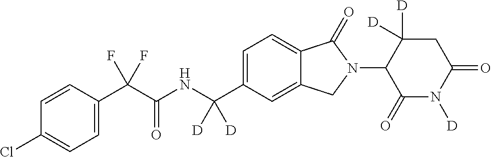 |
| 269 | 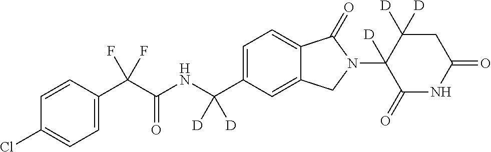 [SMILES: [2H]C([2H])(NC(=O)C(F)(F)c1ccc(Cl)cc1)c1ccc2c(c1)CN(C1([2H])C(=O)NC(=O)CC1([2H])[2H])C2=O] |
| 270 | 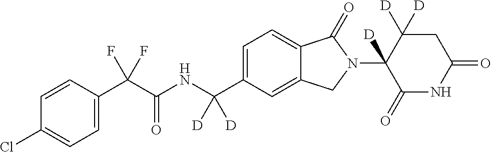 [SMILES: [2H]C([2H])(NC(=O)C(F)(F)c1ccc(Cl)cc1)c1ccc2c(c1)CN([C@@]1([2H])C(=O)NC(=O)CC1([2H])[2H])C2=O] |
| 271 | 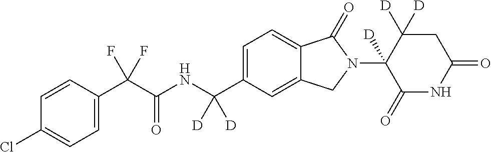 [SMILES: [2H]C([2H])(NC(=O)C(F)(F)c1ccc(Cl)cc1)c1ccc2c(c1)CN([C@]1([2H])C(=O)NC(=O)CC1([2H])[2H])C2=O] |
| 272 | 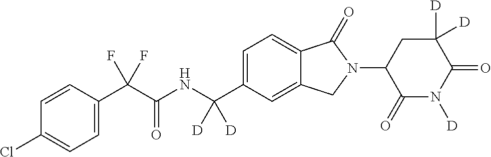 |
| 273 | 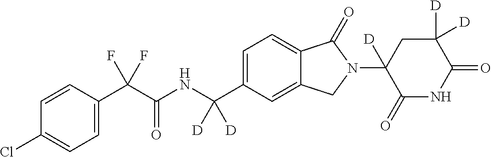 [SMILES: [2H]C1([2H])CC([2H])(N2Cc3cc(C([2H])([2H])NC(=O)C(F)(F)c4ccc(Cl)cc4)ccc3C2=O)C(=O)NC1=O] |
| 274 | 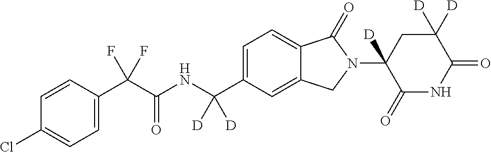 [SMILES: [2H]C1([2H])C[C@@]([2H])(N2Cc3cc(C([2H])([2H])NC(=O)C(F)(F)c4ccc(Cl)cc4)ccc3C2=O)C(=O)NC1=O] |
| 275 | 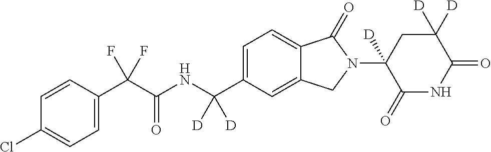 [SMILES: [2H]C1([2H])C[C@]([2H])(N2Cc3cc(C([2H])([2H])NC(=O)C(F)(F)c4ccc(Cl)cc4)ccc3C2=O)C(=O)NC1=O] |
| 276 | 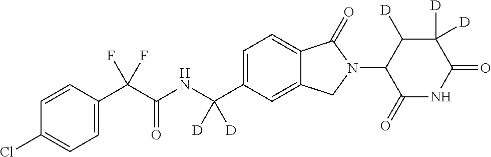 [SMILES: [2H]C1C(N2Cc3cc(C([2H])([2H])NC(=O)C(F)(F)c4ccc(Cl)cc4)ccc3C2=O)C(=O)NC(=O)C1([2H])[2H]] |
| 277 | 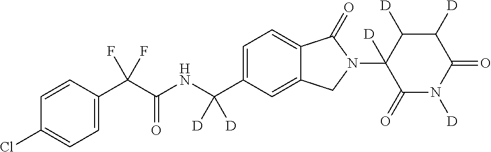 |
| 278 | 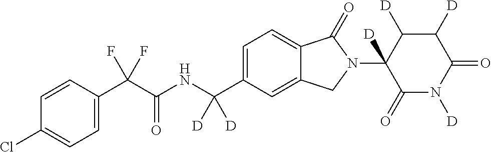 |
| 279 | 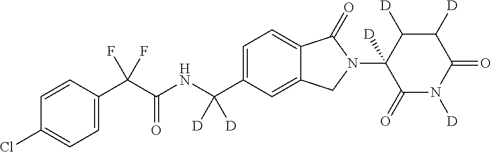 |
| 280 | 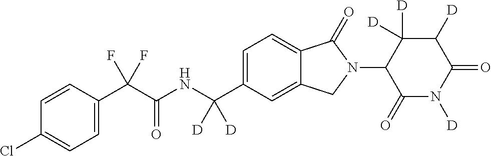 |
| 281 | 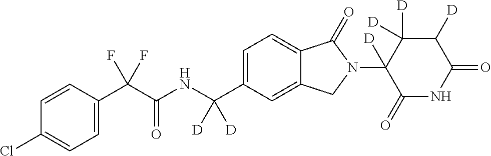 [SMILES: [2H]C1C(=O)NC(=O)C([2H])(N2Cc3cc(C([2H])([2H])NC(=O)C(F)(F)c4ccc(Cl)cc4)ccc3C2=O)C1([2H])[2H]] |
| 282 | 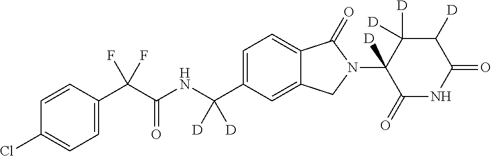 [SMILES: [2H]C1C(=O)NC(=O)[C@]([2H])(N2Cc3cc(C([2H])([2H])NC(=O)C(F)(F)c4ccc(Cl)cc4)ccc3C2=O)C1([2H])[2H]] |
| 283 | 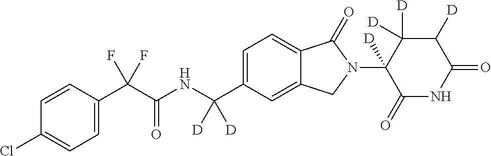 [SMILES: [2H]C1C(=O)NC(=O)[C@@]([2H])(N2Cc3cc(C([2H])([2H])NC(=O)C(F)(F)c4ccc(Cl)cc4)ccc3C2=O)C1([2H])[2H]] |
| 284 | 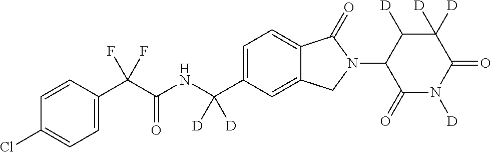 |
| 285 | 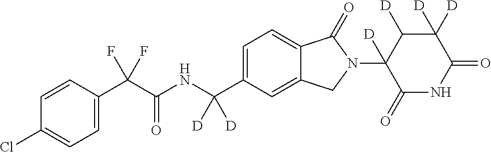 [SMILES: [2H]C1C([2H])([2H])C(=O)NC(=O)C1([2H])N1Cc2cc(C([2H])([2H])NC(=O)C(F)(F)c3ccc(Cl)cc3)ccc2C1=O] |
| 286 | 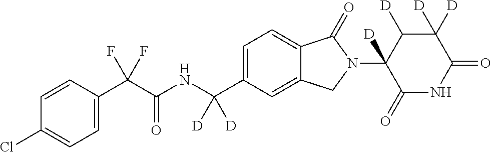 [SMILES: [2H]C1C([2H])([2H])C(=O)NC(=O)[C@]1([2H])N1Cc2cc(C([2H])([2H])NC(=O)C(F)(F)c3ccc(Cl)cc3)ccc2C1=O] |
| 287 | 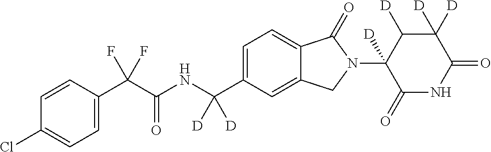 [SMILES: [2H]C1C([2H])([2H])C(=O)NC(=O)[C@@]1([2H])N1Cc2cc(C([2H])([2H])NC(=O)C(F)(F)c3ccc(Cl)cc3)ccc2C1=O] |
| 288 | 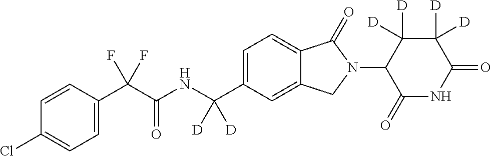 [SMILES: [2H]C([2H])(NC(=O)C(F)(F)c1ccc(Cl)cc1)c1ccc2c(c1)CN(C1C(=O)NC(=O)C([2H])([2H])C1([2H])[2H])C2=O] |
| 289 | 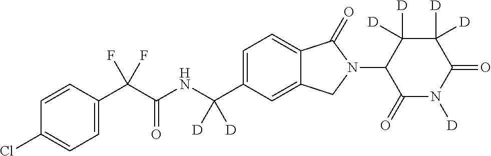 |
| 290 | 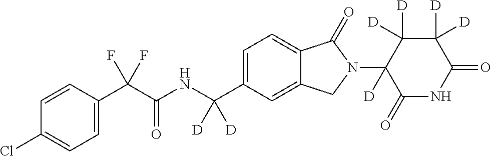 [SMILES: [2H]C([2H])(NC(=O)C(F)(F)c1ccc(Cl)cc1)c1ccc2c(c1)CN(C1([2H])C(=O)NC(=O)C([2H])([2H])C1([2H])[2H])C2=O] |
| 291 | 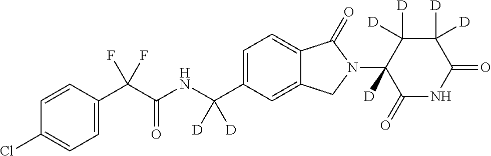 [SMILES: [2H]C([2H])(NC(=O)C(F)(F)c1ccc(Cl)cc1)c1ccc2c(c1)CN([C@@]1([2H])C(=O)NC(=O)C([2H])([2H])C1([2H])[2H])C2=O] |
| 292 | 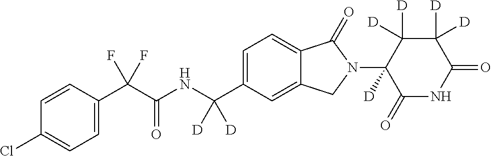 [SMILES: [2H]C([2H])(NC(=O)C(F)(F)c1ccc(Cl)cc1)c1ccc2c(c1)CN([C@]1([2H])C(=O)NC(=O)C([2H])([2H])C1([2H])[2H])C2=O] |
| 293 | 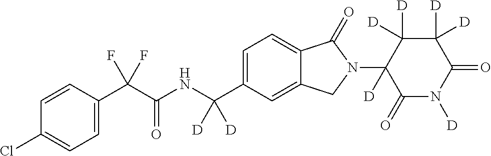 |
| 294 | 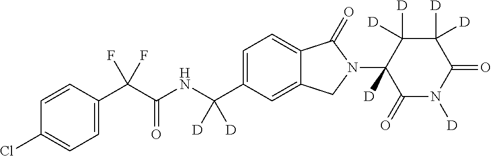 |
| 295 | 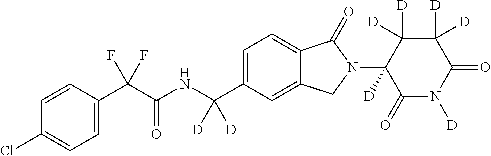 |
| 296 | 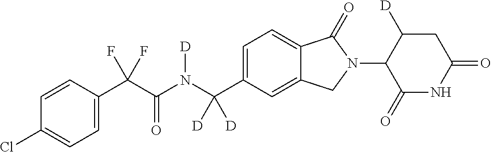 |
| 297 | 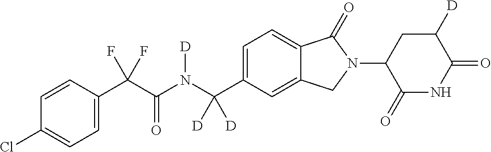 |
| 298 | 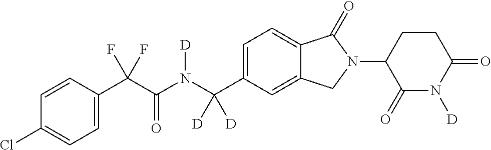 |
| 299 | 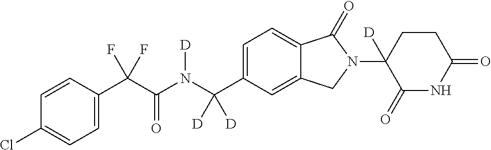 |
| 300 | 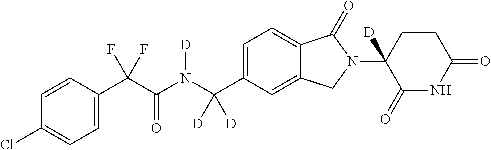 |
| 301 | 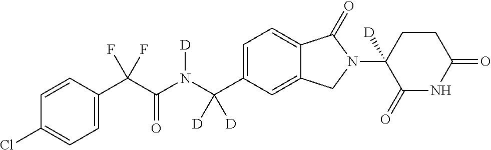 |
| 302 | 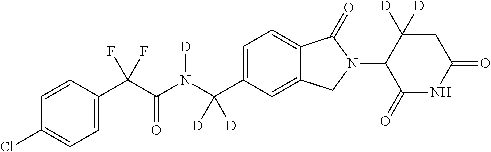 |
| 303 | 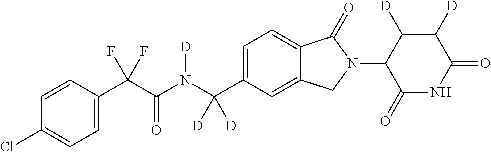 |
| 304 | 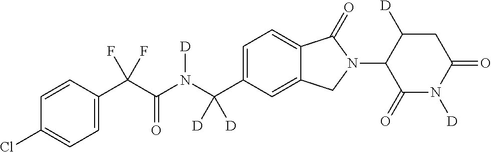 |
| 305 | 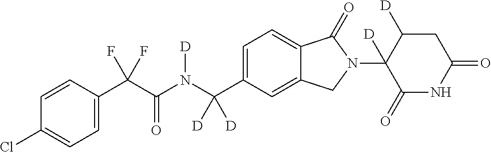 |
| 306 | 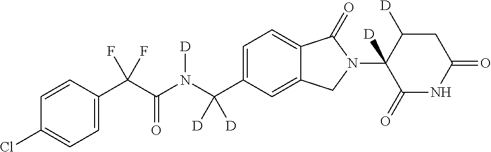 |
| 307 | 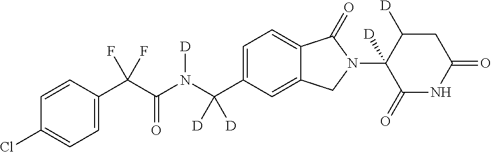 |
| 308 | 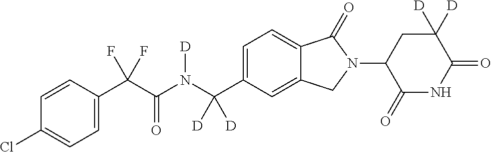 |
| 309 | 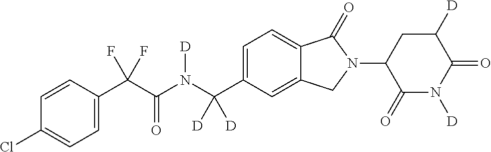 |
| 310 | 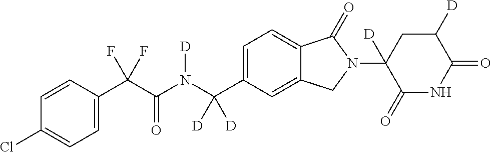 |
| 311 | 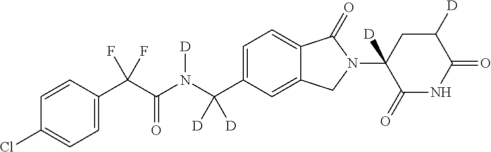 |
| 312 | 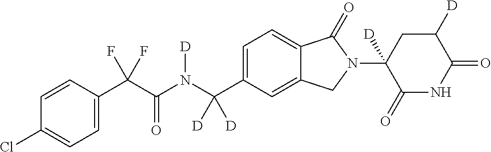 |
| 313 | 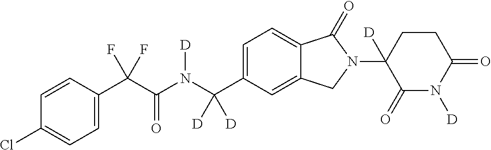 |
| 314 | 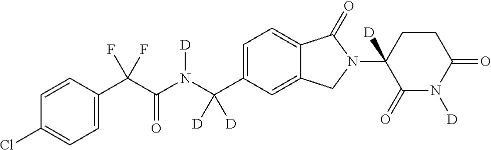 |
| 315 | 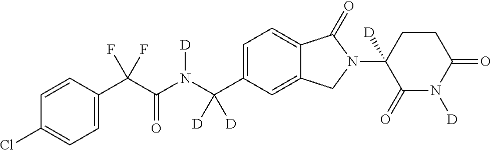 |
| 316 | 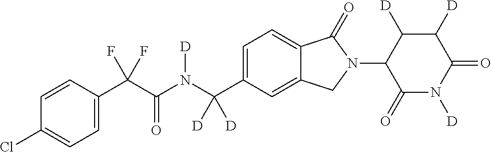 |
| 317 | 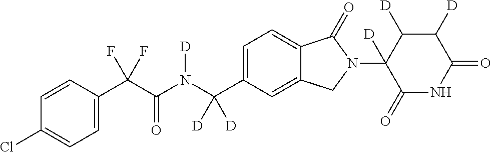 |
| 318 | 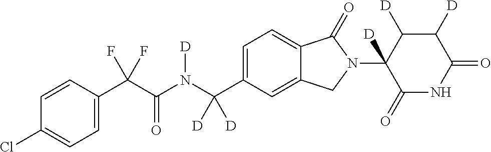 |
| 319 | 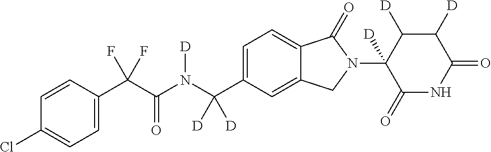 |
| 320 | 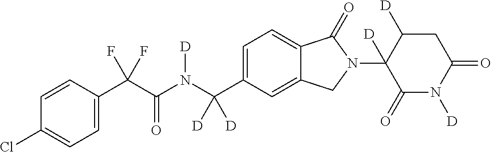 |
| 321 | 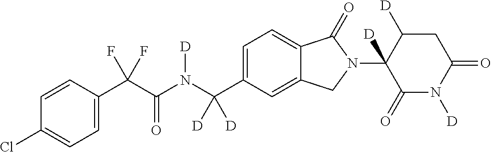 |
| 322 | 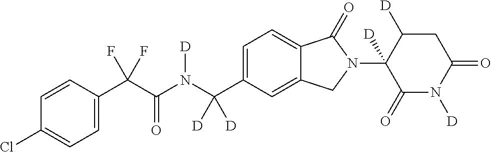 |
| 323 | 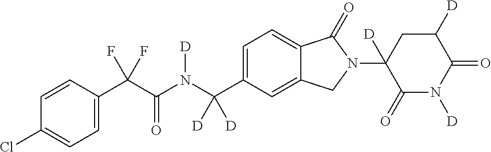 |
| 324 | 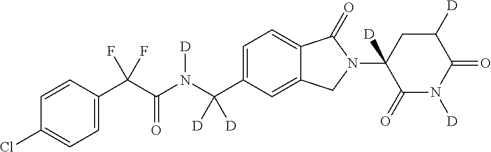 |
| 325 | 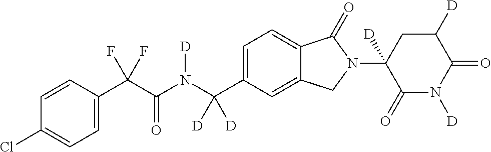 |
| 326 | 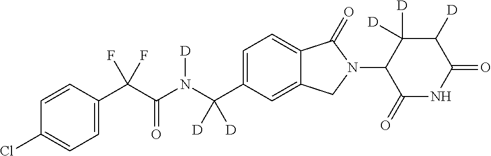 |
| 327 | 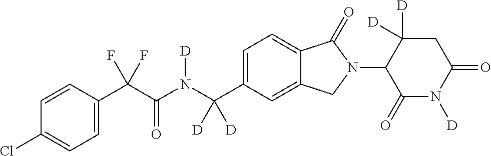 |
| 328 | 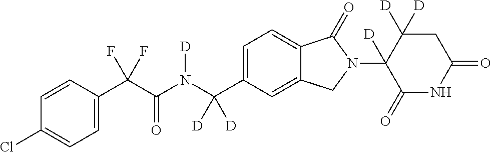 |
| 329 | 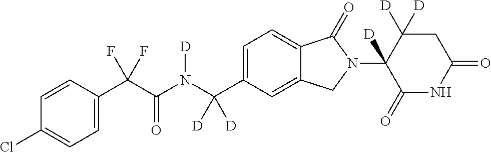 |
| 330 | 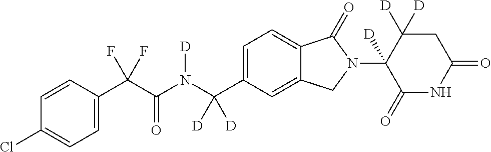 |
| 331 | 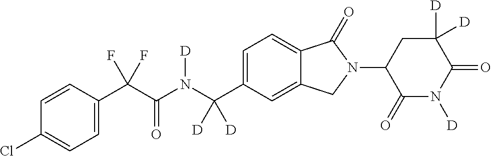 |
| 332 | 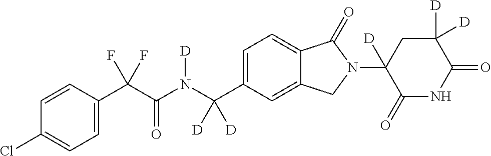 |
| 333 | 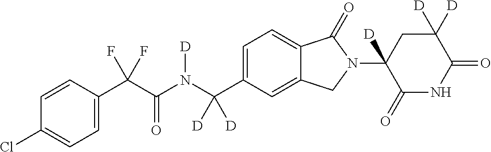 |
| 334 | 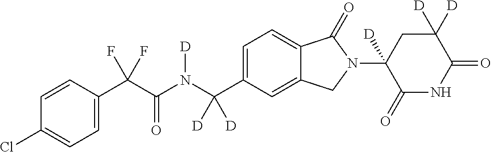 |
| 335 | 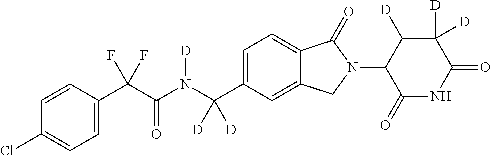 |
| 336 | 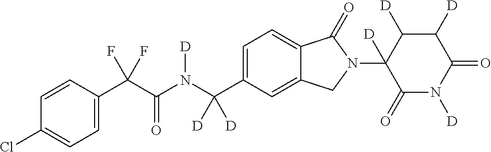 |
| 337 | 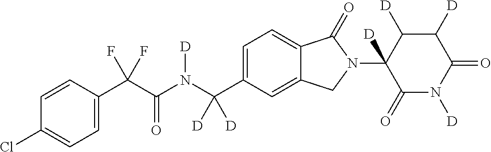 |
| 338 | 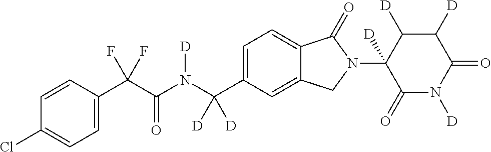 |
| 339 | 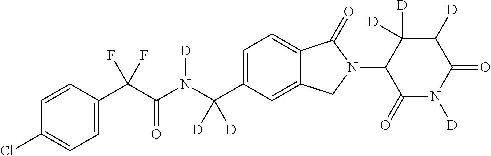 |
| 340 | 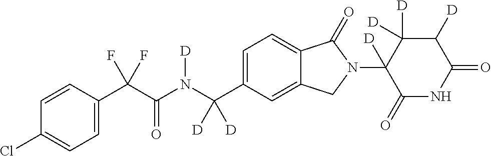 |
| 341 | 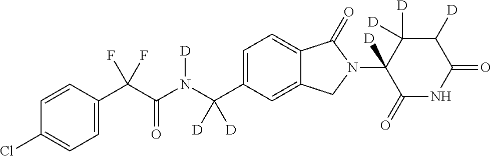 |
| 342 | 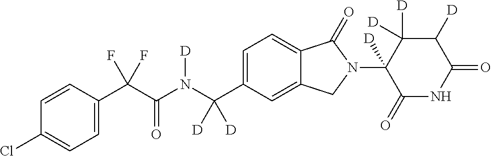 |
| 343 | 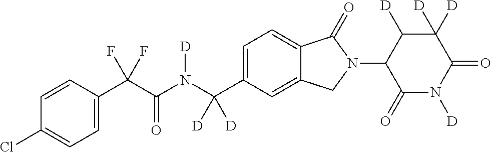 |
| 344 | 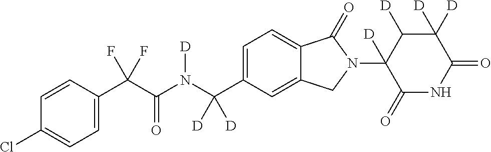 |
| 345 | 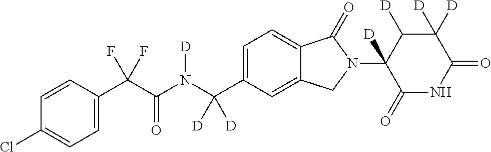 |
| 346 | 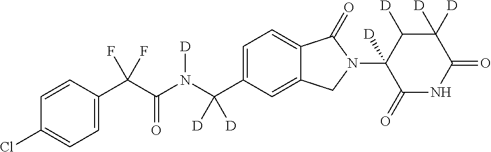 |
| 347 | 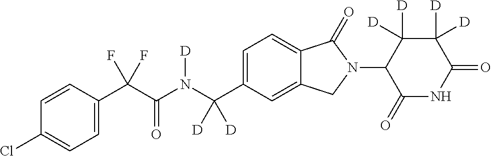 |
| 348 | 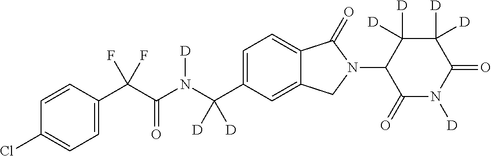 |
| 349 | 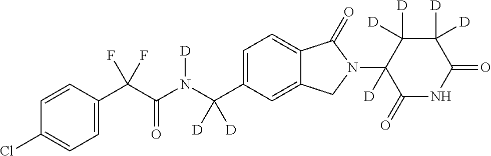 |
| 350 | 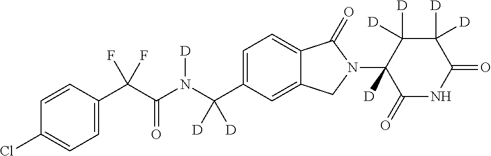 |
| 351 | 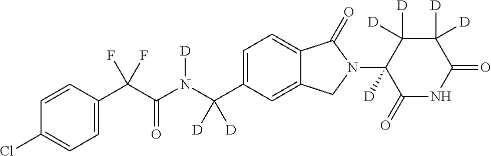 |
| 352 | 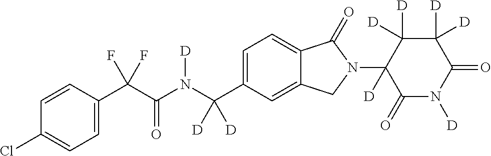 |
| 353 | 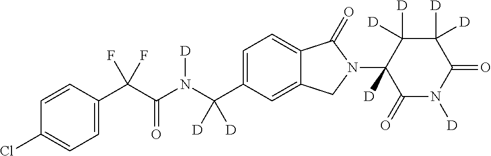 |
| 354 | 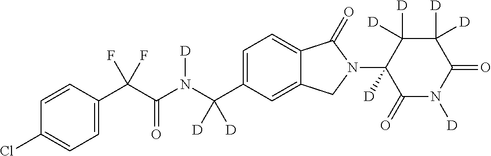 |
| 355 | 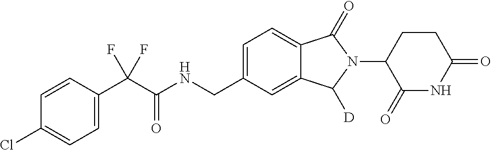 [SMILES: [2H]C1c2cc(CNC(=O)C(F)(F)c3ccc(Cl)cc3)ccc2C(=O)N1C1CCC(=O)NC1=O] |
| 356 | 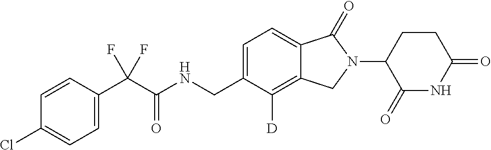 [SMILES: [2H]c1c(CNC(=O)C(F)(F)c2ccc(Cl)cc2)ccc2c1CN(C1CCC(=O)NC1=O)C2=O] |
| 357 | 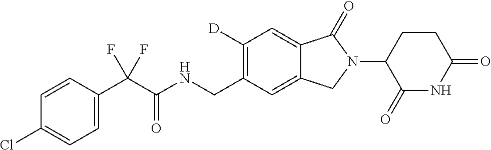 [SMILES: [2H]c1cc2c(cc1CNC(=O)C(F)(F)c1ccc(Cl)cc1)CN(C1CCC(=O)NC1=O)C2=O] |
| 358 | 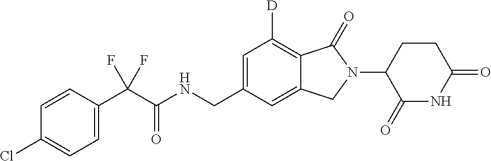 [SMILES: [2H]c1cc(CNC(=O)C(F)(F)c2ccc(Cl)cc2)cc2c1C(=O)N(C1CCC(=O)NC1=O)C2] |
| 359 | 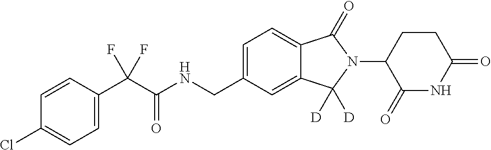 [SMILES: [2H]C1([2H])c2cc(CNC(=O)C(F)(F)c3ccc(Cl)cc3)ccc2C(=O)N1C1CCC(=O)NC1=O] |
| 360 | 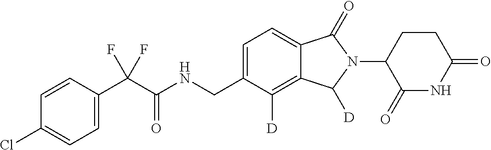 [SMILES: [2H]c1c(CNC(=O)C(F)(F)c2ccc(Cl)cc2)ccc2c1C([2H])N(C1CCC(=O)NC1=O)C2=O] |
| 361 | 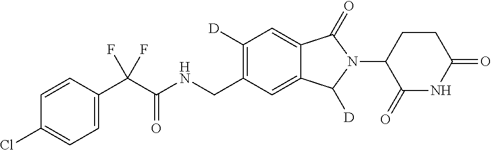 [SMILES: [2H]c1cc2c(cc1CNC(=O)C(F)(F)c1ccc(Cl)cc1)C([2H])N(C1CCC(=O)NC1=O)C2=O] |
| 362 | 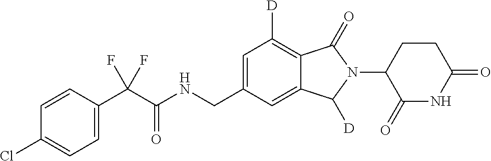 [SMILES: [2H]c1cc(CNC(=O)C(F)(F)c2ccc(Cl)cc2)cc2c1C(=O)N(C1CCC(=O)NC1=O)C2[2H]] |
| 363 | 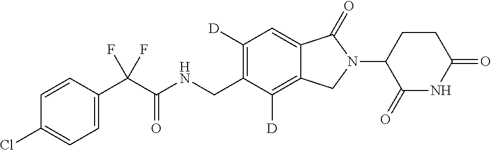 [SMILES: [2H]c1cc2c(c([2H])c1CNC(=O)C(F)(F)c1ccc(Cl)cc1)CN(C1CCC(=O)NC1=O)C2=O] |
| 364 | 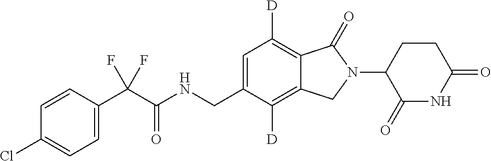 [SMILES: [2H]c1cc(CNC(=O)C(F)(F)c2ccc(Cl)cc2)c([2H])c2c1C(=O)N(C1CCC(=O)NC1=O)C2] |
| 365 | 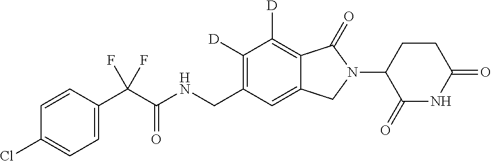 [SMILES: [2H]c1c(CNC(=O)C(F)(F)c2ccc(Cl)cc2)cc2c(c1[2H])C(=O)N(C1CCC(=O)NC1=O)C2] |
| 366 | 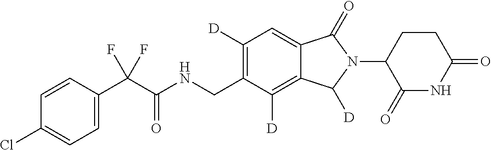 [SMILES: [2H]c1cc2c(c([2H])c1CNC(=O)C(F)(F)c1ccc(Cl)cc1)C([2H])N(C1CCC(=O)NC1=O)C2=O] |
| 367 | 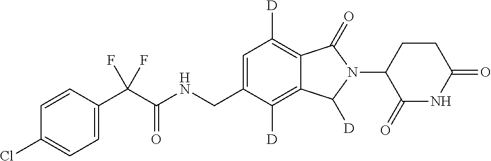 [SMILES: [2H]c1cc(CNC(=O)C(F)(F)c2ccc(Cl)cc2)c([2H])c2c1C(=O)N(C1CCC(=O)NC1=O)C2[2H]] |
| 368 | 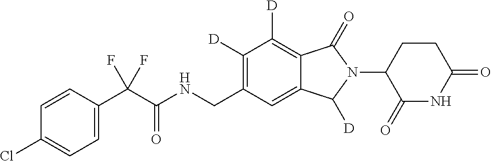 [SMILES: [2H]c1c(CNC(=O)C(F)(F)c2ccc(Cl)cc2)cc2c(c1[2H])C(=O)N(C1CCC(=O)NC1=O)C2[2H]] |
| 369 | 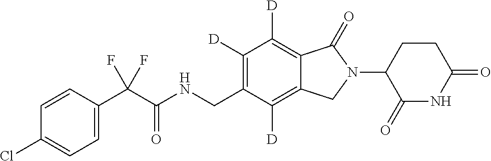 [SMILES: [2H]c1c([2H])c2c(c([2H])c1CNC(=O)C(F)(F)c1ccc(Cl)cc1)CN(C1CCC(=O)NC1=O)C2=O] |
| 370 | 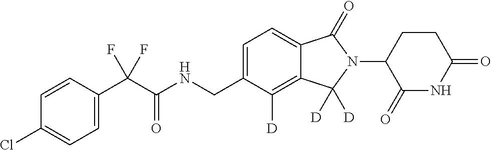 [SMILES: [2H]c1c(CNC(=O)C(F)(F)c2ccc(Cl)cc2)ccc2c1C([2H])([2H])N(C1CCC(=O)NC1=O)C2=O] |
| 371 | 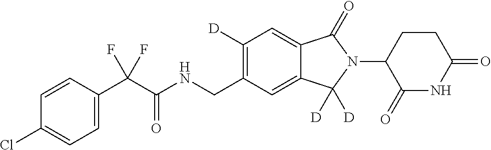 [SMILES: [2H]c1cc2c(cc1CNC(=O)C(F)(F)c1ccc(Cl)cc1)C([2H])([2H])N(C1CCC(=O)NC1=O)C2=O] |
| 372 | 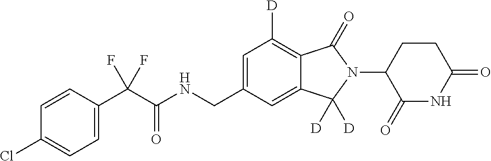 [SMILES: [2H]c1cc(CNC(=O)C(F)(F)c2ccc(Cl)cc2)cc2c1C(=O)N(C1CCC(=O)NC1=O)C2([2H])[2H]] |
| 373 | 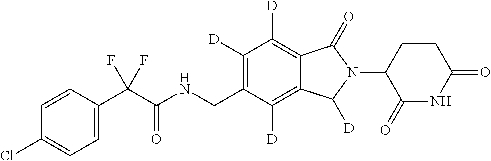 [SMILES: [2H]c1c([2H])c2c(c([2H])c1CNC(=O)C(F)(F)c1ccc(Cl)cc1)C([2H])N(C1CCC(=O)NC1=O)C2=O] |
| 374 | 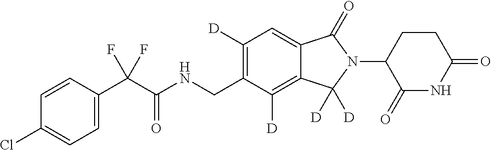 [SMILES: [2H]c1cc2c(c([2H])c1CNC(=O)C(F)(F)c1ccc(Cl)cc1)C([2H])([2H])N(C1CCC(=O)NC1=O)C2=O] |
| 375 | 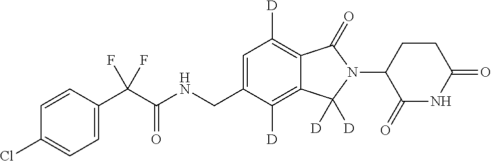 [SMILES: [2H]c1cc(CNC(=O)C(F)(F)c2ccc(Cl)cc2)c([2H])c2c1C(=O)N(C1CCC(=O)NC1=O)C2([2H])[2H]] |
| 376 | 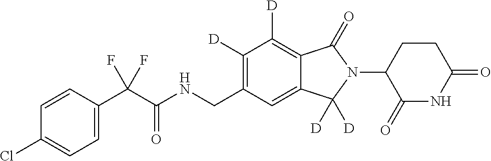 [SMILES: [2H]c1c(CNC(=O)C(F)(F)c2ccc(Cl)cc2)cc2c(c1[2H])C(=O)N(C1CCC(=O)NC1=O)C2([2H])[2H]] |
| 377 | 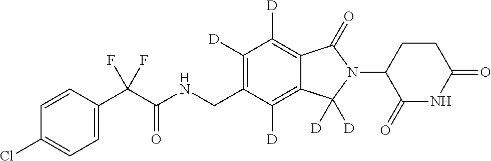 [SMILES: [2H]c1c([2H])c2c(c([2H])c1CNC(=O)C(F)(F)c1ccc(Cl)cc1)C([2H])([2H])N(C1CCC(=O)NC1=O)C2=O] |
| 378 | 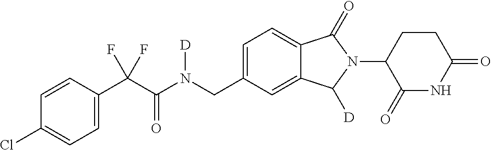 |
| 379 | 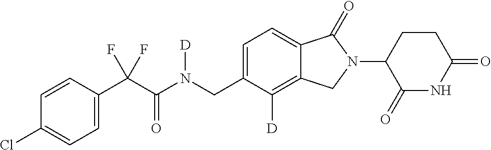 |
| 380 | 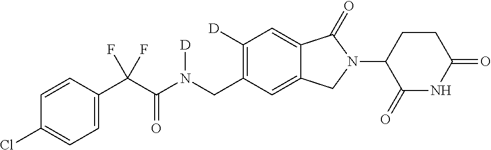 |
| 381 | 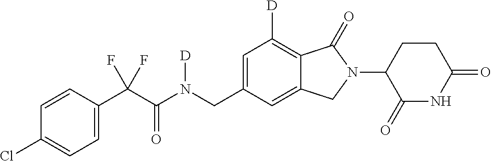 |
| 382 | 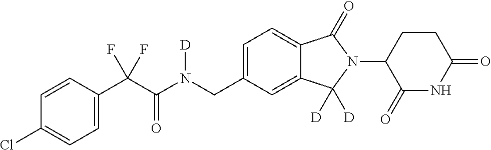 |
| 383 | 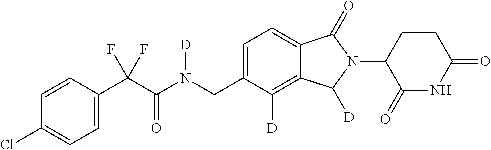 |
| 384 | 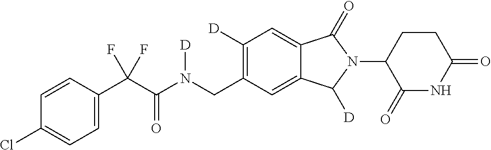 |
| 385 | 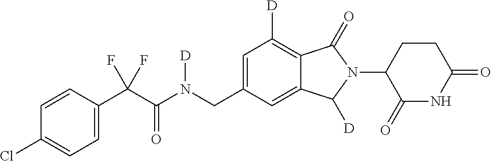 |
| 386 | 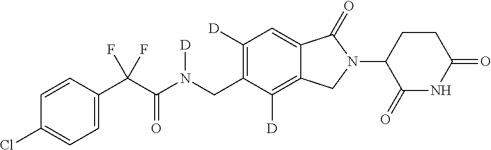 |
| 387 | 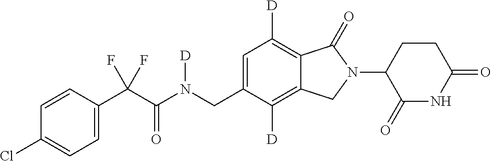 |
| 388 | 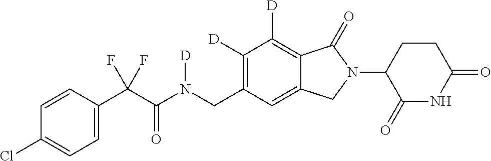 |
| 389 | 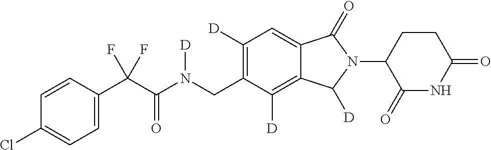 |
| 390 | 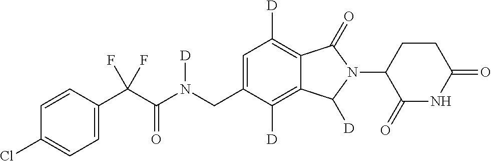 |
| 391 | 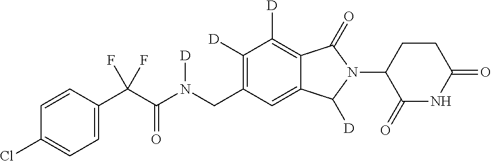 |
| 392 | 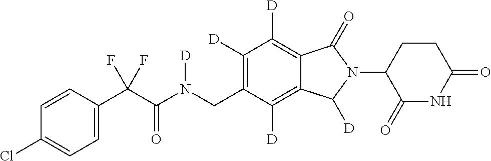 |
| 393 | 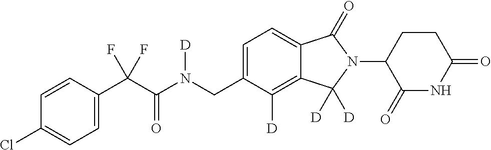 |
| 394 | 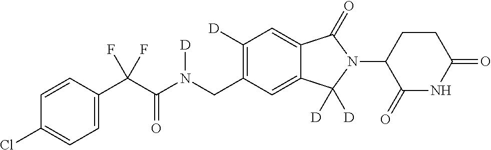 |
| 395 | 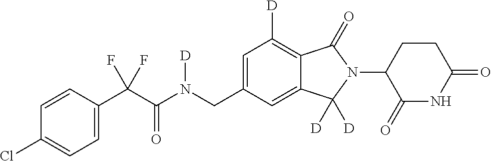 |
| 396 | 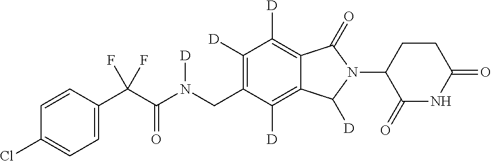 |
| 397 | 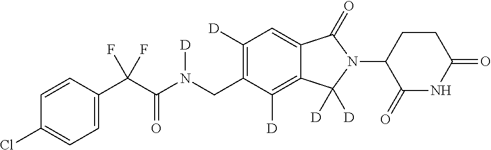 |
| 398 | 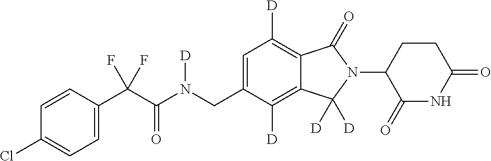 |
| 399 | 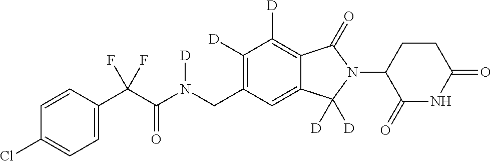 |
| 400 | 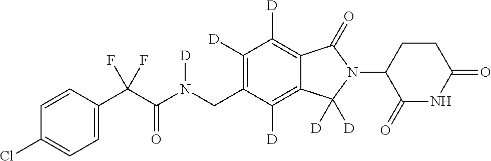 |
| 401 | 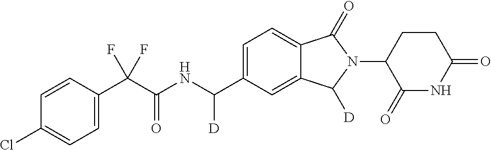 [SMILES: [2H]C(NC(=O)C(F)(F)c1ccc(Cl)cc1)c1ccc2c(c1)C([2H])N(C1CCC(=O)NC1=O)C2=O] |
| 402 | 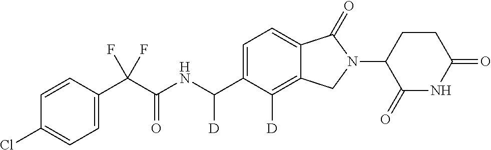 [SMILES: [2H]c1c(C([2H])NC(=O)C(F)(F)c2ccc(Cl)cc2)ccc2c1CN(C1CCC(=O)NC1=O)C2=O] |
| 403 | 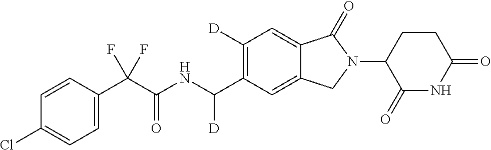 [SMILES: [2H]c1cc2c(cc1C([2H])NC(=O)C(F)(F)c1ccc(Cl)cc1)CN(C1CCC(=O)NC1=O)C2=O] |
| 404 | 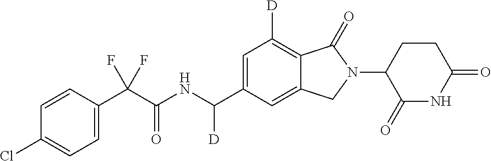 [SMILES: [2H]c1cc(C([2H])NC(=O)C(F)(F)c2ccc(Cl)cc2)cc2c1C(=O)N(C1CCC(=O)NC1=O)C2] |
| 405 | 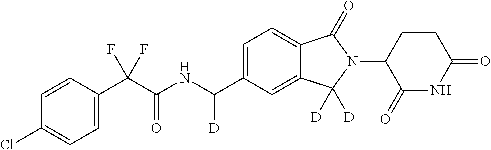 [SMILES: [2H]C(NC(=O)C(F)(F)c1ccc(Cl)cc1)c1ccc2c(c1)C([2H])([2H])N(C1CCC(=O)NC1=O)C2=O] |
| 406 | 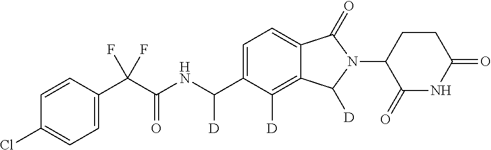 [SMILES: [2H]c1c(C([2H])NC(=O)C(F)(F)c2ccc(Cl)cc2)ccc2c1C([2H])N(C1CCC(=O)NC1=O)C2=O] |
| 407 | 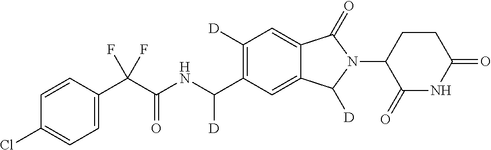 [SMILES: [2H]c1cc2c(cc1C([2H])NC(=O)C(F)(F)c1ccc(Cl)cc1)C([2H])N(C1CCC(=O)NC1=O)C2=O] |
| 408 | 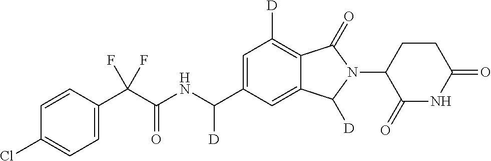 [SMILES: [2H]c1cc(C([2H])NC(=O)C(F)(F)c2ccc(Cl)cc2)cc2c1C(=O)N(C1CCC(=O)NC1=O)C2[2H]] |
| 409 | 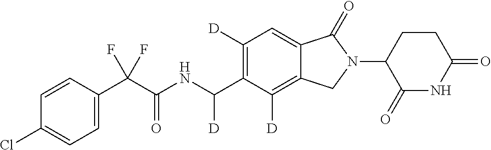 [SMILES: [2H]c1cc2c(c([2H])c1C([2H])NC(=O)C(F)(F)c1ccc(Cl)cc1)CN(C1CCC(=O)NC1=O)C2=O] |
| 410 | 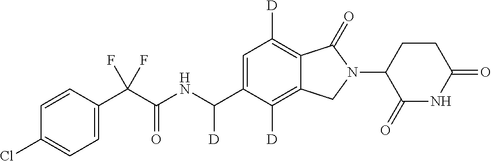 [SMILES: [2H]c1cc(C([2H])NC(=O)C(F)(F)c2ccc(Cl)cc2)c([2H])c2c1C(=O)N(C1CCC(=O)NC1=O)C2] |
| 411 | 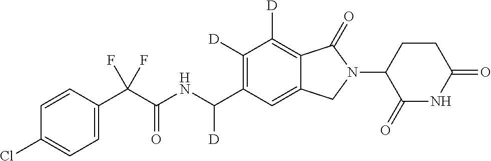 [SMILES: [2H]c1c(C([2H])NC(=O)C(F)(F)c2ccc(Cl)cc2)cc2c(c1[2H])C(=O)N(C1CCC(=O)NC1=O)C2] |
| 412 | 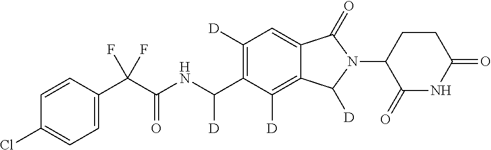 [SMILES: [2H]c1cc2c(c([2H])c1C([2H])NC(=O)C(F)(F)c1ccc(Cl)cc1)C([2H])N(C1CCC(=O)NC1=O)C2=O] |
| 413 | 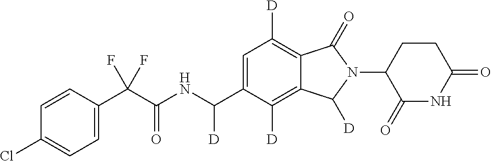 [SMILES: [2H]c1cc(C([2H])NC(=O)C(F)(F)c2ccc(Cl)cc2)c([2H])c2c1C(=O)N(C1CCC(=O)NC1=O)C2[2H]] |
| 414 | 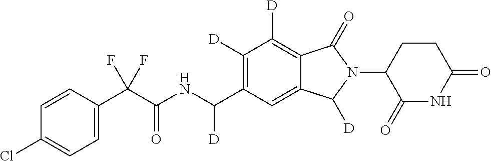 [SMILES: [2H]c1c(C([2H])NC(=O)C(F)(F)c2ccc(Cl)cc2)cc2c(c1[2H])C(=O)N(C1CCC(=O)NC1=O)C2[2H]] |
| 415 | 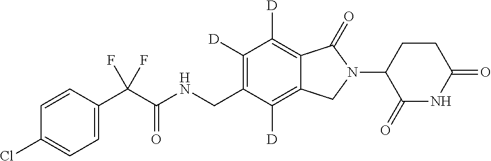 [SMILES: [2H]c1c([2H])c2c(c([2H])c1CNC(=O)C(F)(F)c1ccc(Cl)cc1)CN(C1CCC(=O)NC1=O)C2=O] |
| 416 | 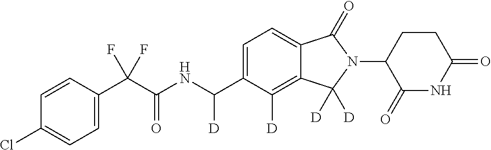 [SMILES: [2H]c1c(C([2H])NC(=O)C(F)(F)c2ccc(Cl)cc2)ccc2c1C([2H])([2H])N(C1CCC(=O)NC1=O)C2=O] |
| 417 | 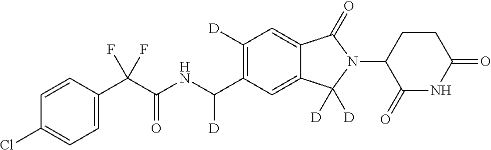 [SMILES: [2H]c1cc2c(cc1C([2H])NC(=O)C(F)(F)c1ccc(Cl)cc1)C([2H])([2H])N(C1CCC(=O)NC1=O)C2=O] |
| 418 | 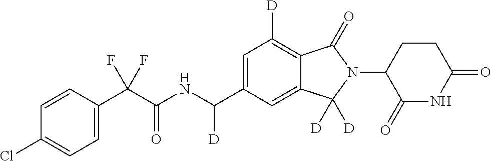 [SMILES: [2H]c1cc(C([2H])NC(=O)C(F)(F)c2ccc(Cl)cc2)cc2c1C(=O)N(C1CCC(=O)NC1=O)C2([2H])[2H]] |
| 419 | 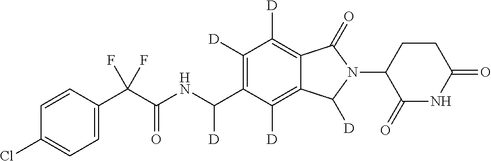 [SMILES: [2H]c1c([2H])c(C([2H])NC(=O)C(F)(F)c2ccc(Cl)cc2)c([2H])c2c1C(=O)N(C1CCC(=O)NC1=O)C2[2H]] |
| 420 | 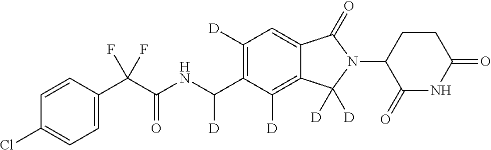 [SMILES: [2H]c1cc2c(c([2H])c1C([2H])NC(=O)C(F)(F)c1ccc(Cl)cc1)C([2H])([2H])N(C1CCC(=O)NC1=O)C2=O] |
| 421 | 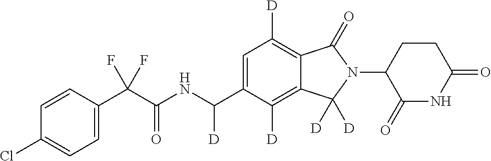 [SMILES: [2H]c1cc(C([2H])NC(=O)C(F)(F)c2ccc(Cl)cc2)c([2H])c2c1C(=O)N(C1CCC(=O)NC1=O)C2([2H])[2H]] |
| 422 | 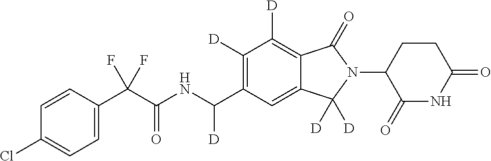 [SMILES: [2H]c1c(C([2H])NC(=O)C(F)(F)c2ccc(Cl)cc2)cc2c(c1[2H])C(=O)N(C1CCC(=O)NC1=O)C2([2H])[2H]] |
| 423 | 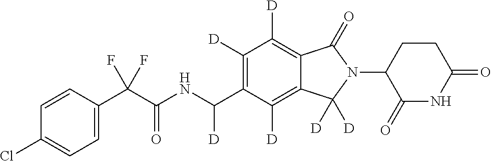 [SMILES: [2H]c1c([2H])c(C([2H])NC(=O)C(F)(F)c2ccc(Cl)cc2)c([2H])c2c1C(=O)N(C1CCC(=O)NC1=O)C2([2H])[2H]] |
| 424 | 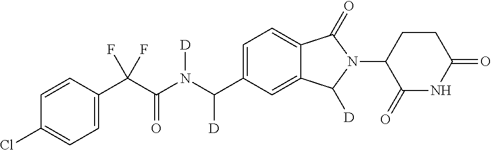 |
| 425 | 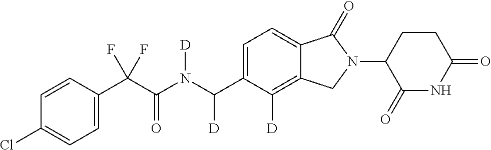 |
| 426 | 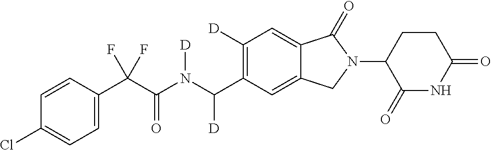 |
| 427 | 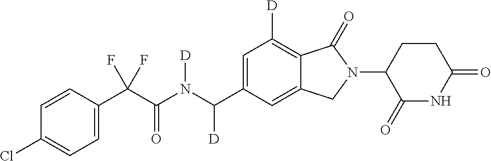 |
| 428 | 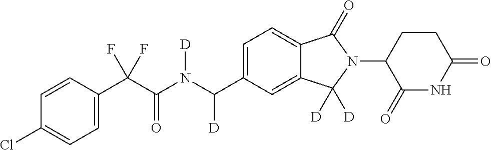 |
| 429 | 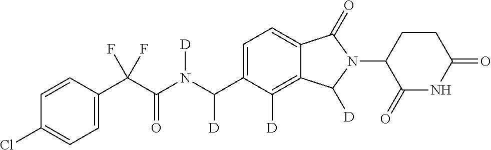 |
| 430 | 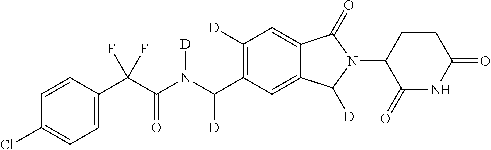 |
| 431 | 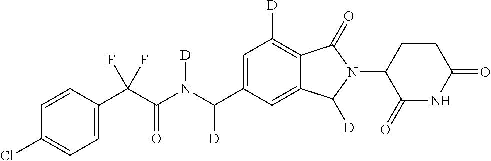 |
| 432 | 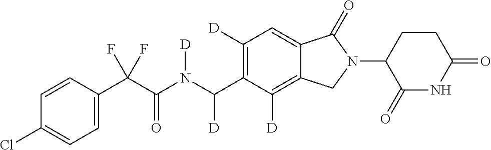 |
| 433 | 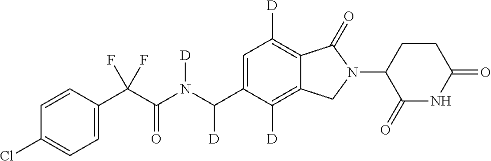 |
| 434 | 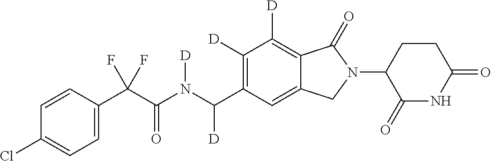 |
| 435 | 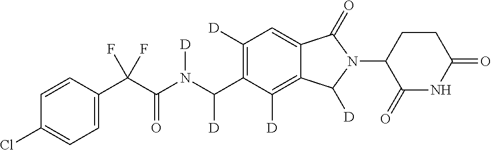 |
| 436 | 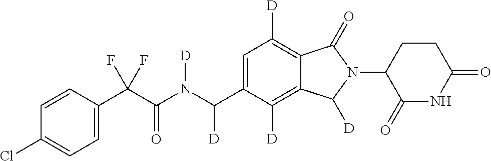 |
| 437 | 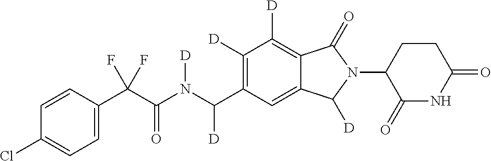 |
| 438 | 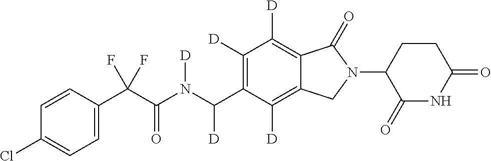 |
| 439 | 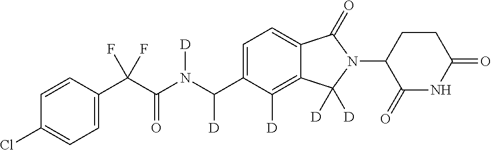 |
| 440 | 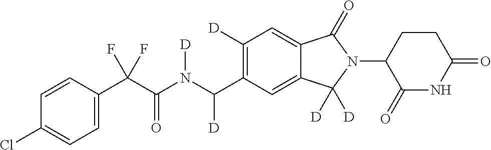 |
| 441 | 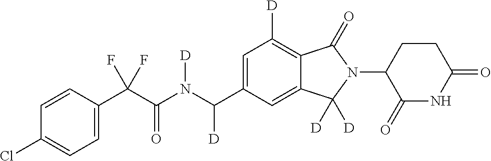 |
| 442 | 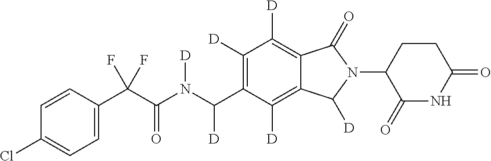 |
| 443 | 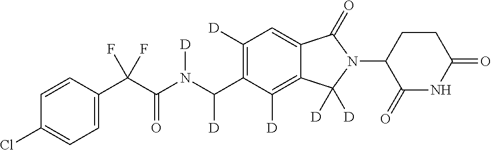 |
| 444 | 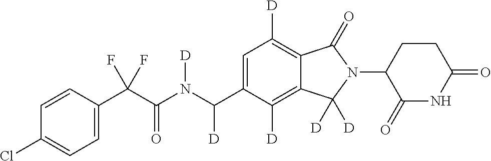 |
| 445 | 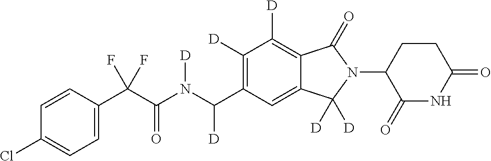 |
| 446 | 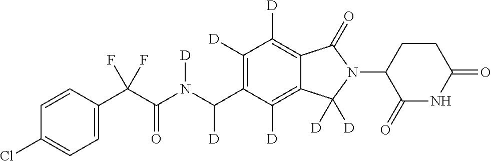 |
| 447 | 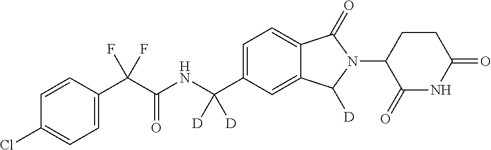 [SMILES: [2H]C1c2cc(C([2H])([2H])NC(=O)C(F)(F)c3ccc(Cl)cc3)ccc2C(=O)N1C1CCC(=O)NC1=O] |
| 448 | 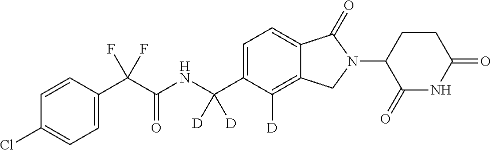 [SMILES: [2H]c1c(C([2H])([2H])NC(=O)C(F)(F)c2ccc(Cl)cc2)ccc2c1CN(C1CCC(=O)NC1=O)C2=O] |
| 449 | 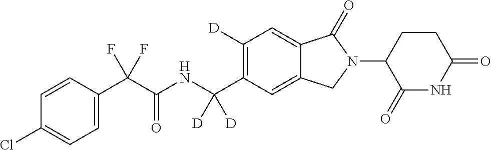 [SMILES: [2H]c1cc2c(cc1C([2H])([2H])NC(=O)C(F)(F)c1ccc(Cl)cc1)CN(C1CCC(=O)NC1=O)C2=O] |
| 450 | 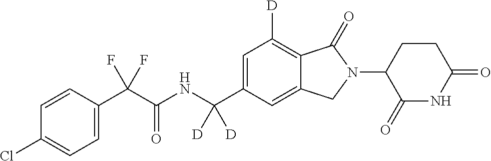 [SMILES: [2H]c1cc(C([2H])([2H])NC(=O)C(F)(F)c2ccc(Cl)cc2)cc2c1C(=O)N(C1CCC(=O)NC1=O)C2] |
| 451 | 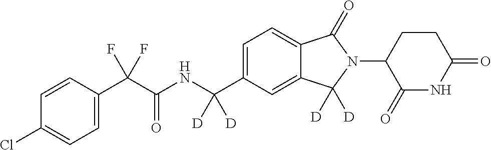 [SMILES: [2H]C([2H])(NC(=O)C(F)(F)c1ccc(Cl)cc1)c1ccc2c(c1)C([2H])([2H])N(C1CCC(=O)NC1=O)C2=O] |
| 452 | 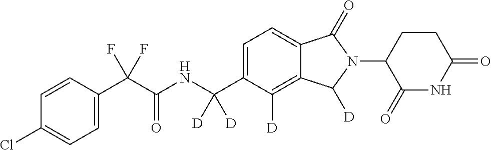 [SMILES: [2H]c1c(C([2H])([2H])NC(=O)C(F)(F)c2ccc(Cl)cc2)ccc2c1C([2H])N(C1CCC(=O)NC1=O)C2=O] |
| 453 | 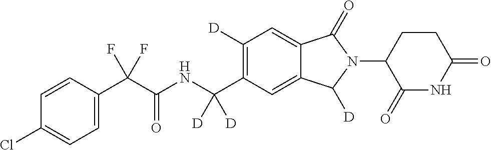 [SMILES: [2H]c1cc2c(cc1C([2H])([2H])NC(=O)C(F)(F)c1ccc(Cl)cc1)C([2H])N(C1CCC(=O)NC1=O)C2=O] |
| 454 | 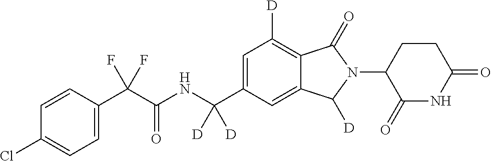 [SMILES: [2H]c1cc(C([2H])([2H])NC(=O)C(F)(F)c2ccc(Cl)cc2)cc2c1C(=O)N(C1CCC(=O)NC1=O)C2[2H]] |
| 455 | 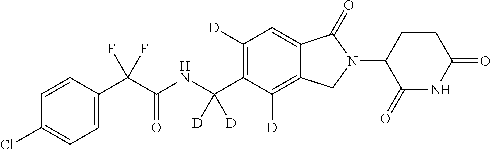 [SMILES: [2H]c1cc2c(c([2H])c1C([2H])([2H])NC(=O)C(F)(F)c1ccc(Cl)cc1)CN(C1CCC(=O)NC1=O)C2=O] |
| 456 | 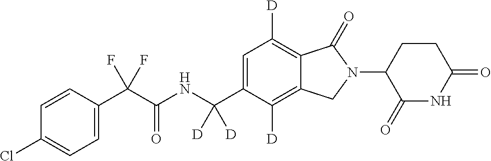 [SMILES: [2H]c1cc(C([2H])([2H])NC(=O)C(F)(F)c2ccc(Cl)cc2)c([2H])c2c1C(=O)N(C1CCC(=O)NC1=O)C2] |
| 457 | 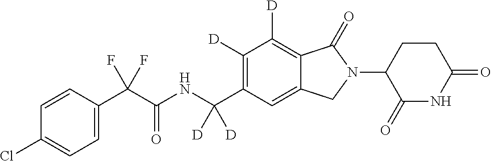 [SMILES: [2H]c1c(C([2H])([2H])NC(=O)C(F)(F)c2ccc(Cl)cc2)cc2c(c1[2H])C(=O)N(C1CCC(=O)NC1=O)C2] |
| 458 | 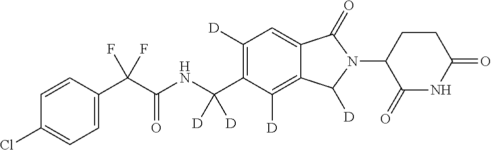 [SMILES: [2H]c1cc2c(c([2H])c1C([2H])([2H])NC(=O)C(F)(F)c1ccc(Cl)cc1)C([2H])N(C1CCC(=O)NC1=O)C2=O] |
| 459 | 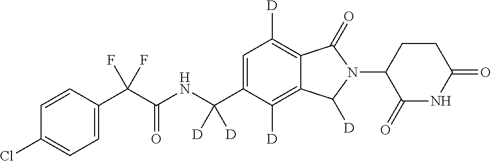 [SMILES: [2H]c1cc(C([2H])([2H])NC(=O)C(F)(F)c2ccc(Cl)cc2)c([2H])c2c1C(=O)N(C1CCC(=O)NC1=O)C2[2H]] |
| 460 | 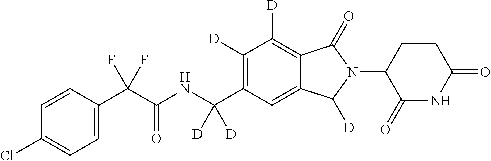 [SMILES: [2H]c1c(C([2H])([2H])NC(=O)C(F)(F)c2ccc(Cl)cc2)cc2c(c1[2H])C(=O)N(C1CCC(=O)NC1=O)C2[2H]] |
| 461 | 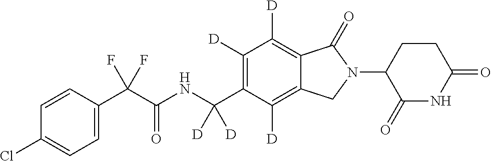 [SMILES: [2H]c1c([2H])c(C([2H])([2H])NC(=O)C(F)(F)c2ccc(Cl)cc2)c([2H])c2c1C(=O)N(C1CCC(=O)NC1=O)C2] |
| 461 | 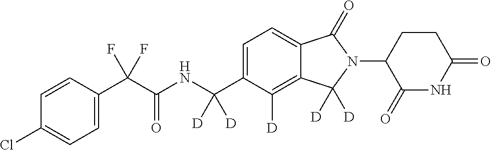 [SMILES: [2H]c1c(C([2H])([2H])NC(=O)C(F)(F)c2ccc(Cl)cc2)ccc2c1C([2H])([2H])N(C1CCC(=O)NC1=O)C2=O] |
| 463 | 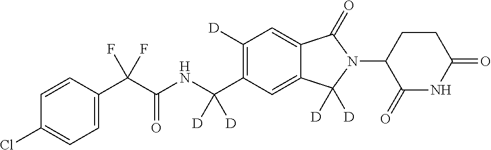 [SMILES: [2H]c1cc2c(cc1C([2H])([2H])NC(=O)C(F)(F)c1ccc(Cl)cc1)C([2H])([2H])N(C1CCC(=O)NC1=O)C2=O] |
| 464 | 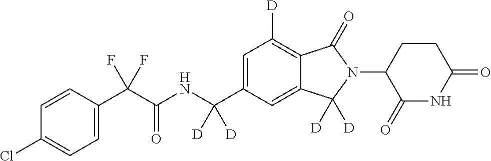 [SMILES: [2H]c1cc(C([2H])([2H])NC(=O)C(F)(F)c2ccc(Cl)cc2)cc2c1C(=O)N(C1CCC(=O)NC1=O)C2([2H])[2H]] |
| 465 | 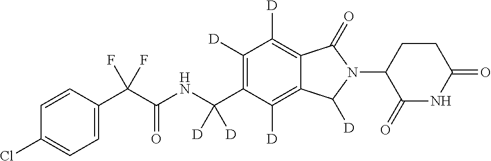 [SMILES: [2H]c1c([2H])c(C([2H])([2H])NC(=O)C(F)(F)c2ccc(Cl)cc2)c([2H])c2c1C(=O)N(C1CCC(=O)NC1=O)C2[2H]] |
| 466 | 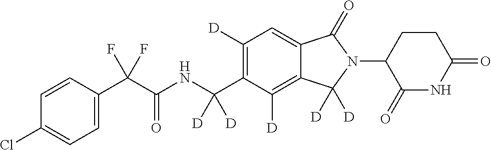 [SMILES: [2H]c1cc2c(c([2H])c1C([2H])([2H])NC(=O)C(F)(F)c1ccc(Cl)cc1)C([2H])([2H])N(C1CCC(=O)NC1=O)C2=O] |
| 467 | 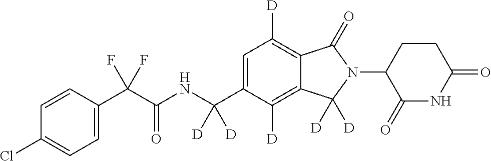 [SMILES: [2H]c1cc(C([2H])([2H])NC(=O)C(F)(F)c2ccc(Cl)cc2)c([2H])c2c1C(=O)N(C1CCC(=O)NC1=O)C2([2H])[2H]] |
| 468 | 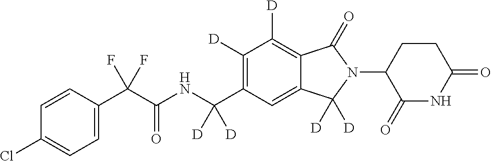 [SMILES: [2H]c1c(C([2H])([2H])NC(=O)C(F)(F)c2ccc(Cl)cc2)cc2c(c1[2H])C(=O)N(C1CCC(=O)NC1=O)C2([2H])[2H]] |
| 469 | 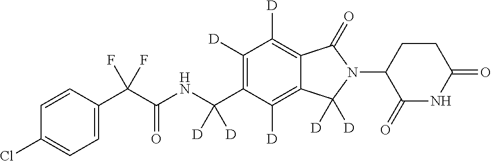 [SMILES: [2H]c1c([2H])c(C([2H])([2H])NC(=O)C(F)(F)c2ccc(Cl)cc2)c([2H])c2c1C(=O)N(C1CCC(=O)NC1=O)C2([2H])[2H]] |
| 470 | 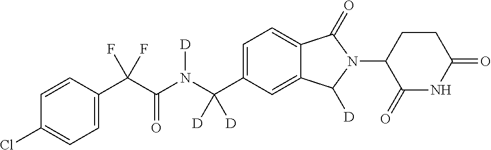 |
| 471 | 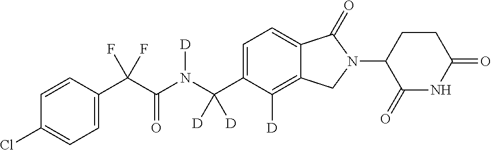 |
| 472 | 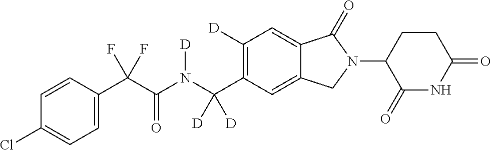 |
| 473 | 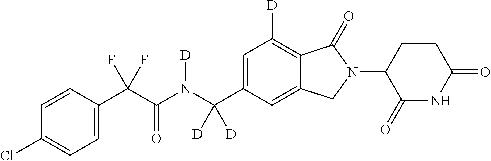 |
| 474 | 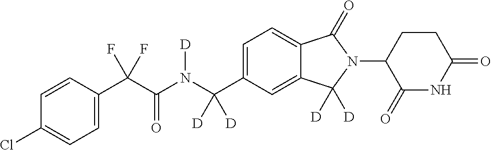 |
| 475 | 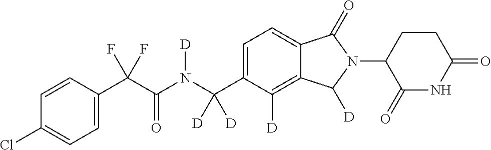 |
| 476 | 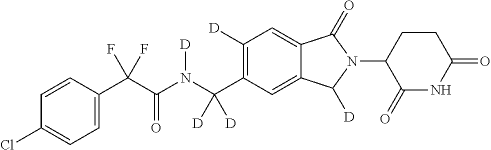 |
| 477 | 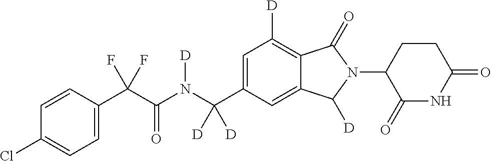 |
| 478 | 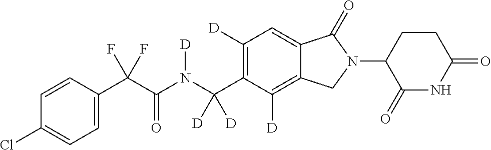 |
| 479 | 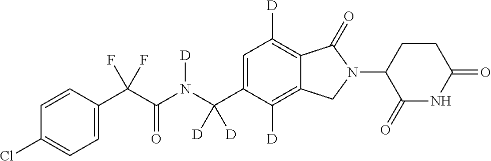 |
| 480 | 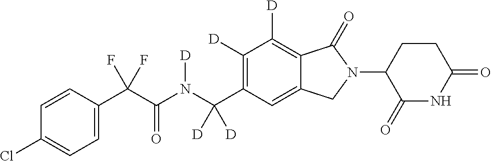 |
| 481 | 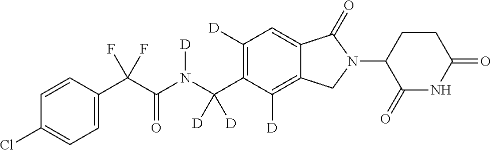 |
| 482 | 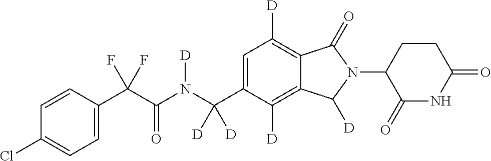 |
| 483 | 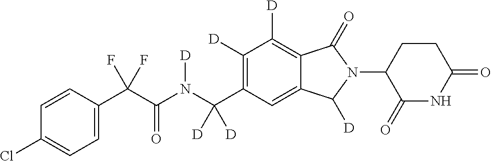 |
| 484 | 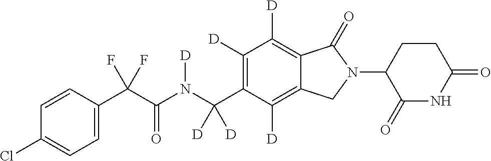 |
| 485 | 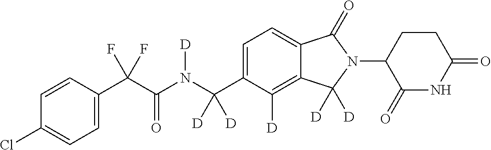 |
| 486 | 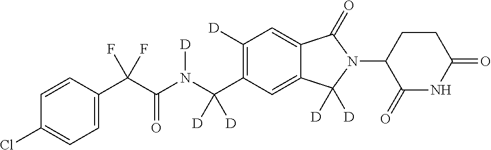 |
| 487 | 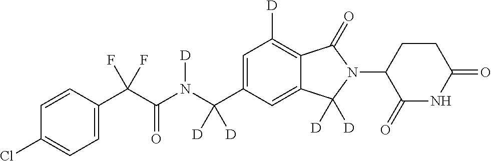 |
| 488 | 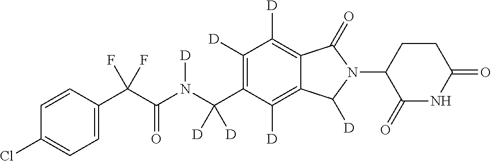 |
| 489 | 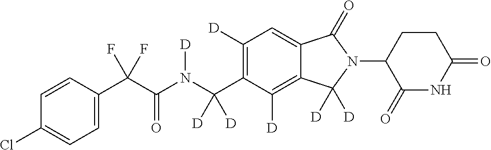 |
| 490 | 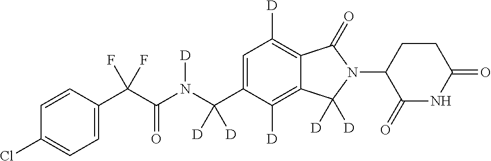 |
| 491 | 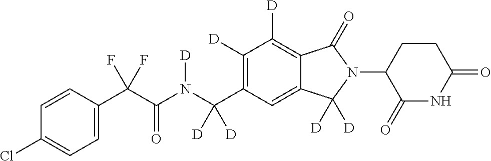 |
| 492 | 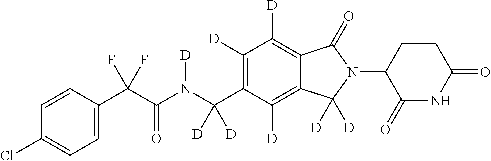 |
| 493 | 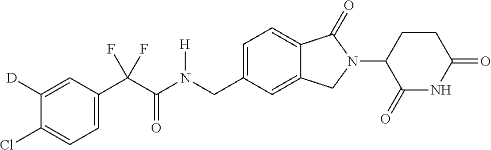 [SMILES: [2H]c1cc(C(F)(F)C(=O)NCc2ccc3c(c2)CN(C2CCC(=O)NC2=O)C3=O)ccc1Cl] |
| 494 | 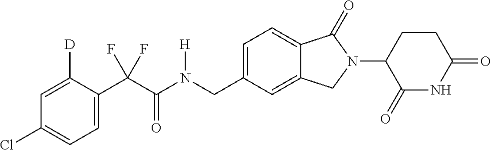 [SMILES: [2H]c1cc(Cl)ccc1C(F)(F)C(=O)NCc1ccc2c(c1)CN(C1CCC(=O)NC1=O)C2=O] |
| 495 | 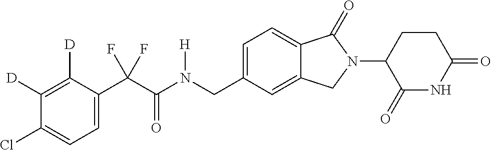 [SMILES: [2H]c1c(Cl)ccc(C(F)(F)C(=O)NCc2ccc3c(c2)CN(C2CCC(=O)NC2=O)C3=O)c1[2H]] |
| 496 | 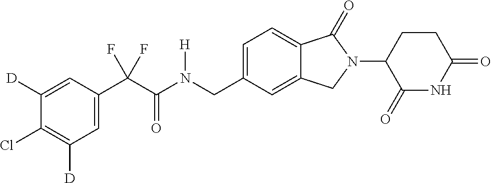 [SMILES: [2H]c1cc(C(F)(F)C(=O)NCc2ccc3c(c2)CN(C2CCC(=O)NC2=O)C3=O)cc([2H])c1Cl] |
| 497 | 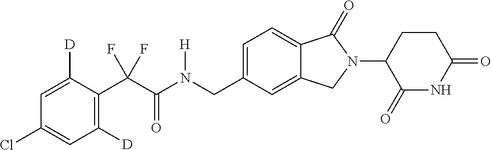 [SMILES: [2H]c1cc(Cl)cc([2H])c1C(F)(F)C(=O)NCc1ccc2c(c1)CN(C1CCC(=O)NC1=O)C2=O] |
| 498 | 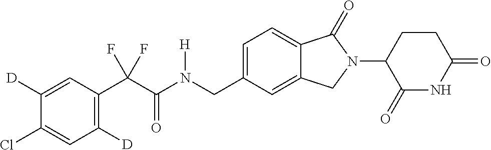 [SMILES: [2H]c1cc(C(F)(F)C(=O)NCc2ccc3c(c2)CN(C2CCC(=O)NC2=O)C3=O)c([2H])cc1Cl] |
| 499 | 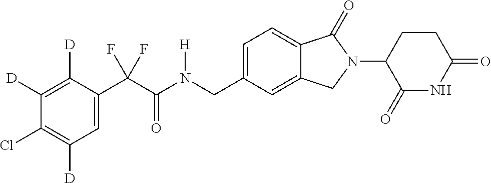 [SMILES: [2H]c1cc(C(F)(F)C(=O)NCc2ccc3c(c2)CN(C2CCC(=O)NC2=O)C3=O)c([2H])c([2H])c1Cl] |
| 500 | 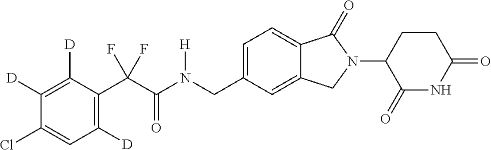 [SMILES: [2H]c1cc(Cl)c([2H])c([2H])c1C(F)(F)C(=O)NCc1ccc2c(c1)CN(C1CCC(=O)NC1=O)C2=O] |
| 501 | 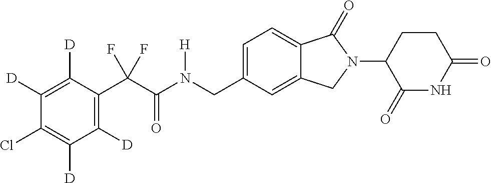 [SMILES: [2H]c1c([2H])c(C(F)(F)C(=O)NCc2ccc3c(c2)CN(C2CCC(=O)NC2=O)C3=O)c([2H])c([2H])c1Cl] |
| 502 | 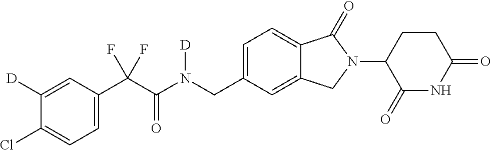 |
| 503 | 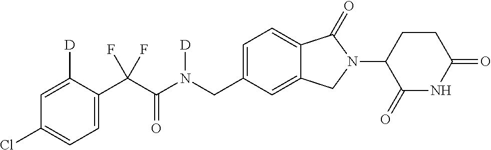 |
| 504 | 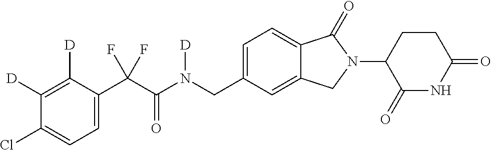 |
| 505 | 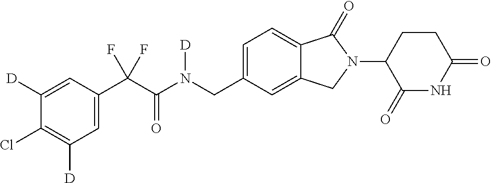 |
| 506 | 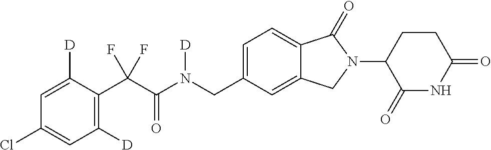 |
| 507 | 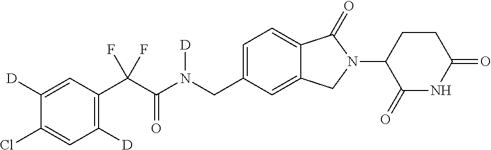 |
| 508 | 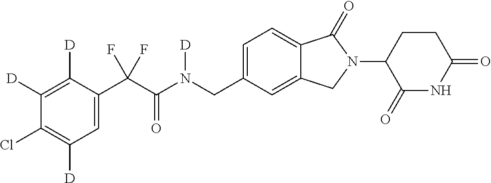 |
| 509 | 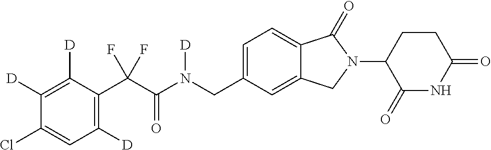 |
| 510 | 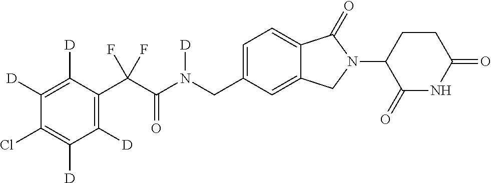 |
| 511 | 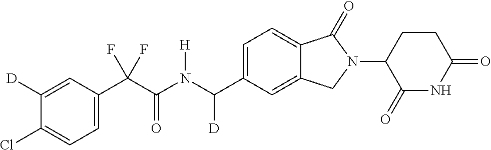 [SMILES: [2H]c1cc(C(F)(F)C(=O)NC([2H])c2ccc3c(c2)CN(C2CCC(=O)NC2=O)C3=O)ccc1Cl] |
| 512 | 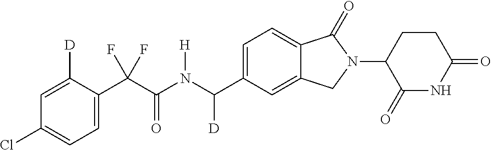 [SMILES: [2H]c1cc(Cl)ccc1C(F)(F)C(=O)NC([2H])c1ccc2c(c1)CN(C1CCC(=O)NC1=O)C2=O] |
| 513 | 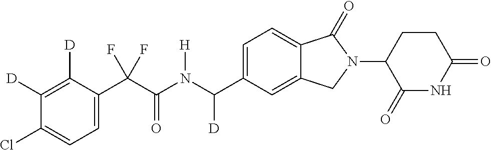 [SMILES: [2H]c1c(Cl)ccc(C(F)(F)C(=O)NC([2H])c2ccc3c(c2)CN(C2CCC(=O)NC2=O)C3=O)c1[2H]] |
| 514 | 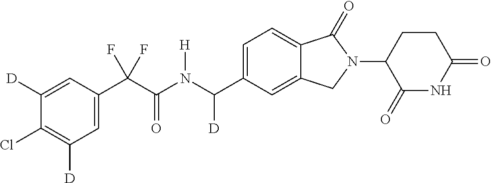 [SMILES: [2H]c1cc(C(F)(F)C(=O)NC([2H])c2ccc3c(c2)CN(C2CCC(=O)NC2=O)C3=O)cc([2H])c1Cl] |
| 515 | 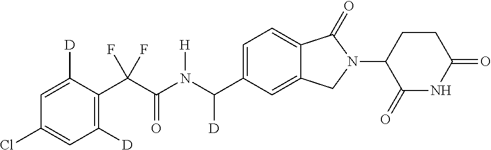 [SMILES: [2H]c1cc(Cl)cc([2H])c1C(F)(F)C(=O)NC([2H])c1ccc2c(c1)CN(C1CCC(=O)NC1=O)C2=O] |
| 516 | 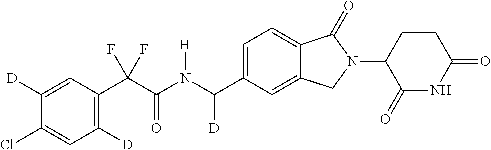 [SMILES: [2H]c1cc(C(F)(F)C(=O)NC([2H])c2ccc3c(c2)CN(C2CCC(=O)NC2=O)C3=O)c([2H])cc1Cl] |
| 517 | 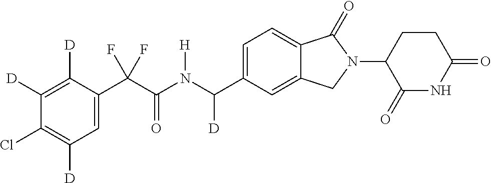 [SMILES: [2H]c1cc(C(F)(F)C(=O)NC([2H])c2ccc3c(c2)CN(C2CCC(=O)NC2=O)C3=O)c([2H])c([2H])c1Cl] |
| 518 | 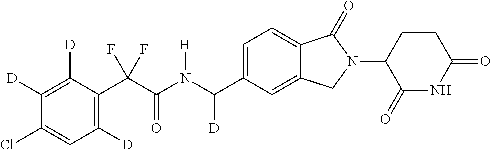 [SMILES: [2H]c1cc(Cl)c([2H])c([2H])c1C(F)(F)C(=O)NC([2H])c1ccc2c(c1)CN(C1CCC(=O)NC1=O)C2=O] |
| 519 | 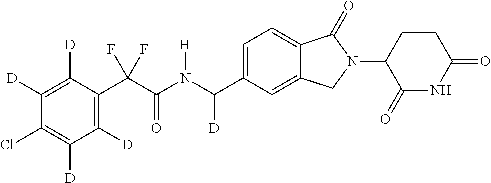 [SMILES: [2H]c1c([2H])c(C(F)(F)C(=O)NC([2H])c2ccc3c(c2)CN(C2CCC(=O)NC2=O)C3=O)c([2H])c([2H])c1Cl] |
| 520 | 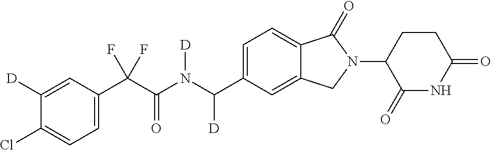 |
| 521 | 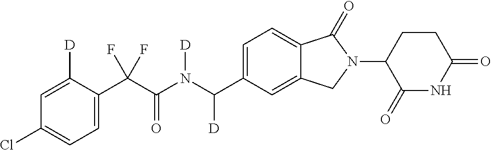 |
| 522 | 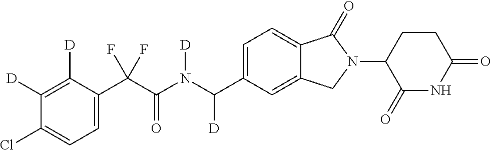 |
| 523 | 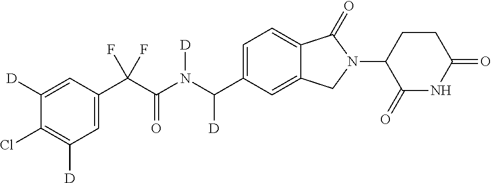 |
| 524 | 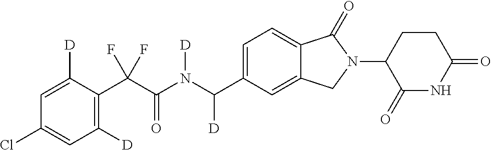 |
| 525 | 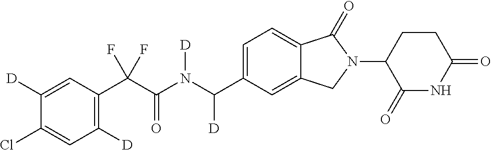 |
| 526 | 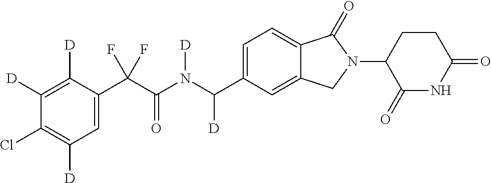 |
| 527 | 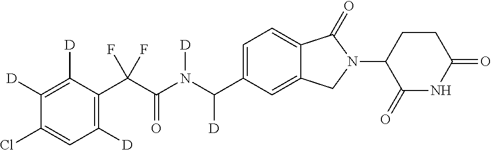 |
| 528 | 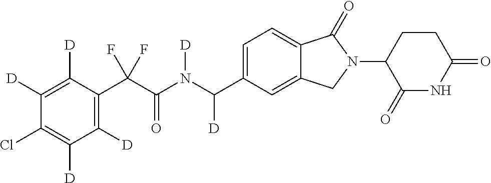 |
| 529 | 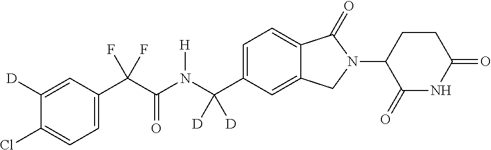 [SMILES: [2H]c1cc(C(F)(F)C(=O)NC([2H])([2H])c2ccc3c(c2)CN(C2CCC(=O)NC2=O)C3=O)ccc1Cl] |
| 530 | 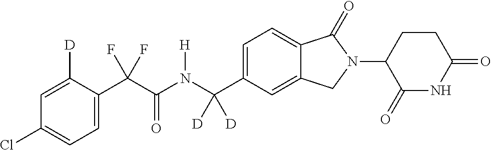 [SMILES: [2H]c1cc(Cl)ccc1C(F)(F)C(=O)NC([2H])([2H])c1ccc2c(c1)CN(C1CCC(=O)NC1=O)C2=O] |
| 531 | 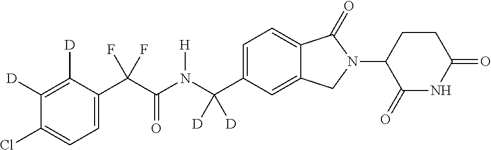 [SMILES: [2H]c1c(Cl)ccc(C(F)(F)C(=O)NC([2H])([2H])c2ccc3c(c2)CN(C2CCC(=O)NC2=O)C3=O)c1[2H]] |
| 532 | 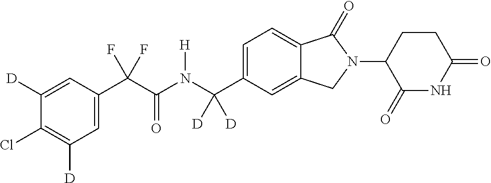 [SMILES: [2H]c1cc(C(F)(F)C(=O)NC([2H])([2H])c2ccc3c(c2)CN(C2CCC(=O)NC2=O)C3=O)cc([2H])c1Cl] |
| 533 | 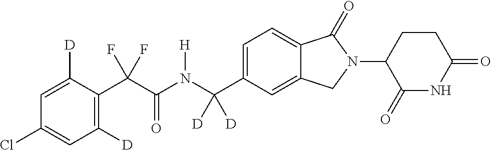 [SMILES: [2H]c1cc(Cl)cc([2H])c1C(F)(F)C(=O)NC([2H])([2H])c1ccc2c(c1)CN(C1CCC(=O)NC1=O)C2=O] |
| 534 | 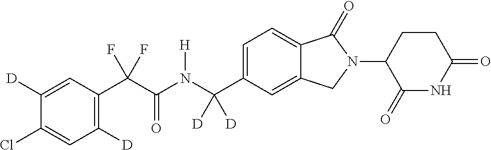 [SMILES: [2H]c1cc(C(F)(F)C(=O)NC([2H])([2H])c2ccc3c(c2)CN(C2CCC(=O)NC2=O)C3=O)c([2H])cc1Cl] |
| 535 | 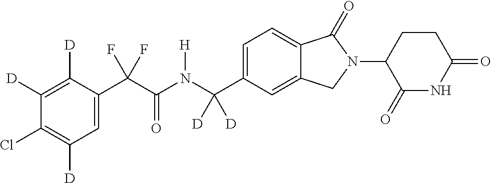 [SMILES: [2H]c1cc(C(F)(F)C(=O)NC([2H])([2H])c2ccc3c(c2)CN(C2CCC(=O)NC2=O)C3=O)c([2H])c([2H])c1Cl] |
| 536 | 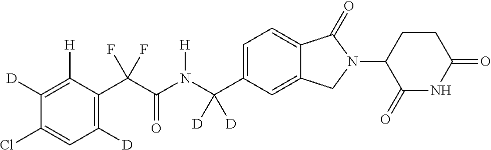 [SMILES: [2H]c1cc(C(F)(F)C(=O)NC([2H])([2H])c2ccc3c(c2)CN(C2CCC(=O)NC2=O)C3=O)c([2H])cc1Cl] |
| 537 | 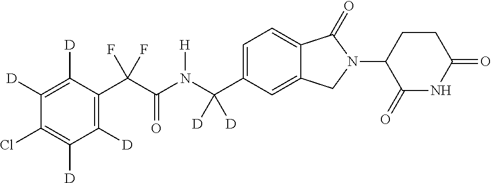 [SMILES: [2H]c1c([2H])c(C(F)(F)C(=O)NC([2H])([2H])c2ccc3c(c2)CN(C2CCC(=O)NC2=O)C3=O)c([2H])c([2H])c1Cl] |
| 538 | 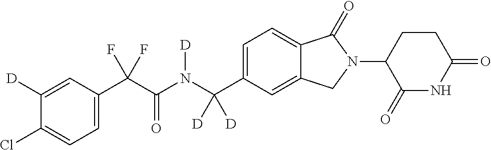 |
| 539 | 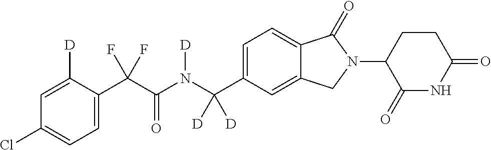 |
| 540 | 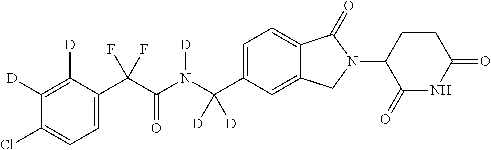 |
| 541 | 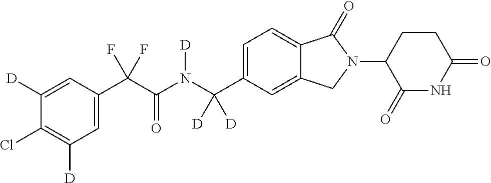 |
| 542 | 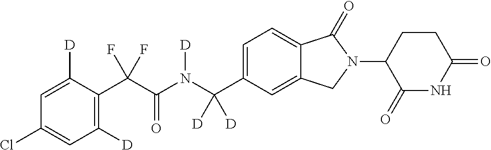 |
| 543 | 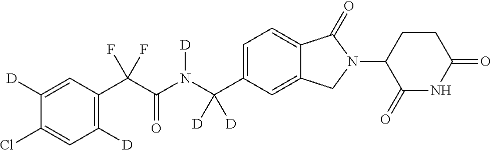 |
| 544 | 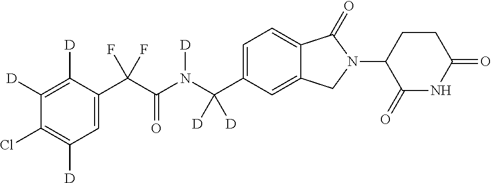 |
| 545 | 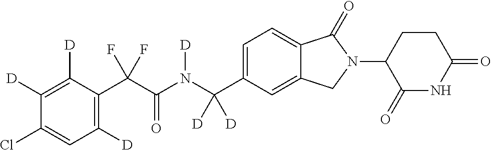 |
| 546 | 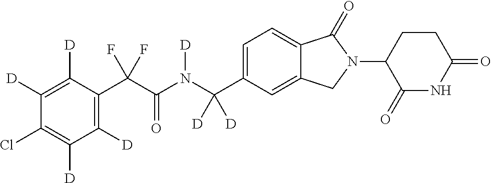 |
| 547 | 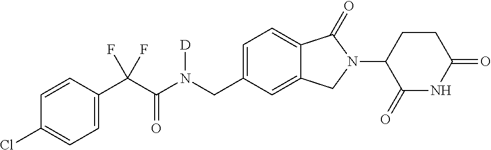 |
| 548 | 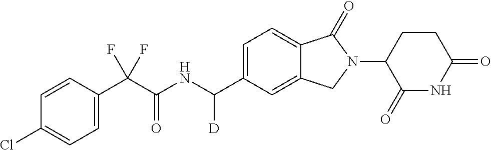 [SMILES: [2H]C(NC(=O)C(F)(F)c1ccc(Cl)cc1)c1ccc2c(c1)CN(C1CCC(=O)NC1=O)C2=O] |
| 549 | 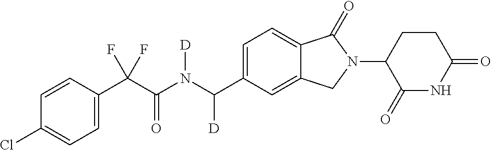 |
| 550 | 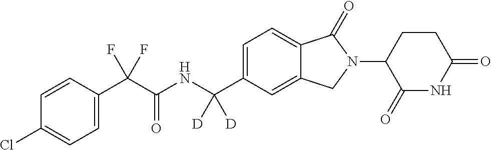 [SMILES: [2H]C([2H])(NC(=O)C(F)(F)c1ccc(Cl)cc1)c1ccc2c(c1)CN(C1CCC(=O)NC1=O)C2=O] |
| 551 | 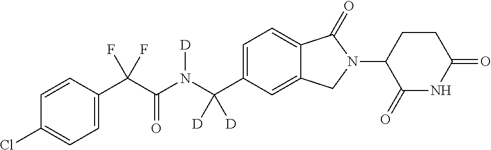 |
| 552 | 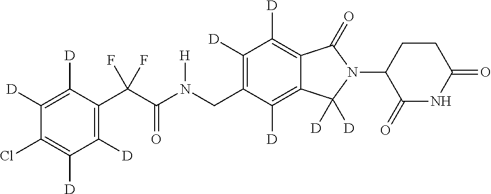 [SMILES: [2H]c1c([2H])c(C(F)(F)C(=O)NCc2c([2H])c([2H])c3c(c2[2H])C([2H])([2H])N(C2CCC(=O)NC2=O)C3=O)c([2H])c([2H])c1Cl] |
| 553 | 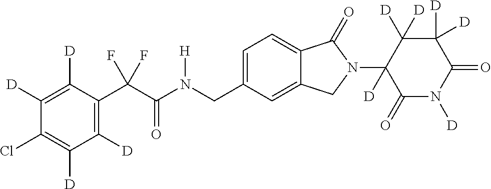 |
| 554 | 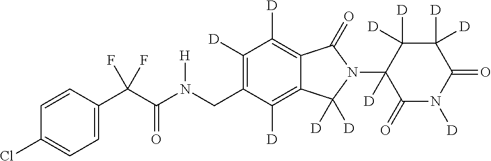 |
| 555 | 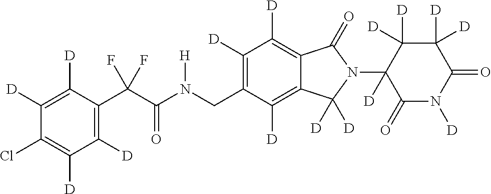 |
| 556 | 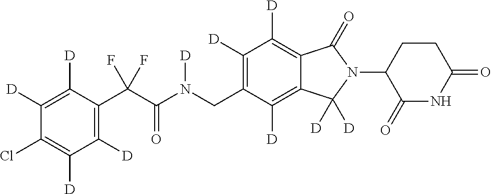 |
| 557 | 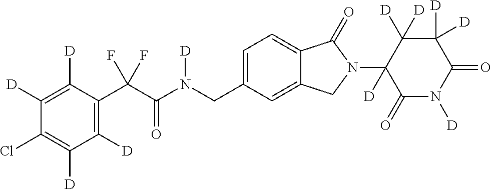 |
| 558 | 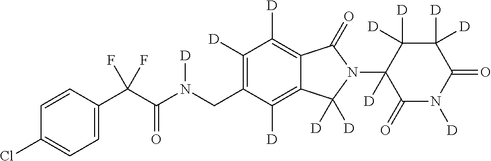 |
| 559 | 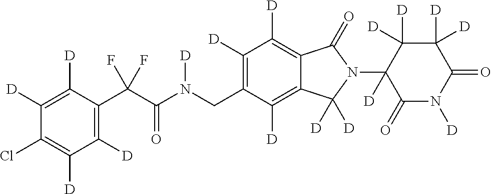 |
| 560 | 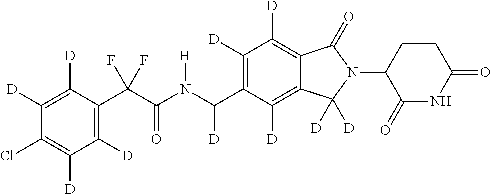 [SMILES: [2H]c1c([2H])c(C(F)(F)C(=O)NC([2H])c2c([2H])c([2H])c3c(c2[2H])C([2H])([2H])N(C2CCC(=O)NC2=O)C3=O)c([2H])c([2H])c1Cl] |
| 561 | 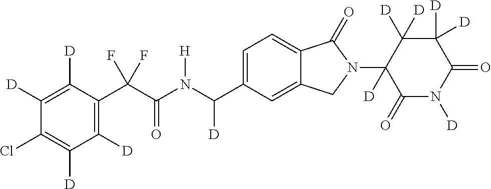 |
| 562 | 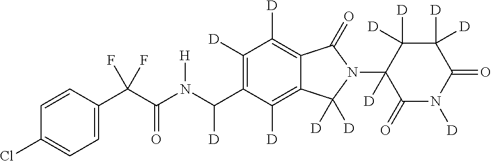 |
| 563 | 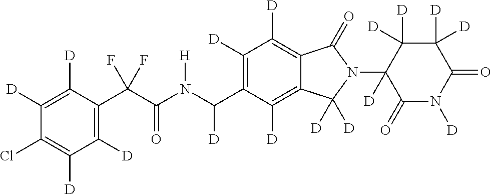 |
| 564 | 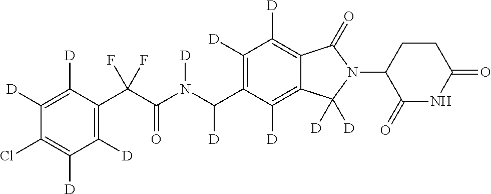 |
| 565 | 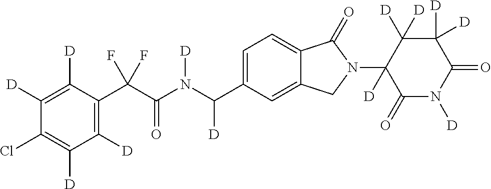 |
| 566 | 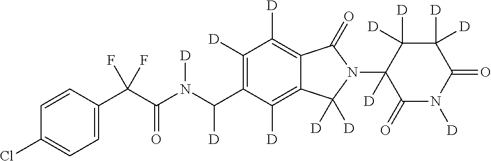 |
| 567 | 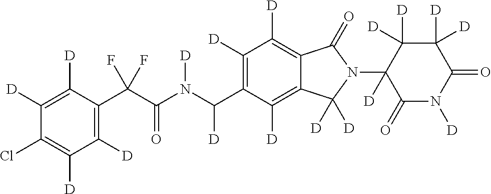 |
| 568 | 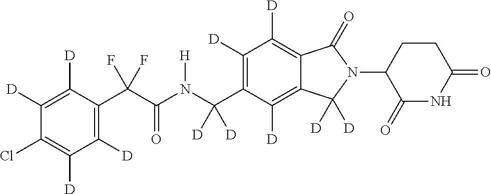 [SMILES: [2H]c1c([2H])c(C(F)(F)C(=O)NC([2H])([2H])c2c([2H])c([2H])c3c(c2[2H])C([2H])([2H])N(C2CCC(=O)NC2=O)C3=O)c([2H])c([2H])c1Cl] |
| 569 | 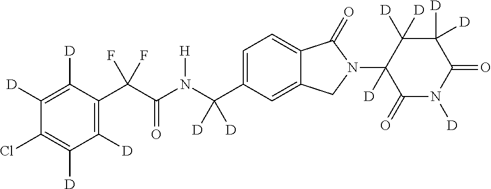 |
| 570 | 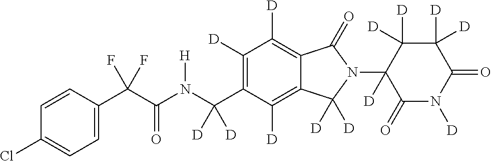 |
| 571 | 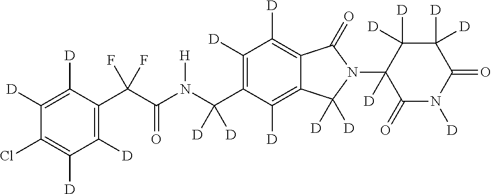 |
| 572 | 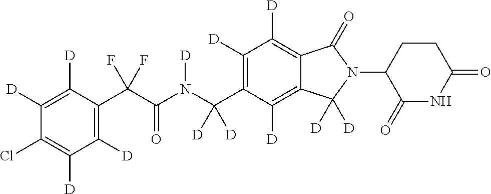 |
| 573 | 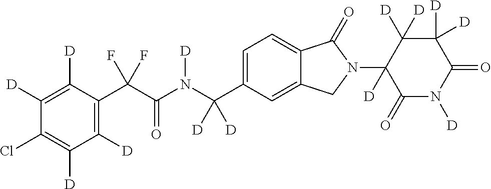 |
| 574 | 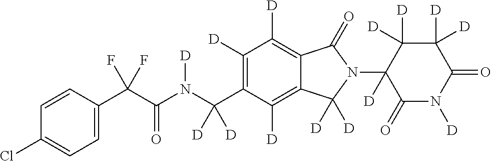 |
| 575 | 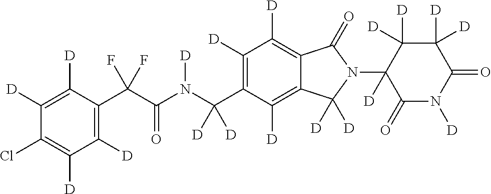 |
| 576 | 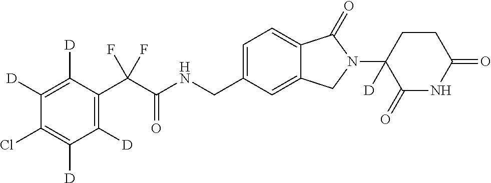 [SMILES: [2H]c1c([2H])c(C(F)(F)C(=O)NCc2ccc3c(c2)CN(C2([2H])CCC(=O)NC2=O)C3=O)c([2H])c([2H])c1Cl] |

or a stereoisomer, a mixture of stereoisomers, a pharmaceutically acceptable salt, a tautomer, a solvate, a hydrate, a co-crystal, a clathrate, or a polymorph thereof.

11. The compound of claim 1, wherein the compound is 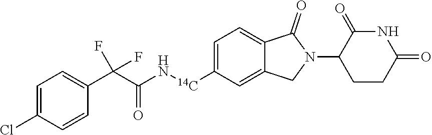 [SMILES: O=C1CCC(N2Cc3cc([14CH2]NC(=O)C(F)(F)c4ccc(Cl)cc4)ccc3C2=O)C(=O)N1]
or a stereoisomer, a mixture of stereoisomers, a pharmaceutically acceptable salt, a tautomer, a solvate, a hydrate, a co-crystal, a clathrate, or a polymorph thereof.

12. A pharmaceutical composition comprising a compound of claim 1, or a stereoisomer, a mixture of stereoisomers, a pharmaceutically acceptable salt, a tautomer, a solvate, a hydrate, a co-crystal, a clathrate, or a polymorph thereof and a pharmaceutically acceptable carrier, diluent and/or excipient.

13. A method of treating cancer comprising administering to a mammal having cancer a therapeutically effective amount of the compound of claim 1.

14. The method of claim 13, wherein the cancer is leukemia.

15. The method of claim 14, wherein the leukemia is chronic lymphocytic leukemia, chronic myelocytic leukemia, acute lymphoblastic leukemia or acute myeloid leukemia.

16. The method of claim 14, wherein the leukemia is an acute myeloid leukemia.

17. The method of claim 14, wherein the leukemia is relapsed, refractory or resistant to conventional therapy.

18. A method of treating a myeloproliferative neoplasm comprising administering to a mammal having cancer a therapeutically effective amount of the compound of claim 1.

19. The method of claim 13, further comprising administering a therapeutically effective amount of a second active agent or a support care therapy.

20. The method of claim 19, wherein the second active agent is a therapeutic antibody that specifically binds to a cancer antigen, hematopoietic growth factor, cytokine, anti-cancer agent, antibiotic, cox-2 inhibitor, immunomodulatory agent, immunosuppressive agent, corticosteroid or a pharmacologically active mutant or derivative thereof.

21. The method of claim 20, wherein the second agent is selected from a JAK inhibitor, a FLT3 inhibitor, an mTOR inhibitor, a spliceosome inhibitor, an ERK inhibitor, an LSD1 inhibitor, an SMG1 inhibitor, a BH3 mimetic, and a topoisomerase inhibitor.

22-30. (canceled)
